(19) United States
(12) Patent Application Publication
(10) Pub. No.: US 2023/0090600 A1
(43) Pub. Date: Mar. 23, 2023

(54) MICROBIAL FERMENTATION FOR THE PRODUCTION OF ISOPRENOID ALCOHOLS AND DERIVATIVES

(71) Applicant: LanzaTech, Inc., Skokie, IL (US)

(72) Inventors: Sean Dennis Simpson, Evanston, IL (US); Michael Koepke, Chicago, IL (US); Rupert Oliver John Norman, Chicago, IL (US); Shivani Garg, Park Ridge, IL (US)

(21) Appl. No.: 17/821,404

(22) Filed: Aug. 22, 2022

Related U.S. Application Data
(60) Provisional application No. 63/260,534, filed on Aug. 24, 2021.

(51) Int. Cl. C12P 7/04; C12P 5/00; C12N 15/52; C12N 15/74

(52) U.S. Cl. CPC C12P 7/04; C12P 5/007; C12N 15/52; C12Y 402/03027; C12Y 503/03002; C12Y 402/03046; C12N 15/74; C12N 2800/101

(57) ABSTRACT

The disclosure provides a method for producing an isoprenoid alcohol, isoprenoid alcohol derivative, or a terpene precursor thereof by microbial fermentation. Typically, the method involves culturing a recombinant bacterium in the presence of a gaseous substrate whereby the bacterium produces an isoprenoid alcohol, isoprenoid alcohol derivative, terpene or a precursor thereof. The microorganism may comprise one or more exogenous enzymes.

### CROSS REFERENCE TO RELATED APPLICATIONS

This application claims the benefit of U.S. Provisional Patent Application No. 63/260,534, filed Aug. 24, 2021, the entirety of which is incorporated herein by reference.

### REFERENCE TO A SEQUENCE LISTING

The application contains a Sequence Listing which has been submitted electronically in ST.26 Sequence listing XML format and is hereby incorporated by reference in its entirety. Said ST.26 Sequence listing XML, created on Oct. 21, 2022, is named LT219US1-Sequences_Corrected.xml and is 306,188 bytes in size.

### FIELD

The present disclosure relates to recombinant microorganisms and methods for the production of isoprenoid alcohols, isoprenoid alcohol derivatives, terpenes and/or precursors thereof by microbial fermentation of a gaseous substrate.

### BACKGROUND

Isoprenoid alcohols are key intermediary products for the production of isoprenoid precursors in these novel synthetic metabolic pathways. Terpenes are a diverse class of naturally occurring chemicals composed of five-carbon isoprene units. Terpene derivatives include terpenoids (also known as isoprenoids) which may be formed by oxidation or rearrangement of the carbon backbone or a number of functional group additions or rearrangements.

Examples of terpenes include: isoprene (C5 hemiterpene), farnesene (C15 Sesquiterpenes), artemisinin (C15 Sesquiterpenes), citral (C10 Monoterpenes), carotenoids (C40 Tetraterpenes), menthol (C10 Monoterpenes), Camphor (C10 Monoterpenes), and cannabinoids.

Isoprenoid acyl-CoAs, such as 3-methyl-but-2-enoyl-CoA and 3-methyl-but-3-enoyl-CoA, and isoprenoid alcohols, such as prenol and isoprenol, are key pathway intermediates that can be converted to isoprenoid precursors, such as isopentenyl phosphate (IP), dimethylallyl phosphate (DMAP), IPP and DMAPP, through phosphorylation enzymes. Any of these products can be further modified if desired. Terpenes are valuable commercial products used in a diverse number of industries. The highest tonnage uses of terpenes are as resins, solvents, fragrances and vitamins. For example, isoprene is used in the production of synthetic rubber (cis-1,4-polyisoprene) for example in the tyre industry; farnesene is used as an energy dense drop-in fuel used for transportation or as jet-fuel; artemisinin is used as a malaria drug; and citral, carotenoids, menthol, camphor, and cannabinoids are used in the manufacture of pharmaceuticals, butadiene, and as aromatic ingredients.

Terpenes may be produced from petrochemical sources and from terpene feed-stocks, such as turpentine. For example, isoprene is produced petrochemically as a by-product of naphtha or oil cracking in the production of ethylene. Many terpenes are also extracted in relatively small quantities from natural sources. However, these production methods are expensive, unsustainable and often cause environmental problems including contributing to climate change.

Due to the extremely flammable nature of isoprene, known methods of production require extensive safeguards to limit potential for fire and explosions.

It is an object of the disclosure to overcome one or more of the disadvantages of the prior art, or at least to provide the public with an alternative means for producing isoprenoid alcohols, isoprenoid alcohol derivatives, terpenes and other related products.

### SUMMARY

Microbial fermentation provides an alternative option for the production of isoprenoid alcohols, isoprenoid alcohol derivatives, and/or terpenes. The reactions of the disclosure serve as a platform for the synthesis of isoprenoid precursors when utilized in combination with a variety of metabolic pathways and enzymes for carbon rearrangement and the addition/removal of functional groups. Isoprenoid alcohols are key intermediary products for the production of isoprenoid precursors in these novel synthetic metabolic pathways. Terpenes are ubiquitous in nature, for example they are involved in bacterial cell wall biosynthesis, and they are produced by some trees (for example poplar) to protect leaves from UV light exposure. However, not all bacteria comprise the necessary cellular machinery to produce terpenes and/or their precursors as metabolic products. For example, carboxydotrophic acetogens, such as C. autoethanogenum or C. ljungdahlii, which are able to ferment substrates comprising carbon monoxide to produce products such as ethanol, are not known to produce and emit any terpenes and/or their precursors as metabolic products. In addition, most bacteria are not known to produce any isoprenoid alcohols or terpenes which are of commercial value.

The disclosure generally provides, inter alia, methods for the production of one or more isoprenoid alcohols, isoprenoid alcohol derivatives, terpenes and/or precursors thereof by microbial fermentation of a substrate comprising CO, and recombinant microorganisms of use in such methods.

The disclosure provides a genetically engineered microorganism capable of producing a product from a gaseous substrate, the microorganism comprising a nucleic acid encoding a group of exogenous enzymes comprising at least acetyl-CoA synthase and at least one of the following:
a) a nucleic acid encoding a group of exogenous enzymes comprising i) keto-acyl-CoA thiolase (KAT1), ii) 3-hydroxy-3-methylglutaryl coenzyme A (HMG-CoA) synthase, iii) methylglutaconyl-CoA hydratase (MGCH), iv) 3-methylcrotonyl-CoA carboxylase (MCCC), v) acyl-CoA reductase (ACOAR), and vi) alcohol dehydrogenase (ADH);
b) a nucleic acid encoding a group of exogenous enzymes comprising i) KAT1, ii) HMG-CoA synthase, iii) MGCH, iv) MCCC, v) phosphotransbutyrase butyrate kinase (Ptb-buk), vi) acetaldehyde-ferredoxin oxidoreductase (AOR), and vii) (ADH);
c) a nucleic acid encoding a group of exogenous enzymes comprising i) KAT1 or PTAr and ACKr, ii) CoA transferase A/B (CtfAB), iii) acetoacetate decarboxylase (ADC) or ADC and hydroxyisovalerate synthase (HIVS), iv) hydroxyisovalerate thioesterase (3HBZCT), v) hydroxyisopentyl-CoA hydro-lyase (HPHL), vi) ACOAR, and vii) ADH;
d) a nucleic acid encoding a group of exogenous enzymes comprising i) KAT1 or PTAr and ACKr, ii) CoA transferase A/B (CtfAB), iii) ADC or ADC and HIVS, iv) 3HBZCT, v) HPHL, vi) Ptb-buk, vii) AOR, and ADH;
e) a nucleic acid encoding a group of exogenous enzymes comprising i) KAT1, ii) HMG-CoA synthase, iii) 3-hydroxy-3-methylglutaryl coenzyme A (HMG-CoA) reductase, iv) mevalonate kinase (MK), v) phosphomevalonate kinase (PMK), vi) diphosphomevalonate decarboxylase (DMD), vii) iso-pentenyldiphosphate isomerase (IDI), viii) dimethylallyl diphosphate kinase (DMPKK), and ix) dimethylallyl phosphate kinase (DMPK);
f) a nucleic acid encoding a group of exogenous enzymes comprising i) KAT1, ii) HMG-CoA synthase, iii) 3-hydroxy-3-methylglutaryl coenzyme A (HMG-CoA) reductase, iv) mevalonate kinase (MK), v) phosphomevalonate decarboxylase (PMVD), vi) iso-pentenylphosphate isomerase (IPI), and vii) prenylphosphatase (DMPase);
g) a nucleic acid encoding a group of exogenous enzymes comprising i) thiolase, acyl-CoA acetyltransferase, or polyketide synthase, ii) β-Ketoacyl-CoA reductase or a β-hydroxyacyl-CoA dehydrogenase, iii) β-hydroxyacyl-CoA dehydratase,
iv) trans-Enoyl-CoA reductase or butyryl-CoA dehydrogenase/electron transferring flavoprotein AB (Bcd-EtfAB), v) an alcohol forming acyl-CoA reductase or aldehyde forming acyl-CoA carboxylate reductase, vi) a hydrolysis enzyme or ADH, and vii) an alcohol dehydratase; and
   wherein the microorganism is a C1-fixing microorganism and the product is an isoprenoid alcohol.

The microorganism of an embodiment, wherein the isoprenoid alcohol is prenol.

The microorganism of an embodiment, further comprising a nucleic acid encoding a group of exogenous enzymes capable of converting prenol to isoprenol.

The microorganism of an embodiment, further comprising a nucleic acid encoding a group of enzymes capable of converting prenol to dimethylallyl pyrophosphate (DMAPP).

The microorganism of an embodiment, further comprising a nucleic acid encoding a group of exogenous enzymes capable of converting isoprenol to isopentenyl diphosphate (IPP).

The microorganism of an embodiment, further comprising a nucleic acid encoding an exogenous enzyme selected from the group consisting of isopentenyl diphosphate isomerase and geranyltranstransferase.

The microorganism of an embodiment, wherein the group of enzymes capable of converting prenol to DMAPP is alcohol diphosphokinase.

The microorganism of an embodiment, wherein the group of enzymes capable of converting isoprenol to IPP is alcohol diphosphokinase.

The microorganism of an embodiment, further comprising three or more enzymes capable of producing the isoprenoid alcohol(s) selected from acetohydroxy acid isomeroreductase, an acetoacetate decarboxylate, an acyl-CoA dehydrogenase, an acyl-CoA reductase, an acyl-CoA synthase, an acyl-CoA transferase, an alcohol dehydratase, an alcohol dehydrogenase, an aldehyde decarboxylase, an alpha-keto acid decarboxylase, an alpha-keto acid dehydrogenase, a carboxylate kinase, a carboxylate reductase, a dehydratase, a dihydroxy acid dehydratase, a diol dehydratase, an enoate hydratase, an enoyl-CoA hydratase, an enoyl-CoA reductase, a glutaconyl-CoA decarboxylase, an hydroxy acid dehydratase, an hydroxy acid dehydrogenase, an hydroxyacyl-CoA dehydratase, an hydroxyacyl-CoA dehydrogenase, an hydroxymethylacyl-CoA synthase, an isomeroreductase, an isopropylmalate dehydrogenase, an isopropylmalate isomerase, an isopropylmalate synthase, a mutase, an omega-oxidation enzyme, a phosphotransacylase, a thioesterase, or a thiolase, where said production optionally proceeds through an isoprenoid acyl-CoA.

The microorganism of an embodiment, further comprising one or more phosphorylation enzyme(s) to convert said isoprenoid alcohol(s) to an isoprenoid precursor(s); and d) optionally one or more enzyme(s) to convert said isoprenoid precursor(s) to another isoprenoid precursor(s) or an isoprenoid(s) or a derivative(s) thereof; wherein one or more of said enzyme(s) is heterologous.

The microorganism of an embodiment, a recombinant microorganism producing an isoprenoid precursor(s), or optionally an isoprenoid(s) or a derivative(s) thereof, said recombinant microorganism comprising: a) a thiolase or a ketoacetyl-CoA synthase enzyme catalyzing a condensation of an acyl-CoA plus a second acyl-CoA to form a beta-ketoacyl CoA, each said acyl-CoA selected from acetyl-CoA, glycolyl-CoA, propionyl-CoA, malonyl-CoA, an unsubstituted acyl-CoA, or a functionalized acyl-CoA; b) optionally one or more iteration(s) wherein said beta-ketoacyl CoA is modified using one or more enzymes and then used as an acyl-CoA primer unit for a new condensation iteration of step a); c) three or more enzyme(s) to convert said beta-ketoacyl CoA to an isoprenoid alcohol, said enzyme(s) comprising a beta-reduction enzyme(s), and an alcohol forming termination enzyme(s); d) one or more phosphorylation enzyme(s) to convert said isoprenoid alcohol(s) to an isoprenoid precursor(s); and e) optionally one or more enzyme(s) to convert said isoprenoid precursor(s) to another isoprenoid precursor(s) or an isoprenoid(s) or a derivative(s) thereof; wherein one or more said enzyme(s) is heterologous.

The microorganism of an embodiment, further comprising a nucleic acid encoding both exogenous enzymes isopentenyl diphosphate isomerase and geranyltranstransferase.

The microorganism of an embodiment, further comprising a nucleic acid encoding a group of exogenous enzymes selected from limonene synthase, pinene synthase, farnesene synthase, or any combination thereof.

The microorganism of an embodiment, further comprising a nucleic acid encoding an exogenous enzyme comprising isoprene synthase.

The microorganism of an embodiment, having carbon monoxide dehydrogenase.

The microorganism of an embodiment, further comprising a disruptive mutation to DXS pathway.

The microorganism of an embodiment, wherein the disruptive mutation is a knockout.

The microorganism of an embodiment, wherein the exogenous enzymes comprise at least e) in combination with any one or more of a), b), c), d), f) and g) in tandem.

The microorganism of an embodiment, wherein the nucleic acids encoding exogenous enzymes are codon optimized.

The microorganism of an embodiment, wherein the nucleic acids encoding exogenous enzymes are integrated into the genome of the microorganism.

The microorganism of an embodiment, wherein the nucleic acids encoding exogenous enzymes are incorporated in a plasmid.

The microorganism of an embodiment, wherein the nucleic acids encoding exogenous enzymes are regulated by a constitutive promoter

The disclosure provides a method for producing an isoprenoid alcohol, by culturing the microorganism according to claim 1 using at least one C1 compound selected from the group consisting of carbon monoxide and carbon dioxide as a carbon source, to allow the microorganism to produce the isoprenoid alcohol.

The disclosure provides a method for producing an isoprenoid alcohol, isoprenoid alcohol derivative, or terpene precursor by providing at least one C1 compound selected from the group consisting of carbon monoxide and carbon dioxide into contact with the microorganism according to claim 1, to allow the microorganism to produce the isoprenoid alcohol, isoprenoid alcohol derivative, or terpene precursor from the C1 compound.

The method of an embodiment, wherein the microorganism is provided with a gas comprising hydrogen.

The method of an embodiment, wherein the isoprenoid alcohol, is recovered.

The method of an embodiment, wherein the microorganism is provided with a gas comprising hydrogen.

The method of an embodiment, wherein the terpene precursor is recovered.

The method of an embodiment, wherein the C1 compound is derived from an industrial process selected from the group consisting of ferrous metal products manufacturing, non-ferrous products manufacturing, petroleum refining, coal gasification, electric power production, carbon black production, ammonia production, methanol production, and coke manufacturing.

The method of an embodiment, wherein the C1 compound is syngas.

The microorganism of an embodiment, wherein the microorganism is selected from the group consisting of Clostridium autoethanogenum, Clostridium ljungdahlii, Clostridium ragsdalei, Clostridium carboxidivorans, Clostridium drakei, Clostridium scatologenes, Clostridium aceticum, Clostridium formicoaceticum, Clostridium magnum, Cupriavidus necator, Moorella thermoacetica, Moorella thermautotrophica, and any combination thereof.

The microorganism of one embodiment, wherein the isoprenoid alcohol is converted to a terpene selected from the group consisting of terpenoids, vitamin A, lycopene, squalene, isoprene, pinene, nerol, citral, camphor, menthol, limonene, nerolidol, farnesol, farnesene, phytol, carotene, linalool, and any combination thereof.

In engineering the microorganisms of the disclosure, the inventors have surprisingly been able to genetically engineer a microorganism capable of producing a product from a gaseous substrate, wherein the microorganism comprises an iterative pathway comprising catalyzing the conversion of (Cₙ)-acyl CoA to β-ketoacyl-CoA; catalyzing the conversion of β-ketoacyl-CoA to β-hydroxyacyl-CoA; catalyzing the conversion of β-hydroxyacyl-CoA to trans-Δ²-Enoyl-CoA; and catalyzing the conversion of trans-Δ²-Enoyl-CoA to (Cₙ₊₂) acyl-CoA; and one or more termination enzymes; and wherein the microorganism is a C1-fixing bacteria comprising a disruptive mutation in a thioesterase. This pathway can be further extended using the same enzymes or engineered variants thereof that have specificity for higher chain length to produce, including but not limited to, a range of C4, C6, C8, C10, C12, C14 alcohols, ketones, enols or diols. Different type of molecules can be obtained also by using primer or extender units different than acetyl-CoA in a thiolase step. This provides for sustainable fermentation to produce primary alcohols using a substrate comprising CO and/or a substrate comprising CO₂.

Primers and extenders are selected from oxalyl-CoA, acetyl-CoA, malonyl CoA, succinyl-CoA, hydoxyacetyl-CoA, 3-hydroxyproprionyl-CoA, 4-hydroxybutyryl-CoA, 2-aminoacetyl-CoA, 3-aminopropionyl-CoA, 4-aminobutyryl-CoA, isobutyryl-CoA, 3-methyl-butyryl-CoA, 2-hydroxyproprionyl-CoA, 3-hydroxybutyryl-CoA, 2-aminoproprionyl-CoA, propionyl-CoA, and valeryl-CoA. Moreover, the bacteria express the group of enzymes in the reverse β-oxidation pathway and the bacteria acquire the ability to generate primary alcohols, trans Δ² fatty alcohols, β-keto alcohols, 1,3-diols, 1,4-diols, 1,6-diols, diacids, β-hydroxy acids, carboxylic acids, or hydrocarbons. In one embodiment, acetyl-CoA is the primer/starter molecule, which leads to synthesis of even-chained n-alcohols and/or carboxylic acids. In another embodiment, propionyl-CoA is the starter/primer molecule, which enables the synthesis of odd-chained n-alcohols and/or carboxylic acids.

In one embodiment, the primers may be one other than acetyl-CoA or propionyl-CoA, although acetyl-CoA may condense with the primer, acting as an extender unit, to add two carbon units thereto. In another embodiment, these primers in combination with different termination enzymes lead to the synthesis of other products.

In one embodiment, the disclosure describes the one or more termination enzymes are selected from alcohol-forming coenzyme-A thioester reductase, an aldehyde-forming CoA thioester reductase, an alcohol dehydrogenase, a thioesterase, an acyl-CoA:acetyl-CoA transferase, a phosphotransacylase and a carboxylate kinase; aldehyde ferredoxin oxidoreductase; an aldehyde-forming CoA thioester reductase, an aldehyde decarbonylase, alcohol dehydrogenase; aldehyde dehydrogenase, an acyl-CoA reductase, or any combination thereof.

In one embodiment, the disclosure describes operation of multiple turns of a reversal of the beta oxidation cycle, requires the condensation of the acyl-CoA generated from a turn(s) of the cycle with an additional acetyl-CoA molecule to lengthen the acyl-CoA by two carbons each cycle turn. In another embodiment, the initiation and extension of multiple cycle turns requires the use of a thiolase(s) with specificity for longer chain acyl-CoA molecules combined with other pathway enzymes capable of acting on pathway intermediates of increasing carbon number.

While the inventors have demonstrated the efficacy of the disclosure in Clostridium autoethanogenum, the disclosure is applicable to the wider group of anaerobic acetogenic microorganisms and fermentation on substrates comprising CO and/or CO₂, as discussed above and further herein.

The disclosure provides a CoA-dependent elongation platform, which accept functionalized acyl-CoAs as primers and extender units in a reverse beta-oxidation like pathway. Products can be pulled out at any point, and further modified if desired. In other aspects of the invention, reactions to enable product synthesis from central carbon metabolites such as pyruvate through various enzyme combinations is possible. Isoprenoid acyl-CoAs, such as 3-methyl-but-2-enoyl-CoA and 3-methyl-but-3-enoyl-CoA, and isoprenoid alcohols, such as prenol and isoprenol, are key pathway intermediates that can be converted to isoprenoid precursors, such as isopentenyl phosphate (IP), dimethylallyl phosphate (DMAP), IPP and DMAPP, through phosphorylation enzymes. As above, any of the products can be further modified if desired.

Another aspect of the disclosure provides a pathway employing beta-oxidation reversal via 3-methyl-3-butenol (isoprenol) instead of prenol. This pathway starts from a primer and an extender unit, catalyzed by thiolase. After three beta-reduction steps catalyzed by hydroxyacyl-CoA dehydrogenase, enoyl-CoA hydratase and enoyl-CoA reductases, 4-hydroxy-2-methylbutanoyl-CoA is generated. 4-hydroxy-2-methylbutanoyl-CoA is converted to 2-methyl-1,4-butanediol by alcohol-forming acyl-CoA reductase or aldehyde forming acyl-CoA reductase and alcohol dehydrogenase or carboxylate reductase and the hydrolysis enzyme selected from the group consisting thioesterase, acyl-CoA synthase, acyl-CoA transferase and carboxylate kinase plus phosphotransacylase. Then, an alcohol dehydratase converts 2-methyl-1,4-butanediol to 3-methyl-3-butenol (isoprenol). Isoprenol is then converted to IPP by one or two steps of phosphorylation. If phosphorylated by two steps, the first step is catalyzed by alcohol kinase and the second step is catalyzed by phosphate kinase. The one step phosphorylation is catalyzed by alcohol diphosphokinase. Isopentenyl pyrophosphate isomerase (IDI) converts DMAPP to IPP. DMAPP and IPP are condensed to GPP catalyzed by GPP synthase.

The disclosure provides a carboxydotrophic acetogenic recombinant microorganism capable of producing one or more terpenes and/or precursors thereof and optionally one or more other products by fermentation of a substrate comprising CO.

In one particular embodiment, the microorganism is adapted to express one or more enzymes in the mevalonate (MVA) pathway not present in a parental microorganism from which the recombinant microorganism is derived (may be referred to herein as an exogenous enzyme).

In another embodiment, the microorganism is adapted to over-express one or more enzymes in the mevalonate (MVA) pathway which are present in a parental microorganism from which the recombinant microorganism is derived (may be referred to herein as an endogenous enzyme).

In a further embodiment, the microorganism is adapted to:
a) express one or more exogenous enzymes in the mevalonate (MVA) pathway and/or overexpress one or more endogenous enzyme in the mevalonate (MVA) pathway; and
b) express one or more exogenous enzymes in the DXS pathway and/or overexpress one or more endogenous enzymes in the DXS pathway.

In one embodiment, the one or more enzymes from the mevalonate (MVA) pathway is selected from the group consisting of:

a) thiolase (EC 2.3.1.9),

b) HMG-CoA synthase (EC 2.3.3.10),

c) HMG-CoA reductase (EC 1.1.1.88),

d) Mevalonate kinase (EC 2.7.1.36),

e) Phosphomevalonate kinase (EC 2.7.4.2),

f) Mevalonate Diphosphate decarboxylase (EC 4.1.1.33), and

g) a functionally equivalent variant of any one thereof.

In a further embodiment, the one or more enzymes from the DXS pathway is selected from the group consisting of:

a) 1-deoxy-D-xylulose-5-phosphate synthase DXS (EC:2.2.1.7),

b) 1-deoxy-D-xylulose 5-phosphate reductoisomerase DXR (EC:1.1.1.267),

c) 2-C-methyl-D-erythritol 4-phosphate cytidylyltransferase IspD (EC:2.7.7.60), d) 4-diphosphocytidyl-2-C-methyl-D-erythritol kinase IspE (EC:2.7.1.148),

e) 2-C-methyl-D-erythritol 2,4-cyclodiphosphate synthase IspF (EC:4.6.1.12),

f) 4-hydroxy-3-methylbut-2-en-1-yl diphosphate synthase IspG (EC:1.17.7.1),

g) 4-hydroxy-3-methylbut-2-enyl diphosphate reductase (EC:1.17.1.2), and

h) a functionally equivalent variant of any one thereof.

In a further embodiment, one or more further exogenous or endogenous enzymes are expressed or over-expressed to result in the production of a terpene compound or a precursor thereof wherein the exogenous enzyme that is expressed, or the endogenous enzyme that is overexpressed, is selected from the group consisting of:

a) geranyltranstransferase Fps (EC:2.5.1.10),

b) heptaprenyl diphosphate synthase (EC:2.5.1.10),

c) octaprenyl-diphosphate synthase (EC:2.5.1.90),

d) isoprene synthase (EC 4.2.3.27),

e) isopentenyl-diphosphate delta-isomerase (EC 5.3.3.2),

f) farnesene synthase (EC 4.2.3.46/EC 4.2.3.47), and

g) a functionally equivalent variant of any one thereof.

In one embodiment, the parental microorganism is capable of fermenting a substrate comprising CO to produce Acetyl CoA, but not of converting Acetyl CoA to mevalonic acid or isopentenyl pyrophosphate (IPP) and the recombinant microorganism is adapted to express one or more enzymes involved in the mevalonate pathway.

In one embodiment, the one or more terpene and/or precursor thereof is chosen from mevalonic acid, IPP, dimethylallyl pyrophosphate (DMAPP), isoprene, geranyl pyrophosphate (GPP), farnesyl pyrophosphate (FPP) and farnesene.

In one embodiment, the microorganism comprises one or more exogenous nucleic acids adapted to increase expression of one or more endogenous nucleic acids and which one or more endogenous nucleic acids encode one or more of the enzymes referred to herein before.

In one embodiment, the one or more exogenous nucleic acids adapted to increase expression is a regulatory element. In one embodiment, the regulatory element is a promoter. In one embodiment, the promoter is a constitutive promoter. In one embodiment, the promoter is selected from the group comprising Wood-Ljungdahl gene cluster or Phosphotransacetylase/Acetate kinase operon promoters.

In one embodiment, the microorganism comprises one or more exogenous nucleic acids encoding and adapted to express one or more of the enzymes referred to hereinbefore. In one embodiment, the microorganisms comprise one or more exogenous nucleic acids encoding and adapted to express at least two of the enzymes. In other embodiments, the microorganism comprises one or more exogenous nucleic acids encoding and adapted to express at least three, at least four, at least five, at least six, at least seven, at least eight, at least nine or more of the enzymes.

In one embodiment, the one or more exogenous nucleic acid is a nucleic acid construct or vector, in one particular embodiment a plasmid, encoding one or more of the enzymes referred to hereinbefore in any combination.

In one embodiment, the exogenous nucleic acid is an expression plasmid.

In one particular embodiment, the parental microorganism is selected from the group of carboxydotrophic acetogenic bacteria. In certain embodiments the microorganism is selected from the group comprising Clostridium autoethanogenum, Clostridium ljungdahlii, Clostridium ragsdalei, Clostridium carboxidivorans, Clostridium drakei, Clostridium scatologenes, Clostridium aceticum, Clostridium formicoaceticum, Clostridium magnum, Butyribacterium methylotrophicum, Acetobacterium woodii, Alkalibaculum bacchii, Blautia producta, Eubacterium limosum, Moorella thermoacetica, Moorella thermautotrophica, Sporomusa ovata, Sporomusa silvacetica, Sporomusa sphaeroides, Oxobacter pfennigii, and Thermoanaerobacter kivui.

In some aspects of the microorganism disclosed herein, the microorganism is a member of a genus selected from the group consisting of Acetobacterium, Alkalibaculum, Blautia, Butyribacterium, Clostridium, Cupriavidus, Eubacterium, Moorella, Oxobacter, Sporomusa, and Thermoanaerobacter.

In some aspects of the microorganism disclosed herein, the microorganism is derived from a parental microorganism selected from the group consisting of Acetobacterium woodii, Alkalibaculum bacchii, Blautia producta, Butyribacterium methylotrophicum, Clostridium aceticum, Clostridium autoethanogenum, Clostridium carboxidivorans, Clostridium coskatii, Clostridium drakei, Clostridium formicoaceticum, Clostridium ljungdahlii, Clostridium magnum, Clostridium ragsdalei, Clostridium scatologenes, Cupriavidus necator, Eubacterium limosum, Moorella thermautotrophica, Moorella thermoacetica, Oxobacter pfennigii, Sporomusa ovata, Sporomusa silvacetica, Sporomusa sphaeroides, and Thermoanaerobacter kiuvi.

In one embodiment the parental microorganism is Clostridium autoethanogenum or Clostridium ljungdahlii. In one particular embodiment, the microorganism is Clostridium autoethanogenum DSM23693. In another particular embodiment, the microorganism is Clostridium ljungdahlii DSM13528 (or ATCC55383).

In one embodiment, the parental microorganism lacks one or more genes in the DXS pathway and/or the mevalonate (MVA) pathway. In one embodiment, the parental microorganism lacks one or more genes encoding an enzyme selected from the group consisting of:

a) thiolase (EC 2.3.1.9),

b) HMG-CoA synthase (EC 2.3.3.10),

c) HMG-CoA reductase (EC 1.1.1.88),

d) Mevalonate kinase (EC 2.7.1.36),

e) Phosphomevalonate kinase (EC 2.7.4.2),

f) Mevalonate Diphosphate decarboxylase (EC 4.1.1.33),

g) 1-deoxy-D-xylulose-5-phosphate synthase DXS (EC:2.2.1.7),

h) 1-deoxy-D-xylulose 5-phosphate reductoisomerase DXR (EC:1.1.1.267),

i) 2-C-methyl-D-erythritol 4-phosphate cytidylyltransferase IspD (EC:2.7.7.60),

j) 4-diphosphocytidyl-2-C-methyl-D-erythritol kinase IspE (EC:2.7.1.148),

k) 2-C-methyl-D-erythritol 2,4-cyclodiphosphate synthase IspF (EC:4.6.1.12),

l) 4-hydroxy-3-methylbut-2-en-1-yl diphosphate synthase IspG (EC:1.17.7.1),

m) 4-hydroxy-3-methylbut-2-enyl diphosphate reductase (EC:1.17.1.2), and

n) a functionally equivalent variant of any one thereof.

In a second aspect, the disclosure provides a nucleic acid encoding one or more enzymes which when expressed in a microorganism allows the microorganism to produce one or more terpenes and/or precursors thereof by fermentation of a substrate comprising CO.

In one embodiment, the nucleic acid encodes two or more enzymes which when expressed in a microorganism allows the microorganism to produce one or more terpenes and/or precursors thereof by fermentation of a substrate comprising CO. In one embodiment, a nucleic acid of the disclosure encodes at least three, at least four, at least five, at least six, at least seven, at least eight, at least nine or more of such enzymes.

In one embodiment, the nucleic acid encodes one or more enzymes in the mevalonate (MVA) pathway. In one embodiment, the one or more enzymes is chosen from the group consisting of:

a) thiolase (EC 2.3.1.9),

b) HMG-CoA synthase (EC 2.3.3.10),

c) HMG-CoA reductase (EC 1.1.1.88),

d) Mevalonate kinase (EC 2.7.1.36),

e) Phosphomevalonate kinase (EC 2.7.4.2),

f) Mevalonate Diphosphate decarboxylase (EC 4.1.1.33), and

g) a functionally equivalent variant of any one thereof.

In a particular embodiment, the nucleic acid encodes thiolase (which may be an acetyl CoA c-acetyltransferase), HMG-CoA synthase and HMG-CoA reductase,

In a further embodiment, the nucleic acid encodes one or more enzymes in the mevalonate (MVA) pathway and one or more further nucleic acids in the DXS pathway. In one embodiment, the one or more enzymes from the DXS pathway is selected from the group consisting of:

a) 1-deoxy-D-xylulose-5-phosphate synthase DXS (EC:2.2.1.7),

b) 1-deoxy-D-xylulose 5-phosphate reductoisomerase DXR (EC:1.1.1.267),

c) 2-C-methyl-D-erythritol 4-phosphate cytidylyltransferase IspD (EC:2.7.7.60),

d) 4-diphosphocytidyl-2-C-methyl-D-erythritol kinase IspE (EC:2.7.1.148),

e) 2-C-methyl-D-erythritol 2,4-cyclodiphosphate synthase IspF (EC:4.6.1.12),

f) 4-hydroxy-3-methylbut-2-en-1-yl diphosphate synthase IspG (EC:1.17.7.1),

g) 4-hydroxy-3-methylbut-2-enyl diphosphate reductase (EC:1.17.1.2), and

h) a functionally equivalent variant of any one thereof.

In a further embodiment, the nucleic acid encodes one or more further exogenous or endogenous enzymes are expressed or over-expressed to result in the production of a terpene compound or a precursor thereof wherein the exogenous nucleic acid that is expressed, or the endogenous enzyme that is overexpressed, encodes and enzyme selected from the group consisting of:

a) geranyltranstransferase Fps (EC:2.5.1.10),

b) heptaprenyl diphosphate synthase (EC:2.5.1.10),

c) octaprenyl-diphosphate synthase (EC:2.5.1.90),

d) isoprene synthase (EC 4.2.3.27),

e) isopentenyl-diphosphate delta-isomerase (EC 5.3.3.2),

f) farnesene synthase (EC 4.2.3.46/EC 4.2.3.47), and

g) a functionally equivalent variant of any one thereof.

In one embodiment, the nucleic acid encoding thiolase (EC 2.3.1.9) has the sequence SEQ ID NO: 40 or is a functionally equivalent variant thereof.

In one embodiment, the nucleic acid encoding thiolase (EC 2.3.1.9) is acetyl CoA c-acetyl transferase that has the sequence SEQ ID NO: 41 or is a functionally equivalent variant thereof.

In one embodiment, the nucleic acid encoding HMG-CoA synthase (EC 2.3.3.10) has the sequence SEQ ID NO: 42 or is a functionally equivalent variant thereof.

In one embodiment, the nucleic acid encoding HMG-CoA reductase (EC 1.1.1.88) has the sequence SEQ ID NO: 43 or is a functionally equivalent variant thereof.

In one embodiment, the nucleic acid encoding Mevalonate kinase (EC 2.7.1.36) has the sequence SEQ ID NO: 51 or is a functionally equivalent variant thereof.

In one embodiment, the nucleic acid encoding Phosphomevalonate kinase (EC 2.7.4.2) has the sequence SEQ ID NO: 52 or is a functionally equivalent variant thereof.

In one embodiment, the nucleic acid encoding Mevalonate Diphosphate decarboxylase (EC 4.1.1.33) has the sequence SEQ ID NO: 53 or is a functionally equivalent variant thereof.

In one embodiment, the nucleic acid encoding 1-deoxy-D-xylulose-5-phosphate synthase DXS (EC:2.2.1.7) has the sequence SEQ ID NO: 1 or is a functionally equivalent variant thereof.

In one embodiment, the nucleic acid encoding 1-deoxy-D-xylulose 5-phosphate reductoisomerase DXR (EC:1.1.1.267) has the sequence SEQ ID NO: 3 or is a functionally equivalent variant thereof.

In one embodiment, the nucleic acid encoding 2-C-methyl-D-erythritol 4-phosphate cytidylyltransferase IspD (EC:2.7.7.60) has the sequence SEQ ID NO: 5 or is a functionally equivalent variant thereof.

In one embodiment, the nucleic acid encoding 4-diphosphocytidyl-2-C-methyl-D-erythritol kinase IspE (EC:2.7.1.148) has the sequence SEQ ID NO: 7 or is a functionally equivalent variant thereof.

In one embodiment, the nucleic acid encoding 2-C-methyl-D-erythritol 2,4-cyclodiphosphate synthase IspF (EC:4.6.1.12) has the sequence SEQ ID NO: 9 or is a functionally equivalent variant thereof.

In one embodiment, the nucleic acid encoding 4-hydroxy-3-methylbut-2-en-1-yl diphosphate synthase IspG (EC:1.17.7.1) has the sequence SEQ ID NO: 11 or is a functionally equivalent variant thereof.

In one embodiment, the nucleic acid encoding 4-hydroxy-3-methylbut-2-enyl diphosphate reductase (EC:1.17.1.2) has the sequence SEQ ID NO: 13 or is a functionally equivalent variant thereof.

In one embodiment, the nucleic acid encoding geranyltranstransferase Fps has the sequence SEQ ID NO: 15, or it is a functionally equivalent variant thereof.

In one embodiment, the nucleic acid encoding heptaprenyl diphosphate synthase has the sequence SEQ ID NO: 17, or it is a functionally equivalent variant thereof.

In one embodiment, the nucleic acid encoding octaprenyl-diphosphate synthase (EC:2.5.1.90) wherein the octaprenyl-diphosphate synthase is polyprenyl synthetase is encoded by sequence SEQ ID NO: 19, or it is a functionally equivalent variant thereof.

In one embodiment, the nucleic acid encoding isoprene synthase (ispS) has the sequence SEQ ID NO: 21, or it is a functionally equivalent variant thereof.

In one embodiment, the nucleic acid encoding Isopentenyl-diphosphate delta-isomerase (idi) has the sequence SEQ ID NO: 54, or it is a functionally equivalent variant thereof.

In one embodiment, the nucleic acid encoding farnesene synthase has the sequence SEQ ID NO: 57, or it is a functionally equivalent variant thereof.

In one embodiment, the nucleic acid encodes the following enzymes:

a) isoprene synthase;

b) Isopentenyl-diphosphate delta-isomerase (idi); and

c) 1-deoxy-D-xylulose-5-phosphate synthase DXS;

or functionally equivalent variants thereof.

In one embodiment, the nucleic acid encodes the following enzymes:

a) Thiolase;

b) HMG-CoA synthase;

c) HMG-CoA reductase;

d) Mevalonate kinase;

e) Phosphomevalonate kinase;

f) Mevalonate Diphosphate decarboxylase;

g) Isopentenyl-diphosphate delta-isomerase (idi); and

h) isoprene synthase;

or functionally equivalent variants thereof.

In one embodiment, the nucleic acid encodes the following enzymes:

a) geranyltranstransferase Fps; and

b) farnesene synthase

or functionally equivalent variants thereof.

In one embodiment, the nucleic acids of the disclosure further comprise a promoter. In one embodiment, the promoter allows for constitutive expression of the genes under its control. In a particular embodiment a Wood-Ljungdahl cluster promoter is used. In another particular embodiment, a Phosphotransacetylase/Acetate kinase operon promoter is used. In one particular embodiment, the promoter is from C. autoethanogenum.

In a third aspect, the disclosure provides a nucleic acid construct or vector comprising one or more nucleic acid of the second aspect.

In one particular embodiment, the nucleic acid construct or vector is an expression construct or vector. In one particular embodiment, the expression construct or vector is a plasmid.

In a fourth aspect, the disclosure provides host organisms comprising any one or more of the nucleic acids of the second aspect or vectors or constructs of the third aspect.

In a fifth aspect, the disclosure provides a composition comprising an expression construct or vector as referred to in the third aspect of the disclosure and a methylation construct or vector.

Preferably, the composition is able to produce a recombinant microorganism according to the first aspect of the disclosure.

In one particular embodiment, the expression construct/vector and/or the methylation construct/vector is a plasmid.

In a sixth aspect, the disclosure provides a method for the production of one or more terpenes and/or precursors thereof and optionally one or more other products by microbial fermentation comprising fermenting a substrate comprising CO using a recombinant microorganism of the first aspect of the disclosure.

In one embodiment the method comprises the steps of:

(a) providing a substrate comprising CO to a bioreactor containing a culture of one or more microorganisms of the first aspect of the disclosure; and
(b) anaerobically fermenting the culture in the bioreactor to produce at least one terpene and/or precursor thereof.

In one embodiment the method comprises the steps of:

(a) capturing CO-containing gas produced as a result of the industrial process;
(b) anaerobic fermentation of the CO-containing gas to produce at least one terpene and/or precursor thereof by a culture containing one or more microorganism of the first aspect of the disclosure.

In particular embodiments of the method aspects, the microorganism is maintained in an aqueous culture medium.

In particular embodiments of the method aspects, the fermentation of the substrate takes place in a bioreactor.

In one embodiment, the one or more terpene and/or precursor thereof is chosen from mevalonic acid, IPP, dimethylallyl pyrophosphate (DMAPP), isoprene, geranyl pyrophosphate (GPP), farnesyl pyrophosphate (FPP) and farnesene.

Preferably, the substrate comprising CO is a gaseous substrate comprising CO. In one embodiment, the substrate comprises an industrial waste gas. In certain embodiments, the gas is steel mill waste gas or syngas.

In one embodiment, the substrate will typically contain a major proportion of CO, such as at least about 20% to about 100% CO by volume, from 20% to 70% CO by volume, from 30% to 60% CO by volume, and from 40% to 55% CO by volume. In particular embodiments, the substrate comprises about 25%, or about 30%, or about 35%, or about 40%, or about 45%, or about 50% CO, or about 55% CO, or about 60% CO by volume.

In certain embodiments the methods further comprise the step of recovering a terpene and/or precursor thereof and optionally one or more other products from the fermentation broth.

In a seventh aspect, the disclosure provides one or more terpene and/or precursor thereof when produced by the method of the sixth aspect. In one embodiment, the one or more terpene and/or precursor thereof is chosen from the group consisting of mevalonic acid, IPP, dimethylallyl pyrophosphate (DMAPP), isoprene, geranyl pyrophosphate (GPP), farnesyl pyrophosphate (FPP) and farnesene.

In another aspect, the disclosure provides a method for the production of a microorganism of the first aspect of the disclosure comprising transforming a carboxydotrophic acetogenic parental microorganism by introduction of one or more nucleic acids such that the microorganism is capable of producing, or increasing the production of, one or more terpenes and/or precursors thereof and optionally one or more other products by fermentation of a substrate comprising CO, wherein the parental microorganism is not capable of producing, or produces at a lower level, the one or more terpene and/or precursor thereof by fermentation of a substrate comprising CO.

In one particular embodiment, a parental microorganism is transformed by introducing one or more exogenous nucleic acids adapted to express one or more enzymes in the mevalonate (MVA) pathway and optionally the DXS pathway. In another embodiment, a parental microorganism is transformed with one or more nucleic acids adapted to over-express one or more enzymes in the mevalonate (MVA) pathway and optionally the DXS pathway which are naturally present in the parental microorganism.

In certain embodiments, the one or more enzymes are as herein before described.

In one embodiment an isolated, genetically engineered, carboxydotrophic, acetogenic bacteria are provided which comprise an exogenous nucleic acid encoding an enzyme in a mevalonate pathway or in a DXS pathway or in a terpene biosynthesis pathway, whereby the bacteria express the enzyme. The enzyme is selected from the group consisting of:
a) thiolase (EC 2.3.1.9);
b) HMG-CoA synthase (EC 2.3.3.10);
c) HMG-CoA reductase (EC 1.1.1.88);
d) Mevalonate kinase (EC 2.7.1.36);
e) Phosphomevalonate kinase (EC 2.7.4.2);
f) Mevalonate Diphosphate decarboxylase (EC 4.1.1.33); 1-deoxy-D-xylulose-5-phosphate synthase DXS (EC:2.2.1.7);
g) 1-deoxy-D-xylulose 5-phosphate reductoisomerase DXR (EC:1.1.1.267);
h) 2-C-methyl-D-erythritol 4-phosphate cytidylyltransferase IspD (EC:2.7.7.60);
i) 4-diphosphocytidyl-2-C-methyl-D-erythritol kinase IspE (EC:2.7.1.148);
j) 2-C-methyl-D-erythritol 2;4-cyclodiphosphate synthase IspF (EC:4.6.1.12);
k) 4-hydroxy-3-methylbut-2-en-1-yl diphosphate synthase IspG (EC:1.17.7.1);
l) 4-hydroxy-3-methylbut-2-enyl diphosphate reductase (EC:1.17.1.2); geranyltranstransferase Fps (EC:2.5.1.10);
m) heptaprenyl diphosphate synthase (EC:2.5.1.10);
n) octaprenyl-diphosphate synthase (EC:2.5.1.90);
o) isoprene synthase (EC 4.2.3.27);
p) isopentenyl-diphosphate delta-isomerase (EC 5.3.3.2); and
q) farnesene synthase (EC 4.2.3.46/EC 4.2.3.47).

In some aspects the bacteria do not express the enzyme in the absence of said nucleic acid. In some aspects the bacteria which express the enzyme under anaerobic conditions.

One embodiment provides a plasmid which can replicate in a carboxydotrophic, acetogenic bacteria. The plasmid comprises a nucleic acid encoding an enzyme in a mevalonate pathway or in a DXS pathway or in a terpene biosynthesis pathway, whereby when the plasmid is in the bacteria, the enzyme is expressed by said bacteria. The enzyme is selected from the group consisting of:
a) thiolase (EC 2.3.1.9);
b) HMG-CoA synthase (EC 2.3.3.10);
c) HMG-CoA reductase (EC 1.1.1.88);
d) Mevalonate kinase (EC 2.7.1.36);
e) Phosphomevalonate kinase (EC 2.7.4.2);
f) Mevalonate Diphosphate decarboxylase (EC 4.1.1.33); 1-deoxy-D-xylulose-5-phosphate synthase DXS (EC:2.2.1.7);
g) 1-deoxy-D-xylulose 5-phosphate reductoisomerase DXR (EC:1.1.1.267);
h) 2-C-methyl-D-erythritol 4-phosphate cytidylyltransferase IspD (EC:2.7.7.60);
i) 4-diphosphocytidyl-2-C-methyl-D-erythritol kinase IspE (EC:2.7.1.148);
j) 2-C-methyl-D-erythritol 2;4-cyclodiphosphate synthase IspF (EC:4.6.1.12);
k) 4-hydroxy-3-methylbut-2-en-1-yl diphosphate synthase IspG (EC:1.17.7.1);
l) 4-hydroxy-3-methylbut-2-enyl diphosphate reductase (EC:1.17.1.2); geranyltranstransferase Fps (EC:2.5.1.10);
m) heptaprenyl diphosphate synthase (EC:2.5.1.10);
n) octaprenyl-diphosphate synthase (EC:2.5.1.90);
o) isoprene synthase (EC 4.2.3.27);
p) isopentenyl-diphosphate delta-isomerase (EC 5.3.3.2); and
q) farnesene synthase (EC 4.2.3.46/EC 4.2.3.47).

A process is provided in another embodiment for converting CO and/or CO₂ into isoprene. The process comprises: passing a gaseous CO-containing and/or CO₂₋containing substrate to a bioreactor containing a culture of carboxydotrophic, acetogenic bacteria in a culture medium such that the bacteria convert the CO and/or CO₂ to isoprene, and recovering the isoprene from the bioreactor. The carboxydotrophic acetogenic bacteria are genetically engineered to express an isoprene synthase.

Another embodiment provides an isolated, genetically engineered, carboxydotrophic, acetogenic bacteria which comprise a nucleic acid encoding an isoprene synthase. The bacteria express the isoprene synthase, and the bacteria are able to convert dimethylallyl diphosphate to isoprene. In one aspect the isoprene synthase is a Populus tremuloides enzyme. In another aspect the nucleic acid is codon optimized. In still another aspect, expression of the isoprene synthase is under the transcriptional control of a promoter for a pyruvate: ferredoxin oxidoreductase gene from Clostridium autoethanogenum.

Another embodiment provides a process for converting CO and/or CO₂ into isopentyl diphosphate (IPP). The process comprises: passing a gaseous CO-containing and/or CO₂₋containing substrate to a bioreactor containing a culture of carboxydotrophic, acetogenic bacteria in a culture medium such that the bacteria convert the CO and/or CO₂ to isopentyl diphosphate (IPP), and recovering the IPP from the bioreactor. The carboxydotrophic acetogenic bacteria are genetically engineered to express a isopentyl diphosphate delta isomerase.

Still another embodiment provides isolated, genetically engineered, carboxydotrophic, acetogenic bacteria which comprise a nucleic acid encoding an isopentyl diphosphate delta isomerase. The bacteria express the isopentyl diphosphate delta isomerase and the bacteria are able to convert dimethylallyl diphosphate to isopentyl diphosphate. In some aspects the nucleic acid encodes a Clostridium beijerinckii isopentyl diphosphate delta isomerase. In other aspects, the nucleic acid is under the transcriptional control of a promoter for a pyruvate: ferredoxin oxidoreductase gene from Clostridium autoethanogenum. In still other aspects, the nucleic acid is under the transcriptional control of a promoter for a pyruvate: ferredoxin oxidoreductase gene from Clostridium autoethanogenum and downstream of a second nucleic acid encoding an isoprene synthase.

Still another embodiment provides a process for converting CO and/or CO₂ into isopentyl diphosphate (IPP) and/or isoprene. The process comprises: passing a gaseous CO-containing and/or CO₂₋containing substrate to a bioreactor containing a culture of carboxydotrophic, acetogenic bacteria in a culture medium such that the bacteria convert the CO and/or CO₂ to isopentyl diphosphate (IPP) and/or isoprene, and recovering the IPP and/or isoprene from the bioreactor. The carboxydotrophic acetogenic bacteria are genetically engineered to have an increased copy number of a nucleic acid encoding a deoxyxylulose 5-phosphate synthase (DXS) enzyme, wherein the increased copy number is greater than 1 per genome.

Yet another embodiment provides isolated, genetically engineered, carboxydotrophic, acetogenic bacteria which comprise a copy number of greater than 1 per genome of a nucleic acid encoding a deoxyxylulose 5-phosphate synthase (DXS) enzyme. In some aspects, the isolated, genetically engineered, carboxydotrophic, acetogenic bacteria may further comprise a nucleic acid encoding an isoprene synthase. In other aspects, the isolated, genetically engineered, carboxydotrophic, acetogenic bacteria of may further comprise a nucleic acid encoding an isopentyl diphosphate delta isomerase. In still other aspects the isolated, genetically engineered, carboxydotrophic, acetogenic bacteria may further comprise a nucleic acid encoding an isopentyl diphosphate delta isomerase and a nucleic acid encoding an isoprene synthase.

Another embodiment provides isolated, genetically engineered, carboxydotrophic, acetogenic bacteria which comprise a nucleic acid encoding a phosphomevalonate kinase (PMK). The bacteria express the encoded enzyme, and the enzyme is not native to the bacteria. In some aspects the enzymes are Staphylococcus aureus enzymes. In some aspects the enzyme is expressed under the control of one or more C. autoethanogenum promoters. In some aspects the bacteria further comprise a nucleic acid encoding thiolase (thlA/vraB), a nucleic acid encoding an HMG-CoA synthase (HMGS), and a nucleic acid encoding an HMG-CoA reductase (HMGR). In some aspects the thiolase is Clostridium acetobutylicum thiolase. In some aspects the bacteria further comprise a nucleic acid encoding a mevalonate diphosphate decarboxylase (PMD).

Still another embodiment provides isolated, genetically engineered, carboxydotrophic, acetogenic bacteria which comprise an exogenous nucleic acid encoding alpha-farnesene synthase. In some aspects the nucleic acid is codon optimized for expression in C. autoethanogenum. In some aspects the alpha-farnesene synthase is a Malus x domestica alpha-farnesene synthase. In some aspects the bacteria further comprise a nucleic acid segment encoding geranyltranstransferase. In some aspects the geranyltranstransferase is an E. coli geranyltranstransferase.

Suitable isolated, genetically engineered, carboxydotrophic, acetogenic bacteria for any of the aspects or embodiments of the disclosure may be selected from the group consisting of Clostridium autoethanogenum, Clostridium ljungdahlii, Clostridium ragsdalei, Clostridium carboxidivorans, Clostridium drakei, Clostridium scatologenes, Clostridium aceticum, Clostridium formicoaceticum, Clostridium magnum, Butyribacterium methylotrophicum, Acetobacterium woodii, Alkalibaculum bacchii, Blautia producta, Eubacterium limosum, Moorella thermoacetica, Moorella thermautotrophica, Sporomusa ovata, Sporomusa silvacetica, Sporomusa sphaeroides, Oxobacter pfennigii, and Thermoanaerobacter kivui.

The disclosure may also be said broadly to consist in the parts, elements and features referred to or indicated in the specification of the application, individually or collectively, in any or all combinations of two or more of said parts, elements or features, and where specific integers are mentioned herein which have known equivalents in the art to which the disclosure relates, such known equivalents are deemed to be incorporated herein as if individually set forth.

### BRIEF DESCRIPTION OF THE FIGURES

These and other aspects of the present disclosure, which should be considered in all its novel aspects, will become apparent from the following description, which is given by way of example only, with reference to the accompanying figures.

FIG. 1: Pathway diagram for production of terpenes, gene targets described in this application are highlighted with bold arrows.

FIG. 2: Genetic map of plasmid pMTL 85146-ispS

FIG. 3: Genetic map of plasmid pMTL 85246-ispS-idi

FIG. 4: Genetic map of plasmid pMTL 85246-ispS-idi-dxs

FIG. 5: Sequencing results for plasmid pMTL 85246-ispS-idi-dxs

FIG. 6: Comparison of energetics for production of terpenes from CO via DXS and mevalonate pathway

FIG. 7: Mevalonate pathway

FIG. 8: Agarose gel electrophoresis image confirming presence of isoprene expression plasmid pMTL 85246-ispS-idi in C. autoethanogenum transformants. Lanes 1, and 20 show 100 bp Plus DNA Ladder. Lane 3-6, 9-12, 15-18 show PCR with isolated plasmids from 4 different clones as template, each in the following order: colE1, ermB, and idi. Lanes 2, 8, and 14 show PCR without template as negative control, each in the following order: colE1, ermB, and idi. Lanes 7, 13, and 19 show PCR with pMTL 85246-ispS-idi from E. coli as positive control, each in the following order: colE1, ermB, and idi.

FIG. 9: Mevalonate expression plasmid pMTL8215-Pptaack-thlA-HMGS-Patp-HMGR

FIG. 10: Isoprene expression plasmid pMTL 8314-Pptaack-thlA-HMGS-Patp-HMGR-Prnf-MK-PMK-PMD-Pfor-idi-ispS

FIG. 11: Farnesene expression plasmid pMTL8314-Pptaack-thlA-HMGS-Patp-HMGR-Prnf-MK-PMK-PMD-Pfor-idi-ispA-FS

FIG. 12: Genetic map of plasmid pMTL 85246-ispS-idi-dxs

FIG. 13: Amplification chart for gene expression experiment with C. autoethanogenum carrying plasmid pMTL 85146-ispS

FIG. 14: Amplification chart for gene expression experiment with C. autoethanogenum carrying plasmid pMTL 85246-ispS-idi

FIG. 15: Amplification chart for gene expression experiment with C. autoethanogenum carrying plasmid pMTL 85246-ispS-idi-dxs

FIG. 16: PCR check for the presence of the plasmid pMTL8314Prnf-MK-PMK-PMD-Pfor-idi-ispA-FS. Expected band size 1584 bp. The DNA marker Fermentas 1 kb DNA ladder.

FIG. 17: Growth curve for transformed C. autoethanogenum carrying plasmid pMTL8314Prnf-MK-PMK-PMD-Pfor-idi-ispA-FS.

FIG. 18: RT-PRC data showing the expression of the genes Mevalonate kinase (MK SEQ ID NO: 51), Phosphomevalonate Kinase (PMK SEQ ID NO: 52), Mevalonate Diphosphate Decarboxylase (PMD SEQ ID NO: 53), Isopentyl-diphosphate Delta-isomerase (idi SEQ ID NO: 54), Geranyltranstransferase (ispA SEQ ID NO: 56) and Farnesene synthase (FS SEQ ID NO: 57).

FIG. 19: GC-MS detection and conformation of the presence of farnesene in 1 mM mevalonate spiked cultures carrying pMTL8314Prnf-MK-PMK-PMD-Pfor-idi-ispA-FS. GC-MS chromatogram scanned for peaks containing ions with a mass of 93. Chromatograms 1 and 2 are transformed C. autoethanogenum, 3 is beta-farnesene standard run at the same time as the C. autoethanogenum samples. 4 is E. coli carrying the plasmids pMTL8314Prnf-MK-PMK-PMD-Pfor-idi-ispA-FS grown on M9 Glucose showing alpha-farnesene production and 5 is beta-farnesene standard run at the time of the E. coli samples. The difference in retention time between the E. coli and the C. autoethanogenum samples are due to minor changes to the instrument. However, the difference in retention time between the beta-farnesene standard and the produced alpha-farnesene are the exact same in both cases, which together with the match in mass spectra's confirm the production of alpha-farnesene in C. autoethanogenum.

FIG. 20: MS spectrums for peaks labeled 1A and 2A in FIG. 19. The MS spectra's matches up with the NIST database spectra (FIG. 21) confirming the peak is alpha-farnesene.

FIG. 21: MS spectrum for alpha-farnesene from the NIST Mass Spectral Database.

FIG. 22: Practical maximum isoprenol selectivity calculations.

FIG. 23: Pathway 1: Isoprenoid Alcohol (IPA) pathway.

FIG. 24: Pathway 2: IPA pathway+Ptb-buk.

FIG. 25: Pathway 3: IPA pathway via acetone.

FIG. 26: Pathway 4: IPA pathway via acetone+Ptb-buk.

FIG. 27: Pathway 5: Mevalonate pathway.

FIG. 28: Pathway 6: Mevalonate pathway+IPP bypass.

FIG. 29: Metabolites of Pathways 1-6.

### DETAILED DESCRIPTION OF THE DISCLOSURE

The following is a description of the present disclosure, including preferred embodiments thereof, given in general terms. The disclosure is further elucidated from the disclosure given under the heading “Examples” herein below, which provides experimental data supporting the disclosure, specific examples of various aspects of the disclosure, and means of performing the disclosure.

The inventors have surprisingly been able to engineer a carboxydotrophic acetogenic microorganism to produce isoprenoid alcohols, isoprenoid alcohol derivatives, terpenes and precursors thereof including isoprene and farnesene by fermentation of a gas substrate. This offers an alternative means for the production of these products which may have benefits over the current methods for their production. In addition, it offers a means of using carbon monoxide from industrial processes which would otherwise be released into the atmosphere and pollute the environment.

The term “non-naturally occurring” when used in reference to a microorganism is intended to mean that the microorganism has at least one genetic modification not found in a naturally occurring strain of the referenced species, including wild-type strains of the referenced species. Non-naturally occurring microorganisms are typically developed in a laboratory or research facility. The microorganisms of the disclosure are non-naturally occurring.

The terms “genetic modification,” “genetic alteration,” or “genetic engineering” broadly refer to manipulation of the genome or nucleic acids of a microorganism by the hand of man. Likewise, the terms “genetically modified,” “genetically altered,” or “genetically engineered” refers to a microorganism containing such a genetic modification, genetic alteration, or genetic engineering. These terms may be used to differentiate a lab-generated microorganism from a naturally-occurring microorganism. Methods of genetic modification of include, for example, heterologous gene expression, gene or promoter insertion or deletion, nucleic acid mutation, altered gene expression or inactivation, enzyme engineering, directed evolution, knowledge-based design, random mutagenesis methods, gene shuffling, and codon optimization. The microorganisms of the disclosure are genetically engineered.

“Recombinant” indicates that a nucleic acid, protein, or microorganism is the product of genetic modification, engineering, or recombination. Generally, the term “recombinant” refers to a nucleic acid, protein, or microorganism that contains or is encoded by genetic material derived from multiple sources, such as two or more different strains or species of microorganisms. The microorganisms of the disclosure are generally recombinant.

“Wild type” refers to the typical form of an organism, strain, gene, or characteristic as it occurs in nature, as distinguished from mutant or variant forms.

“Endogenous” refers to a nucleic acid or protein that is present or expressed in the wild-type or parental microorganism from which the microorganism of the disclosure is derived. For example, an endogenous gene is a gene that is natively present in the wild-type or parental microorganism from which the microorganism of the disclosure is derived. In one embodiment, the expression of an endogenous gene may be controlled by an exogenous regulatory element, such as an exogenous promoter.

“Exogenous” refers to a nucleic acid or protein that originates outside the microorganism of the disclosure. For example, an exogenous gene or enzyme may be artificially or recombinantly created and introduced to or expressed in the microorganism of the disclosure. An exogenous gene or enzyme may also be isolated from a heterologous microorganism and introduced to or expressed in the microorganism of the disclosure. Exogenous nucleic acids may be adapted to integrate into the genome of the microorganism of the disclosure or to remain in an extra-chromosomal state in the microorganism of the disclosure, for example, in a plasmid.

“Heterologous” refers to a nucleic acid or protein that is not present in the wild-type or parental microorganism from which the microorganism of the disclosure is derived. For example, a heterologous gene or enzyme may be derived from a different strain or species and introduced to or expressed in the microorganism of the disclosure. The heterologous gene or enzyme may be introduced to or expressed in the microorganism of the disclosure in the form in which it occurs in the different strain or species. Alternatively, the heterologous gene or enzyme may be modified in some way, e.g., by codon-optimizing it for expression in the microorganism of the disclosure or by engineering it to alter function, such as to reverse the direction of enzyme activity or to alter substrate specificity.

In particular, a heterologous nucleic acid or protein expressed in the microorganism described herein may be derived from Bacillus, Clostridium, Cupriavidus, Escherichia, Gluconobacter, Hyphomicrobium, Lysinibacillus, Paenibacillus, Pseudomonas, Sedimenticola, Sporosarcina, Streptomyces, Thermithiobacillus, Thermotoga, Zea, Klebsiella, Mycobacterium, Salmonella, Mycobacteroides, Staphylococcus, Burkholderia, Listeria, Acinetobacter, Shigella, Neisseria, Bordetella, Streptococcus, Enterobacter, Vibrio, Legionella, Xanthomonas, Serratia, Cronobacter, Cupriavidus, Helicobacter, Yersinia, Cutibacterium, Francisella, Pectobacterium, Arcobacter, Lactobacillus, Shewanella, Erwinia, Sulfurospirillum, Peptococcaceae, Thermococcus, Saccharomyces, Pyrococcus, Glycine, Homo, Ralstonia, Brevibacterium, Methylobacterium, Geobacillus, bos, gallus, Anaerococcus, Xenopus, Amblyrhynchus, rattus, mus, sus, Rhodococcus, Rhizobium, Megasphaera, Mesorhizobium, Peptococcus, Agrobacterium, Campylobacter, Acetobacterium, Alkalibaculum, Blautia, Butyribacterium, Eubacterium, Moorella, Oxobacter, Sporomusa, Thermoanaerobacter, Schizosaccharomyces, Paenibacillus, Fictibacillus, Lysinibacillus, Ornithinibacillus, Halobacillus, Kurthia, Lentibacillus, Anoxybacillus, Solibacillus, Virgibacillus, Alicyclobacillus, Sporosarcina, Salimicrobium, Sporosarcina, Planococcus, Corynebacterium, Thermaerobacter, Sulfobacillus, or Symbiobacterium.

The terms “polynucleotide,” “nucleotide,” “nucleotide sequence,” “nucleic acid,” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides or nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene products.”

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, by disulfide bond formation, glycosylation, lipidation, acetylation, phosphorylation, or any other manipulation, such as conjugation with a labeling component. As used herein, the term “amino acid” includes natural and/or unnatural or synthetic amino acids, including glycine and both the D or L optical isomers, and amino acid analogs and peptidomimetics.

“Enzyme activity,” or simply “activity,” refers broadly to enzymatic activity, including, but not limited, to the activity of an enzyme, the amount of an enzyme, or the availability of an enzyme to catalyze a reaction. Accordingly, “increasing” enzyme activity includes increasing the activity of an enzyme, increasing the amount of an enzyme, or increasing the availability of an enzyme to catalyze a reaction. Similarly, “decreasing” enzyme activity includes decreasing the activity of an enzyme, decreasing the amount of an enzyme, or decreasing the availability of an enzyme to catalyze a reaction.

“Mutated” refers to a nucleic acid or protein that has been modified in the microorganism of the disclosure compared to the wild-type or parental microorganism from which the microorganism of the disclosure is derived. In one embodiment, the mutation may be a deletion, insertion, or substitution in a gene encoding an enzyme. In another embodiment, the mutation may be a deletion, insertion, or substitution of one or more amino acids in an enzyme.

“Disrupted gene” refers to a gene that has been modified in some way to reduce or eliminate expression of the gene, regulatory activity of the gene, or activity of an encoded protein or enzyme. The disruption may partially inactivate, fully inactivate, or delete the gene or enzyme. The disruption may be a knockout (KO) mutation that fully eliminates the expression or activity of a gene, protein, or enzyme. The disruption may also be a knock-down that reduces, but does not entirely eliminate, the expression or activity of a gene, protein, or enzyme. The disruption may be anything that reduces, prevents, or blocks the biosynthesis of a product produced by an enzyme. The disruption may include, for example, a mutation in a gene encoding a protein or enzyme, a mutation in a genetic regulatory element involved in the expression of a gene encoding an enzyme, the introduction of a nucleic acid which produces a protein that reduces or inhibits the activity of an enzyme, or the introduction of a nucleic acid (e.g., antisense RNA, RNAi, TALEN, siRNA, CRISPR, or CRISPRi) or protein which inhibits the expression of a protein or enzyme. The disruption may be introduced using any method known in the art. For the purposes of the present disclosure, disruptions are laboratory-generated, not naturally occurring.

A “parental microorganism” is a microorganism used to generate a microorganism of the disclosure. The parental microorganism may be a naturally-occurring microorganism (i.e., a wild-type microorganism) or a microorganism that has been previously modified (i.e., a mutant or recombinant microorganism). The microorganism of the disclosure may be modified to express or overexpress one or more enzymes that were not expressed or overexpressed in the parental microorganism. Similarly, the microorganism of the disclosure may be modified to contain one or more genes that were not contained by the parental microorganism. The microorganism of the disclosure may also be modified to not express or to express lower amounts of one or more enzymes that were expressed in the parental microorganism.

The microorganism of the disclosure may be derived from essentially any parental microorganism. In one embodiment, the microorganism of the disclosure may be derived from a parental microorganism selected from the group consisting of Clostridium acetobutylicum, Clostridium beijerinckii, Escherichia coli, and Saccharomyces cerevisiae. In other embodiments, the microorganism is derived from a parental microorganism selected from the group consisting of Acetobacterium woodii, Alkalibaculum bacchii, Blautia product, Butyribacterium methylotrophicum, Clostridium aceticum, Clostridium autoethanogenum, Clostridium carboxidivorans, Clostridium coskatii, Clostridium drakei, Clostridium formicoaceticum, Clostridium ljungdahlii, Clostridium magnum, Clostridium ragsdalei, Clostridium scatologenes, Eubacterium limosum, Moorella thermautotrophica, Moorella thermoacetica, Oxobacter pfennigii, Sporomusa ovata, Sporomusa silvacetica, Sporomusa sphaeroides, and Thermoanaerobacter kivui. In a preferred embodiment, the parental microorganism is Clostridium autoethanogenum, Clostridium ljungdahlii, or Clostridium ragsdalei. In an especially preferred embodiment, the parental microorganism is Clostridium autoethanogenum LZ1561, which was deposited on Jun. 7, 2010, with Deutsche Sammlung von Mikroorganismen and Zellkulturen GmbH (DSMZ) located at Inhoffenstraße 7B, D-38124 Braunschweig, Germany on Jun. 7, 2010, under the terms of the Budapest Treaty and accorded accession number DSM23693. This strain is described in International Patent Application No. PCT/NZ2011/000144, which published as WO 2012/015317.

The term “derived from” indicates that a nucleic acid, protein, or microorganism is modified or adapted from a different (e.g., a parental or wild-type) nucleic acid, protein, or microorganism, so as to produce a new nucleic acid, protein, or microorganism. Such modifications or adaptations typically include insertion, deletion, mutation, or substitution of nucleic acids or genes. Generally, the microorganism of the disclosure is derived from a parental microorganism. In one embodiment, the microorganism of the disclosure is derived from Clostridium autoethanogenum, Clostridium ljungdahlii, or Clostridium ragsdalei. In a preferred embodiment, the microorganism of the disclosure is derived from Clostridium autoethanogenum LZ1561, which is deposited under DSMZ accession number DSM23693.

The microorganism of the disclosure may be further classified based on functional characteristics. For example, the microorganism of the disclosure may be or may be derived from a C1-fixing microorganism, an anaerobe, an acetogen, an ethanologen, a carboxydotroph, and/or a methanotroph.

Table 1 provides a representative list of microorganisms and identifies their functional characteristics.

TABLE 1

| | Wood- | C1- | | | | | |
|---|---|---|---|---|---|---|---|
| | Ljungdahl | fixing | Anaerobe | Acetogen | Ethanologen | Autotroph | Carboxydotroph |
| Acetobacterium woodii | + | + | + | + | +/− ¹ | + | − |
| Alkalibaculum bacchii | + | + | + | + | + | + | + |
| Blautia producta | + | + | + | + | − | + | + |
| Butyribacterium methylotrophicum | + | + | + | + | + | + | + |
| Clostridium aceticum | + | + | + | + | − | + | + |
| Clostridium autoethanogenum | + | + | + | + | + | + | + |
| Clostridium carboxidivorans | + | + | + | + | + | + | + |
| Clostridium coskatii | + | + | + | + | + | + | + |
| Clostridium drakei | + | + | + | + | − | + | + |
| Clostridium formicoaceticum | + | + | + | + | − | + | + |
| Clostridium ljungdahlii | + | + | + | + | + | + | + |
| Clostridium magnum | + | + | + | + | − | + | +/− ² |
| Clostridium ragsdalei | + | + | + | + | + | + | + |
| Clostridium scatologenes | + | + | + | + | − | + | + |
| Eubacterium limosum | + | + | + | + | − | + | + |
| Moorella thermautotrophica | + | + | + | + | + | + | + |
| Moorella thermoacetica (formerly | + | + | + | + | − ³ | + | + |
| Clostridium thermoaceticum) | | | | | | | |
| Oxobacter pfennigii | + | + | + | + | − | + | + |
| Sporomusa ovata | + | + | + | + | − | + | +/− ⁴ |
| Sporomusa silvacetica | + | + | + | + | − | + | +/− ⁵ |
| Sporomusa sphaeroides | + | + | + | + | − | + | +/− ⁶ |
| Thermoanaerobacter kivui | + | + | + | + | − | + | − |

¹ Acetobacterium woodii can produce ethanol from fructose, but not from gas.
² It has not been investigated whether Clostridium magnum can grow on CO.
³ One strain of Moorella thermoacetica, Moorella sp. HUC22-1, has been reported to produce ethanol from gas.
⁴ It has not been investigated whether Sporomusa ovata can grow on CO.
⁵ It has not been investigated whether Sporomusa silvacetica can grow on CO.
⁶ It has not been investigated whether Sporomusa sphaeroides can grow on CO.

“Wood-Ljungdahl” refers to the Wood-Ljungdahl pathway of carbon fixation as described, e.g., by Ragsdale, Biochim Biophys Acta, 1784: 1873-1898, 2008. “Wood-Ljungdahl microorganisms” refers, predictably, to microorganisms containing the Wood-Ljungdahl pathway. Often, the microorganism of the disclosure contains a native Wood-Ljungdahl pathway. Herein, a Wood-Ljungdahl pathway may be a native, unmodified Wood-Ljungdahl pathway or it may be a Wood-Ljungdahl pathway with some degree of genetic modification (e.g., overexpression, heterologous expression, knockout, etc.) so long as it still functions to convert CO, CO₂, and/or H₂ to acetyl-CoA.

“C1” refers to a one-carbon molecule, for example, CO, CO₂, CH₄, or CH₃OH. “C1-oxygenate” refers to a one-carbon molecule that also comprises at least one oxygen atom, for example, CO, CO₂, or CH₃OH. “C1-carbon source” refers a one carbon-molecule that serves as a partial or sole carbon source for the microorganism of the disclosure. For example, a C1-carbon source may comprise one or more of CO, CO₂, CH₄, CH₃OH, or CH₂O₂. Preferably, the C1-carbon source comprises one or both of CO and CO₂. A “C1-fixing microorganism” is a microorganism that has the ability to produce one or more products from a C1-carbon source. Often, the microorganism of the disclosure is a C1-fixing bacterium. In a preferred embodiment, the microorganism of the disclosure is derived from a C1-fixing microorganism identified in Table 1.

An “anaerobe” is a microorganism that does not require oxygen for growth. An anaerobe may react negatively or even die if oxygen is present above a certain threshold. However, some anaerobes are capable of tolerating low levels of oxygen (e.g., 0.000001-5% oxygen), sometimes referred to as “microoxic conditions.” Often, the microorganism of the disclosure is an anaerobe. In a preferred embodiment, the microorganism of the disclosure is derived from an anaerobe identified in Table 1.

“Acetogens” are obligately anaerobic bacteria that use the Wood-Ljungdahl pathway as their main mechanism for energy conservation and for synthesis of acetyl-CoA and acetyl-CoA-derived products, such as acetate (Ragsdale, Biochim Biophys Acta, 1784: 1873-1898, 2008). In particular, acetogens use the Wood-Ljungdahl pathway as a (1) mechanism for the reductive synthesis of acetyl-CoA from CO₂, (2) terminal electron-accepting, energy conserving process, (3) mechanism for the fixation (assimilation) of CO₂ in the synthesis of cell carbon (Drake, Acetogenic Prokaryotes, In: The Prokaryotes, 3^{rd} edition, p. 354, New York, N.Y., 2006). All naturally occurring acetogens are C1-fixing, anaerobic, autotrophic, and non-methanotrophic. Often, the microorganism of the disclosure is an acetogen. In a preferred embodiment, the microorganism of the disclosure is derived from an acetogen identified in Table 1.

An “ethanologen” is a microorganism that produces or is capable of producing ethanol. Often, the microorganism of the disclosure is an ethanologen. In a preferred embodiment, the microorganism of the disclosure is derived from an ethanologen identified in Table 1.

An “autotroph” is a microorganism capable of growing in the absence of organic carbon. Instead, autotrophs use inorganic carbon sources, such as CO and/or CO₂. Often, the microorganism of the disclosure is an autotroph. In a preferred embodiment, the microorganism of the disclosure is derived from an autotroph identified in Table 1.

A “carboxydotroph” is a microorganism capable of utilizing CO as a sole source of carbon and energy. Often, the microorganism of the disclosure is a carboxydotroph. In a preferred embodiment, the microorganism of the disclosure is derived from a carboxydotroph identified in Table 1.

A “methanotroph” is a microorganism capable of utilizing methane as a sole source of carbon and energy. In certain embodiments, the microorganism of the disclosure is a methanotroph or is derived from a methanotroph. In other embodiments, the microorganism of the disclosure is not a methanotroph or is not derived from a methanotroph.

In a preferred embodiment, the microorganism of the disclosure is derived from the cluster of Clostridia comprising the species Clostridium autoethanogenum, Clostridium ljungdahlii, and Clostridium ragsdalei. These species were first reported and characterized by Abrini, Arch Microbiol, 161: 345-351, 1994 (Clostridium autoethanogenum), Tanner, Int J System Bacteriol, 43: 232-236, 1993 (Clostridium ljungdahlii), and Huhnke, WO 2008/028055 (Clostridium ragsdalei).

These three species have many similarities. In particular, these species are all C1-fixing, anaerobic, acetogenic, ethanologenic, and carboxydotrophic members of the genus Clostridium. These species have similar genotypes and phenotypes and modes of energy conservation and fermentative metabolism. Moreover, these species are clustered in clostridial rRNA homology group I with 16S rRNA DNA that is more than 99% identical, have a DNA G+C content of about 22-30 mol %, are gram-positive, have similar morphology and size (logarithmic growing cells between 0.5-0.7×3-5 μm), are mesophilic (grow optimally at 30-37° C.), have similar pH ranges of about 4-7.5 (with an optimal pH of about 5.5-6), lack cytochromes, and conserve energy via an Rnf complex. Also, reduction of carboxylic acids into their corresponding alcohols has been shown in these species (Perez, Biotechnol Bioeng, 110:1066-1077, 2012). Importantly, these species also all show strong autotrophic growth on CO-containing gases, produce ethanol and acetate (or acetic acid) as main fermentation products, and produce small amounts of 2,3-butanediol and lactic acid under certain conditions.

However, these three species also have a number of differences. These species were isolated from different sources: Clostridium autoethanogenum from rabbit gut, Clostridium ljungdahlii from chicken yard waste, and Clostridium ragsdalei from freshwater sediment. These species differ in utilization of various sugars (e.g., rhamnose, arabinose), acids (e.g., gluconate, citrate), amino acids (e.g., arginine, histidine), and other substrates (e.g., betaine, butanol). Moreover, these species differ in auxotrophy to certain vitamins (e.g., thiamine, biotin). These species have differences in nucleic and amino acid sequences of Wood-Ljungdahl pathway genes and proteins, although the general organization and number of these genes and proteins has been found to be the same in all species (Köpke, Curr Opin Biotechnol, 22: 320-325, 2011).

Thus, in summary, many of the characteristics of Clostridium autoethanogenum, Clostridium ljungdahlii, or Clostridium ragsdalei are not specific to that species, but are rather general characteristics for this cluster of C1-fixing, anaerobic, acetogenic, ethanologenic, and carboxydotrophic members of the genus Clostridium. However, since these species are, in fact, distinct, the genetic modification or manipulation of one of these species may not have an identical effect in another of these species. For instance, differences in growth, performance, or product production may be observed.

The microorganism of the disclosure may also be derived from an isolate or mutant of Clostridium autoethanogenum, Clostridium ljungdahlii, or Clostridium ragsdalei. Isolates and mutants of Clostridium autoethanogenum include JA1-1 (DSM10061) (Abrini, Arch Microbiol, 161: 345-351, 1994), LBS1560 (DSM19630) (WO 2009/064200), and LZ1561 (DSM23693) (WO 2012/015317). Isolates and mutants of Clostridium ljungdahlii include ATCC 49587 (Tanner, Int J Syst Bacteriol, 43: 232-236, 1993), PETCT (DSM13528, ATCC 55383), ERI-2 (ATCC 55380) (U.S. Pat. No. 5,593,886), C-01 (ATCC 55988) (U.S. Pat. No. 6,368,819), O-52 (ATCC 55989) (U.S. Pat. No. 6,368,819), and OTA-1 (Tirado-Acevedo, Production of bioethanol from synthesis gas using Clostridium ljungdahlii, PhD thesis, North Carolina State University, 2010). Isolates and mutants of Clostridium ragsdalei include PI 1 (ATCC BAA-622, ATCC PTA-7826) (WO 2008/028055).

As described above, however, the microorganism of the disclosure may also be derived from essentially any parental microorganism, such as a parental microorganism selected from the group consisting of Clostridium acetobutylicum, Clostridium beijerinckii, Escherichia coli, and Saccharomyces cerevisiae.

Introduction of a disruptive mutation results in a microorganism of the disclosure that produces no target product or substantially no target product or a reduced amount of target product compared to the parental microorganism from which the microorganism of the disclosure is derived. For example, the microorganism of the disclosure may produce no target product or at least about 1%, 3%, 5%, 10%, 20%, 30%, 40%, 50%, 60%, 70%, 80%, 90%, or 95% less target product than the parental microorganism. For example, the microorganism of the disclosure may produce less than about 0.001, 0.01, 0.10, 0.30, 0.50, or 1.0 g/L target product.

Although exemplary sequences and sources for enzymes are provided herein, the disclosure is by no means limited to these sequences and sources—it also encompasses variants. The term “variants” includes nucleic acids and proteins whose sequence varies from the sequence of a reference nucleic acid and protein, such as a sequence of a reference nucleic acid and protein disclosed in the prior art or exemplified herein. The disclosure may be practiced using variant nucleic acids or proteins that perform substantially the same function as the reference nucleic acid or protein. For example, a variant protein may perform substantially the same function or catalyze substantially the same reaction as a reference protein. A variant gene may encode the same or substantially the same protein as a reference gene. A variant promoter may have substantially the same ability to promote the expression of one or more genes as a reference promoter.

Such nucleic acids or proteins may be referred to herein as “functionally equivalent variants.” By way of example, functionally equivalent variants of a nucleic acid may include allelic variants, fragments of a gene, mutated genes, polymorphisms, and the like. Homologous genes from other microorganisms are also examples of functionally equivalent variants. These include homologous genes in species such as Clostridium acetobutylicum, Clostridium beijerinckii, or Clostridium ljungdahlii, the details of which are publicly available on websites such as Genbank or NCBI. Functionally equivalent variants also include nucleic acids whose sequence varies as a result of codon optimization for a particular microorganism. A functionally equivalent variant of a nucleic acid will preferably have at least approximately 70%, approximately 80%, approximately 85%, approximately 90%, approximately 95%, approximately 98%, or greater nucleic acid sequence identity (percent homology) with the referenced nucleic acid. A functionally equivalent variant of a protein will preferably have at least approximately 70%, approximately 80%, approximately 85%, approximately 90%, approximately 95%, approximately 98%, or greater amino acid identity (percent homology) with the referenced protein. The functional equivalence of a variant nucleic acid or protein may be evaluated using any method known in the art.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%. 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

Nucleic acids may be delivered to a microorganism of the disclosure using any method known in the art. For example, nucleic acids may be delivered as naked nucleic acids or may be formulated with one or more agents, such as liposomes. The nucleic acids may be DNA, RNA, cDNA, or combinations thereof, as is appropriate. Restriction inhibitors may be used in certain embodiments. Additional vectors may include plasmids, viruses, bacteriophages, cosmids, and artificial chromosomes. In a preferred embodiment, nucleic acids are delivered to the microorganism of the disclosure using a plasmid. By way of example, transformation (including transduction or transfection) may be achieved by electroporation, ultrasonication, polyethylene glycol-mediated transformation, chemical or natural competence, protoplast transformation, prophage induction, or conjugation. In certain embodiments having active restriction enzyme systems, it may be necessary to methylate a nucleic acid before introduction of the nucleic acid into a microorganism.

Furthermore, nucleic acids may be designed to comprise a regulatory element, such as a promoter, to increase or otherwise control expression of a particular nucleic acid. The promoter may be a constitutive promoter or an inducible promoter. Ideally, the promoter is a Wood-Ljungdahl pathway promoter, a ferredoxin promoter, a pyruvate ferredoxin oxidoreductase promoter, an Rnf complex operon promoter, an ATP synthase operon promoter, or a phosphotransacetylase/acetate kinase operon promoter.

It should be appreciated that the disclosure may be practiced using nucleic acids whose sequence varies from the sequences specifically exemplified herein provided they perform substantially the same function. For nucleic acid sequences that encode a protein or peptide this means that the encoded protein or peptide has substantially the same function. For nucleic acid sequences that represent promoter sequences, the variant sequence will have the ability to promote expression of one or more genes. Such nucleic acids may be referred to herein as “functionally equivalent variants.” By way of example, functionally equivalent variants of a nucleic acid include allelic variants, fragments of a gene, genes which include mutations (deletion, insertion, nucleotide substitutions and the like) and/or polymorphisms and the like. Homologous genes from other microorganisms may also be considered as examples of functionally equivalent variants of the sequences specifically exemplified herein.

These include homologous genes in species such as Clostridium ljungdahlii, Chloroflexus aurantiacus, Metallosphaera or Sulfolobus spp, details of which are publicly available on websites such as Genbank or NCBI. The phrase “functionally equivalent variants” should also be taken to include nucleic acids whose sequence varies as a result of codon optimisation for a particular organism. “Functionally equivalent variants” of a nucleic acid herein will preferably have at least approximately 70%, preferably approximately 80%, more preferably approximately 85%, preferably approximately 90%, preferably approximately 95% or greater nucleic acid sequence identity with the nucleic acid identified.

It should also be appreciated that the disclosure may be practiced using polypeptides whose sequence varies from the amino acid sequences specifically exemplified herein. These variants may be referred to herein as “functionally equivalent variants.” A functionally equivalent variant of a protein or a peptide includes those proteins or peptides that share at least 40%, preferably 50%, preferably 60%, preferably 70%, preferably 75%, preferably 80%, preferably 85%, preferably 90%, preferably 95% or greater amino acid identity with the protein or peptide identified and has substantially the same function as the peptide or protein of interest. Such variants include within their scope fragments of a protein or peptide wherein the fragment comprises a truncated form of the polypeptide wherein deletions may be from 1 to 5, to 10, to 15, to 20, to 25 amino acids, and may extend from residue 1 through 25 at either terminus of the polypeptide, and wherein deletions may be of any length within the region; or may be at an internal location. Functionally equivalent variants of the specific polypeptides herein should also be taken to include polypeptides expressed by homologous genes in other species of bacteria, for example as exemplified in the previous paragraph.

The microorganisms of the disclosure may be prepared from a parental microorganism and one or more exogenous nucleic acids using any number of techniques known in the art for producing recombinant microorganisms. By way of example only, transformation (including transduction or transfection) may be achieved by electroporation, ultrasonication, polyethylene glycol-mediated transformation, chemical or natural competence, or conjugation. Suitable transformation techniques are described for example in, Sambrook J, Fritsch E F, Maniatis T: Molecular Cloning: A laboratory Manual, Cold Spring Harbour Laboratory Press, Cold Spring Harbour, 1989.

In certain embodiments, due to the restriction systems which are active in the microorganism to be transformed, it is necessary to methylate the nucleic acid to be introduced into the microorganism. This can be done using a variety of techniques, including those described below, and further exemplified in the Examples section herein after.

By way of example, in one embodiment, a recombinant microorganism of the disclosure is produced by a method comprises the following steps: introduction into a shuttle microorganism of (i) of an expression construct/vector as described herein and (ii) a methylation construct/vector comprising a methyltransferase gene; expression of the methyltransferase gene; isolation of one or more constructs/vectors from the shuttle microorganism; and, introduction of the one or more construct/vector into a destination microorganism.

In one embodiment, the methyltransferase gene of step B is expressed constitutively. In another embodiment, expression of the methyltransferase gene of step B is induced.

The shuttle microorganism is a microorganism, preferably a restriction negative microorganism that facilitates the methylation of the nucleic acid sequences that make up the expression construct/vector. In a particular embodiment, the shuttle microorganism is a restriction negative E. coli, Bacillus subtilis, or Lactococcus lactis.

The methylation construct/vector comprises a nucleic acid sequence encoding a methyltransferase.

Once the expression construct/vector and the methylation construct/vector are introduced into the shuttle microorganism, the methyltransferase gene present on the methylation construct/vector is induced. Induction may be by any suitable promoter system although in one particular embodiment of the disclosure, the methylation construct/vector comprises an inducible lac promoter and is induced by addition of lactose or an analogue thereof, more preferably isopropyl-β-D-thiogalactoside (IPTG). Other suitable promoters include the ara, tet, or T7 system. In a further embodiment of the disclosure, the methylation construct/vector promoter is a constitutive promoter.

In a particular embodiment, the methylation construct/vector has an origin of replication specific to the identity of the shuttle microorganism so that any genes present on the methylation construct/vector are expressed in the shuttle microorganism. Preferably, the expression construct/vector has an origin of replication specific to the identity of the destination microorganism so that any genes present on the expression construct/vector are expressed in the destination microorganism.

Expression of the methyltransferase enzyme results in methylation of the genes present on the expression construct/vector. The expression construct/vector may then be isolated from the shuttle microorganism according to any one of a number of known methods. By way of example only, the methodology described in the Examples section described hereinafter may be used to isolate the expression construct/vector.

In one particular embodiment, both construct/vector are concurrently isolated.

The expression construct/vector may be introduced into the destination microorganism using any number of known methods. However, by way of example, the methodology described in the Examples section hereinafter may be used. Since the expression construct/vector is methylated, the nucleic acid sequences present on the expression construct/vector are able to be incorporated into the destination microorganism and successfully expressed.

It is envisaged that a methyltransferase gene may be introduced into a shuttle microorganism and over-expressed. Thus, in one embodiment, the resulting methyltransferase enzyme may be collected using known methods and used in vitro to methylate an expression plasmid. The expression construct/vector may then be introduced into the destination microorganism for expression. In another embodiment, the methyltransferase gene is introduced into the genome of the shuttle microorganism followed by introduction of the expression construct/vector into the shuttle microorganism, isolation of one or more constructs/vectors from the shuttle microorganism and then introduction of the expression construct/vector into the destination microorganism.

It is envisaged that the expression construct/vector and the methylation construct/vector as defined above may be combined to provide a composition of matter. Such a composition has particular utility in circumventing restriction barrier mechanisms to produce the recombinant microorganisms of the disclosure.

In one particular embodiment, the expression construct/vector and/or the methylation construct/vector are plasmids.

Persons of ordinary skill in the art will appreciate a number of suitable methyltransferases of use in producing the microorganisms of the disclosure. However, by way of example the Bacillus subtilis phage ΦT1 methyltransferase and the methyltransferase described in the Examples herein after may be used. Nucleic acids encoding suitable methyltransferases will be readily appreciated having regard to the sequence of the desired methyltransferase and the genetic code.

Any number of constructs/vectors adapted to allow expression of a methyltransferase gene may be used to generate the methylation construct/vector.

In one embodiment, the substrate comprises CO. In one embodiment, the substrate comprises CO2 and CO. In another embodiment, the substrate comprises CO2 and H2. In another embodiment, the substrate comprises CO2 and CO and H2.

“Substrate” refers to a carbon and/or energy source for the microorganism of the disclosure. Often, the substrate is gaseous and comprises a C1-carbon source, for example, CO, CO2, and/or CH4. Preferably, the substrate comprises a C1-carbon source of CO or CO+CO2. The substrate may further comprise other non-carbon components, such as H2, N2, or electrons. In other embodiments, however, the substrate may be a carbohydrate, such as sugar, starch, fiber, lignin, cellulose, or hemicellulose or a combination thereof. For example, the carbohydrate may be fructose, galactose, glucose, lactose, maltose, sucrose, xylose, or some combination thereof. In some embodiments, the substrate does not comprise (D)-xylose (Alkim, Microb Cell Fact, 14: 127, 2015). In some embodiments, the substrate does not comprise a pentose such as xylose (Pereira, Metab Eng, 34: 80-87, 2016). In some embodiments, the substrate may comprise both gaseous and carbohydrate substrates (mixotrophic fermentation). The substrate may further comprise other non-carbon components, such as H2, N2, or electrons.

The gaseous substrate generally comprises at least some amount of CO, such as about 1, 2, 5, 10, 20, 30, 40, 50, 60, 70, 80, 90, or 100 mol % CO. The gaseous substrate may comprise a range of CO, such as about 20-80, 30-70, or 40-60 mol % CO. Preferably, the gaseous substrate comprises about 40-70 mol % CO (e.g., steel mill or blast furnace gas), about 20-30 mol % CO (e.g., basic oxygen furnace gas), or about 15-45 mol % CO (e.g., syngas). In some embodiments, the gaseous substrate may comprise a relatively low amount of CO, such as about 1-10 or 1-20 mol % CO. The microorganism of the disclosure typically converts at least a portion of the CO in the gaseous substrate to a product. In some embodiments, the gaseous substrate comprises no or substantially no (<1 mol %) CO.

The gaseous substrate may comprise some amount of H2. For example, the gaseous substrate may comprise about 1, 2, 5, 10, 15, 20, or 30 mol % H2. In some embodiments, the gaseous substrate may comprise a relatively high amount of H2, such as about 60, 70, 80, or 90 mol % H2. In further embodiments, the gaseous substrate comprises no or substantially no (<1 mol %) H2.

The gaseous substrate may comprise some amount of CO2. For example, the gaseous substrate may comprise about 1-80 or 1-30 mol % CO2. In some embodiments, the gaseous substrate may comprise less than about 20, 15, 10, or 5 mol % CO2. In another embodiment, the gaseous substrate comprises no or substantially no (<1 mol %) CO2.

The gaseous substrate may also be provided in alternative forms. For example, the gaseous substrate may be dissolved in a liquid or adsorbed onto a solid support.

The gaseous substrate and/or C1-carbon source may be a waste gas or an off gas obtained as a byproduct of an industrial process or from some other source, such as from automobile exhaust fumes or biomass gasification. In certain embodiments, the industrial process is selected from the group consisting of ferrous metal products manufacturing, such as a steel mill manufacturing, non-ferrous products manufacturing, petroleum refining, coal gasification, electric power production, carbon black production, ammonia production, methanol production, and coke manufacturing. In these embodiments, the gaseous substrate and/or C1-carbon source may be captured from the industrial process before it is emitted into the atmosphere, using any convenient method.

The gaseous substrate and/or C1-carbon source may be syngas, such as syngas obtained by gasification of coal or refinery residues, gasification of biomass or lignocellulosic material, or reforming of natural gas. In another embodiment, the syngas may be obtained from the gasification of municipal solid waste or industrial solid waste.

The substrate and/or C1-carbon source may be a waste gas obtained as a byproduct of an industrial process or from another source, such as automobile exhaust fumes, biogas, landfill gas, direct air capture, or from electrolysis. The substrate and/or C1-carbon source may be syngas generated by pyrolysis, torrefaction, or gasification. In other words, carbon in waste material may be recycled by pyrolysis, torrefaction, or gasification to generate syngas which is used as the substrate and/or C1-carbon source. The substrate and/or C1-carbon source may be a gas comprising methane.

In certain embodiments, the industrial process is selected from ferrous metal products manufacturing, such as a steel manufacturing, non-ferrous products manufacturing, petroleum refining, electric power production, carbon black production, paper and pulp manufacturing, ammonia production, methanol production, coke manufacturing, petrochemical production, carbohydrate fermentation, cement making, aerobic digestion, anaerobic digestion, catalytic processes, natural gas extraction, cellulosic fermentation, oil extraction, geological reservoirs, gas from fossil resources such as natural gas coal and oil, or any combination thereof. Examples of specific processing steps within an industrial process include catalyst regeneration, fluid catalyst cracking, and catalyst regeneration. Air separation and direct air capture are other suitable industrial processes. Specific examples in steel and ferroalloy manufacturing include blast furnace gas, basic oxygen furnace gas, coke oven gas, direct reduction of iron furnace top-gas, and residual gas from smelting iron. In these embodiments, the substrate and/or C1-carbon source may be captured from the industrial process before it is emitted into the atmosphere, using any known method.

The substrate and/or C1-carbon source may be synthesis gas known as syngas, which may be obtained from reforming, partial oxidation, or gasification processes. Examples of gasification processes include gasification of coal, gasification of refinery residues, gasification of petroleum coke, gasification of biomass, gasification of lignocellulosic material, gasification of waste wood, gasification of black liquor, gasification of municipal solid waste, gasification of municipal liquid waste, gasification of industrial solid waste, gasification of industrial liquid waste, gasification of refuse derived fuel, gasification of sewerage, gasification of sewerage sludge, gasification of sludge from wastewater treatment, gasification of biogas. Examples of reforming processes include, steam methane reforming, steam naphtha reforming, reforming of natural gas, reforming of biogas, reforming of landfill gas, naphtha reforming, and dry methane reforming. Examples of partial oxidation processes include thermal and catalytic partial oxidation processes, catalytic partial oxidation of natural gas, partial oxidation of hydrocarbons. Examples of municipal solid waste include tires, plastics, fibers, such as in shoes, apparel, and textiles. Municipal solid waste may be simply landfill-type waste. The municipal solid waste may be sorted or unsorted. Examples of biomass may include lignocellulosic material and may also include microbial biomass. Lignocellulosic material may include agriculture waste and forest waste.

The substrate and/or C1-carbon source may be a gas stream comprising methane. Such a methane containing gas may be obtained from fossil methane emission such as during fracking, wastewater treatment, livestock, agriculture, and municipal solid waste landfills. It is also envisioned that the methane may be burned to produce electricity or heat, and the C1 byproducts may be used as the substrate or carbon source.

The composition of the gaseous substrate may have a significant impact on the efficiency and/or cost of the reaction. For example, the presence of oxygen (O2) may reduce the efficiency of an anaerobic fermentation process. Depending on the composition of the substrate, it may be desirable to treat, scrub, or filter the substrate to remove any undesired impurities, such as toxins, undesired components, or dust particles, and/or increase the concentration of desirable components.

In certain embodiments, the fermentation is performed in the absence of carbohydrate substrates, such as sugar, starch, fiber, lignin, cellulose, or hemicellulose.

In some embodiments, the overall energetics of CO and H2 to ethylene glycol (MEG) are preferable to those from glucose to ethylene glycol, as shown below, wherein the more negative Gibbs free energy, ΔrG′m, values for CO and H2 indicate a larger driving force towards ethylene glycol. Calculations of overall reaction delta G for the comparison of glucose vs CO as a substrate were performed using equilibrator (http://equilibrator.weizmann.ac Ili), which is a standard method for evaluating the overall feasibility of a pathway or individual steps in pathways in biological systems (Flamholz, E. Noor, A. Bar-Even, R. Milo (2012) eQuilibrator—the biochemical thermodynamics calculator Nucleic Acids Res 40:D770-5; Noor, A. Bar-Even, A. Flamholz, Y. Lubling, D. Davidi, R. Milo (2012) An integrated open framework for thermodynamics of reactions that combines accuracy and coverage Bioinformatics 28:2037-2044; Noor, H. S. Haraldsdóttir, R. Milo, R. M. T. Fleming (2013) Consistent Estimation of Gibbs Energy Using Component Contributions PLoS Comput Biol 9(7): e1003098; Noor, A. Bar-Even, A. Flamholz, E. Reznik, W. Liebermeister, R. Milo (2014) Pathway Thermodynamics Highlights Kinetic Obstacles in Central Metabolism PLoS Comput Biol 10(2):e1003483). The calculations are as follows:

Glucose(aq)+3NADH(aq)⇄3MEG(aq)+3NAD+(aq)ΔrG′m−104 kJ/mol

6CO(aq)+3H2(aq)+6NADH(aq)⇄3MEG(aq)+6NAD+(aq)ΔrG′m−192 kJ/mol

Physiological Conditions:

Glucose(aq)+3NADH(aq)⇄3MEG(aq)+3NAD+(aq)ΔrG′m−70 kJ/mol

6CO(aq)+3H2(aq)+6NADH(aq)⇄3MEG(aq)+6NAD+(aq)ΔrG′m−295 kJ/mol

In addition to ethylene glycol, glyoxylate, and/or glycolate, the microorganism of the disclosure may be cultured to produce one or more co products. For instance, the microorganism of the disclosure may produce or may be engineered to produce ethanol (WO 2007/117157), acetate (WO 2007/117157), 1-butanol (WO 2008/115080, WO 2012/053905, and WO 2017/066498), butyrate (WO 2008/115080), 2,3-butanediol (WO 2009/151342 and WO 2016/094334), lactate (WO 2011/112103), butene (WO 2012/024522), butadiene (WO 2012/024522), methyl ethyl ketone (2-butanone) (WO 2012/024522 and WO 2013/185123), ethylene (WO 2012/026833), acetone (WO 2012/115527), isopropanol (WO 2012/115527), lipids (WO 2013/036147), 3-hydroxypropionate (3-HP) (WO 2013/180581), terpenes, including isoprene (WO 2013/180584), fatty acids (WO 2013/191567), 2-butanol (WO 2013/185123), 1,2-propanediol (WO 2014/036152), 1 propanol (WO 2017/066498), 1 hexanol (WO 2017/066498), 1 octanol (WO 2017/066498), chorismate-derived products (WO 2016/191625), 3 hydroxybutyrate (WO 2017/066498), 1,3 butanediol (WO 2017/066498), 2-hydroxyisobutyrate or 2-hydroxyisobutyric acid (WO 2017/066498), isobutylene (WO 2017/066498), adipic acid (WO 2017/066498), 1,3 hexanediol (WO 2017/066498), 3-methyl-2-butanol (WO 2017/066498), 2-buten-1-ol (WO 2017/066498), isovalerate (WO 2017/066498), isoamyl alcohol (WO 2017/066498), and/or monoethylene glycol (WO 2019/126400) in addition to 2-phenylethanol. In some embodiments, in addition to ethylene glycol, the microorganism of the disclosure also produces ethanol, 2,3-butanediol, and/or succinate. In certain embodiments, microbial biomass itself may be considered a product. These products may be further converted to produce at least one component of diesel, jet fuel, and/or gasoline. In certain embodiments, 2-phenylethanol may be used as an ingredient in fragrances, essential oils, flavors, and soaps. Additionally, the microbial biomass may be further processed to produce a single cell protein (SCP) by any method or combination of methods known in the art. In addition to one or more target products, the microorganism of the disclosure may also produce ethanol, acetate, and/or 2,3-butanediol.

A “native product” is a product produced by a genetically unmodified microorganism. For example, ethanol, acetate, and 2,3-butanediol are native products of Clostridium autoethanogenum, Clostridium ljungdahlii, and Clostridium ragsdalei. A “non-native product” is a product that is produced by a genetically modified microorganism but is not produced by a genetically unmodified microorganism from which the genetically modified microorganism is derived. Ethylene glycol is not known to be produced by any naturally-occurring microorganism, such that it is a non-native product of all microorganisms.

“Selectivity” refers to the ratio of the production of a target product to the production of all fermentation products produced by a microorganism. The microorganism of the disclosure may be engineered to produce products at a certain selectivity or at a minimum selectivity. In one embodiment, a target product, such as ethylene glycol, accounts for at least about 5%, 10%, 15%, 20%, 30%, 50%, or 75% of all fermentation products produced by the microorganism of the disclosure. In one embodiment, ethylene glycol accounts for at least 10% of all fermentation products produced by the microorganism of the disclosure, such that the microorganism of the disclosure has a selectivity for ethylene glycol of at least 10%. In another embodiment, ethylene glycol accounts for at least 30% of all fermentation products produced by the microorganism of the disclosure, such that the microorganism of the disclosure has a selectivity for ethylene glycol of at least 30%.

At least one of the one or more fermentation products may be biomass produced by the culture. At least a portion of the microbial biomass may be converted to a single cell protein (SCP). At least a portion of the single cell protein may be utilized as a component of animal feed.

In one embodiment, the disclosure provides an animal feed comprising microbial biomass and at least one excipient, wherein the microbial biomass comprises a microorganism grown on a gaseous substrate comprising one or more of CO, CO2, and H2.

A “single cell protein” (SCP) refers to a microbial biomass that may be used in protein-rich human and/or animal feeds, often replacing conventional sources of protein supplementation such as soymeal or fishmeal. To produce a single cell protein, or other product, the process may comprise additional separation, processing, or treatments steps. For example, the method may comprise sterilizing the microbial biomass, centrifuging the microbial biomass, and/or drying the microbial biomass. In certain embodiments, the microbial biomass is dried using spray drying or paddle drying. The method may also comprise reducing the nucleic acid content of the microbial biomass using any method known in the art, since intake of a diet high in nucleic acid content may result in the accumulation of nucleic acid degradation products and/or gastrointestinal distress. The single cell protein may be suitable for feeding to animals, such as livestock or pets. In particular, the animal feed may be suitable for feeding to one or more beef cattle, dairy cattle, pigs, sheep, goats, horses, mules, donkeys, deer, buffalo/bison, llamas, alpacas, reindeer, camels, bantengs, gayals, yaks, chickens, turkeys, ducks, geese, quail, guinea fowl, squabs/pigeons, fish, shrimp, crustaceans, cats, dogs, and rodents. The composition of the animal feed may be tailored to the nutritional requirements of different animals. Furthermore, the process may comprise blending or combining the microbial biomass with one or more excipients.

“Microbial biomass” refers biological material comprising microorganism cells. For example, microbial biomass may comprise or consist of a pure or substantially pure culture of a bacterium, archaea, virus, or fungus. When initially separated from a fermentation broth, microbial biomass generally contains a large amount of water. This water may be removed or reduced by drying or processing the microbial biomass.

An “excipient” may refer to any substance that may be added to the microbial biomass to enhance or alter the form, properties, or nutritional content of the animal feed. For example, the excipient may comprise one or more of a carbohydrate, fiber, fat, protein, vitamin, mineral, water, flavour, sweetener, antioxidant, enzyme, preservative, probiotic, or antibiotic. In some embodiments, the excipient may be hay, straw, silage, grains, oils or fats, or other plant material. The excipient may be any feed ingredient identified in Chiba, Section 18: Diet Formulation and Common Feed Ingredients, Animal Nutrition Handbook, 3rd revision, pages 575-633, 2014.

A “biopolymer” refers to natural polymers produced by the cells of living organisms. In certain embodiments, the biopolymer is PHA. In certain embodiments, the biopolymer is PHB.

A “bioplastic” refers to plastic materials produced from renewable biomass sources. A bioplastic may be produced from renewable sources, such as vegetable fats and oils, corn starch, straw, woodchips, sawdust, or recycled food waste.

Herein, reference to an acid (e.g., acetic acid or 2-hydroxyisobutyric acid) should be taken to also include the corresponding salt (e.g., acetate or 2-hydroxyisobutyrate).

Typically, the culture is performed in a bioreactor. The term “bioreactor” includes a culture/fermentation device consisting of one or more vessels, towers, or piping arrangements, such as a continuous stirred tank reactor (CSTR), immobilized cell reactor (ICR), trickle bed reactor (TBR), bubble column, gas lift fermenter, static mixer, or other vessel or other device suitable for gas-liquid contact. In some embodiments, the bioreactor may comprise a first growth reactor and a second culture/fermentation reactor. The substrate may be provided to one or both of these reactors. As used herein, the terms “culture” and “fermentation” are used interchangeably. These terms encompass both the growth phase and product biosynthesis phase of the culture/fermentation process.

The culture is generally maintained in an aqueous culture medium that contains nutrients, vitamins, and/or minerals sufficient to permit growth of the microorganism. Preferably the aqueous culture medium is an anaerobic microbial growth medium, such as a minimal anaerobic microbial growth medium. Suitable media are well known in the art.

The culture/fermentation should desirably be carried out under appropriate conditions for production of ethylene glycol. If necessary, the culture/fermentation is performed under anaerobic conditions. Reaction conditions to consider include pressure (or partial pressure), temperature, gas flow rate, liquid flow rate, media pH, media redox potential, agitation rate (if using a continuous stirred tank reactor), inoculum level, maximum gas substrate concentrations to ensure that gas in the liquid phase does not become limiting, and maximum product concentrations to avoid product inhibition. In particular, the rate of introduction of the substrate may be controlled to ensure that the concentration of gas in the liquid phase does not become limiting.

Operating a bioreactor at elevated pressures allows for an increased rate of gas mass transfer from the gas phase to the liquid phase. Accordingly, it is generally preferable to perform the culture/fermentation at pressures higher than atmospheric pressure. Also, since a given gas conversion rate is, in part, a function of the substrate retention time and retention time dictates the required volume of a bioreactor, the use of pressurized systems can greatly reduce the volume of the bioreactor required and, consequently, the capital cost of the culture/fermentation equipment. This, in turn, means that the retention time, defined as the liquid volume in the bioreactor divided by the input gas flow rate, can be reduced when bioreactors are maintained at elevated pressure rather than atmospheric pressure. The optimum reaction conditions will depend partly on the particular microorganism used. However, in general, it is preferable to operate the fermentation at a pressure higher than atmospheric pressure. Also, since a given gas conversion rate is in part a function of substrate retention time and achieving a desired retention time in turn dictates the required volume of a bioreactor, the use of pressurized systems can greatly reduce the volume of the bioreactor required, and consequently the capital cost of the fermentation equipment.

In certain embodiments, the fermentation is performed in the absence of light or in the presence of an amount of light insufficient to meet the energetic requirements of photosynthetic microorganisms. In certain embodiments, the microorganism of the disclosure is a non-photosynthetic microorganism.

Target products may be separated or purified from a fermentation broth using any method or combination of methods known in the art, including, for example, fractional distillation, evaporation, pervaporation, gas stripping, phase separation, and extractive fermentation, including for example, liquid-liquid extraction. In certain embodiments, target products are recovered from the fermentation broth by continuously removing a portion of the broth from the bioreactor, separating microbial cells from the broth (conveniently by filtration), and recovering one or more target products from the broth. Alcohols and/or acetone may be recovered, for example, by distillation. Acids may be recovered, for example, by adsorption on activated charcoal. Separated microbial cells are preferably returned to the bioreactor. The cell-free permeate remaining after target products have been removed is also preferably returned to the bioreactor. Additional nutrients (such as B vitamins) may be added to the cell-free permeate to replenish the medium before it is returned to the bioreactor. Purification techniques may include affinity tag purification (e.g. His, Twin-Strep, and FLAG), bead-based systems, a tip-based approach, and FPLC system for larger scale, automated purifications. Purification methods that do not rely on affinity tags (e.g. salting out, ion exchange, and size exclusion) are also disclosed.

As referred to herein, a “fermentation broth” is a culture medium comprising at least a nutrient media and bacterial cells.

As referred to herein, a “shuttle microorganism” is a microorganism in which a methyltransferase enzyme is expressed and is distinct from the destination microorganism.

As referred to herein, a “destination microorganism” is a microorganism in which the genes included on an expression construct/vector are expressed and is distinct from the shuttle microorganism.

The term “main fermentation product” is intended to mean the one fermentation product which is produced in the highest concentration and/or yield.

The terms “increasing the efficiency”, “increased efficiency” and the like, when used in relation to a fermentation process, include, but are not limited to, increasing one or more of the rate of growth of microorganisms catalysing the fermentation, the growth and/or product production rate at elevated product concentrations, the volume of desired product produced per volume of substrate consumed, the rate of production or level of production of the desired product, and the relative proportion of the desired product produced compared with other by-products of the fermentation.

The phrase “substrate comprising carbon monoxide” and like terms should be understood to include any substrate in which carbon monoxide is available to one or more strains of bacteria for growth and/or fermentation, for example.

The phrase “gaseous substrate comprising carbon monoxide” and like phrases and terms includes any gas which contains a level of carbon monoxide. In certain embodiments the substrate contains at least about 20% to about 100% CO by volume, from 20% to 70% CO by volume, from 30% to 60% CO by volume, and from 40% to 55% CO by volume. In particular embodiments, the substrate comprises about 25%, or about 30%, or about 35%, or about 40%, or about 45%, or about 50% CO, or about 55% CO, or about 60% CO by volume.

While it is not necessary for the substrate to contain any hydrogen, the presence of H2 should not be detrimental to product formation in accordance with methods of the disclosure. In particular embodiments, the presence of hydrogen results in an improved overall efficiency of alcohol production. For example, in particular embodiments, the substrate may comprise an approx. 2:1, or 1:1, or 1:2 ratio of H₂:CO. In one embodiment the substrate comprises about 30% or less H₂ by volume, 20% or less H₂ by volume, about 15% or less H₂ by volume or about 10% or less H₂ by volume. In other embodiments, the substrate stream comprises low concentrations of H2, for example, less than 5%, or less than 4%, or less than 3%, or less than 2%, or less than 1%, or is substantially hydrogen free. The substrate may also contain some CO₂ for example, such as about 1% to about 80% CO₂ by volume, or 1% to about 30% CO₂ by volume. In one embodiment the substrate comprises less than or equal to about 20% CO₂ by volume. In particular embodiments the substrate comprises less than or equal to about 15% CO₂ by volume, less than or equal to about 10% CO₂ by volume, less than or equal to about 5% CO₂ by volume or substantially no CO₂.

In the description which follows, embodiments of the disclosure are described in terms of delivering and fermenting a “gaseous substrate containing CO”. However, it should be appreciated that the gaseous substrate may be provided in alternative forms. For example, the gaseous substrate containing CO may be provided dissolved in a liquid. Essentially, a liquid is saturated with a carbon monoxide containing gas and then that liquid is added to the bioreactor. This may be achieved using standard methodology. By way of example, a microbubble dispersion generator (Hensirisak et. al. Scale-up of microbubble dispersion generator for aerobic fermentation; Applied Biochemistry and Biotechnology Volume 101, Number 3/October 2002) could be used. By way of further example, the gaseous substrate containing CO may be adsorbed onto a solid support. Such alternative methods are encompassed by use of the term “substrate containing CO” and the like.

In particular embodiments of the disclosure, the CO-containing gaseous substrate is an industrial off or waste gas. “Industrial waste or off gases” should be taken broadly to include any gases comprising CO produced by an industrial process and include gases produced as a result of ferrous metal products manufacturing, non-ferrous products manufacturing, petroleum refining processes, gasification of coal, gasification of biomass, electric power production, carbon black production, and coke manufacturing. Further examples may be provided elsewhere herein.

Unless the context requires otherwise, the phrases “fermenting”, “fermentation process” or “fermentation reaction” and the like, as used herein, are intended to encompass both the growth phase and product biosynthesis phase of the process. As will be described further herein, in some embodiments the bioreactor may comprise a first growth reactor and a second fermentation reactor. As such, the addition of metals or compositions to a fermentation reaction should be understood to include addition to either or both of these reactors.

The term “bioreactor” includes a fermentation device consisting of one or more vessels and/or towers or piping arrangement, which includes the Continuous Stirred Tank Reactor (CSTR), Immobilized Cell Reactor (ICR), Trickle Bed Reactor (TBR), Bubble Column, Gas Lift Fermenter, Static Mixer, or other vessel or other device suitable for gas-liquid contact. In some embodiments the bioreactor may comprise a first growth reactor and a second fermentation reactor. As such, when referring to the addition of substrate to the bioreactor or fermentation reaction it should be understood to include addition to either or both of these reactors where appropriate.

“Exogenous nucleic acids” are nucleic acids which originate outside of the microorganism to which they are introduced. Exogenous nucleic acids may be derived from any appropriate source, including, but not limited to, the microorganism to which they are to be introduced (for example in a parental microorganism from which the recombinant microorganism is derived), strains or species of microorganisms which differ from the organism to which they are to be introduced, or they may be artificially or recombinantly created. In one embodiment, the exogenous nucleic acids represent nucleic acid sequences naturally present within the microorganism to which they are to be introduced, and they are introduced to increase expression of or over-express a particular gene (for example, by increasing the copy number of the sequence (for example a gene), or introducing a strong or constitutive promoter to increase expression). In another embodiment, the exogenous nucleic acids represent nucleic acid sequences not naturally present within the microorganism to which they are to be introduced and allow for the expression of a product not naturally present within the microorganism or increased expression of a gene native to the microorganism (for example in the case of introduction of a regulatory element such as a promoter). The exogenous nucleic acid may be adapted to integrate into the genome of the microorganism to which it is to be introduced or to remain in an extra-chromosomal state.

“Exogenous” may also be used to refer to proteins. This refers to a protein that is not present in the parental microorganism from which the recombinant microorganism is derived.

The term “endogenous” as used herein in relation to a recombinant microorganism and a nucleic acid or protein refers to any nucleic acid or protein that is present in a parental microorganism from which the recombinant microorganism is derived.

It should be appreciated that the disclosure may be practised using nucleic acids whose sequence varies from the sequences specifically exemplified herein provided they perform substantially the same function. For nucleic acid sequences that encode a protein or peptide this means that the encoded protein or peptide has substantially the same function. For nucleic acid sequences that represent promoter sequences, the variant sequence will have the ability to promote expression of one or more genes. Such nucleic acids may be referred to herein as “functionally equivalent variants”. By way of example, functionally equivalent variants of a nucleic acid include allelic variants, fragments of a gene, genes which include mutations (deletion, insertion, nucleotide substitutions and the like) and/or polymorphisms and the like. Homologous genes from other microorganisms may also be considered as examples of functionally equivalent variants of the sequences specifically exemplified herein. These include homologous genes in species such as Clostridium acetobutylicum, Clostridium beijerinckii, C. saccharobutylicum and C. saccharoperbutylacetonicum, details of which are publicly available on websites such as Genbank or NCBI. The phrase “functionally equivalent variants” should also be taken to include nucleic acids whose sequence varies as a result of codon optimisation for a particular organism. “Functionally equivalent variants” of a nucleic acid herein will preferably have at least approximately 70%, preferably approximately 80%, more preferably approximately 85%, preferably approximately 90%, preferably approximately 95% or greater nucleic acid sequence identity with the nucleic acid identified.

It should also be appreciated that the disclosure may be practised using polypeptides whose sequence varies from the amino acid sequences specifically exemplified herein. These variants may be referred to herein as “functionally equivalent variants”. A functionally equivalent variant of a protein or a peptide includes those proteins or peptides that share at least 40%, preferably 50%, preferably 60%, preferably 70%, preferably 75%, preferably 80%, preferably 85%, preferably 90%, preferably 95% or greater amino acid identity with the protein or peptide identified and has substantially the same function as the peptide or protein of interest. Such variants include within their scope fragments of a protein or peptide wherein the fragment comprises a truncated form of the polypeptide wherein deletions may be from 1 to 5, to 10, to 15, to 20, to 25 amino acids, and may extend from residue 1 through 25 at either terminus of the polypeptide, and wherein deletions may be of any length within the region; or may be at an internal location. Functionally equivalent variants of the specific polypeptides herein should also be taken to include polypeptides expressed by homologous genes in other species of bacteria, for example as exemplified in the previous paragraph.

“Substantially the same function” as used herein is intended to mean that the nucleic acid or polypeptide is able to perform the function of the nucleic acid or polypeptide of which it is a variant. For example, a variant of an enzyme of the disclosure will be able to catalyse the same reaction as that enzyme. However, it should not be taken to mean that the variant has the same level of activity as the polypeptide or nucleic acid of which it is a variant.

One may assess whether a functionally equivalent variant has substantially the same function as the nucleic acid or polypeptide of which it is a variant using any number of known methods. However, by way of example, the methods described by Silver et al. (1991, Plant Physiol. 97: 1588-1591) or Zhao et al. (2011, Appl Microbiol Biotechnol, 90:1915-1922) for the isoprene synthase enzyme, by Green et al. (2007, Phytochemistry; 68:176-188) for the farnesene synthase enzyme, by Kuzuyama et al. (2000, J. Bacteriol. 182, 891-897) for the 1-deoxy-D-xylulose 5-phosphate synthase Dxs, by Berndt and Schlegel (1975, Arch. Microbiol. 103, 21-30) or by Stim-Herndon et al. (1995, Gene 154: 81-85) for the thiolase, by Cabano et al. (1997, Insect Biochem. Mol. Biol. 27: 499-505) for the HMG-CoA synthase, by Ma et al. (2011, Metab. Engin., 13:588-597) for the HMG-CoA reductase and mevalonate kinase enzyme, by Herdendorf and Miziorko (2007, Biochemistry, 46: 11780-8) for the phosphomevalonate kinase, and by Krepkiy et al. (2004, Protein Sci. 13: 1875-1881) for the mevalonate diphosphate decarboxylase. It is also possible to identify genes of DXS and mevalonate pathway using inhibitors like fosmidomycin or mevinolin as described by Trutko et al. (2005, Microbiology 74: 153-158).

“Over-express”, “over expression” and like terms and phrases when used in relation to the disclosure should be taken broadly to include any increase in expression of one or more proteins (including expression of one or more nucleic acids encoding same) as compared to the expression level of the protein (including nucleic acids) of a parental microorganism under the same conditions. It should not be taken to mean that the protein (or nucleic acid) is expressed at any particular level.

A “parental microorganism” is a microorganism used to generate a recombinant microorganism of the disclosure. The parental microorganism may be one that occurs in nature (i.e. a wild-type microorganism) or one that has been previously modified but which does not express or over-express one or more of the enzymes that are the subject of the present disclosure. Accordingly, the recombinant microorganisms of the disclosure may have been modified to express or over-express one or more enzymes that were not expressed or over-expressed in the parental microorganism.

The terms nucleic acid “constructs” or “vectors” and like terms should be taken broadly to include any nucleic acid (including DNA and RNA) suitable for use as a vehicle to transfer genetic material into a cell. The terms should be taken to include plasmids, viruses (including bacteriophage), cosmids and artificial chromosomes. Constructs or vectors may include one or more regulatory elements, an origin of replication, a multicloning site and/or a selectable marker. In one particular embodiment, the constructs or vectors are adapted to allow expression of one or more genes encoded by the construct or vector. Nucleic acid constructs or vectors include naked nucleic acids as well as nucleic acids formulated with one or more agents to facilitate delivery to a cell (for example, liposome-conjugated nucleic acid, an organism in which the nucleic acid is contained).

A “terpene” as referred to herein should be taken broadly to include any compound made up of C₅ isoprene units joined together including simple and complex terpenes and oxygen-containing terpene compounds such as alcohols, aldehydes and ketones. Simple terpenes are found in the essential oils and resins of plants such as conifers. More complex terpenes include the terpenoids and vitamin A, carotenoid pigments (such as lycopene), squalene, and rubber. Examples of monoterpenes include, but are not limited to isoprene, pinene, nerol, citral, camphor, menthol, limonene. Examples of sesquiterpenes include but are not limited to nerolidol, farnesol. Examples of diterpenes include but are not limited to phytol, vitamin A₁. Squalene is an example of a triterpene, and carotene (provitamin A₁) is a tetraterpene.

A “terpene precursor” is a compound or intermediate produced during the reaction to form a terpene starting from Acetyl CoA and optionally pyruvate. The term refers to a precursor compound or intermediate found in the mevalonate (MVA) pathway and optionally the DXS pathway as well as downstream precursors of longer chain terpenes, such as FPP and GPP. In particular embodiments, it includes but is not limited to mevalonic acid, IPP, dimethylallyl pyrophosphate (DMAPP), geranyl pyrophosphate (GPP) and farnesyl pyrophosphate (FPP).

The “DXS pathway” is the enzymatic pathway from pyruvate and D-glyceraldehyde-3-phosphate to DMAPP or IPP. It is also known as the deoxyxylulose 5-phosphate (DXP/DXPS/DOXP or DXS)/methylerythritol phosphate (MEP) pathway.

The “mevalonate (MVA) pathway” is the enzymatic pathway from acetyl-CoA to IPP.

### Microorganisms

Two pathways for production of terpenes are known, the deoxyxylulose 5-phosphate (DXP/DXPS/DOXP or DXS)/methylerythritol phosphate (MEP) pathway (Hunter et al., 2007, J. Biol. chem. 282: 21573-77) starting from pyruvate and D-glyceraldehyde-3-phosphate (G3P), the two key intermediates in the glycolysis, and the mevalonate (MVA) pathway (Miziorko, 2011, Arch Biochem Biophys, 505: 131-143) starting from acetyl-CoA. Many different classes of microorganisms have been investigated for presence of either of these pathways (Lange et al., 2000, PNAS, 97: 13172-77; Trutko et al., 2005, Microbiology, 74: 153-158; Julsing et al., 2007, Appl Microbiol Biotechnol, 75: 1377-84), but not carboxydotrophic acetogens. The DXS pathway for example was found to be present in E. coli, Bacillus, or Mycobacterium, while the mevalonate pathway is present in yeast Saccharomyces, Cloroflexus, or Myxococcus.

Genomes of carboxydotrophic acetogens C. autoethanogenum, C. ljungdahlii were analysed by the inventors for presence of either of the two pathways. All genes of the DXS pathway were identified in C. autoethanogenum and C. ljungdahlii (Table 1), while the mevalonate pathway is absent. Additionally, carboxydotrophic acetogens such as C. autoethanogenum or C. ljungdahlii are not known to produce any terpenes as metabolic end products.

TABLE 1
Terpene biosynthesis genes of the DXS pathway identified
in C. autoethanogenum and C. ljungdahlii:

| Gene/Enzyme | C. autoethanogenum | C. ljungdahlii |
|---|---|---|
| 1-deoxy-D- | SEQ ID NO: 1-2 | YP_003779286.1; |
| xylulose-5- | | GI: 300854302, |
| phosphate | | CLJU_c11160 |
| synthase DXS | | |
| (EC: 2.2.1.7) | | |
| 1-deoxy-D- | SEQ ID NO: 3-4 | YP_003779478.1; |
| xylulose 5- | | GI: 300854494, |
| phosphate | | CLJU_c13080 |
| reductoisomerase | | |
| DXR (EC: | | |
| 1.1.1.267) | | |
| 2-C-methyl- | SEQ ID NO: 5-6 | YP_003782252.1 |
| D-erythritol 4- | | GI: 300857268, |
| phosphate | | CLJU_c41280 |
| cytidylyl- | | |
| transferase IspD | | |
| (EC: 2.7.7.60) | | |
| 4-diphospho- | SEQ ID NO: 7-8 | YP_003778403.1; |
| cytidyl-2-C- | | GI: 300853419, |
| methyl-D- | | CLJU_c02110 |
| erythritol kinase | | |
| IspE (EC: | | |
| 2.7.1.148) | | |
| 2-C-methyl-D- | SEQ ID NO: 9-10 | YP_003778349.1; |
| erythritol 2,4- | | GI: 300853365, |
| cyclodi- | | CLJU_c01570 |
| phosphate synthase | | |
| IspF (EC: 4.6.1.12) | | |
| 4-hydroxy-3- | SEQ ID NO: 11-12 | YP_003779480.1; |
| methylbut-2-en- | | GI: 300854496, |
| 1-yl diphosphate | | CLJU_c13100 |
| synthase | | |
| IspG (EC: 1.17.7.1) | | |
| 4-hydroxy-3- | SEQ ID NO: 13-14 | YP_003780294.1; |
| methylbut-2- | | GI: 300855310, |
| enyl diphosphate | | CLJU_c21320 |
| reductase | | |
| (EC: 1.17.1.2) | | |

Genes for downstream synthesis of terpenes from isoprene units were also identified in both organisms (Table 2).

| Gene/Enzyme | C. autoethanogenum | C. ljungdahlii |
|---|---|---|
| geranyl- | SEQ ID NO: 15-16 | YP_003779285.1; |
| transtransferase Fps | | GI: 300854301, |
| (EC: 2.5.1.10) | | CLJU_c11150 |
| heptaprenyl diphosphate | SEQ ID NO: 17-18 | YP_003779312.1; |
| synthase (EC: 2.5.1.10) | | GI: 300854328, |
| | | CLJU_c11420 |
| octaprenyl-diphosphate | SEQ ID NO: 19-20 | YP_003782157.1; |
| synthase [EC: 2.5.1.90] | | GI: 300857173, |
| | | CLJU_c40310 |

Terpenes are energy dense compounds, and their synthesis requires the cell to invest energy in the form of nucleoside triphosphates such as ATP. Using sugar as a substrate requires sufficient energy to be supplied from glycolysis to yield several molecules of ATP. The production of terpenes and/or their precursors via the DXS pathway using sugar as a substrate proceeds in a relatively straightforward manner due to the availability of pyruvate and D-glyceraldehyde-3-phosphate (G3P), G3P being derived from C5 pentose and C6 hexose sugars. These C5 and C6 molecules are thus relatively easily converted into C5 isoprene units from which terpenes are composed.

For anaerobic acetogens using a C1 substrate like CO or CO2, it is more difficult to synthesise long molecules such as hemiterpenoids from C1 units. This is especially true for longer chain terpenes like C10 monoterpenes, C15 sesquiterpenes, or C40 tetraterpenes. To date the product with most carbon atoms reported in acetogens (both native and recombinant organisms) are C4 compounds butanol (Köpke et al., 2011, Curr. Opin. Biotechnol. 22: 320-325; Schiel-Bengelsdorf and Dürre, 2012, FEBS Letters: 10.1016/j.febslet.2012.04.043; Köpke et al., 2011, Proc. Nat. Sci. U.S.A. 107: 13087-92; US patent 2011/0236941) and 2,3-butanediol (Köpke et al., 2011, Appl. Environ. Microbiol. 77:5467-75). The inventors have shown that it is surprisingly possible to anaerobically produce these longer chain terpene molecules using the C1 feedstock CO via the acetyl CoA intermediate.

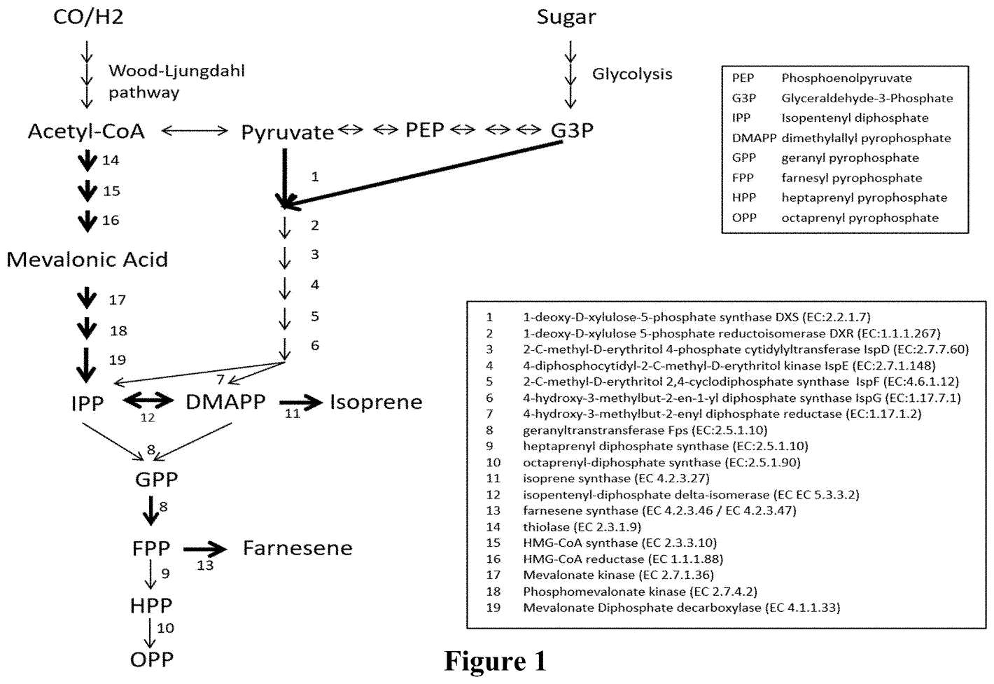

Energetics of the Wood-Ljungdahl pathway of anaerobic acetogens are just emerging, but unlike under aerobic growth conditions or glycolysis of sugar fermenting organisms no ATP is gained in the Wood-Ljungdahl pathway by substrate level phosphorylation, in fact activation of CO₂ to formate actually requires one molecule of ATP and a membrane gradient is required. The inventors note that it is important that a pathway for product formation is energy efficient. The inventors note that in acetogens the substrate CO or CO₂ is channeled directly into acetyl-CoA, which represents the most direct route to terpenes and/or their precursors, especially when compared to sugar based systems, with only six reactions required (FIG. 1). Though less ATP is available in carboxydotrophic acetogens, the inventors believe that this more direct pathway may sustain a higher metabolic flux (owing to higher chemical motive force of intermediate reactions). A highly effective metabolic flux is important as several intermediates in the terpene biosynthesis pathway, such as key intermediates Mevalonate and FPP, are toxic to most bacteria when not turned over efficiently. Despite having a higher ATP availability, this problem of intermediate toxicity can be a bottleneck in production of terpenes from sugar.

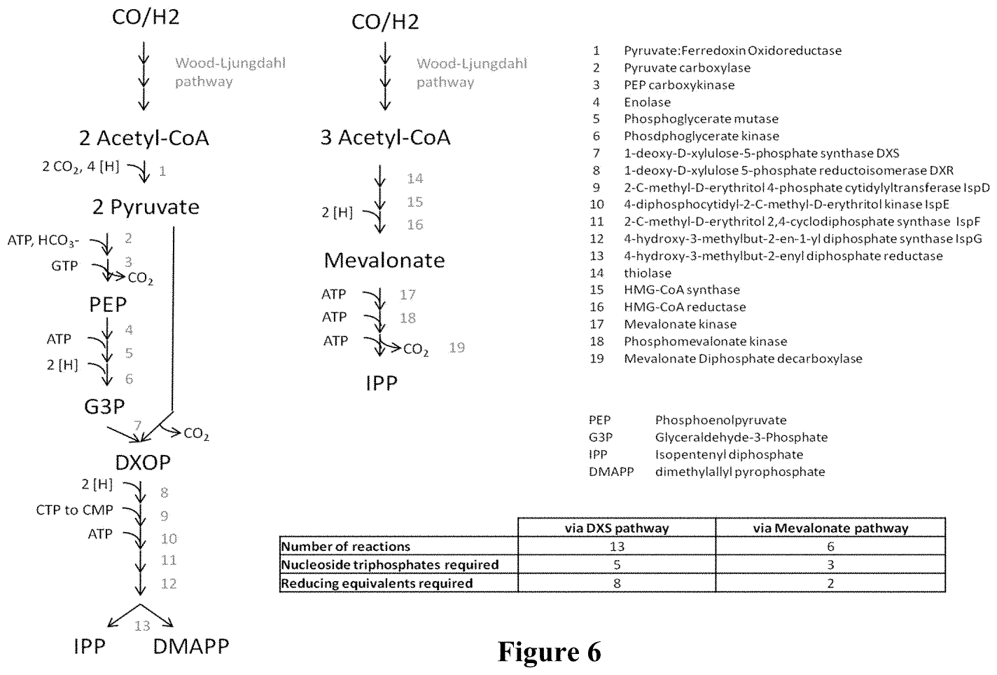

When comparing the energetics of terpene precursor IPP and DMAPP production from CO (FIG. 6) via the mevalonate pathway versus the DXS pathway, the inventors noted that the mevalonate pathway requires less nucleoside triphosphates as ATP, less reducing equivalents, and is also more direct when compared to the DXS pathway with only six necessary reaction steps from acetyl-CoA. This provides advantages in the speed of the reactions and metabolic fluxes and increases overall energy efficiency. Additionally, the lower number of enzymes required simplifies the recombination method required to produce a recombinant microorganism.

No acetogens with a mevalonate pathway have been identified, but the inventors have shown that it is possible to introduce the mevalonate pathway and optionally the DXS pathway into a carboxydotrophic acetogen such as Clostridium autoethanogenum or C. ljungdahlii to efficiently produce terpenes and/or precursors thereof from the C1 carbon substrate CO. They contemplate that this is applicable to all carboxydotrophic acetogenic microorganisms.

Additionally, the production of terpenes and/or precursors thereof has never been shown to be possible using recombinant microorganisms under anaerobic conditions. Anaerobic production of isoprene has the advantage of providing a safer operating environment because isoprene is extremely flammable in the presence of oxygen and has a lower flammable limit (LFL) of 1.5-2.0% and an upper flammable (UFL) limit of 2.0-12% at room temperature and atmospheric pressure. As flames cannot occur in the absence of oxygen, the inventors believe that an anaerobic fermentation process is desirable as it would be safer across all product concentrations, gas compositions, temperature and pressure ranges.

As discussed hereinbefore, the disclosure provides a recombinant microorganism capable of producing one or more terpenes and/or precursors thereof, and optionally one or more other products, by fermentation of a substrate comprising CO.

In a further embodiment, the microorganism is adapted to:

express one or more exogenous enzymes from the mevalonate (MVA) pathway and/or overexpress one or more endogenous enzyme from the mevalonate (MVA) pathway; and
a) express one or more exogenous enzymes from the DXS pathway and/or overexpress one or more endogenous enzymes from the DXS pathway.

In one embodiment, the parental microorganism from which the recombinant microorganism is derived is capable of fermenting a substrate comprising CO to produce Acetyl CoA, but not of converting Acetyl CoA to mevalonic acid or isopentenyl pyrophosphate (IPP) and the recombinant microorganism is adapted to express one or more enzymes involved in the mevalonate pathway.

The microorganism may be adapted to express or over-express the one or more enzymes by any number of recombinant methods including, for example, increasing expression of native genes within the microorganism (for example, by introducing a stronger or constitutive promoter to drive expression of a gene), increasing the copy number of a gene encoding a particular enzyme by introducing exogenous nucleic acids encoding and adapted to express the enzyme, introducing an exogenous nucleic acid encoding and adapted to express an enzyme not naturally present within the parental microorganism.

In one embodiment, the one or more enzymes are from the mevalonate (MVA) pathway and are selected from the group consisting of:

a) thiolase (EC 2.3.1.9),

b) HMG-CoA synthase (EC 2.3.3.10),

c) HMG-CoA reductase (EC 1.1.1.88),

d) Mevalonate kinase (EC 2.7.1.36),

e) Phosphomevalonate kinase (EC 2.7.4.2),

f) Mevalonate Diphosphate decarboxylase (EC 4.1.1.33), and

g) a functionally equivalent variant of any one thereof.

In a further embodiment, the optional one or more enzymes are from the DXS pathway is selected from the group consisting of:

a) 1-deoxy-D-xylulose-5-phosphate synthase DXS (EC:2.2.1.7),

b) 1-deoxy-D-xylulose 5-phosphate reductoisomerase DXR (EC:1.1.1.267),

c) 2-C-methyl-D-erythritol 4-phosphate cytidylyltransferase IspD (EC:2.7.7.60),

d) 4-diphosphocytidyl-2-C-methyl-D-erythritol kinase IspE (EC:2.7.1.148),

e) 2-C-methyl-D-erythritol 2,4-cyclodiphosphate synthase IspF (EC:4.6.1.12),

f) 4-hydroxy-3-methylbut-2-en-1-yl diphosphate synthase IspG (EC:1.17.7.1),

g) 4-hydroxy-3-methylbut-2-enyl diphosphate reductase (EC:1.17.1.2), and

h) a functionally equivalent variant of any one thereof.

In a further embodiment, one or more exogenous or endogenous further enzymes are expressed or over-expressed to result in the production of a terpene compound and/or precursor thereof wherein the exogenous enzyme that is expressed, or the endogenous enzyme that is overexpressed is selected from the group consisting of:

a) geranyltranstransferase Fps (EC:2.5.1.10),

b) heptaprenyl diphosphate synthase (EC:2.5.1.10),

c) octaprenyl-diphosphate synthase (EC:2.5.1.90),

d) isoprene synthase (EC 4.2.3.27),

e) isopentenyl-diphosphate delta-isomerase (EC 5.3.3.2),

f) farnesene synthase (EC 4.2.3.46/EC 4.2.3.47), and

g) a functionally equivalent variant of any one thereof.

By way of example only, sequence information for each of the enzymes is listed in the figures herein.

The enzymes of use in the microorganisms of the disclosure may be derived from any appropriate source, including different genera and species of bacteria, or other organisms. However, in one embodiment, the enzymes are derived from Staphylococcus aureus.

In one embodiment, the enzyme isoprene synthase (ispS) is derived from Poplar tremuloides. In a further embodiment, it has the nucleic acid sequence exemplified in SEQ ID NO: 21 hereinafter, or it is a functionally equivalent variant thereof.

In one embodiment, the enzyme deoxyxylulose 5-phosphate synthase is derived from C. autoethanogenum, encoded by the nucleic acid sequence exemplified in SEQ ID NO: 1 and/or with the amino acid sequence exemplified in SEQ ID NO: 2 hereinafter, or it is a functionally equivalent variant thereof.

In one embodiment, the enzyme 1-deoxy-D-xylulose 5-phosphate reductoisomerase DXR is derived from C. autoethanogenum and is encoded by the nucleic acid sequence exemplified in SEQ ID NO: 3 or is a functionally equivalent variant thereof.

In one embodiment, the enzyme 2-C-methyl-D-erythritol 4-phosphate cytidylyltransferase IspD is derived from C. autoethanogenum and is encoded by the nucleic acid sequence exemplified in SEQ ID NO: 5 or is a functionally equivalent variant thereof.

In one embodiment, the enzyme 4-diphosphocytidyl-2-C-methyl-D-erythritol kinase IspE is derived from C. autoethanogenum and is encoded by the nucleic acid sequence exemplified in SEQ ID NO: 7 or is a functionally equivalent variant thereof.

In one embodiment, the enzyme 2-C-methyl-D-erythritol 2,4-cyclodiphosphate synthase IspF is derived from C. autoethanogenum and is encoded by the nucleic acid sequence exemplified in SEQ ID NO: 9 or is a functionally equivalent variant thereof.

In one embodiment, the enzyme 4-hydroxy-3-methylbut-2-en-1-yl diphosphate synthase IspG is derived from C. autoethanogenum and is encoded by the nucleic acid sequence exemplified in SEQ ID NO: 11 or is a functionally equivalent variant thereof.

In one embodiment, the enzyme 4-hydroxy-3-methylbut-2-enyl diphosphate reductase is derived from C. autoethanogenum and is encoded by the nucleic acid sequence exemplified in SEQ ID NO: 13 or is a functionally equivalent variant thereof.

In one embodiment, the enzyme mevalonate kinase (MK) is derived from Staphylococcus aureus subsp. aureus Mu50 and is encoded by the nucleic acid sequence exemplified in SEQ ID NO: 51 hereinafter, or it is a functionally equivalent variant thereof.

In one embodiment, the enzyme phosphomevalonate kinase (PMK) is derived from Staphylococcus aureus subsp. aureus Mu50 and is encoded by the nucleic acid sequence exemplified in SEQ ID NO: 52 hereinafter, or it is a functionally equivalent variant thereof.

In one embodiment, the enzyme mevalonate diphosphate decarboxylase (PMD) is derived from Staphylococcus aureus subsp. aureus Mu50 and is encoded by the nucleic acid sequence exemplified in SEQ ID NO: 53 hereinafter, or it is a functionally equivalent variant thereof.

In one embodiment, the enzyme Isopentenyl-diphosphate delta-isomerase (idi) is derived from Clostridium beijerinckii and is encoded by the nucleic acid sequence exemplified in SEQ ID NO: 54 hereinafter, or it is a functionally equivalent variant thereof.

In one embodiment, the enzyme thiolase (thlA) is derived from Clostridium acetobutylicum ATCC824 and is encoded by the nucleic acid sequence exemplified in SEQ ID NO: 40 hereinafter, or it is a functionally equivalent variant thereof.

In one embodiment, the enzyme is a thiolase enzyme, and is an acetyl-CoA c-acetyltransferase (vraB) derived from Staphylococcus aureus subsp. aureus Mu50 and is encoded by the nucleic acid sequence exemplified in SEQ ID NO: 41 hereinafter, or it is a functionally equivalent variant thereof.

In one embodiment, the enzyme 3-hydroxy-3-methylglutaryl-CoA synthase (HMGS) is derived from Staphylococcus aureus subsp. aureus Mu50 and is encoded by the nucleic acid sequence exemplified in SEQ ID NO: 42 hereinafter, or it is a functionally equivalent variant thereof.

In one embodiment, the enzyme Hydroxymethylglutaryl-CoA reductase (HMGR) is derived from Staphylococcus aureus subsp. aureus Mu50 and is encoded by the nucleic acid sequence exemplified in SEQ ID NO: 43 hereinafter, or it is a functionally equivalent variant thereof.

In one embodiment, the enzyme Geranyltranstransferase (ispA) is derived from Escherichia coli str. K-12 substr. MG1655 is encoded by the nucleic acid sequence exemplified in SEQ ID NO: 56 hereinafter, or it is a functionally equivalent variant thereof.

In one embodiment, the enzyme heptaprenyl diphosphate synthase is derived from C. autoethanogenum and is encoded by the nucleic acid sequence exemplified in SEQ ID NO: 17 or is a functionally equivalent variant thereof.

In one embodiment, the enzyme polyprenyl synthetase is derived from C. autoethanogenum and is encoded by the nucleic acid sequence exemplified in SEQ ID NO: 19 or is a functionally equivalent variant thereof.

In one embodiment, the enzyme Alpha-farnesene synthase (FS) is derived from Malus x domestica and is encoded by the nucleic acid sequence exemplified in SEQ ID NO: 57 hereinafter, or it is a functionally equivalent variant thereof.

The enzymes and functional variants of use in the microorganisms may be identified by assays known to one of skill in the art. In particular embodiments, the enzyme isoprene synthase may be identified by the method outlined Silver et al. (1991, Plant Physiol. 97: 1588-1591) or Zhao et al. (2011, Appl Microbiol Biotechnol, 90:1915-1922). In a further particular embodiment, the enzyme farnesene synthase may be identified by the method outlined in Green et al., 2007, Phytochemistry; 68:176-188. In further particular embodiments, enzymes from the mevalonate pathway may be identified by the method outlined in Cabano et al. (1997, Insect Biochem. Mol. Biol. 27: 499-505) for the HMG-CoA synthase, Ma et al. (2011, Metab. Engin., 13:588-597) for the HMG-CoA reductase and mevalonate kinase enzyme, Herdendorf and Miziorko (2007, Biochemistry, 46: 11780-8) for the phosphomevalonate kinase, and Krepkiy et al. (2004, Protein Sci. 13: 1875-1881) for the mevalonate diphosphate decarboxylase. Ma et al., 2011, Metab. Engin., 13:588-597. The 1-deoxy-D-xylulose 5-phosphate synthase of the DXS pathway can be assayed using the method outlined in Kuzuyama et al. (2000, J. Bacteriol. 182, 891-897). It is also possible to identify genes of DXS and mevalonate pathway using inhibitors like fosmidomycin or mevinolin as described by Trutko et al. (2005, Microbiology 74: 153-158).

In one embodiment, the microorganism comprises one or more exogenous nucleic acids adapted to increase expression of one or more endogenous nucleic acids and which one or more endogenous nucleic acids encode one or more of the enzymes referred to herein before. In one embodiment, the one or more exogenous nucleic acid adapted to increase expression is a regulatory element. In one embodiment, the regulatory element is a promoter. In one embodiment, the promoter is a constitutive promoter that is preferably highly active under appropriate fermentation conditions. Inducible promoters could also be used. In preferred embodiments, the promoter is selected from the group comprising Wood-Ljungdahl gene cluster or Phosphotransacetylase/Acetate kinase operon promoters. It will be appreciated by those of skill in the art that other promoters which can direct expression, preferably a high level of expression under appropriate fermentation conditions, would be effective as alternatives to the exemplified embodiments.

In one embodiment, the microorganism comprises one or more exogenous nucleic acids encoding and adapted to express one or more of the enzymes referred to herein before. In one embodiment, the microorganisms comprise one or more exogenous nucleic acid encoding and adapted to express at least two, at least of the enzymes. In other embodiments, the microorganism comprises one or more exogenous nucleic acid encoding and adapted to express at least three, at least four, at least five, at least six, at least seven, at least eight, at least nine or more of the enzymes.

In one particular embodiment, the microorganism comprises one or more exogenous nucleic acid encoding an enzyme of the disclosure or a functionally equivalent variant thereof.

The microorganism may comprise one or more exogenous nucleic acids. Where it is desirable to transform the parental microorganism with two or more genetic elements (such as genes or regulatory elements (for example a promoter)) they may be contained on one or more exogenous nucleic acids.

In one embodiment, the one or more exogenous nucleic acid is a nucleic acid construct or vector, in one particular embodiment a plasmid, encoding one or more of the enzymes referred to hereinbefore in any combination.

The exogenous nucleic acids may remain extra-chromosomal upon transformation of the parental microorganism or may integrate into the genome of the parental microorganism. Accordingly, they may include additional nucleotide sequences adapted to assist integration (for example, a region which allows for homologous recombination and targeted integration into the host genome) or expression and replication of an extrachromosomal construct (for example, origin of replication, promoter and other regulatory elements or sequences).

In one embodiment, the exogenous nucleic acids encoding one or enzymes as mentioned herein before will further comprise a promoter adapted to promote expression of the one or more enzymes encoded by the exogenous nucleic acids. In one embodiment, the promoter is a constitutive promoter that is preferably highly active under appropriate fermentation conditions. Inducible promoters could also be used. In preferred embodiments, the promoter is selected from the group comprising Wood-Ljungdahl gene cluster and Phosphotransacetylase/Acetate kinase promoters. It will be appreciated by those of skill in the art that other promoters which can direct expression, preferably a high level of expression under appropriate fermentation conditions, would be effective as alternatives to the exemplified embodiments.

In one embodiment, the exogenous nucleic acid is an expression plasmid.

In one particular embodiment, the parental microorganism is selected from the group of carboxydotrophic acetogenic bacteria. In certain embodiments the microorganism is selected from the group comprising Clostridium autoethanogenum, Clostridium ljungdahlii, Clostridium ragsdalei, Clostridium carboxidivorans, Clostridium drakei, Clostridium scatologenes, Clostridium aceticum, Clostridium formicoaceticum, Clostridium magnum, Butyribacterium methylotrophicum, Acetobacterium woodii, Alkalibaculum bacchii, Blautia producta, Eubacterium limosum, Moorella thermoacetica, Moorella thermautotrophica, Sporomusa ovata, Sporomusa silvacetica, Sporomusa sphaeroides, Oxobacter pfennigii, and Thermoanaerobacter kivui.

In one particular embodiment, the parental microorganism is selected from the cluster of ethanologenic, acetogenic Clostridia comprising the species C. autoethanogenum, C. ljungdahlii, and C. ragsdalei and related isolates. These include but are not limited to strains C. autoethanogenum JAI-1T (DSM10061) [Abrini J, Naveau H, Nyns E-J: Clostridium autoethanogenum, sp. nov., an anaerobic bacterium that produces ethanol from carbon monoxide. Arch Microbiol 1994, 4: 345-351], C. autoethanogenum LBS1560 (DSM19630) [Simpson S D, Forster R L, Tran P T, Rowe M J, Warner I L: Novel bacteria and methods thereof. International patent 2009, WO/2009/064200], C. autoethanogenum LBS1561 (DSM23693), C. ljungdahlii PETC^{T} (DSM13528=ATCC 55383) [Tanner R S, Miller L M, Yang D: Clostridium ljungdahlii sp. nov., an Acetogenic Species in Clostridial rRNA Homology Group I. Int J Syst Bacteriol 1993, 43: 232-236], C. ljungdahlii ERI-2 (ATCC 55380) [Gaddy J L: Clostridium stain which produces acetic acid from waste gases. US patent 1997, 5,593,886], C. ljungdahlii C-01 (ATCC 55988) [Gaddy J L, Clausen E C, Ko C-W: Microbial process for the preparation of acetic acid as well as solvent for its extraction from the fermentation broth. US patent, 2002, 6,368,819], C. ljungdahlii 0-52 (ATCC 55989) [Gaddy J L, Clausen E C, Ko C-W: Microbial process for the preparation of acetic acid as well as solvent for its extraction from the fermentation broth. US patent, 2002, 6,368,819], C. ragsdalei P11^{T} (ATCC BAA-622) [Huhnke R L, Lewis R S, Tanner R S: Isolation and Characterization of novel Clostridial Species. International patent 2008, WO 2008/028055], related isolates such as “C. coskatii” [Zahn et al—Novel ethanologenic species Clostridium coskatii (US Patent Application number U520110229947)] and “Clostridium sp.” (Tyurin et al., 2012, J. Biotech Res. 4: 1-12), or mutated strains such as C. ljungdahlii OTA-1 (Tirado-Acevedo O. Production of Bioethanol from Synthesis Gas Using Clostridium ljungdahlii. PhD thesis, North Carolina State University, 2010). These strains form a subcluster within the Clostridial rRNA cluster I, and their 16S rRNA gene is more than 99% identical with a similar low GC content of around 30%. However, DNA-DNA reassociation and DNA fingerprinting experiments showed that these strains belong to distinct species [Huhnke R L, Lewis R S, Tanner R S: Isolation and Characterization of novel Clostridial Species. International patent 2008, WO 2008/028055].

All species of this cluster have a similar morphology and size (logarithmic growing cells are between 0.5-0.7×3-5 μm), are mesophilic (optimal growth temperature between 30-37° C.) and strictly anaerobe [Tanner R S, Miller L M, Yang D: Clostridium ljungdahlii sp. nov., an Acetogenic Species in Clostridial rRNA Homology Group I. Int J Syst Bacteriol 1993, 43: 232-236; Abrini J, Naveau H, Nyns E-J: Clostridium autoethanogenum, sp. nov., an anaerobic bacterium that produces ethanol from carbon monoxide. Arch Microbiol 1994, 4: 345-351; Huhnke R L, Lewis R S, Tanner R S: Isolation and Characterization of novel Clostridial Species. International patent 2008, WO 2008/028055]. Moreover, they all share the same major phylogenetic traits, such as same pH range (pH 4-7.5, with an optimal initial pH of 5.5-6), strong autotrophic growth on CO containing gases with similar growth rates, and a similar metabolic profile with ethanol and acetic acid as main fermentation end product, and small amounts of 2,3-butanediol and lactic acid formed under certain conditions. [Tanner R S, Miller L M, Yang D: Clostridium ljungdahlii sp. nov., an Acetogenic Species in Clostridial rRNA Homology Group I. Int J Syst Bacteriol 1993, 43: 232-236; Abrini J, Naveau H, Nyns E-J: Clostridium autoethanogenum, sp. nov., an anaerobic bacterium that produces ethanol from carbon monoxide. Arch Microbiol 1994, 4: 345-351; Huhnke R L, Lewis R S, Tanner R S: Isolation and Characterization of novel Clostridial Species. International patent 2008, WO 2008/028055]. Indole production was observed with all three species as well. However, the species differentiate in substrate utilization of various sugars (e.g. rhamnose, arabinose), acids (e.g. gluconate, citrate), amino acids (e.g. arginine, histidine), or other substrates (e.g. betaine, butanol). Moreover, some of the species were found to be auxotroph to certain vitamins (e.g. thiamine, biotin) while others were not.

In one embodiment, the parental carboxydotrophic acetogenic microorganism is selected from the group consisting of Clostridium autoethanogenum, Clostridium ljungdahlii, Clostridium ragsdalei, Clostridium carboxidivorans, Clostridium drakei, Clostridium scatologenes, Butyribacterium limosum, Butyribacterium methylotrophicum, Acetobacterium woodii, Alkalibaculum bacchii, Blautia producta, Eubacterium limosum, Moorella thermoacetica, Moorella thermautotrophica, Oxobacter pfennigii, and Thermoanaerobacter kivui.

In one particular embodiment of the first or second aspects, the parental microorganism is selected from the group of carboxydotrophic Clostridia comprising Clostridium autoethanogenum, Clostridium ljungdahlii, Clostridium ragsdalei, Clostridium carboxidivorans, Clostridium drakei, Clostridium scatologenes, Clostridium aceticum, Clostridium formicoaceticum, Clostridium magnum.

In a one embodiment, the microorganism is selected from a cluster of carboxydotrophic Clostridia comprising the species C. autoethanogenum, C. ljungdahlii, and “C. ragsdalei” and related isolates. These include but are not limited to strains C. autoethanogenum JAI-1^{T} (DSM10061) (Abrini, Naveau, & Nyns, 1994), C. autoethanogenum LBS1560 (DSM19630) (WO/2009/064200), C. autoethanogenum LBS1561 (DSM23693), C. ljungdahlii PETC^{T} (DSM13528=ATCC 55383) (Tanner, Miller, & Yang, 1993), C. ljungdahlii ERI-2 (ATCC 55380) (U.S. Pat. No. 5,593,886), C. ljungdahlii C-01 (ATCC 55988) (U.S. Pat. No. 6,368,819), C. ljungdahlii O-52 (ATCC 55989) (U.S. Pat. No. 6,368,819), or “C. ragsdalei P11^{T}” (ATCC BAA-622) (WO 2008/028055), and related isolates such as “C. coskatii” (US patent 2011/0229947), “Clostridium sp. MT351” (Michael Tyurin & Kiriukhin, 2012) and mutant strains thereof such as C. ljungdahlii OTA-1 (Tirado-Acevedo O. Production of Bioethanol from Synthesis Gas Using Clostridium ljungdahlii. PhD thesis, North Carolina State University, 2010).

These strains form a subcluster within the Clostridial rRNA cluster I (Collins et al., 1994), having at least 99% identity on 16S rRNA gene level, although being distinct species as determined by DNA-DNA reassociation and DNA fingerprinting experiments (WO 2008/028055, US patent 2011/0229947).

The strains of this cluster are defined by common characteristics, having both a similar genotype and phenotype, and they all share the same mode of energy conservation and fermentative metabolism. The strains of this cluster lack cytochromes and conserve energy via an Rnf complex.

All strains of this cluster have a genome size of around 4.2 MBp (Köpke et al., 2010) and a GC composition of around 32% mol (Abrini et al., 1994; Köpke et al., 2010; Tanner et al., 1993) (WO 2008/028055; US patent 2011/0229947), and conserved essential key gene operons encoding for enzymes of Wood-Ljungdahl pathway (Carbon monoxide dehydrogenase, Formyl-tetrahydrofolate synthetase, Methylene-tetrahydrofolate dehydrogenase, Formyl-tetrahydrofolate cyclohydrolase, Methylene-tetrahydrofolate reductase, and Carbon monoxide dehydrogenase/Acetyl-CoA synthase), hydrogenase, formate dehydrogenase, Rnf complex (rnfCDGEAB), pyruvate:ferredoxin oxidoreductase, aldehyde:ferredoxin oxidoreductase (Köpke et al., 2010, 2011). The organization and number of Wood-Ljungdahl pathway genes, responsible for gas uptake, has been found to be the same in all species, despite differences in nucleic and amino acid sequences (Köpke et al., 2011).

The strains all have a similar morphology and size (logarithmic growing cells are between 0.5-0.7×3-5 μm), are mesophilic (optimal growth temperature between 30-37° C.) and strictly anaerobe (Abrini et al., 1994; Tanner et al., 1993)(WO 2008/028055). Moreover, they all share the same major phylogenetic traits, such as same pH range (pH 4-7.5, with an optimal initial pH of 5.5-6), strong autotrophic growth on CO containing gases with similar growth rates, and a metabolic profile with ethanol and acetic acid as main fermentation end product, with small amounts of 2,3-butanediol and lactic acid formed under certain conditions (Abrini et al., 1994; Köpke et al., 2011; Tanner et al., 1993) However, the species differentiate in substrate utilization of various sugars (e.g. rhamnose, arabinose), acids (e.g. gluconate, citrate), amino acids (e.g. arginine, histidine), or other substrates (e.g. betaine, butanol). Some of the species were found to be auxotroph to certain vitamins (e.g. thiamine, biotin) while others were not. Reduction of carboxylic acids into their corresponding alcohols has been shown in a range of these organisms (Perez, Richter, Loftus, & Angenent, 2012).

The traits described are therefore not specific to one organism like C. autoethanogenum or C. ljungdahlii, but rather general traits for carboxydotrophic, ethanol-synthesizing Clostridia. Thus, the disclosure can be anticipated to work across these strains, although there may be differences in performance.

The recombinant carboxydotrophic acetogenic microorganisms of the disclosure may be prepared from a parental carboxydotrophic acetogenic microorganism and one or more exogenous nucleic acids using any number of techniques known in the art for producing recombinant microorganisms. By way of example only, transformation (including transduction or transfection) may be achieved by electroporation, electrofusion, ultrasonication, polyethylene glycol-mediated transformation, conjugation, or chemical and natural competence. Suitable transformation techniques are described for example in Sambrook J, Fritsch E F, Maniatis T: Molecular Cloning: A laboratory Manual, Cold Spring Harbour Laboratory Press, Cold Spring Harbour, 1989.

Electroporation has been described for several carboxydotrophic acetogens as C. ljungdahlii (Köpke et al., 2010; Leang, Ueki, Nevin, & Lovley, 2012) (PCT/NZ2011/000203; WO2012/053905), C. autoethanogenum (PCT/NZ2011/000203; WO2012/053905), Acetobacterium woodii (Strätz, Sauer, Kuhn, & Dürre, 1994) or Moorella thermoacetica (Kita et al., 2012) and is a standard method used in many Clostridia such as C. acetobutylicum (Mermelstein, Welker, Bennett, & Papoutsakis, 1992), C. cellulolyticum (Jennert, Tardif, Young, & Young, 2000) or C. thermocellum (M V Tyurin, Desai, & Lynd, 2004).

Electrofusion has been described for acetogenic Clostridium sp. MT351 (Tyurin and Kiriukhin, 2012).

Prophage Prasanna Tamarapu Parthasarathy induction has been described for carboxydotrophic acetogen as well in case of C. scatologenes (, 2010, Development of a Genetic Modification System in Clostridium scatologenes ATCC 25775 for Generation of Mutants, Masters Project Western Kentucky University).

Conjugation has been described as method of choice for acetogen Clostridium difficile (Herbert, O'Keeffe, Purdy, Elmore, & Minton, 2003) and many other Clostridia including C. acetobutylicum (Williams, Young, & Young, 1990).

In one embodiment, the parental strain uses CO as its sole carbon and energy source.

In one embodiment the parental microorganism is Clostridium autoethanogenum or Clostridium ljungdahlii. In one particular embodiment, the microorganism is Clostridium autoethanogenum DSM23693. In another particular embodiment, the microorganism is Clostridium ljungdahlii DSM13528 (or ATCC55383).

### Nucleic Acids

The disclosure also provides one or more nucleic acids or nucleic acid constructs of use in generating a recombinant microorganism of the disclosure.

In one embodiment, the nucleic acid comprises sequences encoding one or more of the enzymes in the mevalonate (MVA) pathway and optionally the DXS pathway which when expressed in a microorganism allows the microorganism to produce one or more terpenes and/or precursors thereof by fermentation of a substrate comprising CO. In one particular embodiment, the disclosure provides a nucleic acid encoding two or more enzymes which when expressed in a microorganism allows the microorganism to produce one or more terpene and/or precursor thereof by fermentation of substrate comprising CO. In one embodiment, a nucleic acid of the disclosure encodes three, four, five or more of such enzymes.

In one embodiment, the one or more enzymes encoded by the nucleic acid are from the mevalonate (MVA) pathway and are selected from the group consisting of:

a) thiolase (EC 2.3.1.9),

b) HMG-CoA synthase (EC 2.3.3.10),

c) HMG-CoA reductase (EC 1.1.1.88),

d) Mevalonate kinase (EC 2.7.1.36),

e) Phosphomevalonate kinase (EC 2.7.4.2),

f) Mevalonate Diphosphate decarboxylase (EC 4.1.1.33), and

g) a functionally equivalent variant of any one thereof.

In a further embodiment, the one or more optional enzymes encoded by the nucleic acid are from the DXS pathway are selected from the group consisting of:

a) 1-deoxy-D-xylulose-5-phosphate synthase DXS (EC:2.2.1.7),

b) 1-deoxy-D-xylulose 5-phosphate reductoisomerase DXR (EC:1.1.1.267),

c) 2-C-methyl-D-erythritol 4-phosphate cytidylyltransferase IspD (EC:2.7.7.60), d) 4-diphosphocytidyl-2-C-methyl-D-erythritol kinase IspE (EC:2.7.1.148),

e) 2-C-methyl-D-erythritol 2,4-cyclodiphosphate synthase IspF (EC:4.6.1.12),

f) 4-hydroxy-3-methylbut-2-en-1-yl diphosphate synthase IspG (EC:1.17.7.1),

g) 4-hydroxy-3-methylbut-2-enyl diphosphate reductase (EC:1.17.1.2), and

h) a functionally equivalent variant of any one thereof.

In a further embodiment, the nucleic acid encodes one or more further enzymes that are expressed or over-expressed to result in the production of a terpene compound and/or precursor thereof wherein the exogenous enzyme that is expressed, or the endogenous enzyme that is overexpressed is selected from the group consisting of:

a) geranyltranstransferase Fps (EC:2.5.1.10),

b) heptaprenyl diphosphate synthase (EC:2.5.1.10),

c) octaprenyl-diphosphate synthase (EC:2.5.1.90),

d) isoprene synthase (EC 4.2.3.27),

e) isopentenyl-diphosphate delta-isomerase (EC 5.3.3.2),

f) farnesene synthase (EC 4.2.3.46/EC 4.2.3.47), and

g) a functionally equivalent variant of any one thereof.

Exemplary amino acid sequences and nucleic acid sequences encoding each of the above enzymes are provided herein or can be obtained from GenBank as mentioned hereinbefore. However, skilled persons will readily appreciate alternative nucleic acid sequences encoding the enzymes or functionally equivalent variants thereof, having regard to the information contained herein, in GenBank and other databases, and the genetic code.

In a further embodiment, the nucleic acid encoding thiolase (thlA) derived from Clostridium acetobutylicum ATCC824 is encoded by the nucleic acid sequence exemplified in SEQ ID NO: 40 hereinafter, or it is a functionally equivalent variant thereof.

In a further embodiment, the nucleic acid encoding thiolase wherein the thiolase is acetyl-CoA c-acetyltransferase (vraB) derived from Staphylococcus aureus subsp. aureus Mu50 is encoded by the nucleic acid sequence exemplified in SEQ ID NO: 41 hereinafter, or it is a functionally equivalent variant thereof.

In a further embodiment, the nucleic acid encoding 3-hydroxy-3-methylglutaryl-CoA synthase (HMGS) derived from Staphylococcus aureus subsp. aureus Mu50 is encoded by the nucleic acid sequence exemplified in SEQ ID NO: 42 hereinafter, or it is a functionally equivalent variant thereof.

In a further embodiment, the nucleic acid encoding Hydroxymethylglutaryl-CoA reductase (HMGR) derived from Staphylococcus aureus subsp. aureus Mu50 is encoded by the nucleic acid sequence exemplified in SEQ ID NO: 43 hereinafter, or it is a functionally equivalent variant thereof.

In a further embodiment, the nucleic acid encoding mevalonate kinase (MK) derived from Staphylococcus aureus subsp. aureus Mu50 is encoded by the nucleic acid sequence exemplified in SEQ ID NO: 51 hereinafter, or it is a functionally equivalent variant thereof.

In a further embodiment, the nucleic acid encoding phosphomevalonate kinase (PMK) derived from Staphylococcus aureus subsp. aureus Mu50 is encoded by the nucleic acid sequence exemplified in SEQ ID NO: 52 hereinafter, or it is a functionally equivalent variant thereof.

In a further embodiment, the nucleic acid encoding mevalonate diphosphate decarboxylase (PMD) derived from Staphylococcus aureus subsp. aureus Mu50 is encoded by the nucleic acid sequence exemplified in SEQ ID NO: 53 hereinafter, or it is a functionally equivalent variant thereof.

In a further embodiment, the nucleic acid encoding deoxyxylulose 5-phosphate synthase derived from C. autoethanogenum, is encoded by the nucleic acid sequence exemplified in SEQ ID NO: 1 and/or with the amino acid sequence exemplified in SEQ ID NO: 2 hereinafter, or it is a functionally equivalent variant thereof.

In one embodiment, the nucleic acid encoding 1-deoxy-D-xylulose 5-phosphate reductoisomerase DXR (EC:1.1.1.267) has the sequence SEQ ID NO: 3 or is a functionally equivalent variant thereof.

In one embodiment, the nucleic acid encoding 2-C-methyl-D-erythritol 4-phosphate cytidylyltransferase IspD (EC:2.7.7.60) has the sequence SEQ ID NO: 5 or is a functionally equivalent variant thereof.

In one embodiment, the nucleic acid encoding 4-diphosphocytidyl-2-C-methyl-D-erythritol kinase IspE (EC:2.7.1.148) has the sequence SEQ ID NO: 7 or is a functionally equivalent variant thereof.

In one embodiment, the nucleic acid encoding 2-C-methyl-D-erythritol 2,4-cyclodiphosphate synthase IspF (EC:4.6.1.12) has the sequence SEQ ID NO: 9 or is a functionally equivalent variant thereof.

In one embodiment, the nucleic acid encoding 4-hydroxy-3-methylbut-2-en-1-yl diphosphate synthase IspG (EC:1.17.7.1) has the sequence SEQ ID NO: 11 or is a functionally equivalent variant thereof.

In one embodiment, the nucleic acid encoding 4-hydroxy-3-methylbut-2-enyl diphosphate reductase (EC:1.17.1.2) has the sequence SEQ ID NO: 13 or is a functionally equivalent variant thereof.

In a further embodiment, the nucleic acid encoding Geranyltranstransferase (ispA) derived from Escherichia coli str. K-12 substr. MG1655 is encoded by the nucleic acid sequence exemplified in SEQ ID NO: 56 hereinafter, or it is a functionally equivalent variant thereof.

In one embodiment, the nucleic acid encoding heptaprenyl diphosphate synthase has the sequence SEQ ID NO: 17, or it is a functionally equivalent variant thereof.

In one embodiment, the nucleic acid encoding octaprenyl-diphosphate synthase (EC:2.5.1.90) wherein the octaprenyl-diphosphate synthase is polyprenyl synthetase is encoded by sequence SEQ ID NO: 19, or it is a functionally equivalent variant thereof.

In one embodiment, the nucleic acid encoding isoprene synthase (ispS) derived from Poplar tremuloides is exemplified in SEQ ID NO: 21 hereinafter, or it is a functionally equivalent variant thereof.

In a further embodiment, the nucleic acid encoding Isopentenyl-diphosphate delta-isomerase (idi) derived from Clostridium beijerinckii is encoded by the nucleic acid sequence exemplified in SEQ ID NO: 54 hereinafter, or it is a functionally equivalent variant thereof.

In a further embodiment, the nucleic acid encoding Alpha-farnesene synthase (FS) derived from Malus x domestica is encoded by the nucleic acid sequence exemplified in SEQ ID NO: 57 hereinafter, or it is a functionally equivalent variant thereof.

In one embodiment, the nucleic acids of the disclosure will further comprise a promoter. In one embodiment, the promoter allows for constitutive expression of the genes under its control. However, inducible promoters may also be employed. Persons of skill in the art will readily appreciate promoters of use in the disclosure. Preferably, the promoter can direct a high level of expression under appropriate fermentation conditions. In a particular embodiment a Wood-Ljungdahl cluster promoter is used. In another embodiment, a Phosphotransacetylase/Acetate kinase promoter is used. In another embodiment a pyruvate:ferredoxin oxidoreductase promoter, an Rnf complex operon promoter or an ATP synthase operon promoter. In one particular embodiment, the promoter is from C. autoethanogenum.

The nucleic acids of the disclosure may remain extra-chromosomal upon transformation of a parental microorganism or may be adapted for integration into the genome of the microorganism. Accordingly, nucleic acids of the disclosure may include additional nucleotide sequences adapted to assist integration (for example, a region which allows for homologous recombination and targeted integration into the host genome) or stable expression and replication of an extrachromosomal construct (for example, origin of replication, promoter and other regulatory sequences).

In one embodiment, the nucleic acid is nucleic acid construct or vector. In one particular embodiment, the nucleic acid construct or vector is an expression construct or vector, however other constructs and vectors, such as those used for cloning are encompassed by the disclosure. In one particular embodiment, the expression construct or vector is a plasmid.

It will be appreciated that an expression construct/vector of the present disclosure may contain any number of regulatory elements in addition to the promoter as well as additional genes suitable for expression of further proteins if desired. In one embodiment the expression construct/vector includes one promoter. In another embodiment, the expression construct/vector includes two or more promoters. In one particular embodiment, the expression construct/vector includes one promoter for each gene to be expressed. In one embodiment, the expression construct/vector includes one or more ribosomal binding sites, preferably a ribosomal binding site for each gene to be expressed.

It will be appreciated by those of skill in the art that the nucleic acid sequences and construct/vector sequences described herein may contain standard linker nucleotides such as those required for ribosome binding sites and/or restriction sites. Such linker sequences should not be interpreted as being required and do not provide a limitation on the sequences defined.

Nucleic acids and nucleic acid constructs, including expression constructs/vectors of the disclosure may be constructed using any number of techniques standard in the art. For example, chemical synthesis or recombinant techniques may be used. Such techniques are described, for example, in Sambrook et al (Molecular Cloning: A laboratory manual, Cold Spring Harbor Laboratory Press, Cold Spring Harbor, N.Y., 1989). Further exemplary techniques are described in the Examples section herein after. Essentially, the individual genes and regulatory elements will be operably linked to one another such that the genes can be expressed to form the desired proteins. Suitable vectors for use in the disclosure will be appreciated by those of ordinary skill in the art. However, by way of example, the following vectors may be suitable: pMTL80000 vectors, pIMP1, pJIR750, and the plasmids exemplified in the Examples section herein after.

It should be appreciated that nucleic acids of the disclosure may be in any appropriate form, including RNA, DNA, or cDNA.

The disclosure also provides host organisms, particularly microorganisms, and including viruses, bacteria, and yeast, comprising any one or more of the nucleic acids described herein.

### Methods of Producing Organisms

The one or more exogenous nucleic acids may be delivered to a parental microorganism as naked nucleic acids or may be formulated with one or more agents to facilitate the transformation process (for example, liposome-conjugated nucleic acid, an organism in which the nucleic acid is contained). The one or more nucleic acids may be DNA, RNA, or combinations thereof, as is appropriate. Restriction inhibitors may be used in certain embodiments; see, for example Murray, N. E. et al. (2000) Microbial. Molec. Biol. Rev. 64, 412.)

The microorganisms of the disclosure may be prepared from a parental microorganism and one or more exogenous nucleic acids using any number of techniques known in the art for producing recombinant microorganisms. By way of example only, transformation (including transduction or transfection) may be achieved by electroporation, ultrasonication, polyethylene glycol-mediated transformation, chemical or natural competence, or conjugation. Suitable transformation techniques are described for example in, Sambrook J, Fritsch E F, Maniatis T: Molecular Cloning: A laboratory Manual, Cold Spring Harbour Laboratory Press, Cold Spring Harbour, 1989.

In certain embodiments, due to the restriction systems which are active in the microorganism to be transformed, it is necessary to methylate the nucleic acid to be introduced into the microorganism. This can be done using a variety of techniques, including those described below, and further exemplified in the Examples section herein after.

By way of example, in one embodiment, a recombinant microorganism of the disclosure is produced by a method comprises the following steps:
b) introduction into a shuttle microorganism of (i) of an expression construct/vector as described herein and (ii) a methylation construct/vector comprising a methyltransferase gene;
c) expression of the methyltransferase gene;
d) isolation of one or more constructs/vectors from the shuttle microorganism; and,
e) introduction of the one or more construct/vector into a destination microorganism.

In one embodiment, the methyltransferase gene of step B is expressed constitutively. In another embodiment, expression of the methyltransferase gene of step B is induced.

The shuttle microorganism is a microorganism, preferably a restriction negative microorganism, that facilitates the methylation of the nucleic acid sequences that make up the expression construct/vector. In a particular embodiment, the shuttle microorganism is a restriction negative E. coli, Bacillus subtilis, or Lactococcus lactis.

The methylation construct/vector comprises a nucleic acid sequence encoding a methyltransferase.

Once the expression construct/vector and the methylation construct/vector are introduced into the shuttle microorganism, the methyltransferase gene present on the methylation construct/vector is induced. Induction may be by any suitable promoter system although in one particular embodiment of the disclosure, the methylation construct/vector comprises an inducible lac promoter and is induced by addition of lactose or an analogue thereof, more preferably isopropyl-β-D-thio-galactoside (IPTG). Other suitable promoters include the ara, tet, or T7 system. In a further embodiment of the disclosure, the methylation construct/vector promoter is a constitutive promoter.

In a particular embodiment, the methylation construct/vector has an origin of replication specific to the identity of the shuttle microorganism so that any genes present on the methylation construct/vector are expressed in the shuttle microorganism. Preferably, the expression construct/vector has an origin of replication specific to the identity of the destination microorganism so that any genes present on the expression construct/vector are expressed in the destination microorganism.

Expression of the methyltransferase enzyme results in methylation of the genes present on the expression construct/vector. The expression construct/vector may then be isolated from the shuttle microorganism according to any one of a number of known methods. By way of example only, the methodology described in the Examples section described hereinafter may be used to isolate the expression construct/vector.

In one particular embodiment, both construct/vector are concurrently isolated.

The expression construct/vector may be introduced into the destination microorganism using any number of known methods. However, by way of example, the methodology described in the Examples section hereinafter may be used. Since the expression construct/vector is methylated, the nucleic acid sequences present on the expression construct/vector are able to be incorporated into the destination microorganism and successfully expressed.

It is envisaged that a methyltransferase gene may be introduced into a shuttle microorganism and over-expressed. Thus, in one embodiment, the resulting methyltransferase enzyme may be collected using known methods and used in vitro to methylate an expression plasmid. The expression construct/vector may then be introduced into the destination microorganism for expression. In another embodiment, the methyltransferase gene is introduced into the genome of the shuttle microorganism followed by introduction of the expression construct/vector into the shuttle microorganism, isolation of one or more constructs/vectors from the shuttle microorganism and then introduction of the expression construct/vector into the destination microorganism.

It is envisaged that the expression construct/vector and the methylation construct/vector as defined above may be combined to provide a composition of matter. Such a composition has particular utility in circumventing restriction barrier mechanisms to produce the recombinant microorganisms of the disclosure.

In one particular embodiment, the expression construct/vector and/or the methylation construct/vector are plasmids.

Persons of ordinary skill in the art will appreciate a number of suitable methyltransferases of use in producing the microorganisms of the disclosure. However, by way of example the Bacillus subtilis phage ΦT1 methyltransferase and the methyltransferase described in the Examples herein after may be used. In one embodiment, the methyltransferase has the amino acid sequence of SEQ ID NO: 60 or is a functionally equivalent variant thereof. Nucleic acids encoding suitable methyltransferases will be readily appreciated having regard to the sequence of the desired methyltransferase and the genetic code. In one embodiment, the nucleic acid encoding a methyltransferase is as described in the Examples herein after (for example the nucleic acid of SEQ ID NO: 63, or it is a functionally equivalent variant thereof).

Any number of constructs/vectors adapted to allow expression of a methyltransferase gene may be used to generate the methylation construct/vector. However, by way of example, the plasmid described in the Examples section hereinafter may be used.

### Methods of Production

The disclosure provides a method for the production of one or more terpenes and/or precursors thereof, and optionally one or more other products, by microbial fermentation comprising fermenting a substrate comprising CO using a recombinant microorganism of the disclosure. Preferably, the one or more terpene and/or precursor thereof is the main fermentation product. The methods of the disclosure may be used to reduce the total atmospheric carbon emissions from an industrial process.

Preferably, the fermentation comprises the steps of anaerobically fermenting a substrate in a bioreactor to produce at least one or more terpenes and/or a precursor thereof using a recombinant microorganism of the disclosure.

In one embodiment, the one or more terpene and/or precursor thereof is chosen from mevalonic acid, IPP, dimethylallyl pyrophosphate (DMAPP), isoprene, geranyl pyrophosphate (GPP), farnesyl pyrophosphate (FPP) and farnesene.

Instead of producing isoprene directly from terpenoid key intermediates IPP and DMAPP then using this to synthesise longer chain terpenes, it is also possible to synthesise longer chain terpenes, such as C10 Monoterpenoids or C15 Sesquiterpenoids, directly via a geranyltransferase (see Table 6). From C15 Sesquiterpenoid building block farnesyl-PP it is possible to produce farnesene, which, similarly to ethanol, can be used as a transportation fuel.

In one embodiment the method comprises the steps of:

(a) providing a substrate comprising CO to a bioreactor containing a culture of one or more microorganism of the disclosure; and
(b) anaerobically fermenting the culture in the bioreactor to produce at least one or more terpene and/or precursor thereof.

In one embodiment the method comprises the steps of:
a) capturing CO-containing gas produced as a result of the industrial process;
b) anaerobic fermentation of the CO-containing gas to produce the at least one or more terpene and/or precursor thereof by a culture containing one or more microorganism of the disclosure.

In an embodiment of the disclosure, the gaseous substrate fermented by the microorganism is a gaseous substrate containing CO. The gaseous substrate may be a CO-containing waste gas obtained as a by-product of an industrial process, or from some other source such as from automobile exhaust fumes. In certain embodiments, the industrial process is selected from the group consisting of ferrous metal products manufacturing, such as a steel mill, non-ferrous products manufacturing, petroleum refining processes, gasification of coal, electric power production, carbon black production, ammonia production, methanol production and coke manufacturing. In these embodiments, the CO-containing gas may be captured from the industrial process before it is emitted into the atmosphere, using any convenient method. The CO may be a component of syngas (gas comprising carbon monoxide and hydrogen). The CO produced from industrial processes is normally flared off to produce CO₂ and therefore the disclosure has particular utility in reducing CO₂ greenhouse gas emissions and producing a terpene for use as a biofuel. Depending on the composition of the gaseous CO-containing substrate, it may also be desirable to treat it to remove any undesired impurities, such as dust particles before introducing it to the fermentation. For example, the gaseous substrate may be filtered or scrubbed using known methods.

It will be appreciated that for growth of the bacteria and CO-to-at least one or more terpene and/or precursor thereof to occur, in addition to the CO-containing substrate gas, a suitable liquid nutrient medium will need to be fed to the bioreactor. The substrate and media may be fed to the bioreactor in a continuous, batch or batch fed fashion. A nutrient medium will contain vitamins and minerals sufficient to permit growth of the micro-organism used. Anaerobic media suitable for fermentation to produce a terpene and/or a precursor thereof using CO are known in the art. For example, suitable media are described Biebel (2001). In one embodiment of the disclosure the media is as described in the Examples section herein after.

The fermentation should desirably be carried out under appropriate conditions for the CO-to-the at least one or more terpene and/or precursor thereof fermentation to occur. Reaction conditions that should be considered include pressure, temperature, gas flow rate, liquid flow rate, media pH, media redox potential, agitation rate (if using a continuous stirred tank reactor), inoculum level, maximum gas substrate concentrations to ensure that CO in the liquid phase does not become limiting, and maximum product concentrations to avoid product inhibition.

In addition, it is often desirable to increase the CO concentration of a substrate stream (or CO partial pressure in a gaseous substrate) and thus increase the efficiency of fermentation reactions where CO is a substrate. Operating at increased pressures allows a significant increase in the rate of CO transfer from the gas phase to the liquid phase where it can be taken up by the micro-organism as a carbon source for the production of at least one or more terpene and/or precursor thereof. This in turn means that the retention time (defined as the liquid volume in the bioreactor divided by the input gas flow rate) can be reduced when bioreactors are maintained at elevated pressure rather than atmospheric pressure. The optimum reaction conditions will depend partly on the particular micro-organism of the disclosure used. However, in general, it is preferred that the fermentation be performed at pressure higher than ambient pressure. Also, since a given CO-to-at least one or more terpene and/or precursor thereof conversion rate is in part a function of the substrate retention time, and achieving a desired retention time in turn dictates the required volume of a bioreactor, the use of pressurized systems can greatly reduce the volume of the bioreactor required, and consequently the capital cost of the fermentation equipment. According to examples given in U.S. Pat. No. 5,593,886, reactor volume can be reduced in linear proportion to increases in reactor operating pressure, i.e. bioreactors operated at 10 atmospheres of pressure need only be one tenth the volume of those operated at 1 atmosphere of pressure.

By way of example, the benefits of conducting a gas-to-ethanol fermentation at elevated pressures has been described. For example, WO 02/08438 describes gas-to-ethanol fermentations performed under pressures of 30 psig and 75 psig, giving ethanol productivities of 150 g/l/day and 369 g/l/day respectively. However, example fermentations performed using similar media and input gas compositions at atmospheric pressure were found to produce between 10 and 20 times less ethanol per litre per day.

It is also desirable that the rate of introduction of the CO-containing gaseous substrate is such as to ensure that the concentration of CO in the liquid phase does not become limiting. This is because a consequence of CO-limited conditions may be that one or more product is consumed by the culture.

The composition of gas streams used to feed a fermentation reaction can have a significant impact on the efficiency and/or costs of that reaction. For example, 02 may reduce the efficiency of an anaerobic fermentation process. Processing of unwanted or unnecessary gases in stages of a fermentation process before or after fermentation can increase the burden on such stages (e.g. where the gas stream is compressed before entering a bioreactor, unnecessary energy may be used to compress gases that are not needed in the fermentation). Accordingly, it may be desirable to treat substrate streams, particularly substrate streams derived from industrial sources, to remove unwanted components and increase the concentration of desirable components.

In certain embodiments a culture of a bacterium of the disclosure is maintained in an aqueous culture medium. Preferably the aqueous culture medium is a minimal anaerobic microbial growth medium. Suitable media are known in the art and described for example in U.S. Pat. Nos. 5,173,429 and 5,593,886 and WO 02/08438, and as described in the Examples section herein after.

Terpenes and/or precursors thereof, or a mixed stream containing one or more terpenes, precursors thereof and/or one or more other products, may be recovered from the fermentation broth by methods known in the art, such as fractional distillation or evaporation, pervaporation, gas stripping and extractive fermentation, including for example, liquid-liquid extraction.

In certain preferred embodiments of the disclosure, the one or more terpene and/or precursor thereof and one or more products are recovered from the fermentation broth by continuously removing a portion of the broth from the bioreactor, separating microbial cells from the broth (conveniently by filtration), and recovering one or more products from the broth. Alcohols may conveniently be recovered for example by distillation. Acetone may be recovered for example by distillation. Any acids produced may be recovered for example by adsorption on activated charcoal. The separated microbial cells are preferably returned to the fermentation bioreactor. The cell free permeate remaining after any alcohol(s) and acid(s) have been removed is also preferably returned to the fermentation bioreactor. Additional nutrients (such as B vitamins) may be added to the cell free permeate to replenish the nutrient medium before it is returned to the bioreactor.

Also, if the pH of the broth was adjusted as described above to enhance adsorption of acetic acid to the activated charcoal, the pH should be re-adjusted to a similar pH to that of the broth in the fermentation bioreactor, before being returned to the bioreactor.

### Enzymes for Prenol/Isoprenol Pathways (Table 3):

| Abbr. | | | | | | |
|---|---|---|---|---|---|---|
| enzyme | | | | | | |
| names | | | | | | |
| from | | | | Example | | |
| pathway | | # | E.C. | Organism (genome | Gene | Gene locus |
| diagrams | Full name | genes | number | accession) | name | tag(s) |
| KAT1 | 3-ketoacyl-CoA | 1 | 2.3.1.16 or | C. acetobutylicum | thl | CA_C2873 |
| (thiolase) | thiolase | | 2.3.1.9 | (NC_003030.1) | | |
| | (aka acetyl-CoA C- | | | | | |
| | acetyltransferase) | | | | | |
| CtfAB | CoA transferase | 2 | 2.8.3.9 | C. acetobutylicum | ctfA,ctfB | CA_P0163 |
| | | | | (NC_003030.1) | | CA_P0164 |
| HMGCOAS | 3- | 1 | 2.3.3.10 | E. faecalis | mvaS or | YML126C |
| | hydroxymethylglutaryl- | | | (AF290092.1) | hmgS | (for |
| | CoA synthase | | | S. cerevisiae | | S. cerevisiae |
| | | | | (NC_001145.3) | | hmgS) |
| MGCH | Methylglutaconyl- | 1 | 4.2.1.18 | P. putida | liuC | PP_4066 |
| | CoA hydratase | | | (NC_002947.4) | | |
| MCCC | 3-methylcrotonyl- | 2 | 6.4.1.4 | P. aeruginosa | HuBjiuD | PA2014 |
| | CoA carboxylase | | | (AE004091.2) | | PA2012 |
| ACOAR | Acyl-CoA reductase | 1 | 1.2.1.10 | C. beijerinckii | cbjALD | |
| | | | | (AF157306.2) | | |
| ADH | Alcohol | 1 | 1.1.1.2 | E. coli | yahK | b0325 |
| | dehydrogenase | | | (NC_000913.3) | | |
| Ptb-buk | Phosphotransbutyrase- | 2 | 2.3.1.19 | C. acetobutylicum | ptb,buk | CA_C3076 |
| | Butyrate-kinase | | 2.7.2.7 | (NC_003030.1) | | CA_C3075 |
| ADC | Acetoacetate | 1 | 4.1.1.4 | C. acetobutylicum | adc | CA_P0165 |
| | decarboxylase | | | (AE001438.3) | | |
| HIVS | Hydroxyisovalerate | 1 | 2.3.3.10 | S. aureus | mvaS | (BAU36102.1 |
| | synthase. | | | (BAU36102.1) | | is single- |
| | | | | | | gene |
| | | | | | | record) |
| 3HBZCT | hydroxyisovalerate | 1 | 3.1.2.2 | E. coli | yciA | b1253 |
| | thioesterase | (or 2) | | (NC_000913.3) | | |
| HPHL | Hydroxyisopentyl- | 1 | 4.2.1.17 | E. coli | fadB | b3846 |
| | CoA hydrolyase | | | (NC_000913.3) | | |
| HMGCOARx | Hydroxymethylglutaryl- | 1 | 1.1.1.88 | Pseudomonas | mvaA | (M24015.1 |
| | CoA reductase | | | mevalonii | | is single- |
| | | | | (M24015.1) | | gene |
| | | | | | | record) |
| MK | Mevalonate kinase | 1 | 2.7.1.36 | S. cerevisiae | Erg12 | YMR208W |
| | | | | (NC_001145.3) | | |
| DMD | Diphosphomevalonate | 1 | 4.1.1.33 | S. cerevisiae | Mvd1 | YNR043W |
| | decarboxylase | | | (AY693152.1) | | |
| PMK | Phosphomevalonate | 1 | 2.7.4.2 | S. cerevisiae | Erg8 | YMR220W |
| | kinase | | | (NC_001145.3) | | |
| IDI | Isopentenyl- | 1 | 5.33.2 | E. coli | idi | b2889 |
| | diphosphate | | | (NC_000913.3) | | |
| | isomerase | | | | | |
| DMPPK | dimethylallyl | 1 | 2.7.4.26 | Methanocaldococcus | ipkA | MJ0044 |
| | diphosphate kinase | | | jannaschii | | |
| | (aka isopentenyl- | | | (NC_000909.1) | | |
| | diphosphate kinase | | | | | |
| | (IPK)) | | | | | |
| DMPK | dimethylallyl | | | | | |
| | phosphate kinase | | | | | |
| PMVD | Phosphomevalonate | 1 | 4.1.1.99 | Haloferax volcanii | mvaD | HVO_1412 |
| | decarboxylase | | | (CP001956.1) | | |
| DMPase | Phosphatase | 1 | 3.1.3.- | E. coli | aphA | b4055 |
| | | | | (NC_000913.3) | | |

### EXAMPLES

The disclosure will now be described in more detail with reference to the following non-limiting examples.

### Example 1—Expression of Isoprene Synthase in C. autoethanogenum for Production of Isoprene from CO

The inventors have identified terpene biosynthesis genes in carboxydotrophic acetogens such as C. autoethanogenum and C. ljungdahlii. A recombinant organism was engineered to produce isoprene. Isoprene is naturally emitted by some plant such as poplar to protect its leave from UV radiation. Isoprene synthase (EC 4.2.3.27) gene of Poplar was codon optimized and introduced into a carboxydotrophic acetogen C. autoethanogenum to produce isoprene from CO. The enzyme takes key intermediate DMAPP (Dimethylallyl diphosphate) of terpenoid biosynthesis to isoprene in an irreversible reaction (FIG. 1).

### Strains and Growth Conditions:

All subcloning steps were performed in E. coli using standard strains and growth conditions as described earlier (Sambrook et al, Molecular Cloning: A laboratory Manual, Cold Spring Harbour Laboratory Press, Cold Spring Harbour, 1989; Ausubel et al, Current protocols in molecular biology, John Wiley & Sons, Ltd., Hoboken, 1987).

C. autoethanogenum DSM10061 and DSM23693 (a derivative of DSM10061) were obtained from DSMZ (The German Collection of Microorganisms and Cell Cultures, Inhoffenstraße 7 B, 38124 Braunschweig, Germany). Growth was carried out at 37° C. using strictly anaerobic conditions and techniques (Hungate, 1969, Methods in Microbiology, vol. 3B. Academic Press, New York: 117-132; Wolfe, 1971, Adv. Microb. Physiol., 6: 107-146). Chemically defined PETC media without yeast extract (Table 1) and 30 psi carbon monoxide containing steel mill waste gas (collected from New Zealand Steel site in Glenbrook, NZ; composition: 44% CO, 32% N₂, 22% CO₂, 2% H₂) as sole carbon and energy source was used.

TABLE 1

| Media component | Concentration per 1.0 L of media | |
|---|---|---|
| NH₄Cl | 1 | g |
| KCl | 0.1 | g |
| MgSO₄•7H₂O | 0.2 | g |
| NaCl | 0.8 | g |
| KH₂PO₄ | 0.1 | g |
| CaCl₂ | 0.02 | g |
| Trace metal solution | 10 | ml |
| Wolfe's vitamin solution | 10 | ml |
| Resazurin (2 g/L stock) | 0.5 | ml |
| NaHCO₃ | 2 | g |
| Reducing agent | 0.006-0.008% (v/v) | |
| Distilled water | Up to 1 L, pH 5.5 | |
| | (adjusted with HCl) | |
| Wolfe's vitamin solution | per L of Stock | |
| Biotin | 2 | mg |
| Folic acid | 2 | mg |
| Pyridoxine hydrochloride | 10 | mg |
| Riboflavin | 5 | mg |
| Nicotinic acid | 5 | mg |
| Calcium D-(+)-pantothenate | 5 | mg |
| Vitamin B₁₂ | 0.1 | mg |
| p-Aminobenzoic acid | 5 | mg |
| Lipoic acid | 5 | mg |
| Thiamine | 5 | mg |
| Distilled water | To 1 L | |
| Trace metal solution | per L of stock | |
| Nitrilotriacetic Acid | 2 | g |
| MnSO₄•H₂O | 1 | g |
| Fe (SO₄)₂(NH₄)₂•6H₂O | 0.8 | g |
| CoCl₂•6H₂O | 0.2 | g |
| ZnSO₄•7H₂O | 0.2 | mg |
| CuCl₂•2H₂O | 0.02 | g |
| NaMoO₄•2H₂O | 0.02 | g |
| Na₂SeO₃ | 0.02 | g |
| NiCl₂•6H₂O | 0.02 | g |
| Na₂WO₄•2H₂O | 0.02 | g |
| Distilled water | To 1 L | |
| Reducing agent stock | per 100 mL of stock | |
| NaOH | 0.9 | g |
| Cystein•HCl | 4 | g |
| Na₂S | 4 | g |

| | |
|---|---|
| Distilled water | To 100 mL |

### Construction of Expression Plasmid:

Standard Recombinant DNA and molecular cloning techniques were used in this disclosure (Sambrook J, Fritsch E F, Maniatis T: Molecular Cloning: A laboratory Manual, Cold Spring Harbour Laboratory Press, Cold Spring Harbour, 1989; Ausubel F M, Brent R, Kingston R E, Moore D D, Seidman J G, Smith J A, Struhl K: Current protocols in molecular biology. John Wiley & Sons, Ltd., Hoboken, 1987). The isoprene synthase of Poplar tremuloides (AAQ16588.1; GI:33358229) was codon-optimized (SEQ ID NO: 21) and synthesized. A promoter region of the Pyruvate:ferredoxin oxidoreductase of C. autoethanogenum (SEQ ID NO: 22) was used to express the gene.

Genomic DNA from Clostridium autoethanogenum DSM23693 was isolated using a modified method by Bertram and Dürre (1989). A 100-ml overnight culture was harvested (6,000×g, 15 min, 4° C.), washed with potassium phosphate buffer (10 mM, pH 7.5) and suspended in 1.9 ml STE buffer (50 mM Tris-HCl, 1 mM EDTA, 200 mM sucrose; pH 8.0). 300 μl lysozyme (˜100,000 U) was added and the mixture was incubated at 37° C. for 30 min, followed by addition of 280 μl of a 10% (w/v) SDS solution and another incubation for 10 min. RNA was digested at room temperature by addition of 240 μl of an EDTA solution (0.5 M, pH 8), 20 μl Tris-HCl (1 M, pH 7.5), and 10 μl RNase A (Fermentas Life Sciences). Then, 100 μl Proteinase K (0.5 U) was added and proteolysis took place for 1-3 h at 37° C. Finally, 600 μl of sodium perchlorate (5 M) was added, followed by a phenol-chloroform extraction and an isopropanol precipitation. DNA quantity and quality was inspected spectrophotometrically. The Pyruvate:ferredoxin oxidoreductase promoter sequence was amplified by PCR using oligonucleotides Ppfor-NotI-F (SEQ ID NO: 23: AAGCGGCCGCAAAATAGTTGATAATAATGC) and Ppfor-NdeI-R (SEQ ID NO: 24: TACGCATATGAATTCCTCTCCTTTTCAAGC) using iProof High Fidelity DNA Polymerase (Bio-Rad Laboratories) and the following program: initial denaturation at 98° C. for 30 seconds, followed by 32 cycles of denaturation (98° C. for 10 seconds), annealing (50-62° C. for 30-120 seconds) and elongation (72° C. for 30-90 seconds), before a final extension step (72° C. for 10 minutes).

### Construction of Isoprene Synthase Expression Plasmid:

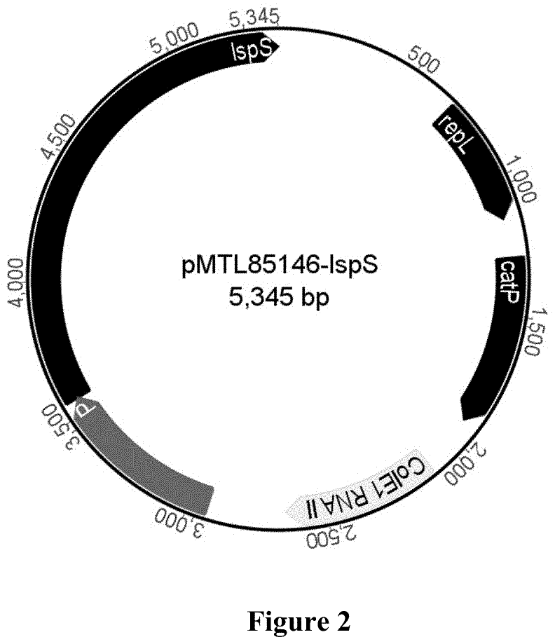

Construction of an expression plasmid was performed in E. coli DH5α-T1^{R} (Invitrogen) and XL1-Blue MRF′ Kan (Stratagene). In a first step, the amplified P_{pfor} promoter region was cloned into the E. coli-Clostridium shuttle vector pMTL85141 (FJ797651.1; Nigel Minton, University of Nottingham; Heap et al., 2009) using NotI and NdeI restriction sites, generating plasmid pMTL85146. As a second step, ispS was cloned into pMTL85146 using restriction sites NdeI and EcoRI, resulting in plasmid pMTL 85146-ispS (FIG. 2, SEQ ID NO: 25).

Transformation and Expression in C. autoethanogenum

Prior to transformation, DNA was methylated in vivo in E. coli using a synthesized hybrid Type II methyltransferase (SEQ ID NO: 63) co-expressed on a methylation plasmid (SEQ ID NO: 64) designed from methyltransferase genes from C. autoethanogenum, C. ragsdalei and C. ljungdahlii as described in US patent 2011/0236941.

Both expression plasmid and methylation plasmid were transformed into same cells of restriction negative E. coli XL1-Blue MRF′ Kan (Stratagene), which is possible due to their compatible Gram-(−) origins of replication (high copy ColE1 in expression plasmid and low copy p15A in methylation plasmid). In vivo methylation was induced by addition of 1 mM IPTG, and methylated plasmids were isolated using QIAGEN Plasmid Midi Kit (QIAGEN). The resulting mixture was used for transformation experiments with C. autoethanogenum DSM23693, but only the abundant (high-copy) expression plasmid has a Gram-(+) replication origin (repL) allowing it to replicate in Clostridia.

Transformation into C. autoethanogenum:

During the complete transformation experiment, C. autoethanogenum DSM23693 was grown in PETC media (Table 1) supplemented with 1 g/L yeast extract and 10 g/l fructose as well as 30 psi steel mill waste gas (collected from New Zealand Steel site in Glenbrook, NZ; composition: 44% CO, 32% N₂, 22% CO₂, 2% H₂) as carbon source.

To make competent cells, a 50 ml culture of C. autoethanogenum DSM23693 was subcultured to fresh media for 3 consecutive days. These cells were used to inoculate 50 ml PETC media containing 40 mM DL-threonine at an OD₆₀₀ₙₘ of 0.05. When the culture reached an OD₆₀₀ₙₘ of 0.4, the cells were transferred into an anaerobic chamber and harvested at 4,700×g and 4° C. The culture was twice washed with ice-cold electroporation buffer (270 mM sucrose, 1 mM MgCl2, 7 mM sodium phosphate, pH 7.4) and finally suspended in a volume of 600 μl fresh electroporation buffer. This mixture was transferred into a pre-cooled electroporation cuvette with a 0.4 cm electrode gap containing 1 μg of the methylated plasmid mixture and immediately pulsed using the Gene pulser Xcell electroporation system (Bio-Rad) with the following settings: 2.5 kV, 600Ω, and 25 g. Time constants of 3.7-4.0 ms were achieved. The culture was transferred into 5 ml fresh media. Regeneration of the cells was monitored at a wavelength of 600 nm using a Spectronic Helios Epsilon Spectrophotometer (Thermo) equipped with a tube holder. After an initial drop in biomass, the cells started growing again. Once the biomass has doubled from that point, the cells were harvested, suspended in 200 μl fresh media and plated on selective PETC plates (containing 1.2% Bacto™ Agar (BD)) with appropriate antibiotics 4 μg/ml Clarithromycin or 15 μg/ml thiamphenicol. After 4-5 days of inoculation with 30 psi steel mill gas at 37° C., colonies were visible.

The colonies were used to inoculate 2 ml PETC media with antibiotics. When growth occurred, the culture was scaled up into a volume of 5 ml and later 50 ml with 30 psi steel mill gas as sole carbon source.

### Confirmation of the Successful Transformation:

To verify the DNA transfer, a plasmid mini prep was performed from 10 ml culture volume using Zyppy plasmid miniprep kit (Zymo). Since the quality of the isolated plasmid was not sufficient for a restriction digest due to Clostridial exonuclease activity [Burchhardt and Dürre, 1990], a PCR was performed with the isolated plasmid with oligonucleotide pairs colE1-F (SEQ ID NO: 65: CGTCAGACCCCGTAGAAA) plus colE1-R (SEQ ID NO: 66: CTCTCCTGTTCCGACCCT). PCR was carried out using iNtRON Maximise Premix PCR kit (Intron Bio Technologies) with the following conditions: initial denaturation at 94° C. for 2 minutes, followed by 35 cycles of denaturation (94° C. for 20 seconds), annealing (55° C. for 20 seconds) and elongation (72° C. for 60 seconds), before a final extension step (72° C. for 5 minutes).

To confirm the identity of the clones, genomic DNA was isolated (see above) from 50 ml cultures of C. autoethanogenum DSM23693. A PCR was performed against the 16s rRNA gene using oligonucleotides fD1 (SEQ ID NO: 67: CCGAATTCGTCGACAACAGAGTTTGATCCTGGCTCAG) and rP2 (SEQ ID NO: 68: CCCGGGATCCAAGCTTACGGCTACCTTGTTACGACTT) [Weisberg et al., 1991] and iNtRON Maximise Premix PCR kit (Intron Bio Technologies) with the following conditions: initial denaturation at 94° C. for 2 minutes, followed by 35 cycles of denaturation (94° C. for 20 seconds), annealing (55° C. for 20 seconds) and elongation (72° C. for 60 seconds), before a final extension step (72° C. for 5 minutes). Sequencing results were at least 99.9% identity against the 16s rRNA gene (rrsA) of C. autoethanogenum (Y18178, GI:7271109).

### Expression of Isoprene Synthase Gene

qRT-PCR experiments were performed to confirm successful expression of introduced isoprene synthase gene in C. autoethanogenum.

A culture harboring isoprene synthase plasmid pMTL 85146-ispS and a control culture without plasmid was grown in 50 mL serum bottles and PETC media (Table 1) with 30 psi steel mill waste gas (collected from New Zealand Steel site in Glenbrook, NZ; composition: 44% CO, 32% N₂, 22% CO₂, 2% H₂) as sole energy and carbon source. 0.8 mL samples were taken during logarithmic growth phase at an OD₆₀₀ₙₘ of around 0.5 and mixed with 1.6 mL RNA protect reagent (Qiagen). The mixture was centrifuged (6,000×g, 5 min, 4° C.), and the cell sediment snap frozen in liquid nitrogen and stored at −80° C. until RNA extraction. Total RNA was isolated using RNeasy Mini Kit (Qiagen) according to protocol 5 of the manual. Disruption of the cells was carried out by passing the mixture through a syringe 10 times and eluted in 50 μL of RNase/DNase-free water. After DNase I treatment using DNA-Free™ Kit (Ambion), the reverse transcription step was then carried out using SuperScript III Reverse Transcriptase Kit (Invitrogen, Carlsbad, Calif., USA). RNA was checked using an Agilent Bioanalyzer 2100 (Agilent Technologies, Santa Clara, Calif., USA), Qubit Fluorometer (Invitrogen, Carlsbad, Calif., USA) and by gel electrophoresis. A non-RT control was performed for every oligonucleotide pair. All qRT-PCR reactions were performed in duplicate using a MyiQ™ Single Colour Detection System (Bio-Rad Laboratories, Carlsbad, Calif., USA) in a total reaction volume of 15 μL with 25 ng of cDNA template, 67 nM of each oligonucleotide (Table 2), and 1×iQ™ SYBR® Green Supermix (Bio-Rad Laboratories, Carlsbad, Calif., USA). The reaction conditions were 95° C. for 3 min, followed by 40 cycles of 95° C. for 15 s, 55° C. for 15 s and 72° C. for 30 s. For detection of oligonucleotide dimerisation or other artifacts of amplification, a melting-curve analysis was performed immediately after completion of the qPCR (38 cycles of 58° C. to 95° C. at 1° C./s). Two housekeeping genes (guanylate kinase and formate tetrahydrofolate ligase) were included for each cDNA sample for normalization. Determination of relative gene expression was conducted using Relative Expression Software Tool (REST©) 2008 V2.0.7 (38). Dilution series of cDNA spanning 4 log units were used to generate standard curves and the resulting amplification efficiencies to calculate concentration of mRNA.

TABLE 2
Oligonucleotides for qRT-PCR

| | Oligo- | | |
|---|---|---|---|
| | nucleotide | | SEQ ID |
| Target | Name | DNA Sequence (5′ to 3′) | NO: |
| Guanylate kinase (gnk) | GnK-F | TCAGGACCTTCTGGAACTGG | 108 |
| | GnK-R | ACCTCCCCTTTTCTTGGAGA | 109 |
| Formate | FoT4L-F | CAGGTTTCGGTGCTGACCTA | 110 |
| tetrahydrofolate ligase | FoT4L-F | AACTCCGCCGTTGTATTTCA | 111 |
| (FoT4L) | | | |
| Isoprene Synthase | ispS-F | AGG CTG AAT TTC TTA CAC TTC | 69 |
| | | TTG A | |
| | ispS-R | GTA ACT CCA TCA AAT CCT | 70 |
| | | CCA CTA C | |

While no amplification was observed with the wild-type strain using oligonucleotide pair ispS, a signal with the ispS oligonucleotide pair was measured for the strain carrying plasmid pMTL 85146-ispS, confirming successful expression of the ispS gene.

### Example 2—Expression of Isopentenyl-Diphosphate Delta-Isomerase to Convert Between Key Terpene Precursors DMAPP (Dimethylallyl Diphosphate) and IPP (Isopentenyl Diphosphate)

Availability and balance of precursors DMAPP (Dimethylallyl diphosphate) and IPP (Isopentenyl diphosphate) is crucial for production of terpenes. While the DXS pathway synthesizes both IPP and DMAPP equally, in the mevalonate pathway the only product is IPP. Production of isoprene requires only the precursor DMAPP to be present in conjunction with an isoprene synthase, while for production of higher terpenes and terpenoids, it is required to have equal amounts of IPP and DMAPP available to produce Geranyl-PP by a geranyltransferase.

### Construction of Isopentenyl-Diphosphate Delta-Isomerase Expression Plasmid:

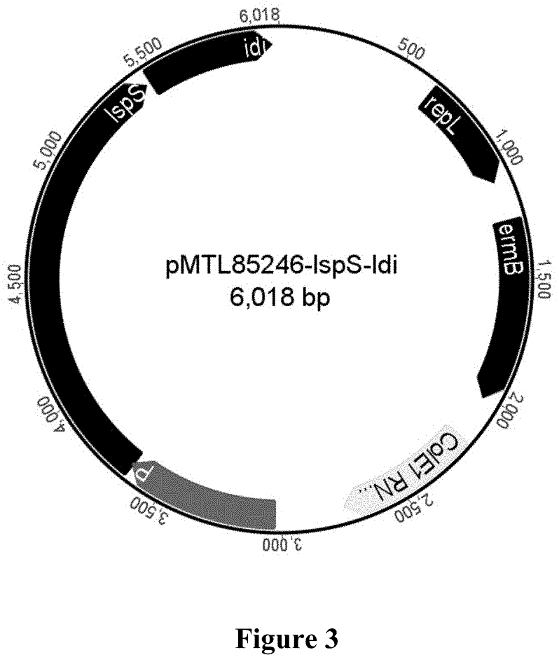

An Isopentenyl-diphosphate delta-isomerase gene idi from C. beijerinckii (Gene ID:5294264), encoding an Isopentenyl-diphosphate delta-isomerase (YP_001310174.1), was cloned downstream of ispS. The gene was amplified using oligonucleotide Idi-Cbei-SacI-F (SEQ ID NO: 26: GTGAGCTCGAAAGGGGAAATTAAATG) and Idi-Cbei-KpnI-R (SEQ ID NO: 27: ATGGTACCCCAAATCTTTATTTAGACG) from genomic DNA of C. beijerinckii NCIMB8052, obtained using the same method as described above for C. autoethanogenum. The PCR product was cloned into vector pMTL 85146-ispS using SacI and KpnI restriction sites to yield plasmid pMTL85146-ispS-idi (SEQ ID NO: 28). The antibiotic resistance marker was exchanged from catP to ermB (released from vector pMTL82254 (FJ797646.1; Nigel Minton, University of Nottingham; Heap et al., 2009) using restriction enzymes PmeI and FseI to form plasmid pMTL85246-ispS-idi (FIG. 3).

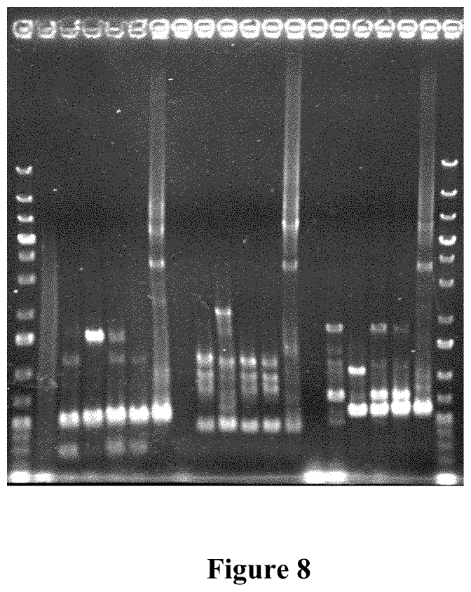

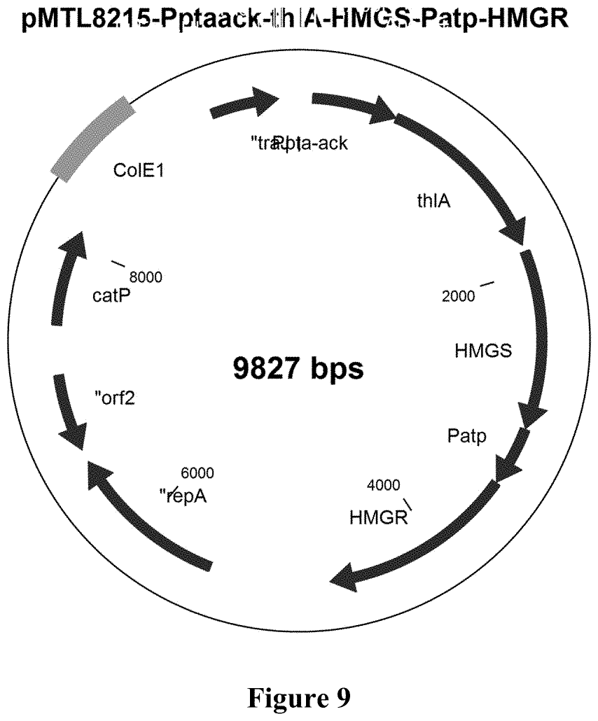

Transformation and expression in C. autoethanogenum was carried out as described for plasmid pMTL 85146-ispS. After successful transformation, growth experiment was carried out in 50 mL 50 mL serum bottles and PETC media (Table 1) with 30 psi steel mill waste gas (collected from New Zealand Steel site in Glenbrook, NZ; composition: 44% CO, 32% N₂, 22% CO₂, 2% H₂) as sole energy and carbon source. To confirm that the plasmid has been successfully introduced, plasmid mini prep DNA was carried out from transformants as described previously. PCR against the isolated plasmid using oligonucleotide pairs that target colE1 (colE1-F: SEQ ID NO: 65: CGTCAGACCCCGTAGAAA and colE1-R: SEQ ID NO: 66: CTCTCCTGTTCCGACCCT), ermB (ermB-F: SEQ ID NO: 106: TTTGTAATTAAGAAGGAG and ermB-R: SEQ ID NO: 107: GTAGAATCCTTCTTCAAC) and idi (Idi-Cbei-SacI-F: SEQ ID NO: 26: GTGAGCTCGAAAGGGGAAATTAAATG and Idi-Cbei-KpnI-R: SEQ ID NO: 27: ATGGTACCCCAAATCTTTATTTAGACG) confirmed transformation success (FIG. 8). Similarly, genomic DNA from these transformants were extracted, and the resulting 16s rRNA amplicon using oligonucleotides fD1 and rP2 (see above) confirmed 99.9% identity against the 16S rRNA gene of C. autoethanogenum (Y18178, GI:7271109).

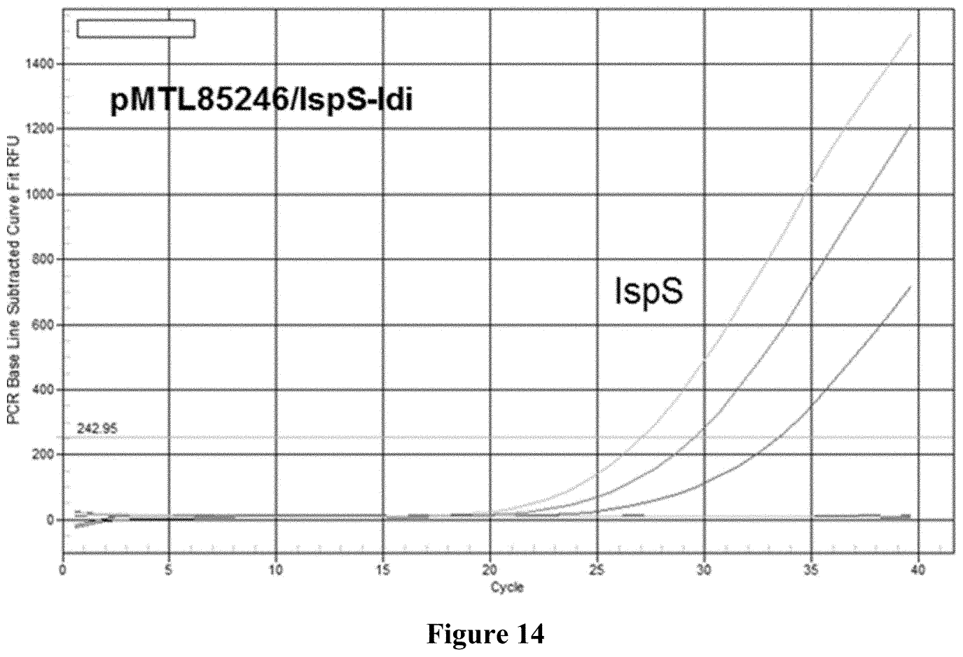

Successful confirmation of gene expression was carried out as described above using a oligonucleotide pair against Isopentenyl-diphosphate delta-isomerase gene idi (idi-F, SEQ ID NO: 71: ATA CGT GCT GTA GTC ATC CAA GAT A and idiR, SEQ ID NO: 72: TCT TCA AGT TCA CAT GTA AAA CCC A) and a sample from a serum bottle growth experiment with C. autoethanogenum carrying plasmid pMTL 85146-ispS-idi. A signal for the isoprene synthase gene ispS was also observed (FIG. 14).

### Example 3—Overexpression of DXS Pathway

To improve flow through the DXS pathway, genes of the pathway were overexpressed. The initial step of the pathway, converting pyruvate and D-glyceraldehyde-3-phosphate (G3P) into deoxyxylulose 5-phosphate (DXP/DXPS/DOXP), is catalyzed by an deoxyxylulose 5-phosphate synthase (DXS).

### Construction of DXS Overexpression Expression Plasmid:

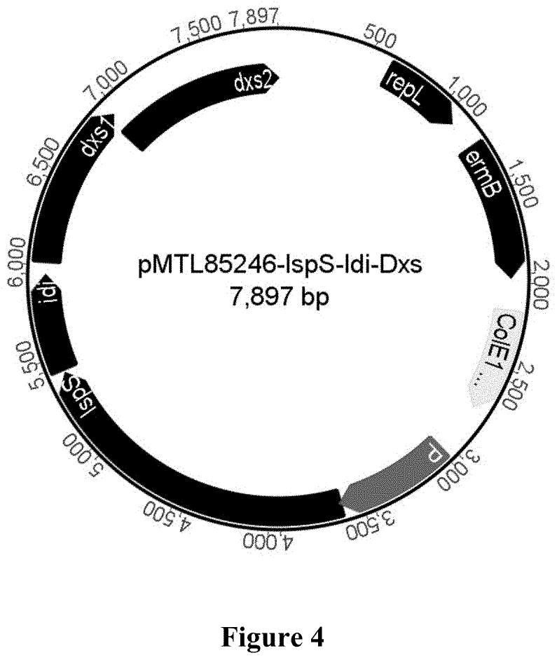

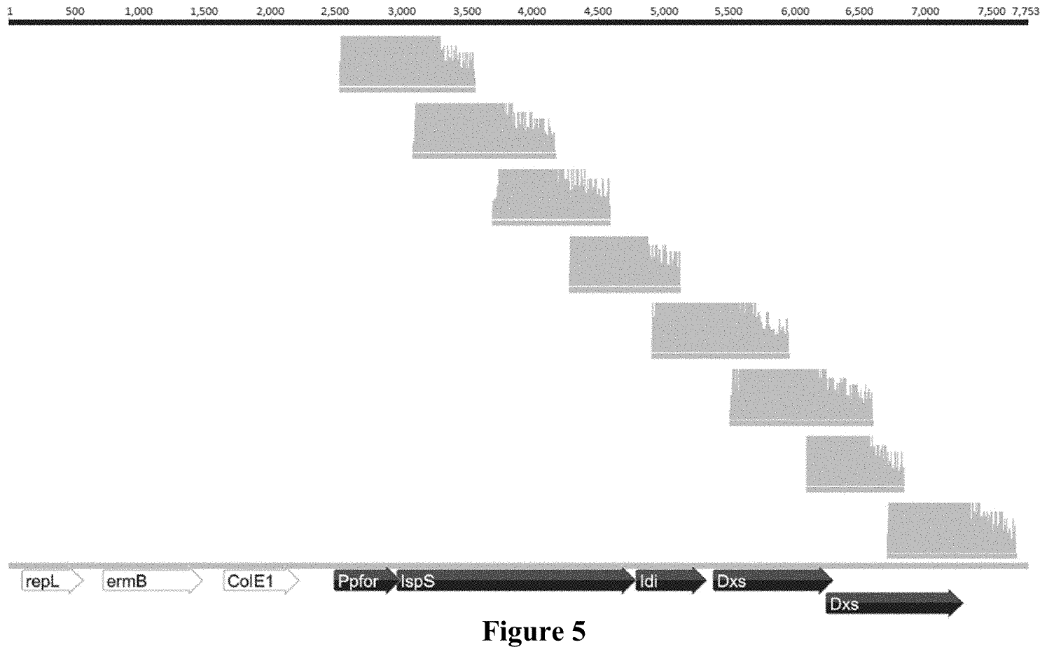

The dxs gene of C. autoethanogenum was amplified from genomic DNA with oligonucleotides Dxs-SalI-F (SEQ ID NO: 29: GCAGTCGACTTTATTAAAGGGATAGATAA) and Dxs-XhoI-R (SEQ ID NO: 30: TGCTCGAGTTAAAATATATGACTTACCTCTG) as described for other genes above. The amplified gene was then cloned into plasmid pMTL85246-ispS-idi with SalI and XhoI to produce plasmid pMTL85246-ispS-idi-dxs (SEQ ID NO: 31 and FIG. 4). DNA sequencing using oligonucleotides given in Table 3 confirmed successful cloning of ispS, idi, and dxs without mutations (FIG. 5). The ispS and idi genes are as described in example 1 and 2 respectively.

TABLE 3
Oligonucleotides for sequencing

| Oligonucleotide | | SEQ ID |
|---|---|---|
| Name | DNA Sequence (5′ to 3′) | NO: |
| M13R | CAGGAAACAGCTATGAC | 32 |
| Isoprene-seq1 | GTTATTCAAGCTACACCTTT | 33 |
| Isoprene-seq2 | GATTGGTAAAGAATTAGCTG | 34 |
| Isoprene-seq3 | TCAAGAAGCTAAGTGGCT | 35 |
| Isoprene-seq4 | CTCACCGTAAAGGAACA | 36 |
| Isoprene-seq5 | GCTAGCTAGAGAAATTAGAA | 37 |
| Isoprene-seq6 | GGAATGGCAAAATATCTTGA | 38 |
| Isoprene-seq7 | GAAACACATCAGGGAATATT | 39 |

Transformation and Expression in C. autoethanogenum

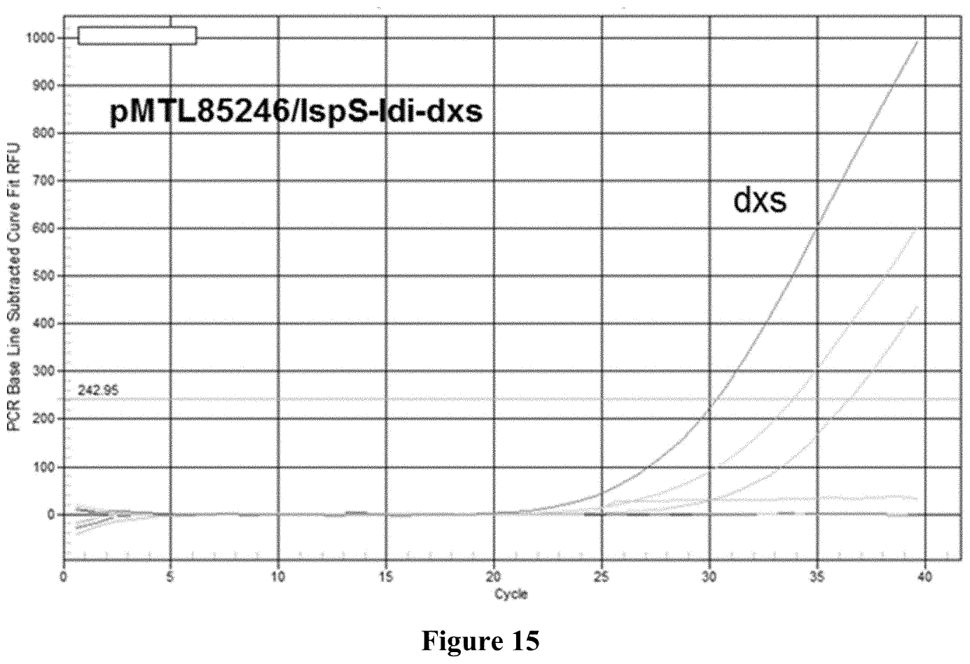

Transformation and expression in C. autoethanogenum was carried out as described for plasmid pMTL 85146-ispS. After successful transformation, a growth experiment was carried out in 50 mL 50 mL serum bottles and PETC media (Table 1) with 30 psi steel mill waste gas (collected from New Zealand Steel site in Glenbrook, NZ; composition: 44% CO, 32% N₂, 22% CO₂, 2% H₂) as sole energy and carbon source. Confirmation of gene expression was carried out as described above from a sample collected at OD₆₀₀ₙₘ=0.75. Oligonucleotide pair dxs-F (SEQ ID NO: 73: ACAAAGTATCTAAGACAGGAGGTCA) and dxs-R (SEQ ID NO: 74: GATGTCCCACATCCCATATAAGTTT) was used to measure expression of gene dxs in both wild-type strain and strain carrying plasmid pMTL 85146-ispS-idi-dxs. mRNA levels in the strain carrying the plasmid were found to be over 3 times increased compared to the wild-type (FIG. 15). Biomass was normalized before RNA extraction.

### Example 4—Introduction and Expression of Mevalonate Pathway

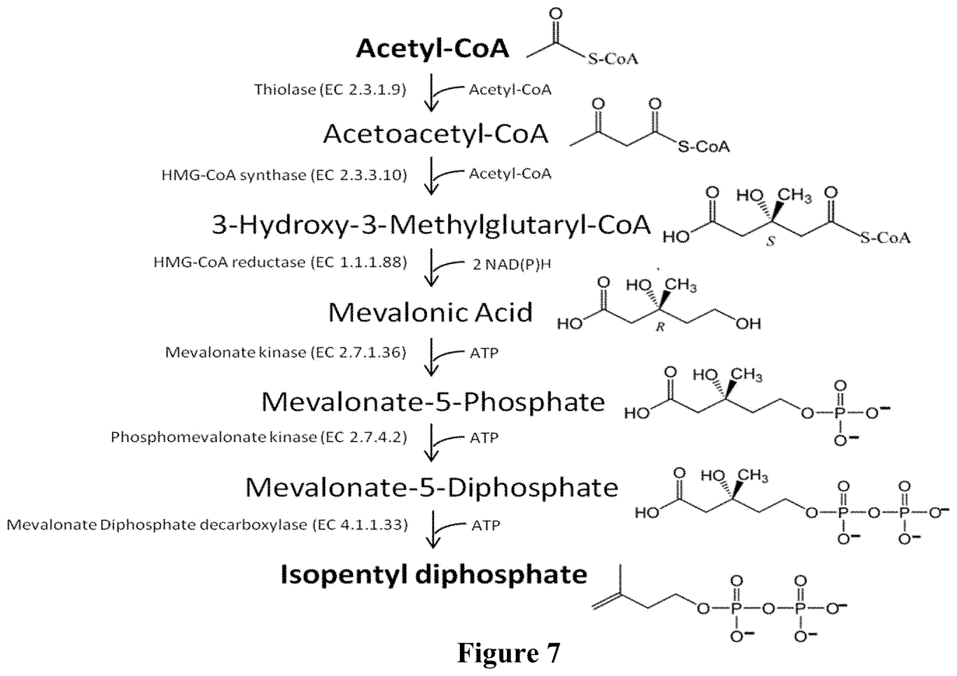

The first step of the mevalonate pathway (FIG. 7) is catalyzed by a thiolase that converts two molecules of acetyl-CoA into acetoacetyl-CoA (and HS-CoA). This enzyme has been successfully expressed in carboxydotrophic acetogens Clostridium autoethanogenum and C. ljungdahlii by the same inventors (US patent 2011/0236941). Constructs for the remaining genes of the mevalonate pathway have been designed.

### Construction of Mevalonate Expression Plasmid:

Standard recombinant DNA and molecular cloning techniques were used (Sambrook, J., and Russell, D., Molecular cloning: A Laboratory Manual 3rd Ed., Cold Spring Harbour Lab Press, Cold Spring Harbour, N Y, 2001). The three genes required for mevalonate synthesis via the upper part of the mevalonate pathway, i.e., thiolase (thlA/vraB), HMG-CoA synthase (HMGS) and HMG-CoA reductase (HMGR), were codon-optimised as an operon (P_{ptaack}-thlA/vraB HMGS-Pₐₜₚ-HMGR).

The Phosphotransacetylase/Acetate kinase operon promoter (P_{pta-ack}) of C. autoethanogenum (SEQ ID NO: 61) was used for expression of the thiolase and HMG-CoA synthase while a promoter region of the ATP synthase (Pₐₜₚ) of C. autoethanogenum was used for expression of the HMG-CoA reductase. Two variants of thiolase, thlA from Clostridium acetobutylicum and vraB from Staphylococcus aureus, were synthesised and flanked by NdeI and EcoRI restriction sites for further sub-cloning. Both HMG-CoA synthase (HMGS) and HMG-CoA reductase (HMGR) were synthesised from Staphylococcus aureus and flanked by EcoRI-SacI and KpnI-XbaI restriction sites respectively for further sub-cloning. All optimized DNA sequences used are given in Table 4.

TABLE 4
Sequences of mevalonate expression plasmid

| | | SEQ |
|---|---|---|
| Description | Source | ID NO: |
| Thiolase (thlA) | Clostridium | 40 |
| | acetobutylicum | |
| | ATCC 824; | |
| | NC_003030.1; | |
| | GI: 1119056 | |
| Acetyl-CoA c- | Staphylococcus aureus | 41 |
| acetyltransferase | subsp. aureus Mu50; | |
| (vraB) | NC_002758.2; region: | |
| | 652965 . . . 654104; | |
| | including GI: 15923566 | |
| 3-hydroxy-3- | Staphylococcus aureus | 42 |
| methylglutaryl- | subsp. aureus Mu50; | |
| CoA synthase (HMGS) | NC_002758.2; region: | |
| | 2689180 . . . 2690346; | |
| | including GI: 15925536 | |
| Hydroxymethyl- | Staphylococcus aureus | 43 |
| glutaryl-CoA | subsp. aureus Mu50; | |
| reductase (HMGR) | NC_002758.2; region: | |
| | complement(2687648 . . . | |
| | 2688925); including | |
| | GI: 15925535 | |
| Phosphotrans- | Clostridium | 44 |
| acetylase-acetate | autoethanogenum | |
| kinase operon (P_{pta-ack}) | DSM10061 | |
| ATP synthase | Clostridium | 45 |
| promoter (Pₐₜₚ) | autoethanogenum | |
| | DSM10061 | |

The ATP synthase promoter (Pₐₜₚ) together with the hydroxymethylglutaryl-CoA reductase (HMGR) was amplified using oligonucleotides pUC57-F (SEQ ID NO: 46: AGCAGATTGTACTGAGAGTGC) and pUC57-R (SEQ ID NO: 47: ACAGCTATGACCATGATTACG) and pUC57-Patp-HMGR as a template. The 2033 bp amplified fragment was digested with SacI and XbaI and ligated into the E. coli-Clostridium shuttle vector pMTL 82151 (FJ7976; Nigel Minton, University of Nottingham, UK; Heap et al., 2009, J Microbiol Methods. 78: 79-85) resulting in plasmid pMTL 82151-Patp-HMGR (SEQ ID NO: 76).

3-hydroxy-3-methylglutaryl-CoA synthase (HMGS) was amplified from the codon-synthesised plasmid pGH-seq3.2 using oligonucleotides EcoRI-HMGS_F (SEQ ID NO: 77: AGCCGTGAATTCGAGGCTTTTACTAAAAACA) and EcoRI-HMGS_R (SEQ ID NO: 78: AGGCGTCTAGATGTTCGTCTCTACAAATAATT). The 1391 bp amplified fragment was digested with SacI and EcoRI and ligated into the previously created plasmid pMTL 82151-Patp-HMGR to give pMTL 82151-HMGS-Patp-HMGR (SEQ ID NO: 79). The created plasmid pMTL 82151-HMGS-Patp-HMGR (SEQ ID NO: 79) and the 1768 bp codon-optimised operon of P_{ptaack}-thlA/vraB were both cut with NotI and EcoRI. A ligation was performed and subsequently transformed into E. coli XL1-Blue MRF′ Kan resulting in plasmid pMTL8215-P_{ptaack}-thlA/vraB-HMGS-Pₐₜₚ-HMGR (SEQ ID NO: 50).

The five genes required for synthesis of terpenoid key intermediates from mevalonate via the bottom part of the mevalonate pathway, i.e., mevalonate kinase (MK), phosphomevalonate kinase (PMK), mevalonate diphosphate decarboxylase (PMD), isopentenyl-diphosphate delta-isomerase (idi) and isoprene synthase (ispS) were codon-optimised by ATG:Biosynthetics GmbH (Merzhausen, Germany). Mevalonate kinase (MK), phosphomevalonate kinase (PMK) and mevalonate diphosphate decarboxylase (PMD) were obtained from Staphylococcus aureus.

The promoter region of the RNF Complex (P_{rnf}) of C. autoethanogenum (SEQ ID NO: 62) was used for expression of mevalonate kinase (MK), phosphomevalonate kinase (PMK) and mevalonate diphosphate decarboxylase (PMD), while the promoter region of the Pyruvate:ferredoxin oxidoreductase (P_{for}) of C. autoethanogenum (SEQ ID NO: 22) was used for expression of isopentenyl-diphosphate delta-isomerase (idi) and isoprene synthase (ispS). All DNA sequences used are given in Table 5. The codon-optimised Prnf-MK was amplified from the synthesised plasmid pGH-Prnf-MK-PMK-PMD with oligonucleotides NotI-XbaI-Prnf-MK_F (SEQ ID NO: 80: ATGCGCGGCCGCTAGGTCTAGAATATCGATACAGATAAAAAAATATATAATACAG) and SalI-Prnf-MK_R (SEQ ID NO: 81: TGGTTCTGTAACAGCGTATTCACCTGC). The amplified gene was then cloned into plasmid pMTL83145 (SEQ ID NO: 49) with NotI and SalI to produce plasmid pMTL8314-Prnf-MK (SEQ ID NO: 82). This resulting plasmid and the 2165 bp codon optimised fragment PMK-PMD was subsequently digested with SalI and HindIII. A ligation was performed resulting in plasmid pMTL 8314-Prnf-MK-PMK-PMD (SEQ ID NO: 83).

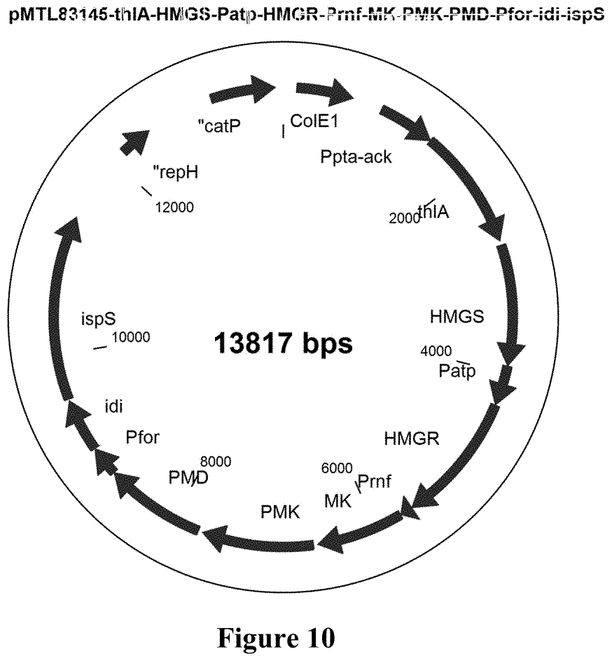

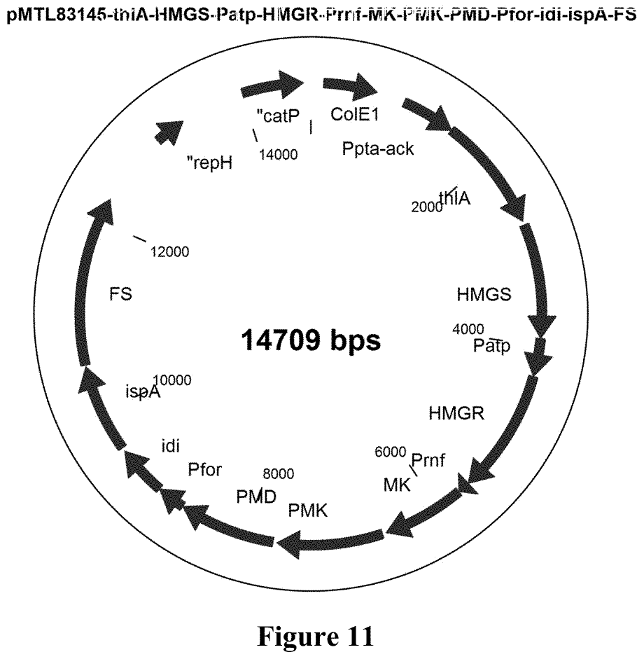

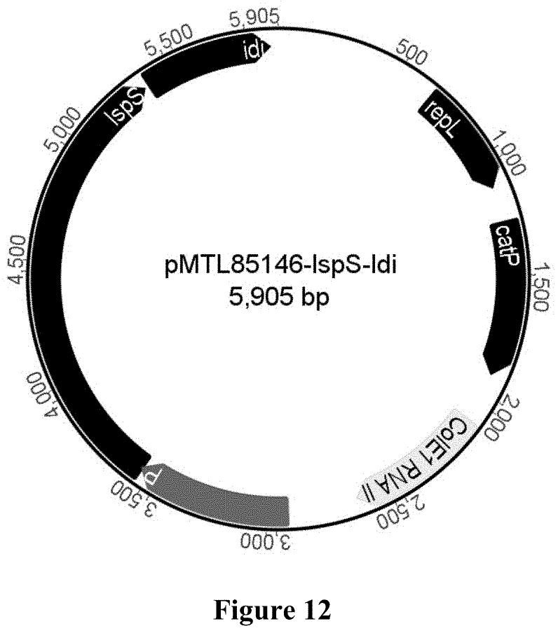

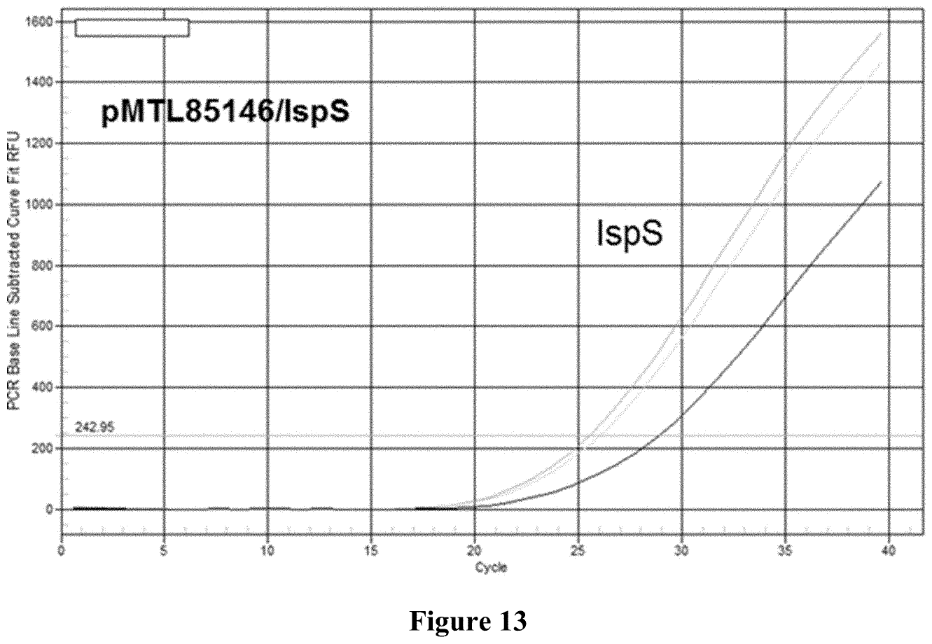

The isoprene expression plasmid without the mevalonate pathway was created by ligating the isoprene synthase (ispS) flanked by restriction sites AgeI and NheI to the previously created farnesene plasmid, pMTL 8314-Prnf-MK-PMK-PMD-Pfor-idi-ispA-FS (SEQ ID NO:91) to result in plasmid pMTL8314-Prnf-MK-PMK-PMD-Pfor-idi-ispS (SEQ ID NO:84). The final isoprene expression plasmid, pMTL 8314-Pptaack-thlA-HMGS-Patp-HMGR-Prnf-MK-PMK-PMD-Pfor-idi-ispS (SEQ ID NO: 58, FIG. 10) is created by ligating the 4630 bp fragment of Pptaack-thlA-HMGS-Patp-HMGR from pMTL8215-Pptaack-thlA-HMGS-Patp-HMGR (SEQ ID NO: 50) with pMTL 8314-Prnf-MK-PMK-PMD-Pfor-idi-ispS (SEQ ID NO: 84) using restriction sites NotI and XbaI.

TABLE 5
Sequences of isoprene expression plasmid
from mevalonate pathway

| | | SEQ ID |
|---|---|---|
| Description | Source | NO: |
| Mevalonate | Staphylococcus aureus | 51 |
| kinase (MK) | subsp. aureus Mu50; | |
| | NC_002758.2; region: | |
| | 665080 . . . 665919; | |
| | including GI: 15923580 | |
| Phosphome- | Staphylococcus aureus | 52 |
| valonate | subsp. aureus Mu50; | |
| kinase (PMK) | NC_002758.2; region: | |
| | 666920 . . . 667996; | |
| | including GI: 15923582 | |
| Mevalonate | Staphylococcus aureus | 53 |
| diphosphate | subsp. aureus Mu50; | |
| decarboxylase | NC_002758.2; region: | |
| (PMD) | 665924 . . . 666907; | |
| | including GI: 15923581 | |
| Isoprene | isoprene synthase of | 21 |
| synthase (isIS) | Poplar tremuloides | |
| | (AAQ16588.1; GI: 33358229) | |
| Isopentenyl- | Clostridium beijerinckii | 54 |
| diphosphate | NCIMB 8052; | |
| delta-isomerase | YP_001310174.1; region: | |
| (idi) | complement(3597793 . . . | |
| | 3598308); including | |
| | GI: 150017920 | |
| RNF Complex | Clostridium autoethanogenum | 55 |
| promoter (P_{rnf}) | DSM10061 | |

### Example 5—Introduction of Farnesene Synthase in C. autoethanogenum for Production of Farnesene from CO Via the Mevalonate Pathway

Instead of producing isoprene directly from terpenoid key intermediates IPP and DMAPP then using this to synthesise longer chain terpenes, it is also possible to synthesise longer chain terpenes, such as C10 Monoterpenoids or C15 Sesquiterpenoids, directly via a geranyltransferase (see Table 6). From C15 Sesquiterpenoid building block farnesyl-PP it is possible to produce farnesene, which, similarly to ethanol, can be used as a transportation fuel.

### Construction of Farnesene Expression Plasmid

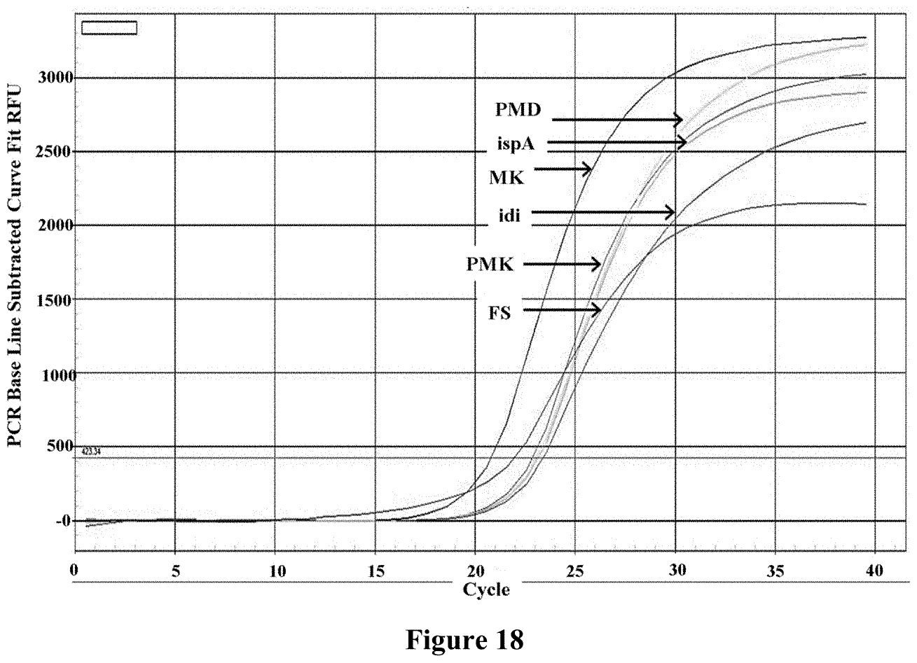

The two genes required for farnesene synthesis from IPP and DMAPP via the mevalonate pathway, i.e., geranyltranstransferase (ispA) and alpha-farnesene synthase (FS) were codon-optimised. Geranyltranstransferase (ispA) was obtained from Escherichia coli str. K-12 substr. MG1655 and alpha-farnesene synthase (FS) was obtained from Malus x domestica. All DNA sequences used are given in Table 6. The codon-optimised idi was amplified from the synthesised plasmid pMTL83245-Pfor-FS-idi (SEQ ID NO: 85) with via the mevalonate pathways idi_F (SEQ ID NO: 86: AGGCACTCGAGATGGCAGAGTATATAATAGCAGTAG) and idi_R2 (SEQ ID NO:87: AGGCGCAAGCTTGGCGCACCGGTTTATTTAAATATCTTATTTTCAGC). The amplified gene was then cloned into plasmid pMTL83245-Pfor with XhoI and HindIII to produce plasmid pMTL83245-Pfor-idi (SEQ ID NO: 88). This resulting plasmid and the 1754 bp codon optimised fragment of farnesene synthase (FS) was subsequently digested with HindIII and NheI. A ligation was performed resulting in plasmid pMTL83245-Pfor-idi-FS (SEQ ID NO: 89). The 946 bp fragment of ispA and pMTL83245-Pfor-idi-FS was subsequently digested with AgeI and HindIII and ligated to create the resulting plasmid pMTL83245-Pfor-idi-ispA-FS (SEQ ID NO: 90). The farnesene expression plasmid without the upper mevalonate pathway was created by ligating the 2516 bp fragment of Pfor-idi-ispA-FS from pMTL83245-Pfor-idi-ispA-FS to pMTL 8314-Prnf-MK-PMK-PMD to result in plasmid pMTL 8314-Prnf-MK-PMK-PMD-Pfor-idi-ispA-FS (SEQ ID NO: 91). The final farnesene expression plasmid pMTL83145-thlA-HMGS-Patp-HMGR-Prnf-MK-PMK-PMD-Pfor-idi-ispA-FS (SEQ ID NO: 59 and FIG. 18) is created by ligating the 4630 bp fragment of Pptaack-thlA-HMGS-Patp-HMGR from pMTL8215-Pptaack-thlA-HMGS-Patp-HMGR (SEQ ID NO: 50) with pMTL 8314-Prnf-MK-PMK-PMD-Pfor-idi-ispA-FS (SEQ ID NO: 91) using restriction sites NotI and XbaI.

TABLE 6
Sequences of farnesene expression plasmid
from mevalonate pathway

| | | SEQ ID |
|---|---|---|
| Description | Source | NO: |
| Geranyl- | Escherichia coli str. K-12 | 56 |
| transtransferase | substr. MG1655; | |
| (ispA) | NC_000913.2; region: | |
| | complement(439426 . . . | |
| | 440325); including | |
| | GI: 16128406 | |
| Alpha- | Malus × domestica; | 57 |
| farnesene | AY787633.1; GI: 60418690 | |
| synthase (FS) | | |

Transformation into C. autoethanogenum

Transformation and expression in C. autoethanogenum was carried out as described in example 1.

### Confirmation of Successful Transformation

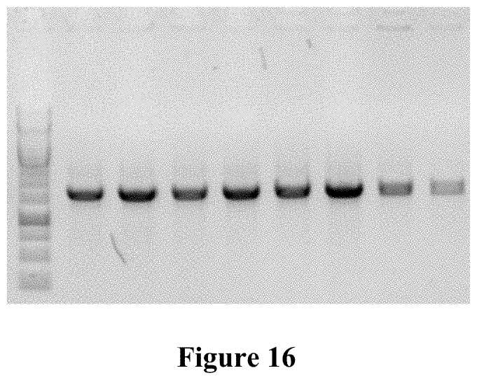

The presence of pMTL8314-Prnf-MK-PMK-PMD-Pfor-idi-ispA-FS (SEQ ID NO: 59) was confirmed by colony PCR using oligonucleotides repHF (SEQ ID NO: 92:AAGAAGGGCGTATATGAAAACTTGT) andcatR (SEQ ID NO: 93: TTCGTTTACAAAACGGCAAATGTGA) which selectively amplifies a portion of the garm +ve perplicon and most of the cat gene on the pMTL83 lxxx series plasmids. Yielding a band of 1584 bp (FIG. 16).

Expression of Lower Mevalonate Pathway in C. autoethanogenum

Confirmation of expression of the lower mevalonate pathway genes Mevalonate kinase (MK SEQ ID NO: 51), Phosphomevalonate Kinase (PMK SEQ ID NO: 52), Mevalonate Diphosphate Decarboxylase (PMD SEQ ID NO: 53), Isopentyl-diphosphate Delta-isomerase (idi; SEQ ID NO: 54), Geranyltranstransferase (ispA; SEQ ID NO: 56) and Farnesene synthase (FS SEQ ID NO: 57) was done as described above in example 1. Using oligonucleotides listed in table 7.

TABLE 7
List of oligonucleotides used for the detection of expression of the genes in
the lower mevalonate pathway carried on plasmid pMTL8314Prnf-MK-PMK-PMD-Pfor-
idi-ispA-FS (SEQ ID NO: 91)

| | Oligonucleotide | | SEQ ID |
|---|---|---|---|
| Target | Name | DNA Sequence (5′ to 3′) | NO: |
| Mevalonate kinase | MK-RTPCR-F | GTGCTGGTAGAGGTGGTTCA | 94 |
| | MK-RTPCR-R | CCAAGTATGTGCTGCACCAG | 95 |
| Phosphomevalonate | PMK-RTPCR-F | ATATCAGACCCACACGCAGC | 96 |
| Kinase | PMK-RTPCR-R | AATGCTTCATTGCTATGTCACATG | 97 |
| Mevalonate | PMD-RTPCR-F | GCAGAAGCAAAGGCAGCAAT | 98 |
| Diphosphate | PMD-RTPCR-R | TTGATCCAAGATTTGTAGCATGC | 99 |
| Decarboxylase | | | |
| Isopentyl-diphosphate | idi-RTPCR-F | GGACAAACACTTGTTGTAGTCACC | 100 |
| Delta-isomerase | idi-RTPCR-R | TCAAGTTCGCAAGTAAATCCCA | 101 |
| Geranyltranstransferase | ispA-RTPCR-F | ACCAGCAATGGATGACGATG | 102 |
| | ispA-RTPCR-R | AGTTTGTAAAGCGTCACCTGC | 103 |
| Farnesene synthase | FS-RTPCR-F | AAGCTAGTAGATGGTGGGCT | 104 |
| | FS-RTPCR-R | AATGCTACACCTACTGCGCA | 105 |

Rt-PCR data confirming expression of all genes in the lower mevalonate pathway is shown in FIG. 18, this data is also summarised in Table 8.

TABLE 8
Average CT values for the genes genes Mevalonate kinase
(MK SEQ ID NO: 51), Phosphomevalonate Kinase (PMK
SEQ ID NO: 52), Mevalonate Diphosphate Decarboxylase
(PMD SEQ ID NO: 53), Isopentyl-diphosphate Delta-isomerase
(idi SEQ ID NO: 54), Geranyltranstransferase (ispA SEQ ID
NO: 56) and Farnesene synthase (FS SEQ ID NO: 57). for
two independent samples taken from the two starter cultures
for the mevalonate feeding experiment (see below).

| Gene | Sample 1 (Ct Mean) | Sample 2 (Ct Mean) |
|---|---|---|
| MK | 21.9 | 20.82 |
| PMK | 23.64 | 22.81 |
| PMD | 24 | 22.83 |
| Idi | 24.23 | 27.54 |
| ispA | 23.92 | 23.22 |
| FS | 21.28 (single Ct) | 21.95 (single Ct) |
| HK (rho) | 31.5 | 28.88 |

Production of Alpha-Farnesene from Mevalonate

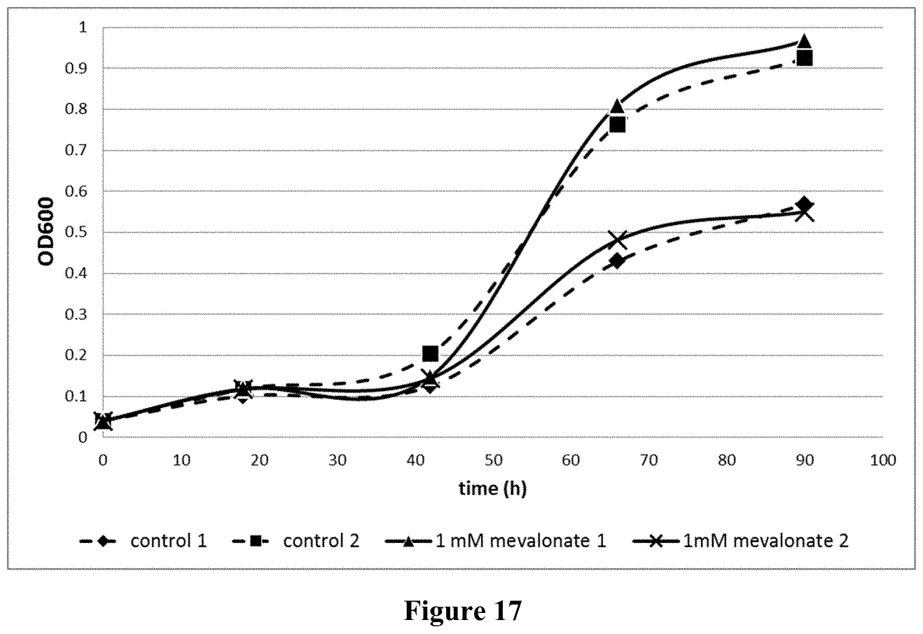

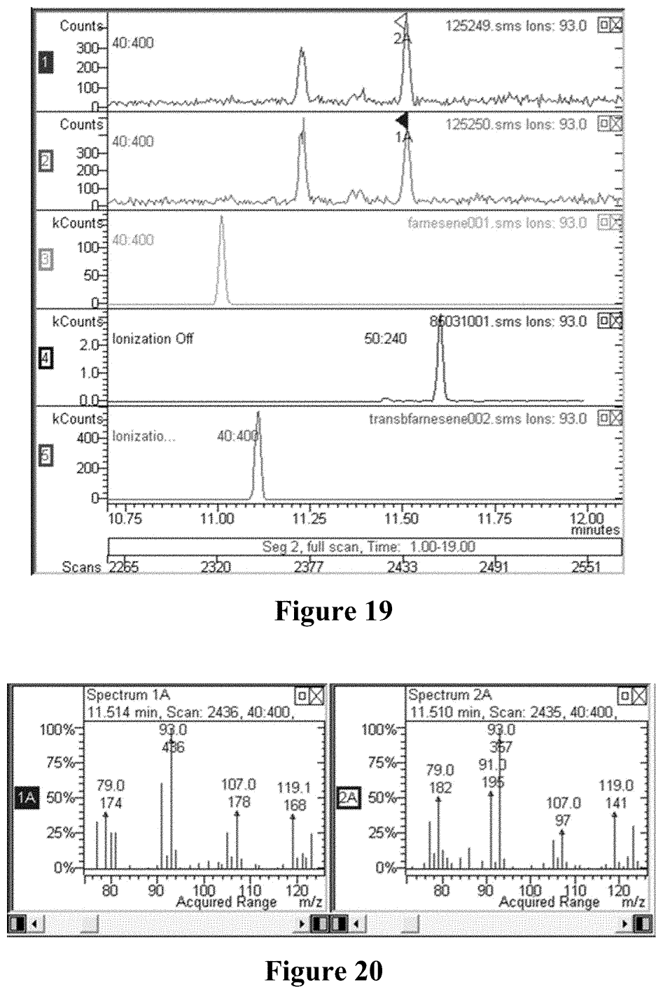

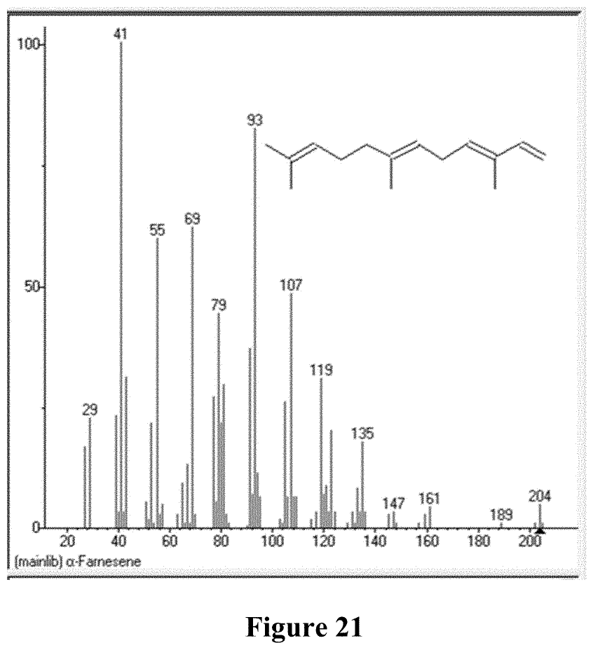

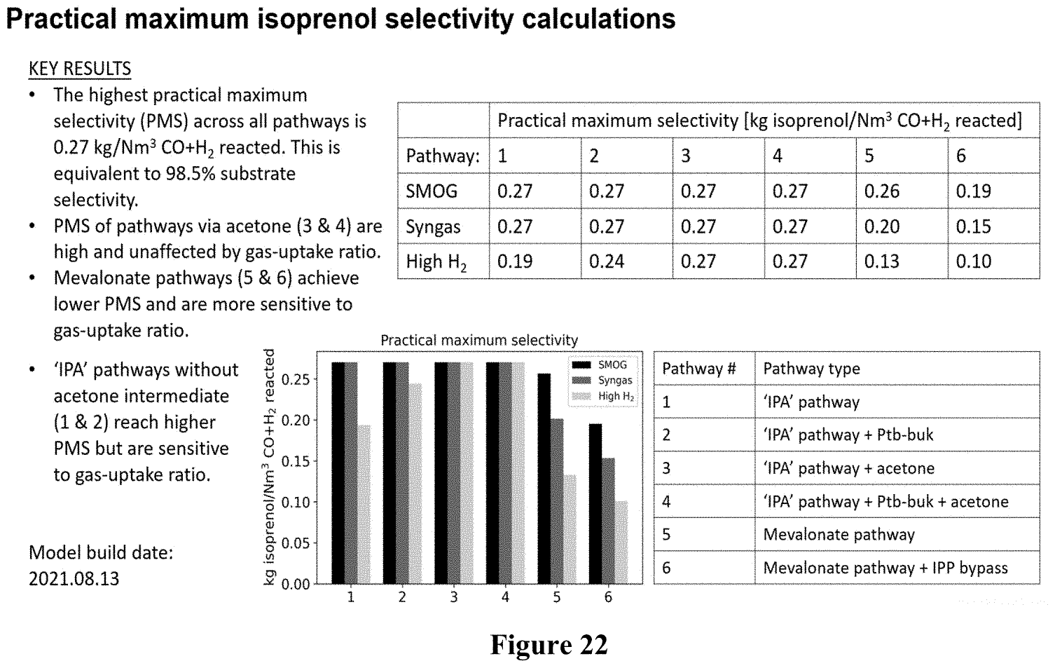

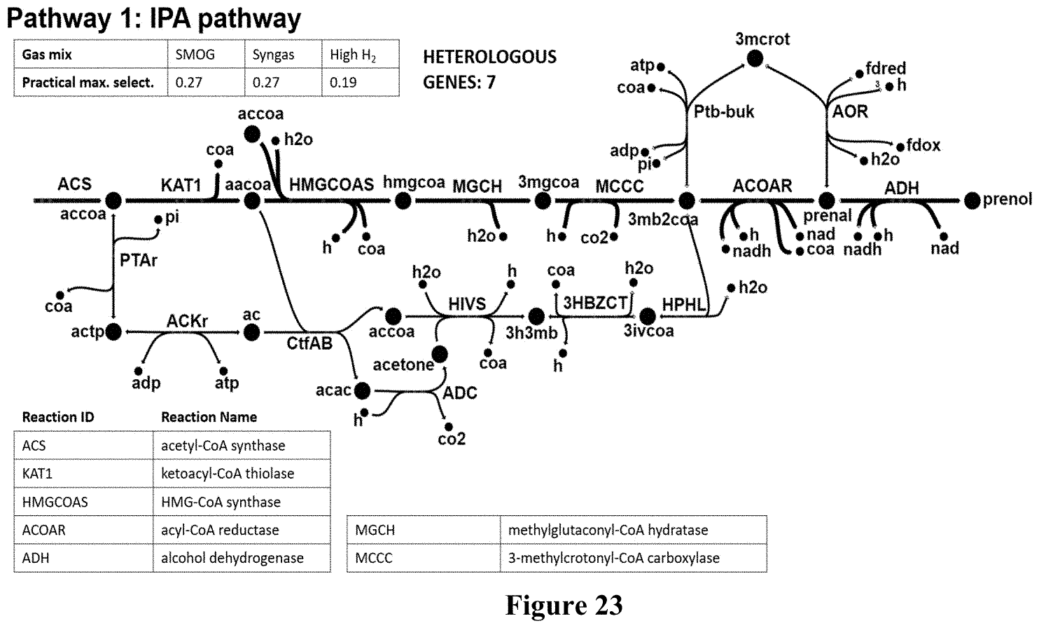

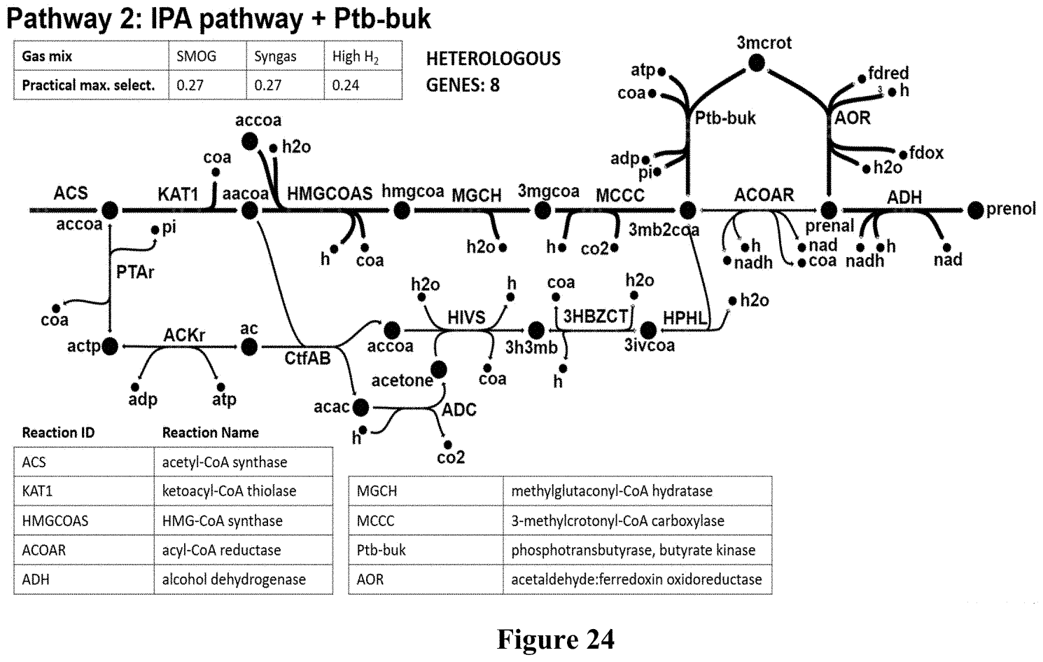

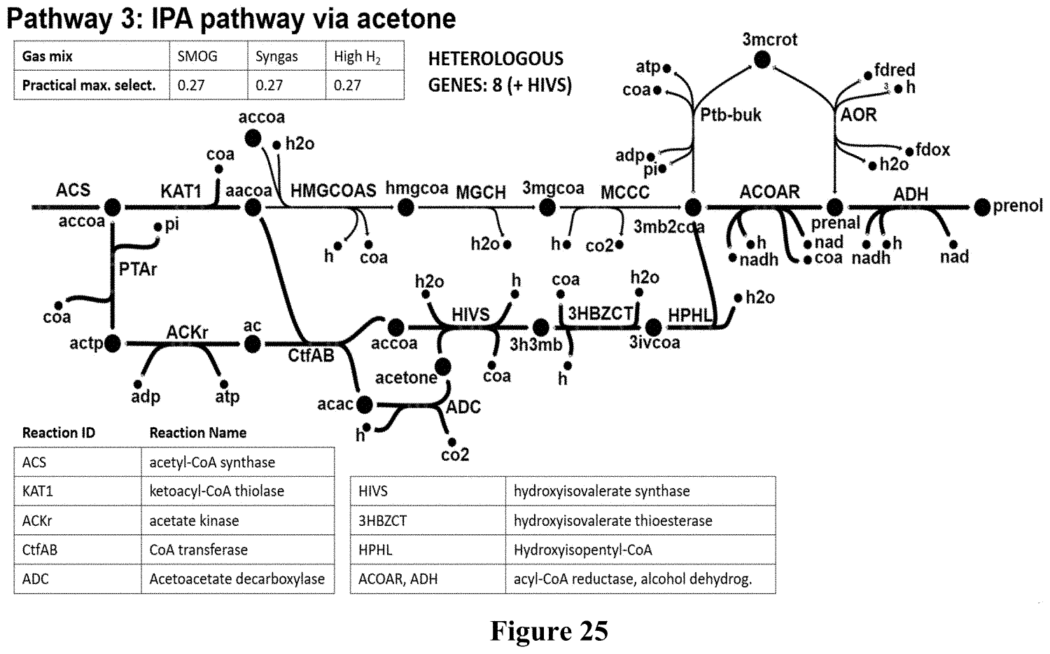

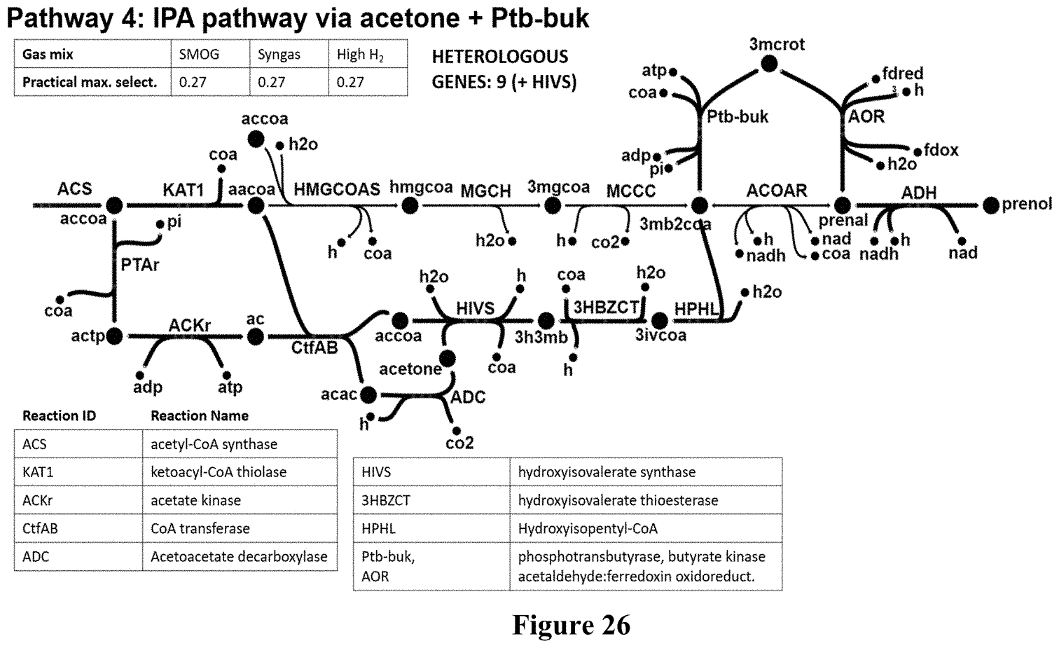

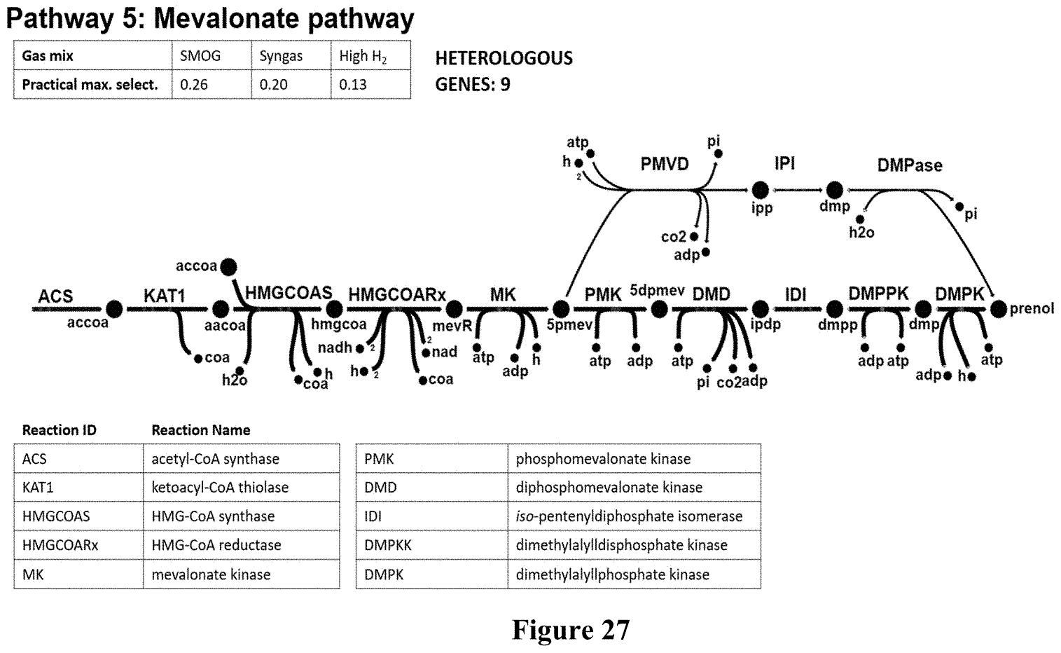

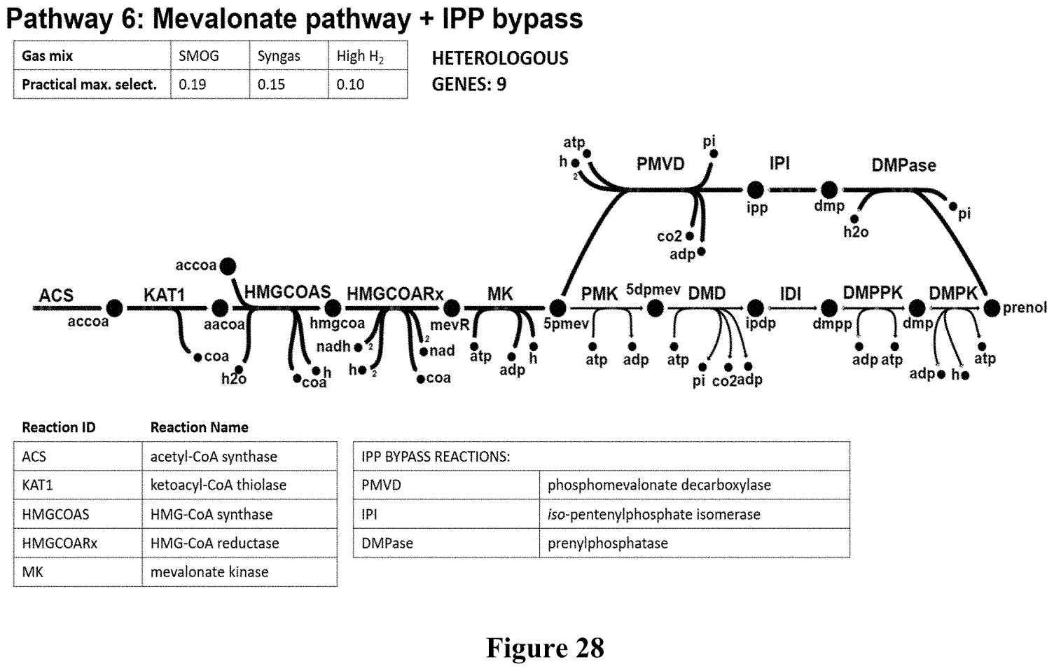

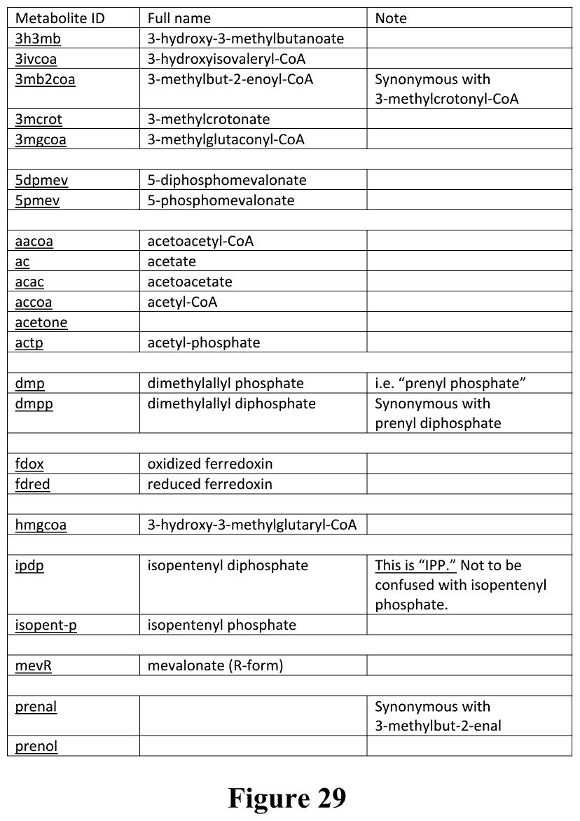

After conformation of successfully transformed of the plasmid pMTL8314-Prnf-MK-PMK-PMD-Pfor-idi-ispA-FS, a growth experiment was carried out in 50 ml PETC media (Table 1) in 250 ml serum bottles with 30 psi Real Mill Gas (collected from New Zealand Steel site in Glenbrook, NZ; composition: 44% CO, 32% N₂, 22% CO₂, 2% H₂) as sole energy and carbon source. All cultures were incubated at 37° C. on an orbital shaker adapted to hold serum bottles. Transformants were first grown up to an OD600 of ˜0.4 before being subcultured into fresh media supplemented with 1 mM mevalonic acid. Controls without mevalonic acid were set up at the same time from the same culture. Samples for GC-MS (Gas Chromatography-Mass Spectroscopy) were taken at each time point. FIG. 17 shows a representative growth curve for 2 control cultures and two cultures fed 1 mM mevalonate. Farnesene was detected in the samples taken at 66 h and 90 h after start of experiment (FIGS. 19-21).

### Detection of Alpha-Farnesene by Gas Chromatography-Mass Spectroscopy

For GC-MS detection of alpha-farnesene hexane extraction was performed on 5 ml of culture by adding 2 ml hexane and shaking vigorously to mix in a sealed glass balch tube. The tubes were then incubated in a sonicating water bath for 5 min to encourage phase separation. 400 μl hexane extract were transferred to a GC vail and loaded on to the auto loader. The samples was analysed on a VARIAN GC3800 MS4000 iontrap GC/MS (Varian Inc, CA, USA. Now Agilent Technologies) with a EC-1000 column 0.25 μm film thickness (Grace Davidson, Oreg., USA) Varian MS workstation (Varian Inc, Ca. Now Agilent Technologies, CA, USA) and NIST MS Search 2.0 (Agilent Technologies, CA, USA). Injection volume of 10 with Helium carrier gas flow rate of 1 ml per min.

The disclosure has been described herein, with reference to certain preferred embodiments, in order to enable the reader to practice the disclosure without undue experimentation. However, a person having ordinary skill in the art will readily recognise that many of the components and parameters may be varied or modified to a certain extent or substituted for known equivalents without departing from the scope of the disclosure. It should be appreciated that such modifications and equivalents are herein incorporated as if individually set forth. Titles, headings, or the like are provided to enhance the reader's comprehension of this document and should not be read as limiting the scope of the present disclosure.

The entire disclosures of all applications, patents and publications, cited above and below, if any, are hereby incorporated by reference. However, the reference to any applications, patents and publications in this specification is not, and should not be taken as, an acknowledgment or any form of suggestion that they constitute valid prior art or form part of the common general knowledge in any country in the world.

Throughout this specification and any claims which follow, unless the context requires otherwise, the words “comprise”, “comprising” and the like, are to be construed in an inclusive sense as opposed to an exclusive sense, that is to say, in the sense of “including, but not limited to.”

All references, including publications, patent applications, and patents, cited herein are hereby incorporated by reference to the same extent as if each reference were individually and specifically indicated to be incorporated by reference and were set forth in its entirety herein. The reference to any prior art in this specification is not, and should not be taken as, an acknowledgement that that prior art forms part of the common general knowledge in the field of endeavour in any country.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. The term “consisting essentially of” limits the scope of a composition, process, or method to the specified materials or steps, or to those that do not materially affect the basic and novel characteristics of the composition, process, or method. The use of the alternative (e.g., “or”) should be understood to mean either one, both, or any combination thereof of the alternatives. As used herein, the term “about” means±20% of the indicated range, value, or structure, unless otherwise indicated.

Recitation of ranges of values herein are merely intended to serve as a shorthand method of referring individually to each separate value falling within the range, unless otherwise indicated herein, and each separate value is incorporated into the specification as if it were individually recited herein. For example, any concentration range, percentage range, ratio range, integer range, size range, or thickness range is to be understood to include the value of any integer within the recited range and, when appropriate, fractions thereof (such as one tenth and one hundredth of an integer), unless otherwise indicated.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A genetically engineered microorganism capable of producing a product from a gaseous substrate, the microorganism comprising a nucleic acid encoding a group of exogenous enzymes comprising at least acetyl-CoA synthase and at least one of the following:
a) a nucleic acid encoding a group of exogenous enzymes comprising i) keto-acyl-CoA thiolase (KAT1), ii) 3-hydroxy-3-methylglutaryl coenzyme A (HMG-CoA) synthase, iii) methylglutaconyl-CoA hydratase (MGCH), iv) 3-methylcrotonyl-CoA carboxylase (MCCC), v) acyl-CoA reductase (ACOAR), and vi) alcohol dehydrogenase (ADH);
b) a nucleic acid encoding a group of exogenous enzymes comprising i) KAT1, ii) HMG-CoA synthase, iii) MGCH, iv) MCCC, v) phosphotransbutyrase butyrate kinase (Ptb-buk), vi) acetaldehyde-ferredoxin oxidoreductase (AOR), and vii) (ADH);
c) a nucleic acid encoding a group of exogenous enzymes comprising i) KAT1 or PTAr and ACKr, ii) CoA transferase A/B (CtfAB), iii) acetoacetate decarboxylase (ADC) or ADC and hydroxyisovalerate synthase (HIVS), iv) hydroxyisovalerate thioesterase (3HBZCT), v) hydroxyisopentyl-CoA hydrolyase (HPHL), vi) ACOAR, and vii) ADH;
d) a nucleic acid encoding a group of exogenous enzymes comprising i) KAT1 or PTAr and ACKr, ii) CoA transferase A/B (CtfAB), iii) ADC or ADC and HIVS, iv) 3HBZCT, v) HPHL, vi) Ptb-buk, vii) AOR, and ADH;
e) a nucleic acid encoding a group of exogenous enzymes comprising i) KAT1, ii) HMG-CoA synthase, iii) 3-hydroxy-3-methylglutaryl coenzyme A (HMG-CoA) reductase, iv) mevalonate kinase (MK), v) phosphomevalonate kinase (PMK), vi) diphosphomevalonate decarboxylase (DMD), vii) iso-pentenyl diphosphate isomerase (IDI), viii) dimethylallyl diphosphate kinase (DMPKK), and ix) dimethylallyl phosphate kinase (DMPK);
f) a nucleic acid encoding a group of exogenous enzymes comprising i) KAT1, ii) HMG-CoA synthase, iii) 3-hydroxy-3-methylglutaryl coenzyme A (HMG-CoA) reductase, iv) mevalonate kinase (MK), v) phosphomevalonate decarboxylase (PMVD), vi) iso-pentenyl phosphate isomerase (IPI), and vii) prenylphosphatase (DMPase);
g) a nucleic acid encoding a group of exogenous enzymes comprising i) thiolase, acyl-CoA acetyltransferase, or polyketide synthase, ii) β-Ketoacyl-CoA reductase or a β-hydroxyacyl-CoA dehydrogenase, iii) β-hydroxyacyl-CoA dehydratase, iv) trans-Enoyl-CoA reductase or butyryl-CoA dehydrogenase/electron transferring flavoprotein AB (Bcd-EtfAB), v) an alcohol forming acyl-CoA reductase or aldehyde forming acyl-CoA carboxylate reductase, vi) a hydrolysis enzyme or ADH, and vii) an alcohol dehydratase; and
wherein the microorganism is a C1-fixing microorganism and the product is an isoprenoid alcohol.

2. The microorganism according to claim 1, wherein the isoprenoid alcohol is prenol.

3. The microorganism according to claim 2, further comprising a nucleic acid encoding a group of exogenous enzymes capable of converting prenol to isoprenol.

4. The microorganism according to claim 1, further comprising a nucleic acid encoding a group of enzymes capable of converting prenol to dimethylallyl pyrophosphate (DMAPP).

5. The microorganism according to claim 3, further comprising a nucleic acid encoding a group of exogenous enzymes capable of converting isoprenol to isopentenyl diphosphate (IPP).

6. The microorganism according to claim 4, further comprising a nucleic acid encoding an exogenous enzyme selected from the group consisting of isopentenyl diphosphate isomerase and geranyltranstransferase.

7. The microorganism according to claim 4, further comprising a nucleic acid encoding both exogenous enzymes isopentenyl diphosphate isomerase and geranyltranstransferase.

8. The microorganism according to claim 7, further comprising a nucleic acid encoding a group of exogenous enzymes selected from limonene synthase, pinene synthase, farnesene synthase, or any combination thereof.

9. The microorganism according to claim 4, further comprising a nucleic acid encoding an exogenous enzyme comprising isoprene synthase.

10. The microorganism according to claim 1, having carbon monoxide dehydrogenase.

11. The microorganism according to claim 1, further comprising a disruptive mutation to DXS pathway.

12. The microorganism according to claim 11, wherein the disruptive mutation is a knockout.

13. The microorganism according to claim 1, wherein the exogenous enzymes comprise at least e) in combination with any one or more of a), b), c), d), f) and g) in tandem.

14. The microorganism according to claim 1, wherein the nucleic acids encoding exogenous enzymes are codon optimized.

15. The microorganism according to claim 1, wherein the nucleic acids encoding exogenous enzymes are integrated into the genome of the microorganism.

16. The microorganism according to claim 1, wherein the nucleic acids encoding exogenous enzymes are incorporated in a plasmid.

17. The microorganism according to claim 1, wherein the nucleic acids encoding exogenous enzymes are regulated by a constitutive promoter.

18. A method for producing an isoprenoid alcohol, by culturing the microorganism according to claim 1 using at least one C1 compound selected from the group consisting of carbon monoxide and carbon dioxide as a carbon source, to allow the microorganism to produce the isoprenoid alcohol.

19. A method for producing an isoprenoid alcohol, isoprenoid alcohol derivative, or terpene precursor by providing at least one C1 compound selected from the group consisting of carbon monoxide and carbon dioxide into contact with the microorganism according to claim 1, to allow the microorganism to produce the isoprenoid alcohol, isoprenoid alcohol derivative, or terpene precursor from the C1 compound.

20. The method according to claim 18, wherein the microorganism is provided with a gas comprising hydrogen.

21. The method according to claim 18, wherein the isoprenoid alcohol, is recovered.

22. The method according to claim 19, wherein the microorganism is provided with a gas comprising hydrogen.

23. The method according to claim 19, wherein the terpene precursor is recovered.

24. The method of claim 18, wherein the C1 compound is derived from an industrial process selected from the group consisting of ferrous metal products manufacturing, non-ferrous products manufacturing, petroleum refining, coal gasification, electric power production, carbon black production, ammonia production, methanol production, and coke manufacturing.

25. The method of claim 18, wherein the C1 compound is syngas.

26. The microorganism according to claim 1, wherein the microorganism is selected from the group consisting of Clostridium autoethanogenum, Clostridium ljungdahlii, Clostridium ragsdalei, Clostridium carboxidivorans, Clostridium drakei, Clostridium scatologenes, Clostridium aceticum, Clostridium formicoaceticum, Clostridium magnum, Cupriavidus necator, Moorella thermoacetica, Moorella thermautotrophica, and any combination thereof.

27. The microorganism according to claim 1, wherein the isoprenoid alcohol is converted to a terpene selected from the group consisting of terpenoids, vitamin A, lycopene, squalene, isoprene, pinene, nerol, citral, camphor, menthol, limonene, nerolidol, farnesol, farnesene, phytol, carotene, linalool, and any combination thereof.


SEQUENCE LISTING (111 sequences)

SEQ ID NO: 1 - DNA, 1875 bp, Clostridium autoethanogenum
  source 1..1875: mol_type = other DNA
atgagtaatttattagataattataaagatataaatgacgtaaagaagatgtcgttaaatgataaaaaaaagctagctagagaaattagaaaatttttaatagacaaagtatctaagacaggaggtcatttggcgtctaacttaggggttgtggagctcactttgagtttatttagtgtatttgatctaaattatgataaacttatatgggatgtgggacatcaggcttatgtgcataaaatcctcacgggaagaaaggataaatttgatactttaaggcaatttggaggattaagtggatttcctaaaaggtgcgaaagtatatatgattttttcgaaacagggcatagtagtacttcaatatctgcagcacttggaatggctagggctagagatttaaagcatgagaaatataatgttgttgcagttataggagatggagcacttactggaggtatggcactagaggccctaaatgatgtaggttatagaaaaactaagcttataataatattaaatgataatcaaatgtctataggaaaaaatgtaggtggagtatctaaatatttaaataaacttagagtggaccctaagtataataaatttaaagcggatgtagaagctaaattaaaaaagatacctaatataggaaaaggaatggcaaaatatcttgaaaaggtaaaaaatggaataaaacaaatggtagttcctggaatgttttttgaagatatgggaattaaatatttaggaccaatagatggtcataatataaaagaacttacagacgtactcgcttctgcaaaagacatacaaggtccagttattatacatataataactaagaaaggaaaaggatatgaatttgcagaaaaaaatccaggtaaattccatggaatagggccttttaattgcgccaatggtgaactggatgctggatcttcaaatacttattccaaggcctttggaaatgaaatggtaaagctagcagaaaaagacgatagaatagtggctataactgcagccatgagggatggaacaggtcttaaaagtttttctcaaaagtttcctgaaaggttttttgatgtgggaatagcagaacagcatgctgtaaccctggcagctggaatggcacaggcaaatttaaaacctgtatttgcagtttactctacttttcttcaaagagcttatgatcaacttattcatgatgtatgtatgcaaaaacttccagtagtttttgctgtagatagggccggcattgtaggagaagatggtgaaacacatcagggaatatttgatttatcttacttaacggaaatgccacatatgacgcttatgtctcctaaatgtatagatgaacttccatatatgttaaaatgggcattaggccagagttttcctgtagctataaggtatccaaggggaggagatagtgtatgtctcaatcccgtagaaaattttaaacttggaaagtgggactgtatttcaaatgaaggcagtgtagcaataattgctcagggtaaaatggtacaaaatgcagtgttagcaggaaaaaaacttaaagaaaagggtatagatgtaaggattataagtgcatgttttattaagccgctggacaaggaaatgttaaacaggttagttgaagaaagtgtaactatcgttactgttgaagacaatgtaataagaggaggattaggatcctatatattagaatatgtaaataaattaaataaaaaagtaaaaataataaacttagggtttgatgataagtttgtacagcatggaaaatccgatattttgtataagctgtatggtttggatcctaaaggtatcgtaaatagtgtacttgaagcagcagaggtaagtcatatattttaa

SEQ ID NO: 2 - protein, 627 aa, Clostridium autoethanogenum
MSNLLDNYKDINDVKKMSLNDKKKLAREIRKFLIDKVSKTGGHLASNLGVVELTLSLFSVFDLNYDKLIWDVGHQAYVHKILTGRKDKFDTLRQFGGLSGFPKRCESIYDFFETGHSSTSISAALGMARARDLKHEKYNVVAVIGDGALTGGMALEALNDVGYRKTKLIIILNDNQMSIGKNVGGVSKYLNKLRVDPKYNKFKADVEAKLKKIPNIGKGMAKYLEKVKNGIKQMVVPGMFFEDMGIKYLGPIDGHNIKELTDVLASAKDIQGPVIIHIITKKGKGYEFAEKNPGKFHGIGPFNCANGELDAGSSNTYSKAFGNEMVKLAEKDDRIVAITAAMRDGTGLKSFSQKFPERFFDVGIAEQHAVTLAAGMAQANLKPVFAVYSTFLQRAYDQLIHDVCMQKLPVVFAVDRAGIVGEDGETHQGIFDLSYLTEMPHMTLMSPKCIDELPYMLKWALGQSFPVAIRYPRGGDSVCLNPVENFKLGKWDCISNEGSVAIIAQGKMVQNAVLAGKKLKEKGIDVRIISACFIKPLDKEMLNRLVEESVTIVTVEDNVIRGGLGSYILEYVNKLNKKVKIINLGFDDKFVQHGKSDILYKLYGLDPKGIVNSVLEAAEVSHIFREF

SEQ ID NO: 3 - DNA, 1158 bp, Clostridium autoethanogenum
  source 1..1158: mol_type = other DNA
atgaagagaatttcaataattggagccacaggttctataggaacccaaactcttgatgtacttagaaaacaaaaaggagattttcagcttataggtgtatctgcaaatagtagtgtagataaacttttacatataatagatgaatttaaccccaaatatgcggtgctaaccgaaaaagaatcttatttaaagataaaagatatttttagtaataaaaaatcaaatacaaaaatattatttggagtagatggattaaatactatagctagtcttcctgaagttgatatggttgtaacatctgtagttggaatgatagggcttgtaccaactataaaagcaattaaagcgaagaaagacatagctttagctaataaggagacattagttgtaggaggagaactggttacaaaattatcgaaagaaaataatataaaaatatttcctgtagattcagagcatagtgctgtttttcaatgccttcagggaaataattttgacgaagttgctaatttgattttaaccgcttcaggtggaccttttaggggaaaaacaaaagatcaactctcaaaagtaactgtaaaagaggcgttgaatcatccaaattggagtatgggaaaaaagctcacaatagattctgctactcttatgaataagggacttgaagttatagaagctcacttcttatttaacttaccttatgaaaatataaaggttgtagttcatccacaaagtatagtacattctatggtggaatatagggatggaagtgttatggcacagcttgccactgcagatatgagattacctatacaatatgcactgaattatccgaaaagaaaggaagctgtaatagataaattggacttctatagcgtaggaaatttaagttttgaaaagcctgatacagatacattcagaccacttaaattagcttatgaagcagggaggataggaggcacaatgccagctatactaaattgtgcaaatgaggaagcagtaagtttattccttgctaataaaataaattttttggatataggcaacatattagaagagtgtatgaataaatttacttcacaaagtacgtatactctggatgatttacttgacctagaaataaaagttaagaaatatgtaaaagataaatttatcaaataa

SEQ ID NO: 4 - protein, 385 aa, Clostridium autoethanogenum
MKRISIIGATGSIGTQTLDVLRKQKGDFQLIGVSANSSVDKLLHIIDEFNPKYAVLTEKESYLKIKDIFSNKKSNTKILFGVDGLNTIASLPEVDMVVTSVVGMIGLVPTIKAIKAKKDIALANKETLVVGGELVTKLSKENNIKIFPVDSEHSAVFQCLQGNNFDEVANLILTASGGPFRGKTKDQLSKVTVKEALNHPNWSMGKKLTIDSATLMNKGLEVIEAHFLFNLPYENIKVVVHPQSIVHSMVEYRDGSVMAQLATADMRLPIQYALNYPKRKEAVIDKLDFYSVGNLSFEKPDTDTFRPLKLAYEAGRIGGTMPAILNCANEEAVSLFLANKINFLDIGNILEECMNKFTSQSTYTLDDLLDLEIKVKKYVKDKFIK

SEQ ID NO: 5 - DNA, 693 bp, Clostridium autoethanogenum
  source 1..693: mol_type = other DNA
atgaatggtaattatgctattattgtagctgccggcaagggaaaaagaatgggaactactattaataagcaatttattaaaattaagggtaagcctatattatattattccataagggcattttccataaatcctcttatagatggaattatactggtatgtgcagaaactgagatagaatattgtaaaagagaagtagtagataaatatgggcttcagaaggtaattaaattagttgctgggggtaaagaacgtcaggattcggtatttaatggactaggagttttagaaaaagaaaactgtagtgttgttctaattcacgatggggctagaccttttgtcactagtaaaattattgatgatggaataaaatattctaataggtatggggcttgtgcttgtggagttaggcctaaggatacactaaaagttagggaagaaagtggattttcttcttctacattagagagaaaaagtttatttgcagttcaaactccgcagtgttttaaatatgatttaatttatgactgtcataaaaaattaatgaatgaaaaaatgtgtgttactgatgatactatggtagtagagcgttatggaaataaggtttatttgtatgaaggtaactatgaaaacataaaagtgaccacaccagaagatttaaatatagctgaaagtatagttgaaaaatattaa

SEQ ID NO: 6 - protein, 230 aa, Clostridium autoethanogenum
MNGNYAIIVAAGKGKRMGTTINKQFIKIKGKPILYYSIRAFSINPLIDGIILVCAETEIEYCKREVVDKYGLQKVIKLVAGGKERQDSVFNGLGVLEKENCSVVLIHDGARPFVTSKIIDDGIKYSNRYGACACGVRPKDTLKVREESGFSSSTLERKSLFAVQTPQCFKYDLIYDCHKKLMNEKMCVTDDTMVVERYGNKVYLYEGNYENIKVTTPEDLNIAESIVEKY

SEQ ID NO: 7 - DNA, 798 bp, Clostridium autoethanogenum
  source 1..798: mol_type = other DNA
gtgggaaaaagaaaagatgggtatcatcttttgaaaatgataatgcagaatatagacttatatgatgttttaaaaatagatgagatcaaaactggaatacagatatgctctaataatagatatattccctgtgacaggagaaatttggtttacagagcagcaaaattatttattgataaatataatataaagaatggaattagtataaacataggtaaaaatatacctgtatcagctggacttgctggtggaagtgcggatgctgcagctatactaaagactatgagaaatatttatactcctgaagtaagtgataaagaattgagcgaattaggcttaaatataggggcagatgttccttattgtataataggaggtacagccttgtgcgaggggataggagagaaggttacaccactcatgccgtttagaaaccatatactcatattaattaaaccaccttttggagtgagcacagcagaggtatataagagtttagacataagtaaaataaaaaggcatcctaatacagaaattttaatagatgcggttaatgaatcaaaattggagatgctgagtaaaaacatgaaaaatgttttggaaaatgtaactttaaaaaaatatcccgtgcttagaaaaataaaaactgatttgatagattttggagcagttggttcacttatgagtggaagcggtccaagcatttttgctttttttgatgatatgctaaaagcacagaaatgttatgataatatgaaaactaggtatagagaggtgtttattacaagaaccatttaa

SEQ ID NO: 8 - protein, 265 aa, Clostridium autoethanogenum
MGKRKDGYHLLKMIMQNIDLYDVLKIDEIKTGIQICSNNRYIPCDRRNLVYRAAKLFIDKYNIKNGISINIGKNIPVSAGLAGGSADAAAILKTMRNIYTPEVSDKELSELGLNIGADVPYCIIGGTALCEGIGEKVTPLMPFRNHILILIKPPFGVSTAEVYKSLDISKIKRHPNTEILIDAVNESKLEMLSKNMKNVLENVTLKKYPVLRKIKTDLIDFGAVGSLMSGSGPSIFAFFDDMLKAQKCYDNMKTRYREVFITRTI

SEQ ID NO: 9 - DNA, 480 bp, Clostridium autoethanogenum
  source 1..480: mol_type = other DNA
gtgaaaatcgggcttggttatgatgtccataaattagtttataatagacctcttattttaggaggcgtaaatatcccttttgaaaaaggtcttatgggacattcagatgcagatgtacttcttcatgcaataatggatagtctccttggagccttgtgtctaggtgatatcggcaagcatttccctgataatgataataaatataagaacatatgtagtcttaaattgctgtcacatgtatcagctttgattaatgaaaaaggatatactatagggaacatagattctattataatagccgaaaagcctaaactttcttcatacatacaagatatgagggtaaatatagctaaaactctaaatgtaactacagccgtaataagtgtaaaagccactacagaggaaggtcttggctttaccggcaaaggagaaggcatagccgctcaaagcatctgtttgttaacagctaattcaaaataa

SEQ ID NO: 10 - protein, 159 aa, Clostridium autoethanogenum
MKIGLGYDVHKLVYNRPLILGGVNIPFEKGLMGHSDADVLLHAIMDSLLGALCLGDIGKHFPDNDNKYKNICSLKLLSHVSALINEKGYTIGNIDSIIIAEKPKLSSYIQDMRVNIAKTLNVTTAVISVKATTEEGLGFTGKGEGIAAQSICLLTANSK

SEQ ID NO: 11 - DNA, 1051 bp, Clostridium autoethanogenum
  source 1..1051: mol_type = other DNA
ttgaatagagtaaaaaagaaaacagtaaaggtaggcaatatatttttaggtggagattttccagtagccgtacaatctatgacaaatacggatactagggatgtagaagccactacagctcagatatttcagctaaaagaagcaggttgtgatatcgtcagatgtgcggtgcctgatgatatagcttgcaattccatgaaaaaaatcatagaaagagtagatattccacttgtagcagatatacattttgattataagttggcgcttaaatctatagaaaatgggatatctgcacttagaataaatcctggaaatattggaagcatagaaagagtacgagaagtggcaagagcagcaaaagaagctaatattccaattagaataggggtaaactctggatcattaaaaaaagatattttaaataaatatggtagagtttgttcggatgcactagtagagagtgctctagaacatgtaaaaattttggaaaacgtaggattttatgatatagttatatccataaaatcttcaaatgtaaatcagatgatagaaagttatagaaaaatatctgaaattgtagattatccacttcaccttggagtaacagaagcaggaactatttggcgaggaactataaaatcaagcataggcataggtactcttttgatggaaggtataggagacactataagagtatctcttacaggaaatccagtggaagaagtaagagtgggaaaagaaatattaaaatcctgtggaattataaaagaaggtgtggaatttatatcatgtcccacctgtggtagaactgaaattgatttaattaaaatagctgagcaagtggaaaaaagacttttaaatatgcataaaaacataaaggttgctgttatgggatgtgtagtaaatggaccaggtgaggctcgggaagcagatattggtatagcaggcggcaaaggtgaaggcattatatttaaaaaaggaaaaatagtaaaaaaggtaagtgaagaaagtttagtagaatcacttatagaagaaatagaaaacatttgar

SEQ ID NO: 12 - protein, 349 aa, Clostridium autoethanogenum
MNRVKKKTVKVGNIFLGGDFPVAVQSMTNTDTRDVEATTAQIFQLKEAGCDIVRCAVPDDIACNSMKKIIERVDIPLVADIHFDYKLALKSIENGISALRINPGNIGSIERVREVARAAKEANIPIRIGVNSGSLKKDILNKYGRVCSDALVESALEHVKILENVGFYDIVISIKSSNVNQMIESYRKISEIVDYPLHLGVTEAGTIWRGTIKSSIGIGTLLMEGIGDTIRVSLTGNPVEEVRVGKEILKSCGIIKEGVEFISCPTCGRTEIDLIKIAEQVEKRLLNMHKNIKVAVMGCVVNGPGEAREADIGIAGGKGEGIIFKKGKIVKKVSEESLVESLIEEIENI

SEQ ID NO: 13 - DNA, 1923 bp, Clostridium autoethanogenum
  source 1..1923: mol_type = other DNA
gtgataaaattgaacattattttagcagacaaatccggattttgctttggagtaaaaagagctgtagacgaatctttaaaggttcaaaaaaaatttaataaaaaaatatatactttaggtcctttgattcataatagtgatgtagtaaataaattaaaggaaaaaggtatatatcctatagaaatagataatatagataatctaagggaagatgatgtggttataatacgttctcatggtgttcccgaaaaaatattttttactttaaaaaataaaaaaataaacatagtaaatgcaacttgcccatatgttttaaatatacaaagaaaagtacaagaatattataaattagggtattctatattaatagtaggagataaaaatcatcctgaagtaattggaataaatggatggtgtgaaaataaagctttaatatctaaagatggcaccaatttagaaaagttaccatcaaaactgtgtatagtttctcaaactacagaaaaacaatctaactgggaaaaagtgcttagtatagtggctaaaaattgtaaagaatttattgcttttaatactatatgcagtgccacagaatttcgtcagaaggcagcagcagatatttctaaagaagtagatatgatggtagtaataggtggtaaaaacagctctaatactactaaactttatgaaatatgtaaagataactgcaataatactatttatgttgaaaattcaggagaaatacctgatgatataagtaattgtaataaaattaaaactataggtgttacagcaggagcttcaacaccagattggataataaaggaggcaattttaaaaatgagtgatgacaaaaatttagaactaaatgagcaactatcttatatggacaaaaatgatacccaaataatattaggtgaaaaaattaagggtacagtaatatctgtaaatccaaaagaggtttttttaaatataggatataaatcagaaggtgtacttccaaaacgtgaaataacaaaaaatgaaagtgacaacttagaagaattaattcattgtggagatgaattatatgttaaagtaataagaagacaaaatgaagatggatatgtggtattatctaagatagaattagaaagagaaaatgcttataaagaattaaaggaagctaatggaaatagtcaggtattaaaggttattgtaaaagaagctgtaaatggaggtcttgttgccaattacaaaggtgctagggtatttatacctgcttctcatgtagaattatatcatgtagatgatctttcacaatatgtagataaagagcttgatgtaactataattgaatttaaagaagaaaagaaaggtaccagaatagtagcttcaagaagagaccttttgagaatggaaagagaaaaaatggaagaacagacttggaatgtgcttgaaaaagatactgtagtagatggtgaagttagaagattgactgattttggcgcatttgttgatgtacaaggagttgacgggcttctacatgtatctgaactttcctggggaagagttggaaaaccaagtgatgttttaaaaatcggagatacgattaaggtttatatcttagacattgataaagaaaaaaagaagttatctttatctttaaaaaagctcatggaagatccatggatcaacgtagacataaaatatcctgttggcaatgtagttcttggtaaagtagttaggtttgcaaattttggtgcatttgttgaattagagccaggtgtagatgcattagttcatatatcacaaataagccataagagaatagataaaccagaagatgtacttaaaataggtcaggaaataaaggctaagatccttgaagtaaacaaagatagcgaaaaaatagctttaagtataaaagaagtagatgaaatctaa

SEQ ID NO: 14 - protein, 640 aa, Clostridium autoethanogenum
MIKLNIILADKSGFCFGVKRAVDESLKVQKKFNKKIYTLGPLIHNSDVVNKLKEKGIYPIEIDNIDNLREDDVVIIRSHGVPEKIFFTLKNKKINIVNATCPYVLNIQRKVQEYYKLGYSILIVGDKNHPEVIGINGWCENKALISKDGTNLEKLPSKLCIVSQTTEKQSNWEKVLSIVAKNCKEFIAFNTICSATEFRQKAAADISKEVDMMVVIGGKNSSNTTKLYEICKDNCNNTIYVENSGEIPDDISNCNKIKTIGVTAGASTPDWIIKEAILKMSDDKNLELNEQLSYMDKNDTQIILGEKIKGTVISVNPKEVFLNIGYKSEGVLPKREITKNESDNLEELIHCGDELYVKVIRRQNEDGYVVLSKIELERENAYKELKEANGNSQVLKVIVKEAVNGGLVANYKGARVFIPASHVELYHVDDLSQYVDKELDVTIIEFKEEKKGTRIVASRRDLLRMEREKMEEQTWNVLEKDTVVDGEVRRLTDFGAFVDVQGVDGLLHVSELSWGRVGKPSDVLKIGDTIKVYILDIDKEKKKLSLSLKKLMEDPWINVDIKYPVGNVVLGKVVRFANFGAFVELEPGVDALVHISQISHKRIDKPEDVLKIGQEIKAKILEVNKDSEKIALSIKEVDEI

SEQ ID NO: 15 - DNA, 882 bp, Clostridium autoethanogenum
  source 1..882: mol_type = other DNA
atggaaattaaaggtgtaattgaaacattaagagaggaattgaataaatacctctatgactatatggagggaaaaggatcttataataagagagtatatgaagctatgcagtatagcttagatgcaggaggaaagagaataagacctctactatttcttttgacatataaactttataagacagattgcaatgaggttatggatatagcagcagctatagaaatgatacacacttattccttaattcatgatgatttacctgctatggacaatgatgatttaagaaggggcaaacctacaaatcataaggtatttggagaagctattgctgtacttgcgggagatggacttttaaatgaagcaatgagtctgatgtttagacactgtattgggaaaaaggataacgctataagggcttgtagcattatttctgaaagtgcaggagctgatgggatggttggcggacagacagtggatattttaagtgaaaacactaagatacctatagatcagctctattacatgcacagtaaaaaaacgggagcgctcataaaaggatctataatatctgcagcagtatatgcgggagcaagtaaagctgaaatagataaattaagctattatggagaaaagttaggattggcatttcaaataaaggatgatatattggatttaacaggagatactgctcttttaggtaaaaagataaaaagtgatctaaataataacaaaactacatttataagtacttatggaataaataaatgcaaagaaatgtgcaattcaattacaagtgaatgtataggagtactgaatgggatgagtgtagatacttcttatctaaaagatttaacatcatttttattaaatagagaaaagtga

SEQ ID NO: 16 - protein, 293 aa, Clostridium autoethanogenum
MEIKGVIETLREELNKYLYDYMEGKGSYNKRVYEAMQYSLDAGGKRIRPLLFLLTYKLYKTDCNEVMDIAAAIEMIHTYSLIHDDLPAMDNDDLRRGKPTNHKVFGEAIAVLAGDGLLNEAMSLMFRHCIGKKDNAIRACSIISESAGADGMVGGQTVDILSENTKIPIDQLYYMHSKKTGALIKGSIISAAVYAGASKAEIDKLSYYGEKLGLAFQIKDDILDLTGDTALLGKKIKSDLNNNKTTFISTYGINKCKEMCNSITSECIGVLNGMSVDTSYLKDLTSFLLNREK

SEQ ID NO: 17 - DNA, 519 bp, Clostridium autoethanogenum
  source 1..519: mol_type = other DNA
atgaataaaacaaggaaaatggtttttttaagctttctaacaagtatggctttagtcatatacataatagaaactcaagttccggttttatttcccggaataaaattaggacttgcaaatacaatttccctagctgcacttatacttataggatggaaagaagccttactaattatgtttttaaggacgcttctaggatctatgtttggtgggacaatgtctacctttatgttcagcatagccggaggaattttaagtaacattgttatgatccttctatacaaatattttaaaaattccttaagtctatggactataagcatatgcggggcaatatttcacaacataggccaacttttagtagcttctatagtaattcaagattttaggatatacatatatctaccggtgcttttaatctctgctataatcacaggatactttataggttggtgcgtgaaattcctaactaataacttatataaaattcctatgtttaaagaattaaaaaataagtaa

SEQ ID NO: 18 - protein, 172 aa, Clostridium autoethanogenum
MNKTRKMVFLSFLTSMALVIYIIETQVPVLFPGIKLGLANTISLAALILIGWKEALLIMFLRTLLGSMFGGTMSTFMFSIAGGILSNIVMILLYKYFKNSLSLWTISICGAIFHNIGQLLVASIVIQDFRIYIYLPVLLISAIITGYFIGWCVKFLTNNLYKIPMFKELKNK

SEQ ID NO: 19 - DNA, 825 bp, Clostridium autoethanogenum
  source 1..825: mol_type = other DNA
atgaacttcgatggaatttcaattccaataataaaagaacttaatcaacttgagttagagttaaaaaatattgcatcaaaattagattctactgttacacaagatatttttacctactttttttcaattccaggtaaaagactaagacctacattaacatttttatctgcaggtgctattagtagcgagcttacttcatctgcaaaacacaacttaattcagttgtcaataagcttagagcttattcacagcgctagtctaattcatgatgatatcatagatggtgacttactaagacgtggtcagaaaaccttaaataagacctttggaaataaaatagcagtacttgccggtgatgctttgtactcaagggcctttactattttctcagatactctgccaagagaatttgcgcaggtaatgggcagagttactgaatcaatgtctgtagctgaaatattaaatgctaacaatccctctcccgatcgtgaaacctattttaaaatcatcttaggaaaaacagcatctttcatgagcgcttgttgtaggcttggtggcagcatagcttatgccccttacgaagagtctaatatgctttctaaatacggtgaaaaccttggtatggcatatcaaatactggatgattatatcgatgaggatcccgttgcaatgaaaaatgtaactattgaagagggatttgaatttgcatataatgccaaagcttctattgaaaatttaaaagactcagcatacaaacaaagcttaataatgttagtagactatgttttagatttttatagtcctaaggtagagaatacattatag

SEQ ID NO: 20 - protein, 274 aa, Clostridium autoethanogenum
MNFDGISIPIIKELNQLELELKNIASKLDSTVTQDIFTYFFSIPGKRLRPTLTFLSAGAISSELTSSAKHNLIQLSISLELIHSASLIHDDIIDGDLLRRGQKTLNKTFGNKIAVLAGDALYSRAFTIFSDTLPREFAQVMGRVTESMSVAEILNANNPSPDRETYFKIILGKTASFMSACCRLGGSIAYAPYEESNMLSKYGENLGMAYQILDDYIDEDPVAMKNVTIEEGFEFAYNAKASIENLKDSAYKQSLIMLVDYVLDFYSPKVENTL

SEQ ID NO: 21 - DNA, 1797 bp, Populus tremuloides
  source 1..1797: mol_type = other DNA
catatggcaacagaattattatgtttacacagacctatatcacttactcacaaactttttaggaatccattacctaaagttattcaagctacacctttaacattaaaacttaggtgtagtgtttctacagaaaatgtatcatttagtgagacagaaactgaaacaagaagatcagcaaattatgaaccaaattcttgggattatgattatcttctttcttctgatactgatgagtcaatagaagtacataaagataaggctaagaaattagaagctgaagttaggagagaaataaataatgagaaggctgaatttcttacacttcttgaacttattgataatgtacaaagacttggattaggatatagatttgagtctgatataagaagagcattagatagatttgtaagtagtggaggatttgatggagttactaaaacttcattacatggaacagcattatcatttaggttattaaggcaacatggttttgaagtatctcaagaagcttttagtggatttaaagatcagaatggaaactttcttgagaatttaaaggaagacataaaagcaattctttctctttatgaagcatcatttttagcattagaaggtgagaatatattagatgaggctaaagtatttgcaatatctcatcttaaagaacttagtgaagaaaagattggtaaagaattagctgaacaagtttcacatgctttagaattaccattacatagaagaacacaaagattagaagcagtttggtcaatagaagcatatagaaagaaagaagacgcaaatcaagtacttttagaacttgcaatacttgactacaatatgattcaaagtgtatatcagagggatttaagagaaacatcaagatggtggagaagagtaggattagcaactaaattacattttgctagagataggcttattgaaagtttttattgggctgttggagttgcttttgaaccacaatattctgattgcagaaatagtgtagcaaagatgttttcatttgttactataattgacgatatttacgatgtatatggaactttagatgaacttgaactttttactgatgcagttgaaagatgggatgtaaatgctattaatgatcttcctgattatatgaagttatgttttcttgcactttacaatactattaacgagatagcttacgataacttaaaagataaaggtgagaacatacttccttatttaacaaaagcatgggcagatttatgtaatgcatttcttcaagaagctaagtggctttataataaatcaacacctacatttgatgattattttggaaatgcatggaaaagttctagtggacctttacagcttatttttgcttattttgctgtagtacagaacattaaaaaggaagagattgagaatcttcagaaatatcatgacataatatcaagacctagtcacatttttaggctttgtaatgatttagcatctgcttcagcagaaatagcaagaggtgaaactgctaattctgtaagttgttatatgagaacaaaaggtatatctgaagaattagctactgaaagtgttatgaatcttatagacgaaacttggaagaaaatgaacaaagaaaaacttggtggatctttatttgcaaaaccttttgttgagactgctataaatttagctagacagtctcattgcacatatcataatggtgatgcacatactagtccagatgaattaactaggaaaagagtacttagtgtaataactgaaccaatattaccatttgaaagataagaattc

SEQ ID NO: 22 - DNA, 617 bp, Clostridium autoethanogenum
  source 1..617: mol_type = other DNA
ggttaatgttaaaaatttatagtataactttaaaaaactgtcttaaaaagttgttatataaaaaatgttgacaattaaacagctatttagtgcaaaacaaccataaaaatttaaaaaataccataaattacttgaaaaatagttgataataatgtagagttataaacaaaggtgaaaagcattacttgtattcttttttatatattattataaattaaaatgaagctgtattagaaaaaatacacacctgtaatataaaattttaaattaatttttaattttttcaaaatgtattttacatgtttagaattttgatgtatattaaaatagtagaatacataagatacttaatttaattaaagatagttaagtacttttcaatgtgcttttttagatgtttaatacaaatctttaattgtaaaagaaatgctgtactatttactgtactagtgacgggattaaactgtattaattataaataaaaaataagtacagttgtttaaaattatattttgtattaaatctaatagtacgatgtaagttattttatactattgctagtttaataaaaagatttaattatatacttgaaaaggagaggaatttttatgcgtaaa

SEQ ID NO: 23 - DNA, 30 bp, synthetic construct
  misc_feature 1..30: note = oligonucleotide Ppfor-NotI-F
  source 1..30: mol_type = other DNA
aagcggccgcaaaatagttgataataatgc

SEQ ID NO: 24 - DNA, 30 bp, synthetic construct
  misc_feature 1..30: note = oligonucleotide Ppfor-NdeI-R
  source 1..30: mol_type = other DNA
tacgcatatgaattcctctccttttcaagc

SEQ ID NO: 25 - DNA, 5345 bp, synthetic construct
  misc_feature 1..5345: note = plasmid pMTL 85146-ispS
  source 1..5345: mol_type = other DNA
aattcgagctcggtacccggggatcctctagagtcgacgtcacgcgtccatggagatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgcattcacttcttttctatataaatatgagcgaagcgaataagcgtcggaaaagcagcaaaaagtttcctttttgctgttggagcatgggggttcagggggtgcagtatctgacgtcaatgccgagcgaaagcgagccgaagggtagcatttacgttagataaccccctgatatgctccgacgctttatatagaaaagaagattcaactaggtaaaatcttaatataggttgagatgataaggtttataaggaatttgtttgttctaatttttcactcattttgttctaatttcttttaacaaatgttcttttttttttagaacagttatgatatagttagaatagtttaaaataaggagtgagaaaaagatgaaagaaagatatggaacagtctataaaggctctcagaggctcatagacgaagaaagtggagaagtcatagaggtagacaagttataccgtaaacaaacgtctggtaacttcgtaaaggcatatatagtgcaattaataagtatgttagatatgattggcggaaaaaaacttaaaatcgttaactatatcctagataatgtccacttaagtaacaatacaatgatagctacaacaagagaaatagcaaaagctacaggaacaagtctacaaacagtaataacaacacttaaaatcttagaagaaggaaatattataaaaagaaaaactggagtattaatgttaaaccctgaactactaatgagaggcgacgaccaaaaacaaaaatacctcttactcgaatttgggaactttgagcaagaggcaaatgaaatagattgacctcccaataacaccacgtagttattgggaggtcaatctatgaaatgcgattaagggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcaggataaaaaaattgtagataaattttataaaatagttttatctacaatttttttatcaggaaacagctatgaccgcggccgcggttaatgttaaaaatttatagtataactttaaaaaactgtcttaaaaagttgttatataaaaaatgttgacaattaaacagctatttagtgcaaaacaaccataaaaatttaaaaaataccataaattacttgaaaaatagttgataataatgtagagttataaacaaaggtgaaaagcattacttgtattcttttttatatattattataaattaaaatgaagctgtattagaaaaaatacacacctgtaatataaaattttaaattaatttttaattttttcaaaatgtattttacatgtttagaattttgatgtatattaaaatagtagaatacataagatacttaatttaattaaagatagttaagtacttttcaatgtgcttttttagatgtttaatacaaatctttaattgtaaaagaaatgctgtactatttactgtactagtgacgggattaaactgtattaattataaataaaaaataagtacagttgtttaaaattatattttgtattaaatctaatagtacgatgtaagttattttatactattgctagtttaataaaaagatttaattatatacttgaaaaggagaggaatttttatgcgtcatatggcaacagaattattatgtttacacagacctatatcacttactcacaaactttttaggaatccattacctaaagttattcaagctacacctttaacattaaaacttaggtgtagtgtttctacagaaaatgtatcatttagtgagacagaaactgaaacaagaagatcagcaaattatgaaccaaattcttgggattatgattatcttctttcttctgatactgatgagtcaatagaagtacataaagataaggctaagaaattagaagctgaagttaggagagaaataaataatgagaaggctgaatttcttacacttcttgaacttattgataatgtacaaagacttggattaggatatagatttgagtctgatataagaagagcattagatagatttgtaagtagtggaggatttgatggagttactaaaacttcattacatggaacagcattatcatttaggttattaaggcaacatggttttgaagtatctcaagaagcttttagtggatttaaagatcagaatggaaactttcttgagaatttaaaggaagacataaaagcaattctttctctttatgaagcatcatttttagcattagaaggtgagaatatattagatgaggctaaagtatttgcaatatctcatcttaaagaacttagtgaagaaaagattggtaaagaattagctgaacaagtttcacatgctttagaattaccattacatagaagaacacaaagattagaagcagtttggtcaatagaagcatatagaaagaaagaagacgcaaatcaagtacttttagaacttgcaatacttgactacaatatgattcaaagtgtatatcagagggatttaagagaaacatcaagatggtggagaagagtaggattagcaactaaattacattttgctagagataggcttattgaaagtttttattgggctgttggagttgcttttgaaccacaatattctgattgcagaaatagtgtagcaaagatgttttcatttgttactataattgacgatatttacgatgtatatggaactttagatgaacttgaactttttactgatgcagttgaaagatgggatgtaaatgctattaatgatcttcctgattatatgaagttatgttttcttgcactttacaatactattaacgagatagcttacgataacttaaaagataaaggtgagaacatacttccttatttaacaaaagcatgggcagatttatgtaatgcatttcttcaagaagctaagtggctttataataaatcaacacctacatttgatgattattttggaaatgcatggaaaagttctagtggacctttacagcttatttttgcttattttgctgtagtacagaacattaaaaaggaagagattgagaatcttcagaaatatcatgacataatatcaagacctagtcacatttttaggctttgtaatgatttagcatctgcttcagcagaaatagcaagaggtgaaactgctaattctgtaagttgttatatgagaacaaaaggtatatctgaagaattagctactgaaagtgttatgaatcttatagacgaaacttggaagaaaatgaacaaagaaaaacttggtggatctttatttgcaaaaccttttgttgagactgctataaatttagctagacagtctcattgcacatatcataatggtgatgcacatactagtccagatgaattaactaggaaaagagtacttagtgtaataactgaaccaatattaccatttgaaagataag

SEQ ID NO: 26 - DNA, 26 bp, synthetic construct
  misc_feature 1..26: note = Oligonucleotide Idi-Cbei-SacI-F
  source 1..26: mol_type = other DNA
gtgagctcgaaaggggaaattaaatg

SEQ ID NO: 27 - DNA, 27 bp, synthetic construct
  misc_feature 1..27: note = Oligonucleotide Idi-Cbei-KpnI-R
  source 1..27: mol_type = other DNA
atggtaccccaaatctttatttagacg

SEQ ID NO: 28 - DNA, 5905 bp, synthetic construct
  misc_feature 1..5905: note = plasmid pMTL85246-ispS-idi
  source 1..5905: mol_type = other DNA
ccggggatcctctagagtcgacgtcacgcgtccatggagatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgcattcacttcttttctatataaatatgagcgaagcgaataagcgtcggaaaagcagcaaaaagtttcctttttgctgttggagcatgggggttcagggggtgcagtatctgacgtcaatgccgagcgaaagcgagccgaagggtagcatttacgttagataaccccctgatatgctccgacgctttatatagaaaagaagattcaactaggtaaaatcttaatataggttgagatgataaggtttataaggaatttgtttgttctaatttttcactcattttgttctaatttcttttaacaaatgttcttttttttttagaacagttatgatatagttagaatagtttaaaataaggagtgagaaaaagatgaaagaaagatatggaacagtctataaaggctctcagaggctcatagacgaagaaagtggagaagtcatagaggtagacaagttataccgtaaacaaacgtctggtaacttcgtaaaggcatatatagtgcaattaataagtatgttagatatgattggcggaaaaaaacttaaaatcgttaactatatcctagataatgtccacttaagtaacaatacaatgatagctacaacaagagaaatagcaaaagctacaggaacaagtctacaaacagtaataacaacacttaaaatcttagaagaaggaaatattataaaaagaaaaactggagtattaatgttaaaccctgaactactaatgagaggcgacgaccaaaaacaaaaatacctcttactcgaatttgggaactttgagcaagaggcaaatgaaatagattgacctcccaataacaccacgtagttattgggaggtcaatctatgaaatgcgattaagggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcaggataaaaaaattgtagataaattttataaaatagttttatctacaatttttttatcaggaaacagctatgaccgcggccgcggttaatgttaaaaatttatagtataactttaaaaaactgtcttaaaaagttgttatataaaaaatgttgacaattaaacagctatttagtgcaaaacaaccataaaaatttaaaaaataccataaattacttgaaaaatagttgataataatgtagagttataaacaaaggtgaaaagcattacttgtattcttttttatatattattataaattaaaatgaagctgtattagaaaaaatacacacctgtaatataaaattttaaattaatttttaattttttcaaaatgtattttacatgtttagaattttgatgtatattaaaatagtagaatacataagatacttaatttaattaaagatagttaagtacttttcaatgtgcttttttagatgtttaatacaaatctttaattgtaaaagaaatgctgtactatttactgtactagtgacgggattaaactgtattaattataaataaaaaataagtacagttgtttaaaattatattttgtattaaatctaatagtacgatgtaagttattttatactattgctagtttaataaaaagatttaattatatacttgaaaaggagaggaatttttatgcgtcatatggcaacagaattattatgtttacacagacctatatcacttactcacaaactttttaggaatccattacctaaagttattcaagctacacctttaacattaaaacttaggtgtagtgtttctacagaaaatgtatcatttagtgagacagaaactgaaacaagaagatcagcaaattatgaaccaaattcttgggattatgattatcttctttcttctgatactgatgagtcaatagaagtacataaagataaggctaagaaattagaagctgaagttaggagagaaataaataatgagaaggctgaatttcttacacttcttgaacttattgataatgtacaaagacttggattaggatatagatttgagtctgatataagaagagcattagatagatttgtaagtagtggaggatttgatggagttactaaaacttcattacatggaacagcattatcatttaggttattaaggcaacatggttttgaagtatctcaagaagcttttagtggatttaaagatcagaatggaaactttcttgagaatttaaaggaagacataaaagcaattctttctctttatgaagcatcatttttagcattagaaggtgagaatatattagatgaggctaaagtatttgcaatatctcatcttaaagaacttagtgaagaaaagattggtaaagaattagctgaacaagtttcacatgctttagaattaccattacatagaagaacacaaagattagaagcagtttggtcaatagaagcatatagaaagaaagaagacgcaaatcaagtacttttagaacttgcaatacttgactacaatatgattcaaagtgtatatcagagggatttaagagaaacatcaagatggtggagaagagtaggattagcaactaaattacattttgctagagataggcttattgaaagtttttattgggctgttggagttgcttttgaaccacaatattctgattgcagaaatagtgtagcaaagatgttttcatttgttactataattgacgatatttacgatgtatatggaactttagatgaacttgaactttttactgatgcagttgaaagatgggatgtaaatgctattaatgatcttcctgattatatgaagttatgttttcttgcactttacaatactattaacgagatagcttacgataacttaaaagataaaggtgagaacatacttccttatttaacaaaagcatgggcagatttatgtaatgcatttcttcaagaagctaagtggctttataataaatcaacacctacatttgatgattattttggaaatgcatggaaaagttctagtggacctttacagcttatttttgcttattttgctgtagtacagaacattaaaaaggaagagattgagaatcttcagaaatatcatgacataatatcaagacctagtcacatttttaggctttgtaatgatttagcatctgcttcagcagaaatagcaagaggtgaaactgctaattctgtaagttgttatatgagaacaaaaggtatatctgaagaattagctactgaaagtgttatgaatcttatagacgaaacttggaagaaaatgaacaaagaaaaacttggtggatctttatttgcaaaaccttttgttgagactgctataaatttagctagacagtctcattgcacatatcataatggtgatgcacatactagtccagatgaattaactaggaaaagagtacttagtgtaataactgaaccaatattaccatttgaaagataagaattcgagctcgaaaggggaaattaaatggcagaatatataatagctgtagatgaatttgataacgaaataggttcaattgaaaaaatggaggctcaccgtaaaggaacattacatagagctttttctatattagtatttaattctaaaaatcaattgttattacagaaaagaaatgtaaaaaaatatcattcgcctggtctctggacaaatacgtgctgtagtcatccaagatacggtgaaagtttacatgatgcgatttatagaaggcttaaggaagaaatgggttttacatgtgaacttgaagaagtatttagttttatttataaagtaaaacttgaagataatctttttgaaaatgaatatgatcatgtattcattgggaaatatgatggagaaataattgtaaacaaagatgaagtagatgattttaagtgggttgatattaatgaggttaagaaggatattatagaaaggccagaagcatacacttattggttcaagtatttagttaataaggcagaaaacaaaatatttaaataagtaagaatttcgtctaaataaagatttggggtac

SEQ ID NO: 29 - DNA, 29 bp, synthetic construct
  misc_feature 1..29: note = Oligonucleotide Dxs-SalI-F
  source 1..29: mol_type = other DNA
gcagtcgactttattaaagggatagataa

SEQ ID NO: 30 - DNA, 31 bp, synthetic construct
  misc_feature 1..31: note = Oligonucleotide Dxs-XhoI-R
  source 1..31: mol_type = other DNA
tgctcgagttaaaatatatgacttacctctg

SEQ ID NO: 31 - DNA, 7784 bp, synthetic construct
  misc_feature 1..7784: note = plasmid pMTL85246-ispS-idi-dxs
  source 1..7784: mol_type = other DNA
tcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgcattcacttcttttctatataaatatgagcgaagcgaataagcgtcggaaaagcagcaaaaagtttcctttttgctgttggagcatgggggttcagggggtgcagtatctgacgtcaatgccgagcgaaagcgagccgaagggtagcatttacgttagataaccccctgatatgctccgacgctttatatagaaaagaagattcaactaggtaaaatcttaatataggttgagatgataaggtttataaggaatttgtttgttctaatttttcactcattttgttctaatttcttttaacaaatgttcttttttttttagaacagttatgatatagttagaatagtttaaaataaggagtgagaaaaagatgaaagaaagatatggaacagtctataaaggctctcagaggctcatagacgaagaaagtggagaagtcatagaggtagacaagttataccgtaaacaaacgtctggtaacttcgtaaaggcatatatagtgcaattaataagtatgttagatatgattggcggaaaaaaacttaaaatcgttaactatatcctagataatgtccacttaagtaacaatacaatgatagctacaacaagagaaatagcaaaagctacaggaacaagtctacaaacagtaataacaacacttaaaatcttagaagaaggaaatattataaaaagaaaaactggagtattaatgttaaaccctgaactactaatgagaggcgacgaccaaaaacaaaaatacctcttactcgaatttgggaactttgagcaagaggcaaatgaaatagattgacctcccaataacaccacgtagttattgggaggtcaatctatgaaatgcgattaagggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcaggataaaaaaattgtagataaattttataaaatagttttatctacaatttttttatcaggaaacagctatgaccgcggccgcggttaatgttaaaaatttatagtataactttaaaaaactgtcttaaaaagttgttatataaaaaatgttgacaattaaacagctatttagtgcaaaacaaccataaaaatttaaaaaataccataaattacttgaaaaatagttgataataatgtagagttataaacaaaggtgaaaagcattacttgtattcttttttatatattattataaattaaaatgaagctgtattagaaaaaatacacacctgtaatataaaattttaaattaatttttaattttttcaaaatgtattttacatgtttagaattttgatgtatattaaaatagtagaatacataagatacttaatttaattaaagatagttaagtacttttcaatgtgcttttttagatgtttaatacaaatctttaattgtaaaagaaatgctgtactatttactgtactagtgacgggattaaactgtattaattataaataaaaaataagtacagttgtttaaaattatattttgtattaaatctaatagtacgatgtaagttattttatactattgctagtttaataaaaagatttaattatatacttgaaaaggagaggaatttttatgcgtcatatggcaacagaattattatgtttacacagacctatatcacttactcacaaactttttaggaatccattacctaaagttattcaagctacacctttaacattaaaacttaggtgtagtgtttctacagaaaatgtatcatttagtgagacagaaactgaaacaagaagatcagcaaattatgaaccaaattcttgggattatgattatcttctttcttctgatactgatgagtcaatagaagtacataaagataaggctaagaaattagaagctgaagttaggagagaaataaataatgagaaggctgaatttcttacacttcttgaacttattgataatgtacaaagacttggattaggatatagatttgagtctgatataagaagagcattagatagatttgtaagtagtggaggatttgatggagttactaaaacttcattacatggaacagcattatcatttaggttattaaggcaacatggttttgaagtatctcaagaagcttttagtggatttaaagatcagaatggaaactttcttgagaatttaaaggaagacataaaagcaattctttctctttatgaagcatcatttttagcattagaaggtgagaatatattagatgaggctaaagtatttgcaatatctcatcttaaagaacttagtgaagaaaagattggtaaagaattagctgaacaagtttcacatgctttagaattaccattacatagaagaacacaaagattagaagcagtttggtcaatagaagcatatagaaagaaagaagacgcaaatcaagtacttttagaacttgcaatacttgactacaatatgattcaaagtgtatatcagagggatttaagagaaacatcaagatggtggagaagagtaggattagcaactaaattacattttgctagagataggcttattgaaagtttttattgggctgttggagttgcttttgaaccacaatattctgattgcagaaatagtgtagcaaagatgttttcatttgttactataattgacgatatttacgatgtatatggaactttagatgaacttgaactttttactgatgcagttgaaagatgggatgtaaatgctattaatgatcttcctgattatatgaagttatgttttcttgcactttacaatactattaacgagatagcttacgataacttaaaagataaaggtgagaacatacttccttatttaacaaaagcatgggcagatttatgtaatgcatttcttcaagaagctaagtggctttataataaatcaacacctacatttgatgattattttggaaatgcatggaaaagttctagtggacctttacagcttatttttgcttattttgctgtagtacagaacattaaaaaggaagagattgagaatcttcagaaatatcatgacataatatcaagacctagtcacatttttaggctttgtaatgatttagcatctgcttcagcagaaatagcaagaggtgaaactgctaattctgtaagttgttatatgagaacaaaaggtatatctgaagaattagctactgaaagtgttatgaatcttatagacgaaacttggaagaaaatgaacaaagaaaaacttggtggatctttatttgcaaaaccttttgttgagactgctataaatttagctagacagtctcattgcacatatcataatggtgatgcacatactagtccagatgaattaactaggaaaagagtacttagtgtaataactgaaccaatattaccatttgaaagataagaattcgagctcgaaaggggaaattaaatggcagaatatataatagctgtagatgaatttgataacgaaataggttcaattgaaaaaatggaggctcaccgtaaaggaacattacatagagctttttctatattagtatttaattctaaaaatcaattgttattacagaaaagaaatgtaaaaaaatatcattcgcctggtctctggacaaatacgtgctgtagtcatccaagatacggtgaaagtttacatgatgcgatttatagaaggcttaaggaagaaatgggttttacatgtgaacttgaagaagtatttagttttatttataaagtaaaacttgaagataatctttttgaaaatgaatatgatcatgtattcattgggaaatatgatggagaaataattgtaaacaaagatgaagtagatgattttaagtgggttgatattaatgaggttaagaaggatattatagaaaggccagaagcatacacttattggttcaagtatttagttaataaggcagaaaacaaaatatttaaataagtaagaatttcgtctaaataaagatttggggtacccggggatcctctagagtcgactttattaaagggatagataaggatgagtaatttattagataattataaagatataaatgacgtaaagaagatgtcgttaaatgataaaaaaaagctagctagagaaattagaaaatttttaatagacaaagtatctaagacaggaggtcatttggcgtctaacttaggggttgtggagctcactttgagtttatttagtgtatttgatctaaattatgataaacttatatgggatgtgggacatcaggcttatgtgcataaaatcctcacgggaagaaaggataaatttgatactttaaggcaatttggaggattaagtggatttcctaaaaggtgcgaaagtatatatgattttttcgaaacagggcatagtagtacttcaatatctgcagcacttggaatggctagggctagagatttaaagcatgagaaatataatgttgttgcagttataggagatggagcacttactggaggtatggcactagaggccctaaatgatgtaggttatagaaaaactaagcttataataatattaaatgataatcaaatgtctataggaaaaaatgtaggtggagtatctaaatatttaaataaacttagagtggaccctaagtataataaatttaaagcggatgtagaagctaaattaaaaaagatacctaatataggaaaaggaatggcaaaatatcttgaaaaggtaaaaaatggaataaaacaaatggtagttcctggaatgttttttgaagatatgggaattaaatatttaggaccaatagatggtcataatataaaagaacttacagacgtactcgcttctgcaaaagacatacaaggtccagttattatacatataataactaagaaaggaaaaggatatgaatttgcaagaaaaaaatccaggtaaattccatggaatagggccttttaattgcgccaatggtgaactggatgctggatcttcaaatacttattccaaggcctttggaaatgaaatggtaaagctagcagaaaaagacgatagaatagtggctataactgcagccatgagggatggaacaggtcttaaaagtttttctcaaaagtttcctgaaaggttttttgatgtgggaatagcagaacagcatgctgtaaccctggcagctggaatggcacaggcaaatttaaaacctgtatttgcagtttactctacttttcttcaaagagcttatgatcaacttattcatgatgtatgtatgcaaaaacttccagtagtttttgctgtagatagggccggcattgtaggagaagatggtgaaacacatcagggaatatttgatttatcttacttaacggaaatgccacatatgacgcttatgtctcctaaatgtatagatgaacttccatatatgttaaaatgggcattaggccagagttttcctgtagctataaggtatccaaggggaggagatagtgtatgtctcaatcccgtagaaaattttaaacttggaaagtgggactgtatttcaaatgaaggcagtgtagcaataattgctcagggtaaaatggtacaaaatgcagtgttagcaggaaaaaaacttaaagaaaagggtatagatgtaaggattataagtgcatgttttattaagccgctggacaaggaaatgttaaacaggttagttgaagaaagtgtaactatcgttactgttgaagacaatgtaataagaggaggattaggatcctatatattagaatatgtaaataaattaaataaaaaagtaaaaataataaacttagggtttgatgataagtttgtacagcatggaaaatccgatattttgtataagctgtatggtttggatcctaaaggtatcgtaaatagtgtacttgaagcagcagaggtaagtcatatattttaac

SEQ ID NO: 32 - DNA, 17 bp, synthetic construct
  misc_feature 1..17: note = Oligonucleotide M13R
  source 1..17: mol_type = other DNA
caggaaacagctatgac

SEQ ID NO: 33 - DNA, 20 bp, synthetic construct
  misc_feature 1..20: note = Oligonucleotides Isoprene-seq1
  source 1..20: mol_type = other DNA
gttattcaagctacaccttt

SEQ ID NO: 34 - DNA, 20 bp, synthetic construct
  misc_feature 1..20: note = Oligonucleotide Isoprene-seq2
  source 1..20: mol_type = other DNA
gattggtaaagaattagctg

SEQ ID NO: 35 - DNA, 18 bp, synthetic construct
  misc_feature 1..18: note = Oligonucleotide Isoprene-seq3
  source 1..18: mol_type = other DNA
tcaagaagctaagtggct

SEQ ID NO: 36 - DNA, 17 bp, synthetic construct
  misc_feature 1..17: note = Oligonucleotide Isoprene-seq4
  source 1..17: mol_type = other DNA
ctcaccgtaaaggaaca

SEQ ID NO: 37 - DNA, 20 bp, synthetic construct
  misc_feature 1..20: note = Oligonucleotide Isoprene-seq5
  source 1..20: mol_type = other DNA
gctagctagagaaattagaa

SEQ ID NO: 38 - DNA, 20 bp, synthetic construct
  misc_feature 1..20: note = Oligonucleotide Isoprene-seq6
  source 1..20: mol_type = other DNA
ggaatggcaaaatatcttga

SEQ ID NO: 39 - DNA, 20 bp, synthetic construct
  misc_feature 1..20: note = Oligonucleotide Isoprene-seq7
  source 1..20: mol_type = other DNA
gaaacacatcagggaatatt

SEQ ID NO: 40 - DNA, 1179 bp, Clostridium acetobutylicum
  source 1..1179: mol_type = other DNA
atgaaagaggttgttattgcatcagctgttagaactgcaataggatcttatggaaaaagtcttaaagatgtaccagcagtagacttaggtgcaactgcaataaaggaagcagtaaagaaagcaggtataaaacctgaagatgttaatgaagttattttaggaaacgtattacaagctggacttggacagaatccagctagacaggcatcattcaaagcaggattaccagtagagatacctgctatgactattaataaagtttgtggttcaggattaagaacagtttctttagctgctcaaattataaaagctggtgacgcagatgtaataatagcaggtggtatggaaaatatgtcaagagcaccataccttgctaataatgctagatggggttatagaatgggaaacgctaaatttgtagacgaaatgataactgatggactttgggatgcatttaacgattatcacatgggaattactgctgaaaatatagctgagagatggaatataagtagagaagaacaagatgagtttgcacttgcatctcagaaaaaggcagaagaagctattaaatcaggacaatttaaagatgaaattgttccagtagtaattaaaggtagaaaaggtgaaacagttgtagacactgatgaacatcctagatttggatctacaatagaaggtttagctaaattaaagcctgcttttaagaaagacggaacagtaactgctggaaacgcatcaggtttaaatgattgtgcagctgttttagttattatgtctgctgaaaaggcaaaggaattaggtgttaaaccacttgctaagatagttagttatggttcagcaggtgtagatcctgctattatgggatatggacctttttatgctacaaaggcagctattgaaaaggctggttggacagttgatgaacttgatcttatagagtcaaatgaggcatttgcagcacaaagtcttgctgttgctaaggatcttaaattcgatatgaataaagtaaatgtaaacggtggtgctatagcacttggtcatccaataggtgctagtggtgctagaattttagttacattagttcatgcaatgcaaaagagagacgctaaaaagggacttgcaactttatgcataggtggtggtcaaggaacagcaatacttcttgaaaaatgttaa

SEQ ID NO: 41 - DNA, 1140 bp, Staphylococcus aureus
  source 1..1140: mol_type = other DNA; sub_species = subsp.aureus
atgaatcaggctgttatagttgctgcaaagagaactgcatttggaaaatatggtggtactttaaaacaccttgaaccagaacaattacttaagccattatttcagcactttaaagaaaaatatcctgaagttatatctaaaatagatgatgtagttttaggaaacgtagttggaaatggtggtaatattgctagaaaagcacttcttgaagcaggattaaaagatagtataccaggtgtaactatagacagacaatgcggatctggattagaatcagtacaatacgcatgtagaatgatacaagcaggtgctggaaaagtttatattgcaggtggtgttgaatctacatcaagagcaccttggaaaataaagagaccacattctgtttatgaaactgcattaccagagttctatgaaagagcatcattcgcacctgaaatgtcagatccaagtatgatacaaggtgctgagaatgtagctaaaatgtatgatgttagtagagaacttcaagatgagtttgcatacagatcacatcaacttacagctgaaaatgtaaagaatggaaatatttcacaagaaattcttccaataacagtaaagggtgaaatattcaatactgatgaaagtttaaaatctcatattccaaaagataatttcggtagatttaaacctgtaataaaaggtggtactgtaacagctgctaatagttgtatgaagaacgatggtgcagtattattacttattatggaaaaggatatggcttatgaacttggatttgagcatggattattatttaaagacggtgtaactgtaggtgtagatagtaactttccaggaataggacctgttcctgctatatcaaatcttttaaagagaaaccaacttacaatagaaaatattgaagtaattgagataaatgaagcattttctgctcaggtagttgcttgtcagcaagctcttaatataagtaatactcagttaaacatttggggtggtgcattagcaagtggtcatccttatggtgcatcaggtgcacagttagttacaagattattttatatgttcgataaagagacaatgattgcttctatgggaataggtggtggtttaggaaatgcagctctttttactagattttaa

SEQ ID NO: 42 - DNA, 1167 bp, Staphylococcus aureus
  source 1..1167: mol_type = other DNA; sub_species = subsp. aureus
atgactataggaattgacaaaataaacttttacgtaccaaaatattatgtagatatggcaaaattagcagaagcaagacaagtagacccaaataaatttcttattggaataggacagactgaaatggcagttagtccagtaaaccaagatatagtatcaatgggtgctaatgctgctaaagatataataactgatgaagacaaaaagaaaataggaatggtaatagtagcaactgagtcagcagtagatgcagcaaaggcagcagcagtacagattcataatttattaggtattcaaccatttgcaagatgtttcgaaatgaaagaagcatgttatgctgctactcctgcaattcagttagctaaggattatttagctacaagaccaaatgagaaagttttagttatagctacagatacagctagatatggacttaattcaggtggtgaacctactcaaggtgctggtgctgttgctatggttatagctcataatcctagtatacttgcattaaatgaagacgctgttgcttatacagaagatgtttatgatttctggagaccaacaggacataagtatccattagtagatggtgctttatcaaaagacgcatatattagatcttttcaacaatcttggaatgaatatgctaagagacaaggaaagagtttagctgattttgctagtctttgctttcatgttccttttactaaaatgggtaaaaaggctttagaatctataatagataacgcagatgaaacaactcaagagagattaagatctggatatgaagatgcagttgattacaatagatatgttggaaatatatacacaggaagtctttatctttctcttataagtcttcttgaaaatagagatttacaggctggtgaaactattggattattttcatacggatcaggttctgttggtgaattttattcagctacacttgtagaaggatataaagatcaccttgatcaggcagcacacaaagcacttttaaacaatagaactgaagtatcagtagatgcatacgaaacatttttcaagagatttgatgatgtagaatttgatgaagagcaggatgcagttcatgaagatagacatatattctatctttcaaacatagagaataatgtaagagaatatcatagacctgaataa

SEQ ID NO: 43 - DNA, 1278 bp, Staphylococcus aureus
  source 1..1278: mol_type = other DNA; sub_species = subsp. aureus
atgcaatcattagacaaaaatttcagacatttatcaagacaacaaaagttacaacaattagttgataaacagtggctttcagaagatcagtttgatattttacttaatcatcctcttatagatgaagaagttgctaatagtcttatagaaaatgtaattgcacagggtgcattaccagttggacttcttcctaatataatagttgatgataaggcttatgttgtaccaatgatggttgaagaacctagtgttgttgcagctgcatcttatggtgctaaattagtaaatcagacaggtggatttaaaactgtatcatcagaaagaataatgattggacagatagtatttgatggtgtagatgacactgaaaaattaagtgcagatattaaagcattagaaaaacaaatacataagattgcagatgaagcatatcctagtataaaagcaagaggtggtggttatcaaagaatagcaatagatacatttccagagcaacaacttttaagtcttaaggtatttgtagatacaaaagatgctatgggtgctaatatgcttaatactatacttgaggcaataactgcattccttaaaaatgaatctcctcaatcagatatattaatgtctatactttcaaaccatgcaactgctagtgtagtaaaagtacaaggtgagatagatgtaaaagatcttgctagaggtgaaagaacaggtgaagaagtagctaagagaatggaaagagcttctgtattagctcaggttgatattcatagagctgcaacacataacaaaggtgttatgaatggaatacatgctgttgttttagctacaggaaatgatactagaggtgctgaagcatctgcacatgcatacgcatcaagagacggacaatatagaggtatagcaacttggagatatgatcagaagagacaaagacttattggaactattgaagttccaatgacacttgctatagtaggtggtggtactaaagtattaccaatagctaaggcatcattagagttattaaatgttgattctgcacaagaacttggacacgtagttgctgctgttggattagcacaaaactttgctgcttgtagagcacttgtttctgaaggtattcaacaaggacacatgtcattacaatataaaagtttagcaatagtagtaggtgcaaaaggtgacgagatagcacaagtagcagaagctcttaaacaggaaccaagagctaatacacaggttgctgaaagaattttacaggaaattagacagcaataa

SEQ ID NO: 44 - DNA, 552 bp, Clostridium autoethanogenum
  source 1..552: mol_type = other DNA
agatagtcataatagttccagaatagttcaatttagaaattagactaaacttcaaaatgtttgttaaatatataccaatctagtatagatattttttaaatactggacttaaacagtagtaatttgcctaaaaaattttttcaattttttttaaaaaatccttttcaagttgtacattgttatggtaatatgtaattgaagaagttatgtagtaatattgtaaacgtttcttgatttttttacatccatgtagtgcttaaaaaaccaaaatatgtcacatgcacttgtatatttcaaataacaatatttattttctcgttaaattcacaaataatttattaataatatcaataaccaagattatacttaaatggatgtttattttttaacacttttatagtaaatatatttattttatgtagtaaaaaggttataattataattgtatttattacaattaattaaaataaaaaatagggttttaggtaaaattaagttattttaagaagtaattacaataaaaattgaagttatttctttaaggaggaaatt

SEQ ID NO: 45 - DNA, 386 bp, Clostridium autoethanogenum
  source 1..386: mol_type = other DNA
gttataattttcaattttcattctttttaaaggagattagcatacattttatcataattatacagacaatatagtaatatatgatgttaaaatatcaatatatggttaaaaatctgtatattttttcccattttaattatttgtactataatattacactgagtgtattgcatatttaaaaaatatttggtacaattagttagttaaataaattctaaattgtaaattatcagaatccttattaaggaaatacatagatttaaggagaaatcataaaaaggtgtaatataaactggctaaaattgagcaaaaattgagcaattaagactttttgattgtatctttttatatatttaaggtatataatcttatttatattgggggaa

SEQ ID NO: 46 - DNA, 21 bp, synthetic construct
  misc_feature 1..21: note = oligonucleotide pUC57-F
  source 1..21: mol_type = other DNA
agcagattgtactgagagtgc

SEQ ID NO: 47 - DNA, 21 bp, synthetic construct
  misc_feature 1..21: note = oligonucleotide pUC57-R
  source 1..21: mol_type = other DNA
acagctatgaccatgattacg

SEQ ID NO: 48 - DNA, 3552 bp, synthetic construct
  misc_feature 1..3552: note = plasmid pMTL 85245
  source 1..3552: mol_type = other DNA
aaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcaggataaaaaaattgtagataaattttataaaatagttttatctacaatttttttatcaggaaacagctatgaccgcggccgcaatatgatatttatgtccattgtgaaagggattatattcaactattattccagttacgttcatagaaattttcctttctaaaatattttattccatgtcaagaactctgtttatttcattaaagaactataagtacaaagtataaggcatttgaaaaaataggctagtatattgattgattatttattttaaaatgcctaagtgaaatatatacatattataacaataaaataagtattagtgtaggatttttaaatagagtatctattttcagattaaatttttgattatttgatttacattatataatattgagtaaagtattgactagcaaaattttttgatactttaatttgtgaaatttcttatcaaaagttatatttttgaataatttttattgaaaaatacaactaaaaaggattatagtataagtgtgtgtaattttgtgttaaatttaaagggaggaaatgaacatgaaacatatggtgaccatgattacgaattcgagctcggtacccggggatcctctagagtcgacgtcacgcgtccatggagatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgcattcacttcttttctatataaatatgagcgaagcgaataagcgtcggaaaagcagcaaaaagtttcctttttgctgttggagcatgggggttcagggggtgcagtatctgacgtcaatgccgagcgaaagcgagccgaagggtagcatttacgttagataaccccctgatatgctccgacgctttatatagaaaagaagattcaactaggtaaaatcttaatataggttgagatgataaggtttataaggaatttgtttgttctaatttttcactcattttgttctaatttcttttaacaaatgttcttttttttttagaacagttatgatatagttagaatagtttaaaataaggagtgagaaaaagatgaaagaaagatatggaacagtctataaaggctctcagaggctcatagacgaagaaagtggagaagtcatagaggtagacaagttataccgtaaacaaacgtctggtaacttcgtaaaggcatatatagtgcaattaataagtatgttagatatgattggcggaaaaaaacttaaaatcgttaactatatcctagataatgtccacttaagtaacaatacaatgatagctacaacaagagaaatagcaaaagctacaggaacaagtctacaaacagtaataacaacacttaaaatcttagaagaaggaaatattataaaaagaaaaactggagtattaatgttaaaccctgaactactaatgagaggcgacgaccaaaaacaaaaatacctcttactcgaatttgggaactttgagcaagaggcaaatgaaatagattgacctcccaataacaccacgtagttattgggaggtcaatctatgaaatgcgattaagggccggccgaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtgttt

SEQ ID NO: 49 - DNA, 4186 bp, synthetic construct
  misc_feature 1..4186: note = plasmid pMTL 83145
  source 1..4186: mol_type = other DNA
aaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcaggataaaaaaattgtagataaattttataaaatagttttatctacaatttttttatcaggaaacagctatgaccgcggccgcaatatgatatttatgtccattgtgaaagggattatattcaactattattccagttacgttcatagaaattttcctttctaaaatattttattccatgtcaagaactctgtttatttcattaaagaactataagtacaaagtataaggcatttgaaaaaataggctagtatattgattgattatttattttaaaatgcctaagtgaaatatatacatattataacaataaaataagtattagtgtaggatttttaaatagagtatctattttcagattaaatttttgattatttgatttacattatataatattgagtaaagtattgactagcaaaattttttgatactttaatttgtgaaatttcttatcaaaagttatatttttgaataatttttattgaaaaatacaactaaaaaggattatagtataagtgtgtgtaattttgtgttaaatttaaagggaggaaatgaacatgaaacatatggtgaccatgattacgaattcgagctcggtacccggggatcctctagagtcgacgtcacgcgtccatggagatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagttt

SEQ ID NO: 50 - DNA, 9827 bp, synthetic construct
  misc_feature 1..9827: note = plasmid pMTL8215-Pptaack-thlA-HMGS-Patp-HMGR
  source 1..9827: mol_type = other DNA
cctgcaggataaaaaaattgtagataaattttataaaatagttttatctacaatttttttatcaggaaacagctatgaccgcggccgcagatagtcataatagttccagaatagttcaatttagaaattagactaaacttcaaaatgtttgttaaatatataccaatctagtatagatattttttaaatactggacttaaacagtagtaatttgcctaaaaaattttttcaattttttttaaaaaatccttttcaagttgtacattgttatggtaatatgtaattgaagaagttatgtagtaatattgtaaacgtttcttgatttttttacatccatgtagtgcttaaaaaaccaaaatatgtcacatgcacttgtatatttcaaataacaatatttattttctcgttaaattcacaaataatttattaataatatcaataaccaagattatacttaaatggatgtttattttttaacacttttatagtaaatatatttattttatgtagtaaaaaggttataattataattgtatttattacaattaattaaaataaaaaatagggttttaggtaaaattaagttattttaagaagtaattacaataaaaattgaagttatttctttaaggaggaaattcatatgaaagaggttgttattgcatcagctgttagaactgcaataggatcttatggaaaaagtcttaaagatgtaccagcagtagacttaggtgcaactgcaataaaggaagcagtaaagaaagcaggtataaaacctgaagatgttaatgaagttattttaggaaacgtattacaagctggacttggacagaatccagctagacaggcatcattcaaagcaggattaccagtagagatacctgctatgactattaataaagtttgtggttcaggattaagaacagtttctttagctgctcaaattataaaagctggtgacgcagatgtaataatagcaggtggtatggaaaatatgtcaagagcaccataccttgctaataatgctagatggggttatagaatgggaaacgctaaatttgtagacgaaatgataactgatggactttgggatgcatttaacgattatcacatgggaattactgctgaaaatatagctgagagatggaatataagtagagaagaacaagatgagtttgcacttgcatctcagaaaaaggcagaagaagctattaaatcaggacaatttaaagatgaaattgttccagtagtaattaaaggtagaaaaggtgaaacagttgtagacactgatgaacatcctagatttggatctacaatagaaggtttagctaaattaaagcctgcttttaagaaagacggaacagtaactgctggaaacgcatcaggtttaaatgattgtgcagctgttttagttattatgtctgctgaaaaggcaaaggaattaggtgttaaaccacttgctaagatagttagttatggttcagcaggtgtagatcctgctattatgggatatggacctttttatgctacaaaggcagctattgaaaaggctggttggacagttgatgaacttgatcttatagagtcaaatgaggcatttgcagcacaaagtcttgctgttgctaaggatcttaaattcgatatgaataaagtaaatgtaaacggtggtgctatagcacttggtcatccaataggtgctagtggtgctagaattttagttacattagttcatgcaatgcaaaagagagacgctaaaaagggacttgcaactttatgcataggtggtggtcaaggaacagcaatacttcttgaaaaatgttaagaattcgaggcttttactaaaaacaataaaaacaggaggaaataatatgactataggaattgacaaaataaacttttacgtaccaaaatattatgtagatatggcaaaattagcagaagcaagacaagtagacccaaataaatttcttattggaataggacagactgaaatggcagttagtccagtaaaccaagatatagtatcaatgggtgctaatgctgctaaagatataataactgatgaagacaaaaagaaaataggaatggtaatagtagcaactgagtcagcagtagatgcagcaaaggcagcagcagtacagattcataatttattaggtattcaaccatttgcaagatgtttcgaaatgaaagaagcatgttatgctgctactcctgcaattcagttagctaaggattatttagctacaagaccaaatgagaaagttttagttatagctacagatacagctagatatggacttaattcaggtggtgaacctactcaaggtgctggtgctgttgctatggttatagctcataatcctagtatacttgcattaaatgaagacgctgttgcttatacagaagatgtttatgatttctggagaccaacaggacataagtatccattagtagatggtgctttatcaaaagacgcatatattagatcttttcaacaatcttggaatgaatatgctaagagacaaggaaagagtttagctgattttgctagtctttgctttcatgttccttttactaaaatgggtaaaaaggctttagaatctataatagataacgcagatgaaacaactcaagagagattaagatctggatatgaagatgcagttgattacaatagatatgttggaaatatatacacaggaagtctttatctttctcttataagtcttcttgaaaatagagatttacaggctggtgaaactattggattattttcatacggatcaggttctgttggtgaattttattcagctacacttgtagaaggatataaagatcaccttgatcaggcagcacacaaagcacttttaaacaatagaactgaagtatcagtagatgcatacgaaacatttttcaagagatttgatgatgtagaatttgatgaagagcaggatgcagttcatgaagatagacatatattctatctttcaaacatagagaataatgtaagagaatatcatagacctgaataagagctcgttataattttcaattttcattctttttaaaggagattagcatacattttatcataattatacagacaatatagtaatatatgatgttaaaatatcaatatatggttaaaaatctgtatattttttcccattttaattatttgtactataatattacactgagtgtattgcatatttaaaaaatatttggtacaattagttagttaaataaattctaaattgtaaattatcagaatccttattaaggaaatacatagatttaaggagaaatcataaaaaggtgtaatataaactggctaaaattgagcaaaaattgagcaattaagactttttgattgtatctttttatatatttaaggtatataatcttatttatattgggggaaggtaccatgcaatcattagacaaaaatttcagacatttatcaagacaacaaaagttacaacaattagttgataaacagtggctttcagaagatcagtttgatattttacttaatcatcctcttatagatgaagaagttgctaatagtcttatagaaaatgtaattgcacagggtgcattaccagttggacttcttcctaatataatagttgatgataaggcttatgttgtaccaatgatggttgaagaacctagtgttgttgcagctgcatcttatggtgctaaattagtaaatcagacaggtggatttaaaactgtatcatcagaaagaataatgattggacagatagtatttgatggtgtagatgacactgaaaaattaagtgcagatattaaagcattagaaaaacaaatacataagattgcagatgaagcatatcctagtataaaagcaagaggtggtggttatcaaagaatagcaatagatacatttccagagcaacaacttttaagtcttaaggtatttgtagatacaaaagatgctatgggtgctaatatgcttaatactatacttgaggcaataactgcattccttaaaaatgaatctcctcaatcagatatattaatgtctatactttcaaaccatgcaactgctagtgtagtaaaagtacaaggtgagatagatgtaaaagatcttgctagaggtgaaagaacaggtgaagaagtagctaagagaatggaaagagcttctgtattagctcaggttgatattcatagagctgcaacacataacaaaggtgttatgaatggaatacatgctgttgttttagctacaggaaatgatactagaggtgctgaagcatctgcacatgcatacgcatcaagagacggacaatatagaggtatagcaacttggagatatgatcagaagagacaaagacttattggaactattgaagttccaatgacacttgctatagtaggtggtggtactaaagtattaccaatagctaaggcatcattagagttattaaatgttgattctgcacaagaacttggacacgtagttgctgctgttggattagcacaaaactttgctgcttgtagagcacttgtttctgaaggtattcaacaaggacacatgtcattacaatataaaagtttagcaatagtagtaggtgcaaaaggtgacgagatagcacaagtagcagaagctcttaaacaggaaccaagagctaatacacaggttgctgaaagaattttacaggaaattagacagcaataatctagagtcgacgtcacgcgtccatggagatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgttctgaatccttagctaatggttcaacaggtaactatgacgaagatagcaccctggataagtctgtaatggattctaaggcatttaatgaagacgtgtatataaaatgtgctaatgaaaaagaaaatgcgttaaaagagcctaaaatgagttcaaatggttttgaaattgattggtagtttaatttaatatattttttctattggctatctcgatacctatagaatcttctgttcacttttgtttttgaaatataaaaaggggctttttagccccttttttttaaaactccggaggagtttcttcattcttgatactatacgtaactattttcgatttgacttcattgtcaattaagctagtaaaatcaatggttaaaaaacaaaaaacttgcatttttctacctagtaatttataattttaagtgtcgagtttaaaagtataatttaccaggaaaggagcaagttttttaataaggaaaaatttttccttttaaaattctatttcgttatatgactaattataatcaaaaaaatgaaaataaacaagaggtaaaaactgctttagagaaatgtactgataaaaaaagaaaaaatcctagatttacgtcatacatagcacctttaactactaagaaaaatattgaaaggacttccacttgtggagattatttgtttatgttgagtgatgcagacttagaacattttaaattacataaaggtaatttttgcggtaatagattttgtccaatgtgtagttggcgacttgcttgtaaggatagtttagaaatatctattcttatggagcatttaagaaaagaagaaaataaagagtttatatttttaactcttacaactccaaatgtaaaaagttatgatcttaattattctattaaacaatataataaatcttttaaaaaattaatggagcgtaaggaagttaaggatataactaaaggttatataagaaaattagaagtaacttaccaaaaggaaaaatacataacaaaggatttatggaaaataaaaaaagattattatcaaaaaaaaggacttgaaattggtgatttagaacctaattttgatacttataatcctcattttcatgtagttattgcagttaataaaagttattttacagataaaaattattatataaatcgagaaagatggttggaattatggaagtttgctactaaggatgattctataactcaagttgatgttagaaaagcaaaaattaatgattataaagaggtttacgaacttgcgaaatattcagctaaagacactgattatttaatatcgaggccagtatttgaaattttttataaagcattaaaaggcaagcaggtattagtttttagtggattttttaaagatgcacacaaattgtacaagcaaggaaaacttgatgtttataaaaagaaagatgaaattaaatatgtctatatagtttattataattggtgcaaaaaacaatatgaaaaaactagaataagggaacttacggaagatgaaaaagaagaattaaatcaagatttaatagatgaaatagaaatagattaaagtgtaactatactttatatatatatgattaaaaaaataaaaaacaacagcctattaggttgttgttttttattttctttattaatttttttaatttttagtttttagttcttttttaaaataagtttcagcctctttttcaatattttttaaagaaggagtatttgcatgaattgccttttttctaacagacttaggaaatattttaacagtatcttcttgcgccggtgattttggaacttcataacttactaatttataattattattttcttttttaattgtaacagttgcaaaagaagctgaacctgttccttcaactagtttatcatcttcaatataatattcttgacctatatagtataaatatatttttattatatttttacttttttctgaatctattattttataatcataaaaagttttaccaccaaaagaaggttgtactccttctggtccaacatatttttttactatattatctaaataatttttgggaactggtgttgtaatttgattaatcgaacaaccagttatacttaaaggaattataactataaaaatatataggattatctttttaaatttcattattggcctcctttttattaaatttatgttaccataaaaaggacataacgggaatatgtagaatatttttaatgtagacaaaattttacataaatataaagaaaggaagtgtttgtttaaattttatagcaaactatcaaaaattagggggataaaaatttatgaaaaaaaggttttcgatgttatttttatgtttaactttaatagtttgtggtttatttacaaattcggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatcgggccc

SEQ ID NO: 51 - DNA, 843 bp, Staphylococcus aureus
  source 1..843: mol_type = other DNA; sub_species = aureus
atgatagctgttccatttaacgctggaaaaataaaagttttaattgaggcattagaatctggaaattattcatcaataaaatcagatgtatatgacggaatgttatatgatgcaccagatcaccttaaatcattagtaaacagatttgtagaacttaataatataactgagccattagcagtaactatacagacaaatcttcctccttcaagaggtcttggatctagtgcagctgttgctgttgcttttgtaagagcaagttatgatttcttaggaaaaagtttaactaaagaagagcttatagaaaaggctaattgggctgaacaaatagctcatggaaagccatctggaatagatacacaaacaatagtatctggaaagcctgtttggtttcaaaagggacatgcagaaacacttaaaactctttcacttgatggatacatggtagtaattgatacaggtgttaaaggaagtacaagacaggctgtagaagatgttcataaactttgcgaagatcctcaatatatgagtcacgtaaaacacataggaaaacttgtacttagagcatctgatgttattgaacatcataactttgaagcacttgctgatatattcaatgaatgtcatgctgatttaaaggctcttacagtaagtcatgacaaaatagaacagttaatgaagataggaaaagaaaatggtgctatagctggtaaattaactggtgctggtagaggtggttcaatgttattacttgcaaaagacttaccaactgcaaagaatatagttaaagcagtagagaaagctggtgcagcacatacttggattgaaaatttaggtggttaa

SEQ ID NO: 52 - DNA, 1077 bp, Staphylococcus aureus
  source 1..1077: mol_type = other DNA; sub_species = subsp. aureus
atgatacaagtaaaggcaccaggaaaattatatatagcaggtgaatacgctgttacagaaccaggatataaatctgttcttatagctcttgatagatttgttacagctactattgaggaagctgatcaatacaaaggaacaatacattcaaaggcattacatcacaatccagtaacttttagtagagatgaagattctattgttatatcagacccacacgcagcaaaacaacttaattatgtagtaactgctatagaaatatttgagcaatatgcaaaatcatgtgacatagcaatgaagcattttcatttaactatagattctaacttagatgatagtaatggacataagtatggacttggatcttctgctgctgttttagtttcagtaattaaagtacttaacgaattttatgatatgaaactttcaaacctttatatatataagttagcagtaattgctaatatgaaattacagagtttatcttcatgcggtgatatagcagtaagtgtttattcaggttggttagcttattctacatttgaccatgaatgggtaaaacaccagatagaagatacaacagttgaagaagtacttattaaaaattggcctggattacacatagagccacttcaagctcctgaaaatatggaagttcttataggttggacaggtagtccagctagtagtcctcattttgtttctgaagttaaaagacttaagtcagatccttcattttacggtgatttcttagaagattcacatagatgtgtagaaaaattaattcatgcattcaaaactaataatattaagggtgttcagaaaatggtaagacagaatagaactattatacaaagaatggataaggaagcaacagttgatatagagactgagaagttaaaatatttatgtgatattgctgaaaaatatcatggtgcaagtaaaacttcaggtgctggtggtggtgattgcggaataactataataaataaggatgtagacaaagagaaaatatatgatgaatggactaaacatggaataaagcctcttaagtttaatatttatcatggacaataa

SEQ ID NO: 53 - DNA, 984 bp, Staphylococcus aureus
  source 1..984: mol_type = other DNA; sub_species = subsp. aureus
atgataaaatctggaaaagcaagagcacacactaatatagcacttataaaatattggggtaagaaagatgaggcattaataataccaatgaataactcaatatcagtaactttagaaaagttttatactgaaacaaaagttacatttaacgatcagcttactcaagatcaattttggcttaatggtgaaaaagtttctggaaaagaattagaaaagatttcaaagtatatggatattgttagaaatagagctggaatagattggtatgctgagatagaatctgataattttgttcctacagctgctggtcttgctagttctgctagtgcttatgcagcattagctgctgcatgtaaccaagcacttgatttacagttaagtgataaagacttaagtagattagctagaattggatcaggatcagcatcaagatcaatatacggtggttttgcagaatgggaaaaaggatataatgacgaaacttcttatgctgttccattagaaagtaatcactttgaagatgatcttgctatgatttttgtagtaataaaccaacattctaaaaaggttccttcaagatatggaatgtctcttacaagaaatacaagtagattctatcaatattggttagaccatattgatgaagatcttgcagaagcaaaggcagcaatacaagataaggattttaagagattaggtgaagttattgaagagaatggacttagaatgcatgctacaaatcttggatcaactccaccttttacttacttagtacaagagtcatacgatgtaatggcattagtacatgagtgtagagaagcaggatatccatgctatttcactatggatgctggacctaatgtaaaaatacttgtagagaagaaaaacaaacaacagataatagataaacttttaactcagttcgataataatcagataatagatagtgatattatagctacaggtattgaaattatagaataa

SEQ ID NO: 54 - DNA, 516 bp, Clostridium beijerinckii
  source 1..516: mol_type = other DNA
atggcagagtatataatagcagtagatgagttcgataacgaaataggatcaatagaaaagatggaagctcatagaaaaggaacacttcatagagcattcagtattttagtttttaactcaaagaatcaacttttattacagaaaagaaatgtaaagaaatatcactctccaggattatggacaaacacttgttgtagtcacccaagatatggtgaatctcttcatgatgctatatacagaagattaaaagaagagatgggatttacttgcgaacttgaagaagtattctcattcatatataaggtaaaacttgaagataatttatttgagaatgaatatgaccatgtatttattggtaaatatgatggtgagataattgttaataaagatgaagttgatgattttaaatgggtagacattaatgaagttaaaaaggacataatagaaagacctgaggcatatacttactggtttaagtatcttgtaaataaagctgaaaataagatatttaaataa

SEQ ID NO: 55 - DNA, 120 bp, Clostridium autoethanogenum
  source 1..120: mol_type = other DNA
atatcgatacagataaaaaaatatataatacagaagaaaaaattataaatttgtggtataatataaagtatagtaatttaagtttaaacctcgtgaaaacgctaacaaataataggaggt

SEQ ID NO: 56 - DNA, 900 bp, Escherichia coli
  source 1..900: mol_type = other DNA
atggatttcccacaacaattagaagcatgtgtaaaacaggctaatcaggcacttagtagatttattgctcctcttccttttcaaaatacaccagtagtagaaactatgcaatacggtgcacttttaggtggtaaaagattaagaccattcttagtatatgctacaggacatatgtttggtgtatcaactaatactttagacgctccagctgctgctgttgaatgtattcatgcatattctttaatacatgatgacttaccagcaatggatgacgatgatttaagaagaggtttacctacatgtcatgttaaatttggtgaagctaatgcaattttagcaggtgacgctttacaaactttagctttttctatactttcagatgcagacatgcctgaagtttcagatagagatagaatttctatgatatcagagcttgcatctgcatcaggaatagctggaatgtgcggtggtcaagcacttgatttagatgcagaaggtaaacacgtaccacttgatgcattagagagaattcatagacataaaacaggtgctcttataagagcagcagtaagattaggtgctttaagtgctggtgacaagggtagaagagcacttccagtacttgataagtatgcagaaagtataggattagcttttcaagttcaagatgacatacttgacgttgttggtgatactgctactttaggaaaaagacagggtgcagatcagcaattaggaaaatctacataccctgctttacttggattagaacaggctagaaagaaagcaagagacttaatagatgacgcaagacaaagtcttaaacagttagctgaacaatcacttgacacaagtgcacttgaagcacttgcagattatattatacagagaaacaagtaa

SEQ ID NO: 57 - DNA, 975 bp, Malus x domestica
  source 1..975: mol_type = other DNA
atggaatttagagtacatttacaggcagacaacgaacagaaaatatttcaaaatcaaatgaaaccagagccagaagcatcatatcttataaatcaaagaagaagtgctaattataaaccaaacatttggaaaaacgattttcttgatcagtctttaatatcaaaatatgatggtgatgaatatagaaaactttcagaaaagttaatagaagaagtaaagatatacatatcagcagagactatggatttagttgctaaattagaacttatagattctgttagaaaacttggacttgctaatctttttgagaaagaaataaaggaagcattagacagtatagcagcaatagaatcagataatttaggaactagagacgatctttatggaacagctcttcattttaagattcttagacagcatggatataaggtaagtcaagatatatttggtagatttatggatgagaaaggaacattagaaaatcatcactttgcacacttaaaaggaatgttagaattatttgaggcaagtaatcttggatttgaaggtgaagacatattagatgaagctaaagcatctcttacacttgctcttagagattcaggacatatttgttatccagactcaaacttaagtagagatgtagttcatagtttagaattacctagtcatagaagagttcaatggttcgatgtaaaatggcagattaatgcatacgaaaaagatatttgtagagtaaatgcaactttattagagttagcaaagttaaattttaatgttgttcaagctcagcttcagaagaatcttagagaagctagtagatggtgggctaatcttggtttcgcagataatttaaagtttgctagagatagacttgtagagtgtttttcatgcgcagtaggtgtagcatttgaaccagagcattcatcttttagaatatgtttaactaaggtaattaatcttgttcttattata

SEQ ID NO: 58 - DNA, 13817 bp, synthetic construct
  misc_feature 1..13817: note = pMTL8314-Pptaack-thlA-HMGS-Patp-HMGR-Prnf-MK-PMK-PM D-Pfor-idi-ispS
  source 1..13817: mol_type = other DNA
aaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcaggataaaaaaattgtagataaattttataaaatagttttatctacaatttttttatcaggaaacagctatgaccgcggccgcagatagtcataatagttccagaatagttcaatttagaaattagactaaacttcaaaatgtttgttaaatatataccaatctagtatagatattttttaaatactggacttaaacagtagtaatttgcctaaaaaattttttcaattttttttaaaaaatccttttcaagttgtacattgttatggtaatatgtaattgaagaagttatgtagtaatattgtaaacgtttcttgatttttttacatccatgtagtgcttaaaaaaccaaaatatgtcacatgcacttgtatatttcaaataacaatatttattttctcgttaaattcacaaataatttattaataatatcaataaccaagattatacttaaatggatgtttattttttaacacttttatagtaaatatatttattttatgtagtaaaaaggttataattataattgtatttattacaattaattaaaataaaaaatagggttttaggtaaaattaagttattttaagaagtaattacaataaaaattgaagttatttctttaaggaggaaattcatatgaaagaggttgttattgcatcagctgttagaactgcaataggatcttatggaaaaagtcttaaagatgtaccagcagtagacttaggtgcaactgcaataaaggaagcagtaaagaaagcaggtataaaacctgaagatgttaatgaagttattttaggaaacgtattacaagctggacttggacagaatccagctagacaggcatcattcaaagcaggattaccagtagagatacctgctatgactattaataaagtttgtggttcaggattaagaacagtttctttagctgctcaaattataaaagctggtgacgcagatgtaataatagcaggtggtatggaaaatatgtcaagagcaccataccttgctaataatgctagatggggttatagaatgggaaacgctaaatttgtagacgaaatgataactgatggactttgggatgcatttaacgattatcacatgggaattactgctgaaaatatagctgagagatggaatataagtagagaagaacaagatgagtttgcacttgcatctcagaaaaaggcagaagaagctattaaatcaggacaatttaaagatgaaattgttccagtagtaattaaaggtagaaaaggtgaaacagttgtagacactgatgaacatcctagatttggatctacaatagaaggtttagctaaattaaagcctgcttttaagaaagacggaacagtaactgctggaaacgcatcaggtttaaatgattgtgcagctgttttagttattatgtctgctgaaaaggcaaaggaattaggtgttaaaccacttgctaagatagttagttatggttcagcaggtgtagatcctgctattatgggatatggacctttttatgctacaaaggcagctattgaaaaggctggttggacagttgatgaacttgatcttatagagtcaaatgaggcatttgcagcacaaagtcttgctgttgctaaggatcttaaattcgatatgaataaagtaaatgtaaacggtggtgctatagcacttggtcatccaataggtgctagtggtgctagaattttagttacattagttcatgcaatgcaaaagagagacgctaaaaagggacttgcaactttatgcataggtggtggtcaaggaacagcaatacttcttgaaaaatgttaagaattcgaggcttttactaaaaacaataaaaacaggaggaaataatatgactataggaattgacaaaataaacttttacgtaccaaaatattatgtagatatggcaaaattagcagaagcaagacaagtagacccaaataaatttcttattggaataggacagactgaaatggcagttagtccagtaaaccaagatatagtatcaatgggtgctaatgctgctaaagatataataactgatgaagacaaaaagaaaataggaatggtaatagtagcaactgagtcagcagtagatgcagcaaaggcagcagcagtacagattcataatttattaggtattcaaccatttgcaagatgtttcgaaatgaaagaagcatgttatgctgctactcctgcaattcagttagctaaggattatttagctacaagaccaaatgagaaagttttagttatagctacagatacagctagatatggacttaattcaggtggtgaacctactcaaggtgctggtgctgttgctatggttatagctcataatcctagtatacttgcattaaatgaagacgctgttgcttatacagaagatgtttatgatttctggagaccaacaggacataagtatccattagtagatggtgctttatcaaaagacgcatatattagatcttttcaacaatcttggaatgaatatgctaagagacaaggaaagagtttagctgattttgctagtctttgctttcatgttccttttactaaaatgggtaaaaaggctttagaatctataatagataacgcagatgaaacaactcaagagagattaagatctggatatgaagatgcagttgattacaatagatatgttggaaatatatacacaggaagtctttatctttctcttataagtcttcttgaaaatagagatttacaggctggtgaaactattggattattttcatacggatcaggttctgttggtgaattttattcagctacacttgtagaaggatataaagatcaccttgatcaggcagcacacaaagcacttttaaacaatagaactgaagtatcagtagatgcatacgaaacatttttcaagagatttgatgatgtagaatttgatgaagagcaggatgcagttcatgaagatagacatatattctatctttcaaacatagagaataatgtaagagaatatcatagacctgaataagagctcgttataattttcaattttcattctttttaaaggagattagcatacattttatcataattatacagacaatatagtaatatatgatgttaaaatatcaatatatggttaaaaatctgtatattttttcccattttaattatttgtactataatattacactgagtgtattgcatatttaaaaaatatttggtacaattagttagttaaataaattctaaattgtaaattatcagaatccttattaaggaaatacatagatttaaggagaaatcataaaaaggtgtaatataaactggctaaaattgagcaaaaattgagcaattaagactttttgattgtatctttttatatatttaaggtatataatcttatttatattgggggaaggtaccatgcaatcattagacaaaaatttcagacatttatcaagacaacaaaagttacaacaattagttgataaacagtggctttcagaagatcagtttgatattttacttaatcatcctcttatagatgaagaagttgctaatagtcttatagaaaatgtaattgcacagggtgcattaccagttggacttcttcctaatataatagttgatgataaggcttatgttgtaccaatgatggttgaagaacctagtgttgttgcagctgcatcttatggtgctaaattagtaaatcagacaggtggatttaaaactgtatcatcagaaagaataatgattggacagatagtatttgatggtgtagatgacactgaaaaattaagtgcagatattaaagcattagaaaaacaaatacataagattgcagatgaagcatatcctagtataaaagcaagaggtggtggttatcaaagaatagcaatagatacatttccagagcaacaacttttaagtcttaaggtatttgtagatacaaaagatgctatgggtgctaatatgcttaatactatacttgaggcaataactgcattccttaaaaatgaatctcctcaatcagatatattaatgtctatactttcaaaccatgcaactgctagtgtagtaaaagtacaaggtgagatagatgtaaaagatcttgctagaggtgaaagaacaggtgaagaagtagctaagagaatggaaagagcttctgtattagctcaggttgatattcatagagctgcaacacataacaaaggtgttatgaatggaatacatgctgttgttttagctacaggaaatgatactagaggtgctgaagcatctgcacatgcatacgcatcaagagacggacaatatagaggtatagcaacttggagatatgatcagaagagacaaagacttattggaactattgaagttccaatgacacttgctatagtaggtggtggtactaaagtattaccaatagctaaggcatcattagagttattaaatgttgattctgcacaagaacttggacacgtagttgctgctgttggattagcacaaaactttgctgcttgtagagcacttgtttctgaaggtattcaacaaggacacatgtcattacaatataaaagtttagcaatagtagtaggtgcaaaaggtgacgagatagcacaagtagcagaagctcttaaacaggaaccaagagctaatacacaggttgctgaaagaattttacaggaaattagacagcaataatctagaatatcgatacagataaaaaaatatataatacagaagaaaaaattataaatttgtggtataatataaagtatagtaatttaagtttaaacctcgtgaaaacgctaacaaataataggaggtcaattgatgatagctgttccatttaacgctggaaaaataaaagttttaattgaggcattagaatctggaaattattcatcaataaaatcagatgtatatgacggaatgttatatgatgcaccagatcaccttaaatcattagtaaacagatttgtagaacttaataatataactgagccattagcagtaactatacagacaaatcttcctccttcaagaggtcttggatctagtgcagctgttgctgttgcttttgtaagagcaagttatgatttcttaggaaaaagtttaactaaagaagagcttatagaaaaggctaattgggctgaacaaatagctcatggaaagccatctggaatagatacacaaacaatagtatctggaaagcctgtttggtttcaaaagggacatgcagaaacacttaaaactctttcacttgatggatacatggtagtaattgatacaggtgttaaaggaagtacaagacaggctgtagaagatgttcataaactttgcgaagatcctcaatatatgagtcacgtaaaacacataggaaaacttgtacttagagcatctgatgttattgaacatcataactttgaagcacttgctgatatattcaatgaatgtcatgctgatttaaaggctcttacagtaagtcatgacaaaatagaacagttaatgaagataggaaaagaaaatggtgctatagctggtaaattaactggtgctggtagaggtggttcaatgttattacttgcaaaagacttaccaactgcaaagaatatagttaaagcagtagagaaagctggtgcagcacatacttggattgaaaatttaggtggttaagtcgacaaagacactaaaaaattataaaagtaaaggaggacattaaatgatacaagtaaaggcaccaggaaaattatatatagcaggtgaatacgctgttacagaaccaggatataaatctgttcttatagctcttgatagatttgttacagctactattgaggaagctgatcaatacaaaggaacaatacattcaaaggcattacatcacaatccagtaacttttagtagagatgaagattctattgttatatcagacccacacgcagcaaaacaacttaattatgtagtaactgctatagaaatatttgagcaatatgcaaaatcatgtgacatagcaatgaagcattttcatttaactatagattctaacttagatgatagtaatggacataagtatggacttggatcttctgctgctgttttagtttcagtaattaaagtacttaacgaattttatgatatgaaactttcaaacctttatatatataagttagcagtaattgctaatatgaaattacagagtttatcttcatgcggtgatatagcagtaagtgtttattcaggttggttagcttattctacatttgaccatgaatgggtaaaacaccagatagaagatacaacagttgaagaagtacttattaaaaattggcctggattacacatagagccacttcaagctcctgaaaatatggaagttcttataggttggacaggtagtccagctagtagtcctcattttgtttctgaagttaaaagacttaagtcagatccttcattttacggtgatttcttagaagattcacatagatgtgtagaaaaattaattcatgcattcaaaactaataatattaagggtgttcagaaaatggtaagacagaatagaactattatacaaagaatggataaggaagcaacagttgatatagagactgagaagttaaaatatttatgtgatattgctgaaaaatatcatggtgcaagtaaaacttcaggtgctggtggtggtgattgcggaataactataataaataaggatgtagacaaagagaaaatatatgatgaatggactaaacatggaataaagcctcttaagtttaatatttatcatggacaataaccatggtcaataatcttacaataaataaaagaaaggaggcaaaaatatgataaaatctggaaaagcaagagcacacactaatatagcacttataaaatattggggtaagaaagatgaggcattaataataccaatgaataactcaatatcagtaactttagaaaagttttatactgaaacaaaagttacatttaacgatcagcttactcaagatcaattttggcttaatggtgaaaaagtttctggaaaagaattagaaaagatttcaaagtatatggatattgttagaaatagagctggaatagattggtatgctgagatagaatctgataattttgttcctacagctgctggtcttgctagttctgctagtgcttatgcagcattagctgctgcatgtaaccaagcacttgatttacagttaagtgataaagacttaagtagattagctagaattggatcaggatcagcatcaagatcaatatacggtggttttgcagaatgggaaaaaggatataatgacgaaacttcttatgctgttccattagaaagtaatcactttgaagatgatcttgctatgatttttgtagtaataaaccaacattctaaaaaggttccttcaagatatggaatgtctcttacaagaaatacaagtagattctatcaatattggttagaccatattgatgaagatcttgcagaagcaaaggcagcaatacaagataaggattttaagagattaggtgaagttattgaagagaatggacttagaatgcatgctacaaatcttggatcaactccaccttttacttacttagtacaagagtcatacgatgtaatggcattagtacatgagtgtagagaagcaggatatccatgctatttcactatggatgctggacctaatgtaaaaatacttgtagagaagaaaaacaaacaacagataatagataaacttttaactcagttcgataataatcagataatagatagtgatattatagctacaggtattgaaattatagaataaactagttgtatattaaaatagtagaatacataagatacttaatttaattaaagatagttaagtacttttcaatgtgcttttttagatgtttaatacaaatctttaattgtaaaagaaatgctgtactatttactgttctagtgacgggattaaactgtattaattataaataaaaaataagtacagttgtttaaaattatattttgtattaaatctaatagtacgatgtaagttattttatactattgctagtttaataaaaagatttaattatatacttgaaaaggagaggaactcgagatggcagagtatataatagcagtagatgagttcgataacgaaataggatcaatagaaaagatggaagctcatagaaaaggaacacttcatagagcattcagtattttagtttttaactcaaagaatcaacttttattacagaaaagaaatgtaaagaaatatcactctccaggattatggacaaacacttgttgtagtcacccaagatatggtgaatctcttcatgatgctatatacagaagattaaaagaagagatgggatttacttgcgaacttgaagaagtattctcattcatatataaggtaaaacttgaagataatttatttgagaatgaatatgaccatgtatttattggtaaatatgatggtgagataattgttaataaagatgaagttgatgattttaaatgggtagacattaatgaagttaaaaaggacataatagaaagacctgaggcatatacttactggtttaagtatcttgtaaataaagctgaaaataagatatttaaataaaccggtgggaggaaatgaacatggcaacagaattattatgtttacacagacctatatcacttactcacaaactttttaggaatccattacctaaagttattcaagctacacctttaacattaaaacttaggtgtagtgtttctacagaaaatgtatcatttagtgagacagaaactgaaacaagaagatcagcaaattatgaaccaaattcttgggattatgattatcttctttcttctgatactgatgagtcaatagaagtacataaagataaggctaagaaattagaagctgaagttaggagagaaataaataatgagaaggctgaatttcttacacttcttgaacttattgataatgtacaaagacttggattaggatatagatttgagtctgatataagaagagcattagatagatttgtaagtagtggaggatttgatggagttactaaaacttcattacatggaacagcattatcatttaggttattaaggcaacatggttttgaagtatctcaagaagcttttagtggatttaaagatcagaatggaaactttcttgagaatttaaaggaagacataaaagcaattctttctctttatgaagcatcatttttagcattagaaggtgagaatatattagatgaggctaaagtatttgcaatatctcatcttaaagaacttagtgaagaaaagattggtaaagaattagctgaacaagtttcacatgctttagaattaccattacatagaagaacacaaagattagaagcagtttggtcaatagaagcatatagaaagaaagaagacgcaaatcaagtacttttagaacttgcaatacttgactacaatatgattcaaagtgtatatcagagggatttaagagaaacatcaagatggtggagaagagtaggattagcaactaaattacattttgctagagataggcttattgaaagtttttattgggctgttggagttgcttttgaaccacaatattctgattgcagaaatagtgtagcaaagatgttttcatttgttactataattgacgatatttacgatgtatatggaactttagatgaacttgaactttttactgatgcagttgaaagatgggatgtaaatgctattaatgatcttcctgattatatgaagttatgttttcttgcactttacaatactattaacgagatagcttacgataacttaaaagataaaggtgagaacatacttccttatttaacaaaagcatgggcagatttatgtaatgcatttcttcaagaagctaagtggctttataataaatcaacacctacatttgatgattattttggaaatgcatggaaaagttctagtggacctttacagcttatttttgcttattttgctgtagtacagaacattaaaaaggaagagattgagaatcttcagaaatatcatgacataatatcaagacctagtcacatttttaggctttgtaatgatttagcatctgcttcagcagaaatagcaagaggtgaaactgctaattctgtaagttgttatatgagaacaaaaggtatatctgaagaattagctactgaaagtgttatgaatcttatagacgaaacttggaagaaaatgaacaaagaaaaacttggtggatctttatttgcaaaaccttttgttgagactgctataaatttagctagacagtctcattgcacatatcataatggtgatgcacatactagtccagatgaattaactaggaaaagagtacttagtgtaataactgaaccaatattaccatttgaaagataagctagcataaaaataagaagcctgcatttgcaggcttcttatttttatggcgcgccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagttt

SEQ ID NO: 59 - DNA, 14709 bp, synthetic construct
  misc_feature 1..14709: note = plasmidpMTL8314-Pptaack-thlA-HMGS-Patp-HMGR-Prnf-MK -PMK-PMD-Pfor-idi-ispA-FS
  source 1..14709: mol_type = other DNA
aaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcaggataaaaaaattgtagataaattttataaaatagttttatctacaatttttttatcaggaaacagctatgaccgcggccgcagatagtcataatagttccagaatagttcaatttagaaattagactaaacttcaaaatgtttgttaaatatataccaatctagtatagatattttttaaatactggacttaaacagtagtaatttgcctaaaaaattttttcaattttttttaaaaaatccttttcaagttgtacattgttatggtaatatgtaattgaagaagttatgtagtaatattgtaaacgtttcttgatttttttacatccatgtagtgcttaaaaaaccaaaatatgtcacatgcacttgtatatttcaaataacaatatttattttctcgttaaattcacaaataatttattaataatatcaataaccaagattatacttaaatggatgtttattttttaacacttttatagtaaatatatttattttatgtagtaaaaaggttataattataattgtatttattacaattaattaaaataaaaaatagggttttaggtaaaattaagttattttaagaagtaattacaataaaaattgaagttatttctttaaggaggaaattcatatgaaagaggttgttattgcatcagctgttagaactgcaataggatcttatggaaaaagtcttaaagatgtaccagcagtagacttaggtgcaactgcaataaaggaagcagtaaagaaagcaggtataaaacctgaagatgttaatgaagttattttaggaaacgtattacaagctggacttggacagaatccagctagacaggcatcattcaaagcaggattaccagtagagatacctgctatgactattaataaagtttgtggttcaggattaagaacagtttctttagctgctcaaattataaaagctggtgacgcagatgtaataatagcaggtggtatggaaaatatgtcaagagcaccataccttgctaataatgctagatggggttatagaatgggaaacgctaaatttgtagacgaaatgataactgatggactttgggatgcatttaacgattatcacatgggaattactgctgaaaatatagctgagagatggaatataagtagagaagaacaagatgagtttgcacttgcatctcagaaaaaggcagaagaagctattaaatcaggacaatttaaagatgaaattgttccagtagtaattaaaggtagaaaaggtgaaacagttgtagacactgatgaacatcctagatttggatctacaatagaaggtttagctaaattaaagcctgcttttaagaaagacggaacagtaactgctggaaacgcatcaggtttaaatgattgtgcagctgttttagttattatgtctgctgaaaaggcaaaggaattaggtgttaaaccacttgctaagatagttagttatggttcagcaggtgtagatcctgctattatgggatatggacctttttatgctacaaaggcagctattgaaaaggctggttggacagttgatgaacttgatcttatagagtcaaatgaggcatttgcagcacaaagtcttgctgttgctaaggatcttaaattcgatatgaataaagtaaatgtaaacggtggtgctatagcacttggtcatccaataggtgctagtggtgctagaattttagttacattagttcatgcaatgcaaaagagagacgctaaaaagggacttgcaactttatgcataggtggtggtcaaggaacagcaatacttcttgaaaaatgttaagaattcgaggcttttactaaaaacaataaaaacaggaggaaataatatgactataggaattgacaaaataaacttttacgtaccaaaatattatgtagatatggcaaaattagcagaagcaagacaagtagacccaaataaatttcttattggaataggacagactgaaatggcagttagtccagtaaaccaagatatagtatcaatgggtgctaatgctgctaaagatataataactgatgaagacaaaaagaaaataggaatggtaatagtagcaactgagtcagcagtagatgcagcaaaggcagcagcagtacagattcataatttattaggtattcaaccatttgcaagatgtttcgaaatgaaagaagcatgttatgctgctactcctgcaattcagttagctaaggattatttagctacaagaccaaatgagaaagttttagttatagctacagatacagctagatatggacttaattcaggtggtgaacctactcaaggtgctggtgctgttgctatggttatagctcataatcctagtatacttgcattaaatgaagacgctgttgcttatacagaagatgtttatgatttctggagaccaacaggacataagtatccattagtagatggtgctttatcaaaagacgcatatattagatcttttcaacaatcttggaatgaatatgctaagagacaaggaaagagtttagctgattttgctagtctttgctttcatgttccttttactaaaatgggtaaaaaggctttagaatctataatagataacgcagatgaaacaactcaagagagattaagatctggatatgaagatgcagttgattacaatagatatgttggaaatatatacacaggaagtctttatctttctcttataagtcttcttgaaaatagagatttacaggctggtgaaactattggattattttcatacggatcaggttctgttggtgaattttattcagctacacttgtagaaggatataaagatcaccttgatcaggcagcacacaaagcacttttaaacaatagaactgaagtatcagtagatgcatacgaaacatttttcaagagatttgatgatgtagaatttgatgaagagcaggatgcagttcatgaagatagacatatattctatctttcaaacatagagaataatgtaagagaatatcatagacctgaataagagctcgttataattttcaattttcattctttttaaaggagattagcatacattttatcataattatacagacaatatagtaatatatgatgttaaaatatcaatatatggttaaaaatctgtatattttttcccattttaattatttgtactataatattacactgagtgtattgcatatttaaaaaatatttggtacaattagttagttaaataaattctaaattgtaaattatcagaatccttattaaggaaatacatagatttaaggagaaatcataaaaaggtgtaatataaactggctaaaattgagcaaaaattgagcaattaagactttttgattgtatctttttatatatttaaggtatataatcttatttatattgggggaaggtaccatgcaatcattagacaaaaatttcagacatttatcaagacaacaaaagttacaacaattagttgataaacagtggctttcagaagatcagtttgatattttacttaatcatcctcttatagatgaagaagttgctaatagtcttatagaaaatgtaattgcacagggtgcattaccagttggacttcttcctaatataatagttgatgataaggcttatgttgtaccaatgatggttgaagaacctagtgttgttgcagctgcatcttatggtgctaaattagtaaatcagacaggtggatttaaaactgtatcatcagaaagaataatgattggacagatagtatttgatggtgtagatgacactgaaaaattaagtgcagatattaaagcattagaaaaacaaatacataagattgcagatgaagcatatcctagtataaaagcaagaggtggtggttatcaaagaatagcaatagatacatttccagagcaacaacttttaagtcttaaggtatttgtagatacaaaagatgctatgggtgctaatatgcttaatactatacttgaggcaataactgcattccttaaaaatgaatctcctcaatcagatatattaatgtctatactttcaaaccatgcaactgctagtgtagtaaaagtacaaggtgagatagatgtaaaagatcttgctagaggtgaaagaacaggtgaagaagtagctaagagaatggaaagagcttctgtattagctcaggttgatattcatagagctgcaacacataacaaaggtgttatgaatggaatacatgctgttgttttagctacaggaaatgatactagaggtgctgaagcatctgcacatgcatacgcatcaagagacggacaatatagaggtatagcaacttggagatatgatcagaagagacaaagacttattggaactattgaagttccaatgacacttgctatagtaggtggtggtactaaagtattaccaatagctaaggcatcattagagttattaaatgttgattctgcacaagaacttggacacgtagttgctgctgttggattagcacaaaactttgctgcttgtagagcacttgtttctgaaggtattcaacaaggacacatgtcattacaatataaaagtttagcaatagtagtaggtgcaaaaggtgacgagatagcacaagtagcagaagctcttaaacaggaaccaagagctaatacacaggttgctgaaagaattttacaggaaattagacagcaataatctagaatatcgatacagataaaaaaatatataatacagaagaaaaaattataaatttgtggtataatataaagtatagtaatttaagtttaaacctcgtgaaaacgctaacaaataataggaggtcaattgatgatagctgttccatttaacgctggaaaaataaaagttttaattgaggcattagaatctggaaattattcatcaataaaatcagatgtatatgacggaatgttatatgatgcaccagatcaccttaaatcattagtaaacagatttgtagaacttaataatataactgagccattagcagtaactatacagacaaatcttcctccttcaagaggtcttggatctagtgcagctgttgctgttgcttttgtaagagcaagttatgatttcttaggaaaaagtttaactaaagaagagcttatagaaaaggctaattgggctgaacaaatagctcatggaaagccatctggaatagatacacaaacaatagtatctggaaagcctgtttggtttcaaaagggacatgcagaaacacttaaaactctttcacttgatggatacatggtagtaattgatacaggtgttaaaggaagtacaagacaggctgtagaagatgttcataaactttgcgaagatcctcaatatatgagtcacgtaaaacacataggaaaacttgtacttagagcatctgatgttattgaacatcataactttgaagcacttgctgatatattcaatgaatgtcatgctgatttaaaggctcttacagtaagtcatgacaaaatagaacagttaatgaagataggaaaagaaaatggtgctatagctggtaaattaactggtgctggtagaggtggttcaatgttattacttgcaaaagacttaccaactgcaaagaatatagttaaagcagtagagaaagctggtgcagcacatacttggattgaaaatttaggtggttaagtcgacaaagacactaaaaaattataaaagtaaaggaggacattaaatgatacaagtaaaggcaccaggaaaattatatatagcaggtgaatacgctgttacagaaccaggatataaatctgttcttatagctcttgatagatttgttacagctactattgaggaagctgatcaatacaaaggaacaatacattcaaaggcattacatcacaatccagtaacttttagtagagatgaagattctattgttatatcagacccacacgcagcaaaacaacttaattatgtagtaactgctatagaaatatttgagcaatatgcaaaatcatgtgacatagcaatgaagcattttcatttaactatagattctaacttagatgatagtaatggacataagtatggacttggatcttctgctgctgttttagtttcagtaattaaagtacttaacgaattttatgatatgaaactttcaaacctttatatatataagttagcagtaattgctaatatgaaattacagagtttatcttcatgcggtgatatagcagtaagtgtttattcaggttggttagcttattctacatttgaccatgaatgggtaaaacaccagatagaagatacaacagttgaagaagtacttattaaaaattggcctggattacacatagagccacttcaagctcctgaaaatatggaagttcttataggttggacaggtagtccagctagtagtcctcattttgtttctgaagttaaaagacttaagtcagatccttcattttacggtgatttcttagaagattcacatagatgtgtagaaaaattaattcatgcattcaaaactaataatattaagggtgttcagaaaatggtaagacagaatagaactattatacaaagaatggataaggaagcaacagttgatatagagactgagaagttaaaatatttatgtgatattgctgaaaaatatcatggtgcaagtaaaacttcaggtgctggtggtggtgattgcggaataactataataaataaggatgtagacaaagagaaaatatatgatgaatggactaaacatggaataaagcctcttaagtttaatatttatcatggacaataaccatggtcaataatcttacaataaataaaagaaaggaggcaaaaatatgataaaatctggaaaagcaagagcacacactaatatagcacttataaaatattggggtaagaaagatgaggcattaataataccaatgaataactcaatatcagtaactttagaaaagttttatactgaaacaaaagttacatttaacgatcagcttactcaagatcaattttggcttaatggtgaaaaagtttctggaaaagaattagaaaagatttcaaagtatatggatattgttagaaatagagctggaatagattggtatgctgagatagaatctgataattttgttcctacagctgctggtcttgctagttctgctagtgcttatgcagcattagctgctgcatgtaaccaagcacttgatttacagttaagtgataaagacttaagtagattagctagaattggatcaggatcagcatcaagatcaatatacggtggttttgcagaatgggaaaaaggatataatgacgaaacttcttatgctgttccattagaaagtaatcactttgaagatgatcttgctatgatttttgtagtaataaaccaacattctaaaaaggttccttcaagatatggaatgtctcttacaagaaatacaagtagattctatcaatattggttagaccatattgatgaagatcttgcagaagcaaaggcagcaatacaagataaggattttaagagattaggtgaagttattgaagagaatggacttagaatgcatgctacaaatcttggatcaactccaccttttacttacttagtacaagagtcatacgatgtaatggcattagtacatgagtgtagagaagcaggatatccatgctatttcactatggatgctggacctaatgtaaaaatacttgtagagaagaaaaacaaacaacagataatagataaacttttaactcagttcgataataatcagataatagatagtgatattatagctacaggtattgaaattatagaataaactagttgtatattaaaatagtagaatacataagatacttaatttaattaaagatagttaagtacttttcaatgtgcttttttagatgtttaatacaaatctttaattgtaaaagaaatgctgtactatttactgttctagtgacgggattaaactgtattaattataaataaaaaataagtacagttgtttaaaattatattttgtattaaatctaatagtacgatgtaagttattttatactattgctagtttaataaaaagatttaattatatacttgaaaaggagaggaactcgagatggcagagtatataatagcagtagatgagttcgataacgaaataggatcaatagaaaagatggaagctcatagaaaaggaacacttcatagagcattcagtattttagtttttaactcaaagaatcaacttttattacagaaaagaaatgtaaagaaatatcactctccaggattatggacaaacacttgttgtagtcacccaagatatggtgaatctcttcatgatgctatatacagaagattaaaagaagagatgggatttacttgcgaacttgaagaagtattctcattcatatataaggtaaaacttgaagataatttatttgagaatgaatatgaccatgtatttattggtaaatatgatggtgagataattgttaataaagatgaagttgatgattttaaatgggtagacattaatgaagttaaaaaggacataatagaaagacctgaggcatatacttactggtttaagtatcttgtaaataaagctgaaaataagatatttaaataaaccggtcagtaacgaatagaattagaaaaacaaaggaggcaagacaatggatttcccacaacaattagaagcatgtgtaaaacaggctaatcaggcacttagtagatttattgctcctcttccttttcaaaatacaccagtagtagaaactatgcaatacggtgcacttttaggtggtaaaagattaagaccattcttagtatatgctacaggacacatgtttggtgtatcaactaatactttagacgctccagctgctgctgttgaatgtattcatgcttattctttaatacatgatgacttaccagcaatggatgacgatgatttaagaagaggtttacctacatgtcatgttaaatttggtgaagctaatgcaattttagcaggtgacgctttacaaactttagctttttctatactttcagatgcagacatgcctgaagtttcagatagagatagaatttctatgatatcagagcttgcatctgcatcaggaatagctggaatgtgcggtggtcaagcacttgatttagatgcagaaggtaaacacgtaccacttgatgctttagagagaatacatagacataaaacaggtgctcttataagagcagcagtaagattaggtgctttaagtgctggtgacaagggtagaagagcacttccagtacttgataagtatgcagaaagtataggattagcttttcaagttcaagatgacatacttgacgttgttggtgatactgctactttaggaaaaagacagggtgcagatcagcaattaggaaaatctacataccctgctttacttggattagaacaggctagaaagaaagcaagagacttaatagatgacgcaagacaaagtcttaaacagttagctgaacaatcacttgacacaagtgcacttgaagcacttgcagattatattatacagagaaacaagtaaaagcttttaaaggaggggaaaaaatggaatttagagtacatttacaggcagacaacgaacagaaaatatttcaaaatcaaatgaaaccagagccagaagcatcatatcttataaatcaaagaagaagtgctaattataaaccaaacatttggaaaaacgattttcttgatcagtctttaatatcaaaatatgatggtgatgaatatagaaaactttcagaaaagttaatagaagaagtaaagatatacatatcagcagagactatggatttagttgctaaattagaacttatagattctgttagaaaacttggacttgctaatctttttgagaaagaaataaaggaagcattagacagtatagcagcaatagaatcagataatttaggaactagagacgatctttatggaacagctcttcattttaagattcttagacagcatggatataaggtaagtcaagatatatttggtagatttatggatgagaaaggaacattagaaaatcatcactttgcacacttaaaaggaatgttagaattatttgaggcaagtaatcttggatttgaaggtgaagacatattagatgaagctaaagcatctcttacacttgctcttagagattcaggacatatttgttatccagactcaaacttaagtagagatgtagttcatagtttagaattacctagtcatagaagagttcaatggttcgatgtaaaatggcagattaatgcatacgaaaaagatatttgtagagtaaatgcaactttattagagttagcaaagttaaattttaatgttgttcaagctcagcttcagaagaatcttagagaagctagtagatggtgggctaatcttggtttcgcagataatttaaagtttgctagagatagacttgtagagtgtttttcatgcgcagtaggtgtagcatttgaaccagagcattcatcttttagaatatgtttaactaaggtaattaatcttgttcttattatagatgatgtatacgatatatatggatctgaagaagagttaaaacattttacaaatgctgttgatagatgggacagtagagaaacagaacagcttcctgaatgcatgaaaatgtgttttcaagtattatataacactacttgcgaaatagcaagagagatagaagaagaaaacggttggaatcaagtattacctcaacttactaaggtttgggctgatttttgtaaggctcttttagttgaagcagagtggtacaataaatcacatattccaacattagaagaatatcttagaaacggatgtatatcaagtagtgtatctgtacttttagttcactctttcttttcaataactcatgaaggtacaaaagaaatggctgatttcttacataaaaatgaagatcttttatacaacataagtcttatagtaagattaaacaatgatttaggtacatcagctgctgaacaggaaagaggtgattctccttcttctatagtttgctatatgagagaagttaatgcttctgaagagactgcaagaaagaatataaagggaatgattgataatgcttggaaaaaggttaatggaaaatgtttcacaactaaccaagttccatttctttcatcattcatgaataatgcaactaacatggcaagagtagcacactcattatataaagacggtgatggttttggtgatcaagaaaaaggacctagaacacatattcttagtttattattccaacctttagtaaattaagctagcataaaaataagaagcctgcatttgcaggcttcttatttttatggcgcgccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagttt

SEQ ID NO: 60 - protein, 601 aa, synthetic construct
  REGION 1..601: note = hybrid Type II methyltransferase
MFPCNAYIEYGDKNMNSFIEDVEQIYNFIKKNIDVEEKMHFIETYKQKSNMKKEISFSEEYYKQKIMNGKNGVVYTPPEMAAFMVKNLINVNDVIGNPFIKIIDPSCGSGNLICKCFLYLNRIFIKNIEVINSKNNLNLKLEDISYHIVRNNLFGFDIDETAIKVLKIDLFLISNQFSEKNFQVKDFLVENIDRKYDVFIGNPPYIGHKSVDSSYSYVLRKIYGSIYRDKGDISYCFFQKSLKCLKEGGKLVFVTSRYFCESCSGKELRKFLIENTSIYKIIDFYGIRPFKRVGIDPMIIFLVRTKNWNNNIEIIRPNKIEKNEKNKFLDSLFLDKSEKCKKFSISQKSINNDGWVFVDEVEKNIIDKIKEKSKFILKDICHSCQGIITGCDRAFIVDRDIINSRKIELRLIKPWIKSSHIRKNEVIKGEKFIIYSNLIENETECPNAIKYIEQYKKRLMERRECKKGTRKWYELQWGRKPEIFEEKKIVFPYKSCDNRFALDKGSYFSADIYSLVLKKNVPFTYEILLNILNSPLYEFYFKTFAKKLGENLYEYYPNNLMKLCIPSIDFGGENNIEKKLYDFFGLTDKEIEIVEKIKDNC

SEQ ID NO: 61 - DNA, 419 bp, Clostridium autoethanogenum
  source 1..419: mol_type = other DNA
agaaattttcctttctaaaatattttattccatgtcaagaactctgtttatttcattaaagaactataagtacaaagtataaggcatttgaaaaaataggctagtatattgattgattatttattttaaaatgcctaagtgaaatatatacatattataacaataaaataagtattagtgtaggatttttaaatagagtatctattttcagattaaatttttgattatttgatttacattatataatattgagtaaagtattgactagcaaaattttttgatactttaatttgtgaaatttcttatcaaaagttatatttttgaataatttttattgaaaaatacaactaaaaaggattatagtataagtgtgtgtaattttgtgttaaatttaaagggaggaaatgaacatgaaattg

SEQ ID NO: 62 - DNA, 567 bp, Clostridium autoethanogenum
  source 1..567: mol_type = other DNA
ctcctaattttgaaatctaatatatctattaaatcatattttcatatgtaaataaataagtttttatgcaattttgaaaaaggtatttgcataaaacggcttgaaatcaatagttaacgcaatagttattcttttagcatacattaagtcaacaaaattagcatgtaataattatgaataattattacatatattcaatattatattaaaaaaaatactttgttttaagtataaagtaaaaaaataggcataaatgtaacaaaaactgttaattttttgtgtcaataatttttgttatattattttaattaaatttttcacatgtataattaaaagtaagatagatattctaatgtacttacttaggtagaaaaacatgtatacaaaattaaaaaactattataacacatagtatcaatattgaaggtaatactgttcaatatcgatacagataaaaaaaatatataatacagaagaaaaaattataaatttgtggtataatataaagtatagtaatttaagtttaaacctcgtgaaaacgctaacaaataataggaggtgtattat

SEQ ID NO: 63 - DNA, 1806 bp, synthetic construct
  misc_feature 1..1806: note = hybrid Type II methyltransferase
  source 1..1806: mol_type = other DNA
atgtttccgtgcaatgcctatatcgaatatggtgataaaaatatgaacagctttatcgaagatgtggaacagatctacaacttcattaaaaagaacattgatgtggaagaaaagatgcatttcattgaaacctataaacagaaaagcaacatgaagaaagagattagctttagcgaagaatactataaacagaagattatgaacggcaaaaatggcgttgtgtacaccccgccggaaatggcggcctttatggttaaaaatctgatcaacgttaacgatgttattggcaatccgtttattaaaatcattgacccgagctgcggtagcggcaatctgatttgcaaatgttttctgtatctgaatcgcatctttattaagaacattgaggtgattaacagcaaaaataacctgaatctgaaactggaagacatcagctaccacatcgttcgcaacaatctgtttggcttcgatattgacgaaaccgcgatcaaagtgctgaaaattgatctgtttctgatcagcaaccaatttagcgagaaaaatttccaggttaaagactttctggtggaaaatattgatcgcaaatatgacgtgttcattggtaatccgccgtatatcggtcacaaaagcgtggacagcagctacagctacgtgctgcgcaaaatctacggcagcatctaccgcgacaaaggcgatatcagctattgtttctttcagaagagcctgaaatgtctgaaggaaggtggcaaactggtgtttgtgaccagccgctacttctgcgagagctgcagcggtaaagaactgcgtaaattcctgatcgaaaacacgagcatttacaagatcattgatttttacggcatccgcccgttcaaacgcgtgggtatcgatccgatgattatttttctggttcgtacgaagaactggaacaataacattgaaattattcgcccgaacaagattgaaaagaacgaaaagaacaaattcctggatagcctgttcctggacaaaagcgaaaagtgtaaaaagtttagcattagccagaaaagcattaataacgatggctgggttttcgtggacgaagtggagaaaaacattatcgacaaaatcaaagagaaaagcaagttcattctgaaagatatttgccatagctgtcaaggcattatcaccggttgtgatcgcgcctttattgtggaccgtgatatcatcaatagccgtaagatcgaactgcgtctgattaaaccgtggattaaaagcagccatatccgtaagaatgaagttattaagggcgaaaaattcatcatctatagcaacctgattgagaatgaaaccgagtgtccgaatgcgattaaatatatcgaacagtacaagaaacgtctgatggagcgccgcgaatgcaaaaagggcacgcgtaagtggtatgaactgcaatggggccgtaaaccggaaatcttcgaagaaaagaaaattgttttcccgtataaaagctgtgacaatcgttttgcactggataagggtagctattttagcgcagacatttatagcctggttctgaagaaaaatgtgccgttcacctatgagatcctgctgaatatcctgaatagcccgctgtacgagttttactttaagaccttcgcgaaaaagctgggcgagaatctgtacgagtactatccgaacaacctgatgaagctgtgcatcccgagcatcgatttcggcggtgagaacaatattgagaaaaagctgtatgatttctttggtctgacggataaagaaattgagattgtggagaagatcaaagataactgctaa

SEQ ID NO: 64 - DNA, 4709 bp, synthetic construct
  misc_feature 1..4709: note = designed methylation plasmid
  source 1..4709: mol_type = other DNA
gtttgccacctgacgtctaagaaaaggaatattcagcaatttgcccgtgccgaagaaaggcccacccgtgaaggtgagccagtgagttgattgctacgtaattagttagttagcccttagtgactcgtaatacgactcactatagggctcgaggcggccgcgcaacgcaattaatgtgagttagctcactcattaggcaccccaggctttacactttatgcttccggctcgtatgttgtgtggaattgtgagcggataacaatttcacacaggaaacacatatgtttccgtgcaatgcctatatcgaatatggtgataaaaatatgaacagctttatcgaagatgtggaacagatctacaacttcattaaaaagaacattgatgtggaagaaaagatgcatttcattgaaacctataaacagaaaagcaacatgaagaaagagattagctttagcgaagaatactataaacagaagattatgaacggcaaaaatggcgttgtgtacaccccgccggaaatggcggcctttatggttaaaaatctgatcaacgttaacgatgttattggcaatccgtttattaaaatcattgacccgagctgcggtagcggcaatctgatttgcaaatgttttctgtatctgaatcgcatctttattaagaacattgaggtgattaacagcaaaaataacctgaatctgaaactggaagacatcagctaccacatcgttcgcaacaatctgtttggcttcgatattgacgaaaccgcgatcaaagtgctgaaaattgatctgtttctgatcagcaaccaatttagcgagaaaaatttccaggttaaagactttctggtggaaaatattgatcgcaaatatgacgtgttcattggtaatccgccgtatatcggtcacaaaagcgtggacagcagctacagctacgtgctgcgcaaaatctacggcagcatctaccgcgacaaaggcgatatcagctattgtttctttcagaagagcctgaaatgtctgaaggaaggtggcaaactggtgtttgtgaccagccgctacttctgcgagagctgcagcggtaaagaactgcgtaaattcctgatcgaaaacacgagcatttacaagatcattgatttttacggcatccgcccgttcaaacgcgtgggtatcgatccgatgattatttttctggttcgtacgaagaactggaacaataacattgaaattattcgcccgaacaagattgaaaagaacgaaaagaacaaattcctggatagcctgttcctggacaaaagcgaaaagtgtaaaaagtttagcattagccagaaaagcattaataacgatggctgggttttcgtggacgaagtggagaaaaacattatcgacaaaatcaaagagaaaagcaagttcattctgaaagatatttgccatagctgtcaaggcattatcaccggttgtgatcgcgcctttattgtggaccgtgatatcatcaatagccgtaagatcgaactgcgtctgattaaaccgtggattaaaagcagccatatccgtaagaatgaagttattaagggcgaaaaattcatcatctatagcaacctgattgagaatgaaaccgagtgtccgaatgcgattaaatatatcgaacagtacaagaaacgtctgatggagcgccgcgaatgcaaaaagggcacgcgtaagtggtatgaactgcaatggggccgtaaaccggaaatcttcgaagaaaagaaaattgttttcccgtataaaagctgtgacaatcgttttgcactggataagggtagctattttagcgcagacatttatagcctggttctgaagaaaaatgtgccgttcacctatgagatcctgctgaatatcctgaatagcccgctgtacgagttttactttaagaccttcgcgaaaaagctgggcgagaatctgtacgagtactatccgaacaacctgatgaagctgtgcatcccgagcatcgatttcggcggtgagaacaatattgagaaaaagctgtatgatttctttggtctgacggataaagaaattgagattgtggagaagatcaaagataactgctaagaattcgatatcacccgggaactagtctgcagccctttagtgagggttaattggagtcactaagggttagttagttagattagcagaaagtcaaaagcctccgaccggaggcttttgactaaaacttcccttggggttatcattggggctcactcaaaggcggtaatcagataaaaaaaatccttagctttcgctaaggatgatttctgctagagatggaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgtttactagaggttgatcgggcacgtaagaggttccaactttcaccataatgaaataagatcactaccgggcgtattttttgagttatcgagattttcaggagctaaggaagctaaaatggagaaaaaaatcacgggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaacgctcacccggagtttcgtatggccatgaaagacggtgagctggtgatctgggatagtgttcacccttgttacaccgttttccatgagcaaactgaaacgttttcgtccctctggagtgaataccacgacgatttccggcagtttctccacatatattcgcaagatgtggcgtgttacggtgaaaacctggcctatttccctaaagggtttattgagaatatgttttttgtctcagccaatccctgggtgagtttcaccagttttgatttaaacgtggccaatatggacaacttcttcgcccccgttttcacgatgggcaaatattatacgcaaggcgacaaggtgctgatgccgctggcgatccaggttcatcatgccgtttgtgatggcttccatgtcggccgcatgcttaatgaattacaacagtactgtgatgagtggcagggcggggcgtaataatactagctccggcaaaaaaacgggcaaggtgtcaccaccctgccctttttctttaaaaccgaaaagattacttcgc

SEQ ID NO: 65 - DNA, 18 bp, synthetic construct
  misc_feature 1..18: note = Oligonucleotide colE1-F
  source 1..18: mol_type = other DNA
cgtcagaccccgtagaaa

SEQ ID NO: 66 - DNA, 18 bp, synthetic construct
  misc_feature 1..18: note = Oligonucleotide colE1-R
  source 1..18: mol_type = other DNA
ctctcctgttccgaccct

SEQ ID NO: 67 - DNA, 37 bp, synthetic construct
  misc_feature 1..37: note = Oligonucleotide fD1
  source 1..37: mol_type = other DNA
ccgaattcgtcgacaacagagtttgatcctggctcag

SEQ ID NO: 68 - DNA, 37 bp, synthetic construct
  misc_feature 1..37: note = Oligonucleotide Rp2
  source 1..37: mol_type = other DNA
cccgggatccaagcttacggctaccttgttacgactt

SEQ ID NO: 69 - DNA, 25 bp, synthetic construct
  misc_feature 1..25: note = Oligonucleotide ispS-F
  source 1..25: mol_type = other DNA
aggctgaatttcttacacttcttga

SEQ ID NO: 70 - DNA, 25 bp, synthetic construct
  misc_feature 1..25: note = Oligonucleotide ispS-R
  source 1..25: mol_type = other DNA
gtaactccatcaaatcctccactac

SEQ ID NO: 71 - DNA, 25 bp, synthetic construct
  misc_feature 1..25: note = Oligonucleotide idi-F
  source 1..25: mol_type = other DNA
atacgtgctgtagtcatccaagata

SEQ ID NO: 72 - DNA, 25 bp, synthetic construct
  misc_feature 1..25: note = Oligonucleotide idiR
  source 1..25: mol_type = other DNA
tcttcaagttcacatgtaaaaccca

SEQ ID NO: 73 - DNA, 25 bp, synthetic construct
  misc_feature 1..25: note = oligonucleotide dxs-F
  source 1..25: mol_type = other DNA
acaaagtatctaagacaggaggtca

SEQ ID NO: 74 - DNA, 25 bp, synthetic construct
  misc_feature 1..25: note = oligonucleotide dxs-R
  source 1..25: mol_type = other DNA
gatgtcccacatcccatataagttt

SEQ ID NO: 75 - DNA, 6018 bp, synthetic construct
  misc_feature 1..6018: note = plasmid pMTL85246-IspS-Idi
  source 1..6018: mol_type = other DNA
ccggggatcctctagagtcgacgtcacgcgtccatggagatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgcattcacttcttttctatataaatatgagcgaagcgaataagcgtcggaaaagcagcaaaaagtttcctttttgctgttggagcatgggggttcagggggtgcagtatctgacgtcaatgccgagcgaaagcgagccgaagggtagcatttacgttagataaccccctgatatgctccgacgctttatatagaaaagaagattcaactaggtaaaatcttaatataggttgagatgataaggtttataaggaatttgtttgttctaatttttcactcattttgttctaatttcttttaacaaatgttcttttttttttagaacagttatgatatagttagaatagtttaaaataaggagtgagaaaaagatgaaagaaagatatggaacagtctataaaggctctcagaggctcatagacgaagaaagtggagaagtcatagaggtagacaagttataccgtaaacaaacgtctggtaacttcgtaaaggcatatatagtgcaattaataagtatgttagatatgattggcggaaaaaaacttaaaatcgttaactatatcctagataatgtccacttaagtaacaatacaatgatagctacaacaagagaaatagcaaaagctacaggaacaagtctacaaacagtaataacaacacttaaaatcttagaagaaggaaatattataaaaagaaaaactggagtattaatgttaaaccctgaactactaatgagaggcgacgaccaaaaacaaaaatacctcttactcgaatttgggaactttgagcaagaggcaaatgaaatagattgacctcccaataacaccacgtagttattgggaggtcaatctatgaaatgcgattaagggccggccgaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtgtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcaggataaaaaaattgtagataaattttataaaatagttttatctacaatttttttatcaggaaacagctatgaccgcggccgcggttaatgttaaaaatttatagtataactttaaaaaactgtcttaaaaagttgttatataaaaaatgttgacaattaaacagctatttagtgcaaaacaaccataaaaatttaaaaaataccataaattacttgaaaaatagttgataataatgtagagttataaacaaaggtgaaaagcattacttgtattcttttttatatattattataaattaaaatgaagctgtattagaaaaaatacacacctgtaatataaaattttaaattaatttttaattttttcaaaatgtattttacatgtttagaattttgatgtatattaaaatagtagaatacataagatacttaatttaattaaagatagttaagtacttttcaatgtgcttttttagatgtttaatacaaatctttaattgtaaaagaaatgctgtactatttactgtactagtgacgggattaaactgtattaattataaataaaaaataagtacagttgtttaaaattatattttgtattaaatctaatagtacgatgtaagttattttatactattgctagtttaataaaaagatttaattatatacttgaaaaggagaggaatttttatgcgtcatatggcaacagaattattatgtttacacagacctatatcacttactcacaaactttttaggaatccattacctaaagttattcaagctacacctttaacattaaaacttaggtgtagtgtttctacagaaaatgtatcatttagtgagacagaaactgaaacaagaagatcagcaaattatgaaccaaattcttgggattatgattatcttctttcttctgatactgatgagtcaatagaagtacataaagataaggctaagaaattagaagctgaagttaggagagaaataaataatgagaaggctgaatttcttacacttcttgaacttattgataatgtacaaagacttggattaggatatagatttgagtctgatataagaagagcattagatagatttgtaagtagtggaggatttgatggagttactaaaacttcattacatggaacagcattatcatttaggttattaaggcaacatggttttgaagtatctcaagaagcttttagtggatttaaagatcagaatggaaactttcttgagaatttaaaggaagacataaaagcaattctttctctttatgaagcatcatttttagcattagaaggtgagaatatattagatgaggctaaagtatttgcaatatctcatcttaaagaacttagtgaagaaaagattggtaaagaattagctgaacaagtttcacatgctttagaattaccattacatagaagaacacaaagattagaagcagtttggtcaatagaagcatatagaaagaaagaagacgcaaatcaagtacttttagaacttgcaatacttgactacaatatgattcaaagtgtatatcagagggatttaagagaaacatcaagatggtggagaagagtaggattagcaactaaattacattttgctagagataggcttattgaaagtttttattgggctgttggagttgcttttgaaccacaatattctgattgcagaaatagtgtagcaaagatgttttcatttgttactataattgacgatatttacgatgtatatggaactttagatgaacttgaactttttactgatgcagttgaaagatgggatgtaaatgctattaatgatcttcctgattatatgaagttatgttttcttgcactttacaatactattaacgagatagcttacgataacttaaaagataaaggtgagaacatacttccttatttaacaaaagcatgggcagatttatgtaatgcatttcttcaagaagctaagtggctttataataaatcaacacctacatttgatgattattttggaaatgcatggaaaagttctagtggacctttacagcttatttttgcttattttgctgtagtacagaacattaaaaaggaagagattgagaatcttcagaaatatcatgacataatatcaagacctagtcacatttttaggctttgtaatgatttagcatctgcttcagcagaaatagcaagaggtgaaactgctaattctgtaagttgttatatgagaacaaaaggtatatctgaagaattagctactgaaagtgttatgaatcttatagacgaaacttggaagaaaatgaacaaagaaaaacttggtggatctttatttgcaaaaccttttgttgagactgctataaatttagctagacagtctcattgcacatatcataatggtgatgcacatactagtccagatgaattaactaggaaaagagtacttagtgtaataactgaaccaatattaccatttgaaagataagaattcgagctcgaaaggggaaattaaatggcagaatatataatagctgtagatgaatttgataacgaaataggttcaattgaaaaaatggaggctcaccgtaaaggaacattacatagagctttttctatattagtatttaattctaaaaatcaattgttattacagaaaagaaatgtaaaaaaatatcattcgcctggtctctggacaaatacgtgctgtagtcatccaagatacggtgaaagtttacatgatgcgatttatagaaggcttaaggaagaaatgggttttacatgtgaacttgaagaagtatttagttttatttataaagtaaaacttgaagataatctttttgaaaatgaatatgatcatgtattcattgggaaatatgatggagaaataattgtaaacaaagatgaagtagatgattttaagtgggttgatattaatgaggttaagaaggatattatagaaaggccagaagcatacacttattggttcaagtatttagttaataaggcagaaaacaaaatatttaaataagtaagaatttcgtctaaataaagatttggggtac

SEQ ID NO: 76 - DNA, 6909 bp, synthetic construct
  misc_feature 1..6909: note = plasmid pMTL 82151-Patp-HMGR
  source 1..6909: mol_type = other DNA
cctgcaggataaaaaaattgtagataaattttataaaatagttttatctacaatttttttatcaggaaacagctatgaccgcggccgctgtatccatatgaccatgattacgaattcgagctcgttataattttcaattttcattctttttaaaggagattagcatacattttatcataattatacagacaatatagtaatatatgatgttaaaatatcaatatatggttaaaaatctgtatattttttcccattttaattatttgtactataatattacactgagtgtattgcatatttaaaaaatatttggtacaattagttagttaaataaattctaaattgtaaattatcagaatccttattaaggaaatacatagatttaaggagaaatcataaaaaggtgtaatataaactggctaaaattgagcaaaaattgagcaattaagactttttgattgtatctttttatatatttaaggtatataatcttatttatattgggggaaggtaccatgcaatcattagacaaaaatttcagacatttatcaagacaacaaaagttacaacaattagttgataaacagtggctttcagaagatcagtttgatattttacttaatcatcctcttatagatgaagaagttgctaatagtcttatagaaaatgtaattgcacagggtgcattaccagttggacttcttcctaatataatagttgatgataaggcttatgttgtaccaatgatggttgaagaacctagtgttgttgcagctgcatcttatggtgctaaattagtaaatcagacaggtggatttaaaactgtatcatcagaaagaataatgattggacagatagtatttgatggtgtagatgacactgaaaaattaagtgcagatattaaagcattagaaaaacaaatacataagattgcagatgaagcatatcctagtataaaagcaagaggtggtggttatcaaagaatagcaatagatacatttccagagcaacaacttttaagtcttaaggtatttgtagatacaaaagatgctatgggtgctaatatgcttaatactatacttgaggcaataactgcattccttaaaaatgaatctcctcaatcagatatattaatgtctatactttcaaaccatgcaactgctagtgtagtaaaagtacaaggtgagatagatgtaaaagatcttgctagaggtgaaagaacaggtgaagaagtagctaagagaatggaaagagcttctgtattagctcaggttgatattcatagagctgcaacacataacaaaggtgttatgaatggaatacatgctgttgttttagctacaggaaatgatactagaggtgctgaagcatctgcacatgcatacgcatcaagagacggacaatatagaggtatagcaacttggagatatgatcagaagagacaaagacttattggaactattgaagttccaatgacacttgctatagtaggtggtggtactaaagtattaccaatagctaaggcatcattagagttattaaatgttgattctgcacaagaacttggacacgtagttgctgctgttggattagcacaaaactttgctgcttgtagagcacttgtttctgaaggtattcaacaaggacacatgtcattacaatataaaagtttagcaatagtagtaggtgcaaaaggtgacgagatagcacaagtagcagaagctcttaaacaggaaccaagagctaatacacaggttgctgaaagaattttacaggaaattagacagcaataatctagagtcgacgtcacgcgtccatggagatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgttctgaatccttagctaatggttcaacaggtaactatgacgaagatagcaccctggataagtctgtaatggattctaaggcatttaatgaagacgtgtatataaaatgtgctaatgaaaaagaaaatgcgttaaaagagcctaaaatgagttcaaatggttttgaaattgattggtagtttaatttaatatattttttctattggctatctcgatacctatagaatcttctgttcacttttgtttttgaaatataaaaaggggctttttagccccttttttttaaaactccggaggagtttcttcattcttgatactatacgtaactattttcgatttgacttcattgtcaattaagctagtaaaatcaatggttaaaaaacaaaaaacttgcatttttctacctagtaatttataattttaagtgtcgagtttaaaagtataatttaccaggaaaggagcaagttttttaataaggaaaaatttttccttttaaaattctatttcgttatatgactaattataatcaaaaaaatgaaaataaacaagaggtaaaaactgctttagagaaatgtactgataaaaaaagaaaaaatcctagatttacgtcatacatagcacctttaactactaagaaaaatattgaaaggacttccacttgtggagattatttgtttatgttgagtgatgcagacttagaacattttaaattacataaaggtaatttttgcggtaatagattttgtccaatgtgtagttggcgacttgcttgtaaggatagtttagaaatatctattcttatggagcatttaagaaaagaagaaaataaagagtttatatttttaactcttacaactccaaatgtaaaaagttatgatcttaattattctattaaacaatataataaatcttttaaaaaattaatggagcgtaaggaagttaaggatataactaaaggttatataagaaaattagaagtaacttaccaaaaggaaaaatacataacaaaggatttatggaaaataaaaaaagattattatcaaaaaaaaggacttgaaattggtgatttagaacctaattttgatacttataatcctcattttcatgtagttattgcagttaataaaagttattttacagataaaaattattatataaatcgagaaagatggttggaattatggaagtttgctactaaggatgattctataactcaagttgatgttagaaaagcaaaaattaatgattataaagaggtttacgaacttgcgaaatattcagctaaagacactgattatttaatatcgaggccagtatttgaaattttttataaagcattaaaaggcaagcaggtattagtttttagtggattttttaaagatgcacacaaattgtacaagcaaggaaaacttgatgtttataaaaagaaagatgaaattaaatatgtctatatagtttattataattggtgcaaaaaacaatatgaaaaaactagaataagggaacttacggaagatgaaaaagaagaattaaatcaagatttaatagatgaaatagaaatagattaaagtgtaactatactttatatatatatgattaaaaaaataaaaaacaacagcctattaggttgttgttttttattttctttattaatttttttaatttttagtttttagttcttttttaaaataagtttcagcctctttttcaatattttttaaagaaggagtatttgcatgaattgccttttttctaacagacttaggaaatattttaacagtatcttcttgcgccggtgattttggaacttcataacttactaatttataattattattttcttttttaattgtaacagttgcaaaagaagctgaacctgttccttcaactagtttatcatcttcaatataatattcttgacctatatagtataaatatatttttattatatttttacttttttctgaatctattattttataatcataaaaagttttaccaccaaaagaaggttgtactccttctggtccaacatatttttttactatattatctaaataatttttgggaactggtgttgtaatttgattaatcgaacaaccagttatacttaaaggaattataactataaaaatatataggattatctttttaaatttcattattggcctcctttttattaaatttatgttaccataaaaaggacataacgggaatatgtagaatatttttaatgtagacaaaattttacataaatataaagaaaggaagtgtttgtttaaattttatagcaaactatcaaaaattagggggataaaaatttatgaaaaaaaggttttcgatgttatttttatgtttaactttaatagtttgtggtttatttacaaattcggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatcgggccc

SEQ ID NO: 77 - DNA, 31 bp, synthetic construct
  misc_feature 1..31: note = oligonucleotide EcoRI-HMGS_F
  source 1..31: mol_type = other DNA
agccgtgaattcgaggcttttactaaaaaca

SEQ ID NO: 78 - DNA, 32 bp, synthetic construct
  misc_feature 1..32: note = oligonucleotide EcoRI-HMGS_R
  source 1..32: mol_type = other DNA
aggcgtctagatgttcgtctctacaaataatt

SEQ ID NO: 79 - DNA, 8116 bp, synthetic construct
  misc_feature 1..8116: note = plasmid pMTL 82151-HMGS-Patp-HMGR
  source 1..8116: mol_type = other DNA
cctgcaggataaaaaaattgtagataaattttataaaatagttttatctacaatttttttatcaggaaacagctatgaccgcggccgctgtatccatatgaccatgattacgaattcgaggcttttactaaaaacaataaaaacaggaggaaataatatgactataggaattgacaaaataaacttttacgtaccaaaatattatgtagatatggcaaaattagcagaagcaagacaagtagacccaaataaatttcttattggaataggacagactgaaatggcagttagtccagtaaaccaagatatagtatcaatgggtgctaatgctgctaaagatataataactgatgaagacaaaaagaaaataggaatggtaatagtagcaactgagtcagcagtagatgcagcaaaggcagcagcagtacagattcataatttattaggtattcaaccatttgcaagatgtttcgaaatgaaagaagcatgttatgctgctactcctgcaattcagttagctaaggattatttagctacaagaccaaatgagaaagttttagttatagctacagatacagctagatatggacttaattcaggtggtgaacctactcaaggtgctggtgctgttgctatggttatagctcataatcctagtatacttgcattaaatgaagacgctgttgcttatacagaagatgtttatgatttctggagaccaacaggacataagtatccattagtagatggtgctttatcaaaagacgcatatattagatcttttcaacaatcttggaatgaatatgctaagagacaaggaaagagtttagctgattttgctagtctttgctttcatgttccttttactaaaatgggtaaaaaggctttagaatctataatagataacgcagatgaaacaactcaagagagattaagatctggatatgaagatgcagttgattacaatagatatgttggaaatatatacacaggaagtctttatctttctcttataagtcttcttgaaaatagagatttacaggctggtgaaactattggattattttcatacggatcaggttctgttggtgaattttattcagctacacttgtagaaggatataaagatcaccttgatcaggcagcacacaaagcacttttaaacaatagaactgaagtatcagtagatgcatacgaaacatttttcaagagatttgatgatgtagaatttgatgaagagcaggatgcagttcatgaagatagacatatattctatctttcaaacatagagaataatgtaagagaatatcatagacctgaataagagctcgttataattttcaattttcattctttttaaaggagattagcatacattttatcataattatacagacaatatagtaatatatgatgttaaaatatcaatatatggttaaaaatctgtatattttttcccattttaattatttgtactataatattacactgagtgtattgcatatttaaaaaatatttggtacaattagttagttaaataaattctaaattgtaaattatcagaatccttattaaggaaatacatagatttaaggagaaatcataaaaaggtgtaatataaactggctaaaattgagcaaaaattgagcaattaagactttttgattgtatctttttatatatttaaggtatataatcttatttatattgggggaaggtaccatgcaatcattagacaaaaatttcagacatttatcaagacaacaaaagttacaacaattagttgataaacagtggctttcagaagatcagtttgatattttacttaatcatcctcttatagatgaagaagttgctaatagtcttatagaaaatgtaattgcacagggtgcattaccagttggacttcttcctaatataatagttgatgataaggcttatgttgtaccaatgatggttgaagaacctagtgttgttgcagctgcatcttatggtgctaaattagtaaatcagacaggtggatttaaaactgtatcatcagaaagaataatgattggacagatagtatttgatggtgtagatgacactgaaaaattaagtgcagatattaaagcattagaaaaacaaatacataagattgcagatgaagcatatcctagtataaaagcaagaggtggtggttatcaaagaatagcaatagatacatttccagagcaacaacttttaagtcttaaggtatttgtagatacaaaagatgctatgggtgctaatatgcttaatactatacttgaggcaataactgcattccttaaaaatgaatctcctcaatcagatatattaatgtctatactttcaaaccatgcaactgctagtgtagtaaaagtacaaggtgagatagatgtaaaagatcttgctagaggtgaaagaacaggtgaagaagtagctaagagaatggaaagagcttctgtattagctcaggttgatattcatagagctgcaacacataacaaaggtgttatgaatggaatacatgctgttgttttagctacaggaaatgatactagaggtgctgaagcatctgcacatgcatacgcatcaagagacggacaatatagaggtatagcaacttggagatatgatcagaagagacaaagacttattggaactattgaagttccaatgacacttgctatagtaggtggtggtactaaagtattaccaatagctaaggcatcattagagttattaaatgttgattctgcacaagaacttggacacgtagttgctgctgttggattagcacaaaactttgctgcttgtagagcacttgtttctgaaggtattcaacaaggacacatgtcattacaatataaaagtttagcaatagtagtaggtgcaaaaggtgacgagatagcacaagtagcagaagctcttaaacaggaaccaagagctaatacacaggttgctgaaagaattttacaggaaattagacagcaataatctagagtcgacgtcacgcgtccatggagatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgttctgaatccttagctaatggttcaacaggtaactatgacgaagatagcaccctggataagtctgtaatggattctaaggcatttaatgaagacgtgtatataaaatgtgctaatgaaaaagaaaatgcgttaaaagagcctaaaatgagttcaaatggttttgaaattgattggtagtttaatttaatatattttttctattggctatctcgatacctatagaatcttctgttcacttttgtttttgaaatataaaaaggggctttttagccccttttttttaaaactccggaggagtttcttcattcttgatactatacgtaactattttcgatttgacttcattgtcaattaagctagtaaaatcaatggttaaaaaacaaaaaacttgcatttttctacctagtaatttataattttaagtgtcgagtttaaaagtataatttaccaggaaaggagcaagttttttaataaggaaaaatttttccttttaaaattctatttcgttatatgactaattataatcaaaaaaatgaaaataaacaagaggtaaaaactgctttagagaaatgtactgataaaaaaagaaaaaatcctagatttacgtcatacatagcacctttaactactaagaaaaatattgaaaggacttccacttgtggagattatttgtttatgttgagtgatgcagacttagaacattttaaattacataaaggtaatttttgcggtaatagattttgtccaatgtgtagttggcgacttgcttgtaaggatagtttagaaatatctattcttatggagcatttaagaaaagaagaaaataaagagtttatatttttaactcttacaactccaaatgtaaaaagttatgatcttaattattctattaaacaatataataaatcttttaaaaaattaatggagcgtaaggaagttaaggatataactaaaggttatataagaaaattagaagtaacttaccaaaaggaaaaatacataacaaaggatttatggaaaataaaaaaagattattatcaaaaaaaaggacttgaaattggtgatttagaacctaattttgatacttataatcctcattttcatgtagttattgcagttaataaaagttattttacagataaaaattattatataaatcgagaaagatggttggaattatggaagtttgctactaaggatgattctataactcaagttgatgttagaaaagcaaaaattaatgattataaagaggtttacgaacttgcgaaatattcagctaaagacactgattatttaatatcgaggccagtatttgaaattttttataaagcattaaaaggcaagcaggtattagtttttagtggattttttaaagatgcacacaaattgtacaagcaaggaaaacttgatgtttataaaaagaaagatgaaattaaatatgtctatatagtttattataattggtgcaaaaaacaatatgaaaaaactagaataagggaacttacggaagatgaaaaagaagaattaaatcaagatttaatagatgaaatagaaatagattaaagtgtaactatactttatatatatatgattaaaaaaataaaaaacaacagcctattaggttgttgttttttattttctttattaatttttttaatttttagtttttagttcttttttaaaataagtttcagcctctttttcaatattttttaaagaaggagtatttgcatgaattgccttttttctaacagacttaggaaatattttaacagtatcttcttgcgccggtgattttggaacttcataacttactaatttataattattattttcttttttaattgtaacagttgcaaaagaagctgaacctgttccttcaactagtttatcatcttcaatataatattcttgacctatatagtataaatatatttttattatatttttacttttttctgaatctattattttataatcataaaaagttttaccaccaaaagaaggttgtactccttctggtccaacatatttttttactatattatctaaataatttttgggaactggtgttgtaatttgattaatcgaacaaccagttatacttaaaggaattataactataaaaatatataggattatctttttaaatttcattattggcctcctttttattaaatttatgttaccataaaaaggacataacgggaatatgtagaatatttttaatgtagacaaaattttacataaatataaagaaaggaagtgtttgtttaaattttatagcaaactatcaaaaattagggggataaaaatttatgaaaaaaaggttttcgatgttatttttatgtttaactttaatagtttgtggtttatttacaaattcggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatcgggccc

SEQ ID NO: 80 - DNA, 55 bp, synthetic construct
  misc_feature 1..55: note = Oligonucleotide NotI-XbaI-Prnf-MK_F
  source 1..55: mol_type = other DNA
atgcgcggccgctaggtctagaatatcgatacagataaaaaaatatataatacag

SEQ ID NO: 81 - DNA, 27 bp, synthetic construct
  misc_feature 1..27: note = Oligonucleotide SalI-Prnf-MK_R
  source 1..27: mol_type = other DNA
tggttctgtaacagcgtattcacctgc

SEQ ID NO: 82 - DNA, 4633 bp, synthetic construct
  misc_feature 1..4633: note = plasmid pMTL8314-Prnf-MK
  source 1..4633: mol_type = other DNA
aaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcaggataaaaaaattgtagataaattttataaaatagttttatctacaatttttttatcaggaaacagctatgaccgcggccgctaggtctagaatatcgatacagataaaaaaatatataatacagaagaaaaaattataaatttgtggtataatataaagtatagtaatttaagtttaaacctcgtgaaaacgctaacaaataataggaggtcaattgatgatagctgttccatttaacgctggaaaaataaaagttttaattgaggcattagaatctggaaattattcatcaataaaatcagatgtatatgacggaatgttatatgatgcaccagatcaccttaaatcattagtaaacagatttgtagaacttaataatataactgagccattagcagtaactatacagacaaatcttcctccttcaagaggtcttggatctagtgcagctgttgctgttgcttttgtaagagcaagttatgatttcttaggaaaaagtttaactaaagaagagcttatagaaaaggctaattgggctgaacaaatagctcatggaaagccatctggaatagatacacaaacaatagtatctggaaagcctgtttggtttcaaaagggacatgcagaaacacttaaaactctttcacttgatggatacatggtagtaattgatacaggtgttaaaggaagtacaagacaggctgtagaagatgttcataaactttgcgaagatcctcaatatatgagtcacgtaaaacacataggaaaacttgtacttagagcatctgatgttattgaacatcataactttgaagcacttgctgatatattcaatgaatgtcatgctgatttaaaggctcttacagtaagtcatgacaaaatagaacagttaatgaagataggaaaagaaaatggtgctatagctggtaaattaactggtgctggtagaggtggttcaatgttattacttgcaaaagacttaccaactgcaaagaatatagttaaagcagtagagaaagctggtgcagcacatacttggattgaaaatttaggtggttaagtcgacgtcacgcgtccatggagatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagttt

SEQ ID NO: 83 - DNA, 6753 bp, synthetic construct
  misc_feature 1..6753: note = plasmid pMTL 8314-Prnf-MK-PMK-PMD
  source 1..6753: mol_type = other DNA
aaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcaggataaaaaaattgtagataaattttataaaatagttttatctacaatttttttatcaggaaacagctatgaccgcggccgctaggtctagaatatcgatacagataaaaaaatatataatacagaagaaaaaattataaatttgtggtataatataaagtatagtaatttaagtttaaacctcgtgaaaacgctaacaaataataggaggtcaattgatgatagctgttccatttaacgctggaaaaataaaagttttaattgaggcattagaatctggaaattattcatcaataaaatcagatgtatatgacggaatgttatatgatgcaccagatcaccttaaatcattagtaaacagatttgtagaacttaataatataactgagccattagcagtaactatacagacaaatcttcctccttcaagaggtcttggatctagtgcagctgttgctgttgcttttgtaagagcaagttatgatttcttaggaaaaagtttaactaaagaagagcttatagaaaaggctaattgggctgaacaaatagctcatggaaagccatctggaatagatacacaaacaatagtatctggaaagcctgtttggtttcaaaagggacatgcagaaacacttaaaactctttcacttgatggatacatggtagtaattgatacaggtgttaaaggaagtacaagacaggctgtagaagatgttcataaactttgcgaagatcctcaatatatgagtcacgtaaaacacataggaaaacttgtacttagagcatctgatgttattgaacatcataactttgaagcacttgctgatatattcaatgaatgtcatgctgatttaaaggctcttacagtaagtcatgacaaaatagaacagttaatgaagataggaaaagaaaatggtgctatagctggtaaattaactggtgctggtagaggtggttcaatgttattacttgcaaaagacttaccaactgcaaagaatatagttaaagcagtagagaaagctggtgcagcacatacttggattgaaaatttaggtggttaagtcgacaaagacactaaaaaattataaaagtaaaggaggacattaaatgatacaagtaaaggcaccaggaaaattatatatagcaggtgaatacgctgttacagaaccaggatataaatctgttcttatagctcttgatagatttgttacagctactattgaggaagctgatcaatacaaaggaacaatacattcaaaggcattacatcacaatccagtaacttttagtagagatgaagattctattgttatatcagacccacacgcagcaaaacaacttaattatgtagtaactgctatagaaatatttgagcaatatgcaaaatcatgtgacatagcaatgaagcattttcatttaactatagattctaacttagatgatagtaatggacataagtatggacttggatcttctgctgctgttttagtttcagtaattaaagtacttaacgaattttatgatatgaaactttcaaacctttatatatataagttagcagtaattgctaatatgaaattacagagtttatcttcatgcggtgatatagcagtaagtgtttattcaggttggttagcttattctacatttgaccatgaatgggtaaaacaccagatagaagatacaacagttgaagaagtacttattaaaaattggcctggattacacatagagccacttcaagctcctgaaaatatggaagttcttataggttggacaggtagtccagctagtagtcctcattttgtttctgaagttaaaagacttaagtcagatccttcattttacggtgatttcttagaagattcacatagatgtgtagaaaaattaattcatgcattcaaaactaataatattaagggtgttcagaaaatggtaagacagaatagaactattatacaaagaatggataaggaagcaacagttgatatagagactgagaagttaaaatatttatgtgatattgctgaaaaatatcatggtgcaagtaaaacttcaggtgctggtggtggtgattgcggaataactataataaataaggatgtagacaaagagaaaatatatgatgaatggactaaacatggaataaagcctcttaagtttaatatttatcatggacaataaccatggtcaataatcttacaataaataaaagaaaggaggcaaaaatatgataaaatctggaaaagcaagagcacacactaatatagcacttataaaatattggggtaagaaagatgaggcattaataataccaatgaataactcaatatcagtaactttagaaaagttttatactgaaacaaaagttacatttaacgatcagcttactcaagatcaattttggcttaatggtgaaaaagtttctggaaaagaattagaaaagatttcaaagtatatggatattgttagaaatagagctggaatagattggtatgctgagatagaatctgataattttgttcctacagctgctggtcttgctagttctgctagtgcttatgcagcattagctgctgcatgtaaccaagcacttgatttacagttaagtgataaagacttaagtagattagctagaattggatcaggatcagcatcaagatcaatatacggtggttttgcagaatgggaaaaaggatataatgacgaaacttcttatgctgttccattagaaagtaatcactttgaagatgatcttgctatgatttttgtagtaataaaccaacattctaaaaaggttccttcaagatatggaatgtctcttacaagaaatacaagtagattctatcaatattggttagaccatattgatgaagatcttgcagaagcaaaggcagcaatacaagataaggattttaagagattaggtgaagttattgaagagaatggacttagaatgcatgctacaaatcttggatcaactccaccttttacttacttagtacaagagtcatacgatgtaatggcattagtacatgagtgtagagaagcaggatatccatgctatttcactatggatgctggacctaatgtaaaaatacttgtagagaagaaaaacaaacaacagataatagataaacttttaactcagttcgataataatcagataatagatagtgatattatagctacaggtattgaaattatagaataaactagttccgctaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagttt

SEQ ID NO: 84 - DNA, 9198 bp, synthetic construct
  misc_feature 1..9198: note = plasmid pMTL8314-Prnf-MK-PMK-PMD-Pfor-idi-ispS
  source 1..9198: mol_type = other DNA
aaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcaggataaaaaaattgtagataaattttataaaatagttttatctacaatttttttatcaggaaacagctatgaccgcggccgctaggtctagaatatcgatacagataaaaaaatatataatacagaagaaaaaattataaatttgtggtataatataaagtatagtaatttaagtttaaacctcgtgaaaacgctaacaaataataggaggtcaattgatgatagctgttccatttaacgctggaaaaataaaagttttaattgaggcattagaatctggaaattattcatcaataaaatcagatgtatatgacggaatgttatatgatgcaccagatcaccttaaatcattagtaaacagatttgtagaacttaataatataactgagccattagcagtaactatacagacaaatcttcctccttcaagaggtcttggatctagtgcagctgttgctgttgcttttgtaagagcaagttatgatttcttaggaaaaagtttaactaaagaagagcttatagaaaaggctaattgggctgaacaaatagctcatggaaagccatctggaatagatacacaaacaatagtatctggaaagcctgtttggtttcaaaagggacatgcagaaacacttaaaactctttcacttgatggatacatggtagtaattgatacaggtgttaaaggaagtacaagacaggctgtagaagatgttcataaactttgcgaagatcctcaatatatgagtcacgtaaaacacataggaaaacttgtacttagagcatctgatgttattgaacatcataactttgaagcacttgctgatatattcaatgaatgtcatgctgatttaaaggctcttacagtaagtcatgacaaaatagaacagttaatgaagataggaaaagaaaatggtgctatagctggtaaattaactggtgctggtagaggtggttcaatgttattacttgcaaaagacttaccaactgcaaagaatatagttaaagcagtagagaaagctggtgcagcacatacttggattgaaaatttaggtggttaagtcgacaaagacactaaaaaattataaaagtaaaggaggacattaaatgatacaagtaaaggcaccaggaaaattatatatagcaggtgaatacgctgttacagaaccaggatataaatctgttcttatagctcttgatagatttgttacagctactattgaggaagctgatcaatacaaaggaacaatacattcaaaggcattacatcacaatccagtaacttttagtagagatgaagattctattgttatatcagacccacacgcagcaaaacaacttaattatgtagtaactgctatagaaatatttgagcaatatgcaaaatcatgtgacatagcaatgaagcattttcatttaactatagattctaacttagatgatagtaatggacataagtatggacttggatcttctgctgctgttttagtttcagtaattaaagtacttaacgaattttatgatatgaaactttcaaacctttatatatataagttagcagtaattgctaatatgaaattacagagtttatcttcatgcggtgatatagcagtaagtgtttattcaggttggttagcttattctacatttgaccatgaatgggtaaaacaccagatagaagatacaacagttgaagaagtacttattaaaaattggcctggattacacatagagccacttcaagctcctgaaaatatggaagttcttataggttggacaggtagtccagctagtagtcctcattttgtttctgaagttaaaagacttaagtcagatccttcattttacggtgatttcttagaagattcacatagatgtgtagaaaaattaattcatgcattcaaaactaataatattaagggtgttcagaaaatggtaagacagaatagaactattatacaaagaatggataaggaagcaacagttgatatagagactgagaagttaaaatatttatgtgatattgctgaaaaatatcatggtgcaagtaaaacttcaggtgctggtggtggtgattgcggaataactataataaataaggatgtagacaaagagaaaatatatgatgaatggactaaacatggaataaagcctcttaagtttaatatttatcatggacaataaccatggtcaataatcttacaataaataaaagaaaggaggcaaaaatatgataaaatctggaaaagcaagagcacacactaatatagcacttataaaatattggggtaagaaagatgaggcattaataataccaatgaataactcaatatcagtaactttagaaaagttttatactgaaacaaaagttacatttaacgatcagcttactcaagatcaattttggcttaatggtgaaaaagtttctggaaaagaattagaaaagatttcaaagtatatggatattgttagaaatagagctggaatagattggtatgctgagatagaatctgataattttgttcctacagctgctggtcttgctagttctgctagtgcttatgcagcattagctgctgcatgtaaccaagcacttgatttacagttaagtgataaagacttaagtagattagctagaattggatcaggatcagcatcaagatcaatatacggtggttttgcagaatgggaaaaaggatataatgacgaaacttcttatgctgttccattagaaagtaatcactttgaagatgatcttgctatgatttttgtagtaataaaccaacattctaaaaaggttccttcaagatatggaatgtctcttacaagaaatacaagtagattctatcaatattggttagaccatattgatgaagatcttgcagaagcaaaggcagcaatacaagataaggattttaagagattaggtgaagttattgaagagaatggacttagaatgcatgctacaaatcttggatcaactccaccttttacttacttagtacaagagtcatacgatgtaatggcattagtacatgagtgtagagaagcaggatatccatgctatttcactatggatgctggacctaatgtaaaaatacttgtagagaagaaaaacaaacaacagataatagataaacttttaactcagttcgataataatcagataatagatagtgatattatagctacaggtattgaaattatagaataaactagttgtatattaaaatagtagaatacataagatacttaatttaattaaagatagttaagtacttttcaatgtgcttttttagatgtttaatacaaatctttaattgtaaaagaaatgctgtactatttactgttctagtgacgggattaaactgtattaattataaataaaaaataagtacagttgtttaaaattatattttgtattaaatctaatagtacgatgtaagttattttatactattgctagtttaataaaaagatttaattatatacttgaaaaggagaggaactcgagatggcagagtatataatagcagtagatgagttcgataacgaaataggatcaatagaaaagatggaagctcatagaaaaggaacacttcatagagcattcagtattttagtttttaactcaaagaatcaacttttattacagaaaagaaatgtaaagaaatatcactctccaggattatggacaaacacttgttgtagtcacccaagatatggtgaatctcttcatgatgctatatacagaagattaaaagaagagatgggatttacttgcgaacttgaagaagtattctcattcatatataaggtaaaacttgaagataatttatttgagaatgaatatgaccatgtatttattggtaaatatgatggtgagataattgttaataaagatgaagttgatgattttaaatgggtagacattaatgaagttaaaaaggacataatagaaagacctgaggcatatacttactggtttaagtatcttgtaaataaagctgaaaataagatatttaaataaaccggtgggaggaaatgaacatggcaacagaattattatgtttacacagacctatatcacttactcacaaactttttaggaatccattacctaaagttattcaagctacacctttaacattaaaacttaggtgtagtgtttctacagaaaatgtatcatttagtgagacagaaactgaaacaagaagatcagcaaattatgaaccaaattcttgggattatgattatcttctttcttctgatactgatgagtcaatagaagtacataaagataaggctaagaaattagaagctgaagttaggagagaaataaataatgagaaggctgaatttcttacacttcttgaacttattgataatgtacaaagacttggattaggatatagatttgagtctgatataagaagagcattagatagatttgtaagtagtggaggatttgatggagttactaaaacttcattacatggaacagcattatcatttaggttattaaggcaacatggttttgaagtatctcaagaagcttttagtggatttaaagatcagaatggaaactttcttgagaatttaaaggaagacataaaagcaattctttctctttatgaagcatcatttttagcattagaaggtgagaatatattagatgaggctaaagtatttgcaatatctcatcttaaagaacttagtgaagaaaagattggtaaagaattagctgaacaagtttcacatgctttagaattaccattacatagaagaacacaaagattagaagcagtttggtcaatagaagcatatagaaagaaagaagacgcaaatcaagtacttttagaacttgcaatacttgactacaatatgattcaaagtgtatatcagagggatttaagagaaacatcaagatggtggagaagagtaggattagcaactaaattacattttgctagagataggcttattgaaagtttttattgggctgttggagttgcttttgaaccacaatattctgattgcagaaatagtgtagcaaagatgttttcatttgttactataattgacgatatttacgatgtatatggaactttagatgaacttgaactttttactgatgcagttgaaagatgggatgtaaatgctattaatgatcttcctgattatatgaagttatgttttcttgcactttacaatactattaacgagatagcttacgataacttaaaagataaaggtgagaacatacttccttatttaacaaaagcatgggcagatttatgtaatgcatttcttcaagaagctaagtggctttataataaatcaacacctacatttgatgattattttggaaatgcatggaaaagttctagtggacctttacagcttatttttgcttattttgctgtagtacagaacattaaaaaggaagagattgagaatcttcagaaatatcatgacataatatcaagacctagtcacatttttaggctttgtaatgatttagcatctgcttcagcagaaatagcaagaggtgaaactgctaattctgtaagttgttatatgagaacaaaaggtatatctgaagaattagctactgaaagtgttatgaatcttatagacgaaacttggaagaaaatgaacaaagaaaaacttggtggatctttatttgcaaaaccttttgttgagactgctataaatttagctagacagtctcattgcacatatcataatggtgatgcacatactagtccagatgaattaactaggaaaagagtacttagtgtaataactgaaccaatattaccatttgaaagataagctagcataaaaataagaagcctgcatttgcaggcttcttatttttatggcgcgccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagttt

SEQ ID NO: 85 - DNA, 6841 bp, synthetic construct
  misc_feature 1..6841: note = plasmid pMTL83245-Pfor-FS-idi
  source 1..6841: mol_type = other DNA
aaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcaggataaaaaaattgtagataaattttataaaatagttttatctacaatttttttatcaggaaacagctatgaccgcggccgcaatatgatatttatgtccattgtgaaagggattatattcaactattattccagttacgttcatagaaattttcctttctaaaatattttattccatgtcaagaactctgtttatttcattaaagaactataagtacaaagtataaggcatttgaaaaaataggctagtatattgattgattatttattttaaaatgcctaagtgaaatatatacatattataacaataaaataagtattagtgtaggatttttaaatagagtatctattttcagattaaatttttgattatttgatttacattatataatattgagtaaagtattgactagcaaaattttttgatactttaatttgtgaaatttcttatcaaaagttatatttttgaataatttttattgaaaaatacaactaaaaaggattatagtataagtgtgtgtaattttgtgttaaatttaaagggaggaaatgaacatgaaacatatggtgaccatgattacgaattcgagctcggtacccggggatcctctagttgtatattaaaatagtagaatacataagatacttaatttaattaaagatagttaagtacttttcaatgtgcttttttagatgtttaatacaaatctttaattgtaaaagaaatgctgtactatttactgttctagtgacgggattaaactgtattaattataaataaaaaataagtacagttgtttaaaattatattttgtattaaatctaatagtacgatgtaagttattttatactattgctagtttaataaaaagatttaattatatacttgaaaaggagaggaactcgagatggaatttagagtacatttacaggcagacaacgaacagaaaatatttcaaaatcaaatgaaaccagagccagaagcatcatatcttataaatcaaagaagaagtgctaattataaaccaaacatttggaaaaacgattttcttgatcagtctttaatatcaaaatatgatggtgatgaatatagaaaactttcagaaaagttaatagaagaagtaaagatatacatatcagcagagactatggatttagttgctaaattagaacttatagattctgttagaaaacttggacttgctaatctttttgagaaagaaataaaggaagcattagacagtatagcagcaatagaatcagataatttaggaactagagacgatctttatggaacagctcttcattttaagattcttagacagcatggatataaggtaagtcaagatatatttggtagatttatggatgagaaaggaacattagaaaatcatcactttgcacacttaaaaggaatgttagaattatttgaggcaagtaatcttggatttgaaggtgaagacatattagatgaagctaaagcatctcttacacttgctcttagagattcaggacatatttgttatccagactcaaacttaagtagagatgtagttcatagtttagaattacctagtcatagaagagttcaatggttcgatgtaaaatggcagattaatgcatacgaaaaagatatttgtagagtaaatgcaactttattagagttagcaaagttaaattttaatgttgttcaagctcagcttcagaagaatcttagagaagctagtagatggtgggctaatcttggtttcgcagataatttaaagtttgctagagatagacttgtagagtgtttttcatgcgcagtaggtgtagcatttgaaccagagcattcatcttttagaatatgtttaactaaggtaattaatcttgttcttattatagatgatgtatacgatatatatggatctgaagaagagttaaaacattttacaaatgctgttgatagatgggacagtagagaaacagaacagcttcctgaatgcatgaaaatgtgttttcaagtattatataacactacttgcgaaatagcaagagagatagaagaagaaaacggttggaatcaagtattacctcaacttactaaggtttgggctgatttttgtaaggctcttttagttgaagcagagtggtacaataaatcacatattccaacattagaagaatatcttagaaacggatgtatatcaagtagtgtatctgtacttttagttcactctttcttttcaataactcatgaaggtacaaaagaaatggctgatttcttacataaaaatgaagatcttttatacaacataagtcttatagtaagattaaacaatgatttaggtacatcagctgctgaacaggaaagaggtgattctccttcttctatagtttgctatatgagagaagttaatgcttctgaagagactgcaagaaagaatataaagggaatgattgataatgcttggaaaaaggttaatggaaaatgtttcacaactaaccaagttccatttctttcatcattcatgaataatgcaactaacatggcaagagtagcacactcattatataaagacggtgatggttttggtgatcaagaaaaaggacctagaacacatattcttagtttattattccaacctttagtaaattaactgcagggttcaaaacatagattaaaaaattaaaggaggggaaaaaatggcagagtatataatagcagtagatgagttcgataacgaaataggatcaatagaaaagatggaagctcatagaaaaggaacacttcatagagcattcagtattttagtttttaactcaaagaatcaacttttattacagaaaagaaatgtaaagaaatatcactctccaggattatggacaaacacttgttgtagtcacccaagatatggtgaatctcttcatgatgctatatacagaagattaaaagaagagatgggatttacttgcgaacttgaagaagtattctcattcatatataaggtaaaacttgaagataatttatttgagaatgaatatgaccatgtatttattggtaaatatgatggtgagataattgttaataaagatgaagttgatgattttaaatgggtagacattaatgaagttaaaaaggacataatagaaagacctgaggcatatacttactggtttaagtatcttgtaaataaagctgaaaataagatatttaaataaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggccggccgaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtgttt

SEQ ID NO: 86 - DNA, 36 bp, synthetic construct
  misc_feature 1..36: note = Oligonucleotide idi_F2
  source 1..36: mol_type = other DNA
aggcactcgagatggcagagtatataatagcagtag

SEQ ID NO: 87 - DNA, 47 bp, synthetic construct
  misc_feature 1..47: note = Oligonucleotide idi_R2
  source 1..47: mol_type = other DNA
aggcgcaagcttggcgcaccggtttatttaaatatcttattttcagc

SEQ ID NO: 88 - DNA, 5075 bp, synthetic construct
  misc_feature 1..5075: note = plasmid pMTL83245-Pfor-idi
  source 1..5075: mol_type = other DNA
aaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcaggataaaaaaattgtagataaattttataaaatagttttatctacaatttttttatcaggaaacagctatgaccgcggccgcaatatgatatttatgtccattgtgaaagggattatattcaactattattccagttacgttcatagaaattttcctttctaaaatattttattccatgtcaagaactctgtttatttcattaaagaactataagtacaaagtataaggcatttgaaaaaataggctagtatattgattgattatttattttaaaatgcctaagtgaaatatatacatattataacaataaaataagtattagtgtaggatttttaaatagagtatctattttcagattaaatttttgattatttgatttacattatataatattgagtaaagtattgactagcaaaattttttgatactttaatttgtgaaatttcttatcaaaagttatatttttgaataatttttattgaaaaatacaactaaaaaggattatagtataagtgtgtgtaattttgtgttaaatttaaagggaggaaatgaacatgaaacatatggtgaccatgattacgaattcgagctcggtacccggggatcctctagttgtatattaaaatagtagaatacataagatacttaatttaattaaagatagttaagtacttttcaatgtgcttttttagatgtttaatacaaatctttaattgtaaaagaaatgctgtactatttactgttctagtgacgggattaaactgtattaattataaataaaaaataagtacagttgtttaaaattatattttgtattaaatctaatagtacgatgtaagttattttatactattgctagtttaataaaaagatttaattatatacttgaaaaggagaggaactcgagatggcagagtatataatagcagtagatgagttcgataacgaaataggatcaatagaaaagatggaagctcatagaaaaggaacacttcatagagcattcagtattttagtttttaactcaaagaatcaacttttattacagaaaagaaatgtaaagaaatatcactctccaggattatggacaaacacttgttgtagtcacccaagatatggtgaatctcttcatgatgctatatacagaagattaaaagaagagatgggatttacttgcgaacttgaagaagtattctcattcatatataaggtaaaacttgaagataatttatttgagaatgaatatgaccatgtatttattggtaaatatgatggtgagataattgttaataaagatgaagttgatgattttaaatgggtagacattaatgaagttaaaaaggacataatagaaagacctgaggcatatacttactggtttaagtatcttgtaaataaagctgaaaataagatatttaaataaaccggtgcgccaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggccggccgaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtgttt

SEQ ID NO: 89 - DNA, 6662 bp, synthetic construct
  misc_feature 1..6662: note = plasmid pMTL83245-Pfor-idi-FS
  source 1..6662: mol_type = other DNA
aaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcaggataaaaaaattgtagataaattttataaaatagttttatctacaatttttttatcaggaaacagctatgaccgcggccgcaatatgatatttatgtccattgtgaaagggattatattcaactattattccagttacgttcatagaaattttcctttctaaaatattttattccatgtcaagaactctgtttatttcattaaagaactataagtacaaagtataaggcatttgaaaaaataggctagtatattgattgattatttattttaaaatgcctaagtgaaatatatacatattataacaataaaataagtattagtgtaggatttttaaatagagtatctattttcagattaaatttttgattatttgatttacattatataatattgagtaaagtattgactagcaaaattttttgatactttaatttgtgaaatttcttatcaaaagttatatttttgaataatttttattgaaaaatacaactaaaaaggattatagtataagtgtgtgtaattttgtgttaaatttaaagggaggaaatgaacatgaaacatatggtgaccatgattacgaattcgagctcggtacccggggatcctctagttgtatattaaaatagtagaatacataagatacttaatttaattaaagatagttaagtacttttcaatgtgcttttttagatgtttaatacaaatctttaattgtaaaagaaatgctgtactatttactgttctagtgacgggattaaactgtattaattataaataaaaaataagtacagttgtttaaaattatattttgtattaaatctaatagtacgatgtaagttattttatactattgctagtttaataaaaagatttaattatatacttgaaaaggagaggaactcgagatggcagagtatataatagcagtagatgagttcgataacgaaataggatcaatagaaaagatggaagctcatagaaaaggaacacttcatagagcattcagtattttagtttttaactcaaagaatcaacttttattacagaaaagaaatgtaaagaaatatcactctccaggattatggacaaacacttgttgtagtcacccaagatatggtgaatctcttcatgatgctatatacagaagattaaaagaagagatgggatttacttgcgaacttgaagaagtattctcattcatatataaggtaaaacttgaagataatttatttgagaatgaatatgaccatgtatttattggtaaatatgatggtgagataattgttaataaagatgaagttgatgattttaaatgggtagacattaatgaagttaaaaaggacataatagaaagacctgaggcatatacttactggtttaagtatcttgtaaataaagctgaaaataagatatttaaataaaccggtgcgccaagcttttaaaggaggggaaaaaatggaatttagagtacatttacaggcagacaacgaacagaaaatatttcaaaatcaaatgaaaccagagccagaagcatcatatcttataaatcaaagaagaagtgctaattataaaccaaacatttggaaaaacgattttcttgatcagtctttaatatcaaaatatgatggtgatgaatatagaaaactttcagaaaagttaatagaagaagtaaagatatacatatcagcagagactatggatttagttgctaaattagaacttatagattctgttagaaaacttggacttgctaatctttttgagaaagaaataaaggaagcattagacagtatagcagcaatagaatcagataatttaggaactagagacgatctttatggaacagctcttcattttaagattcttagacagcatggatataaggtaagtcaagatatatttggtagatttatggatgagaaaggaacattagaaaatcatcactttgcacacttaaaaggaatgttagaattatttgaggcaagtaatcttggatttgaaggtgaagacatattagatgaagctaaagcatctcttacacttgctcttagagattcaggacatatttgttatccagactcaaacttaagtagagatgtagttcatagtttagaattacctagtcatagaagagttcaatggttcgatgtaaaatggcagattaatgcatacgaaaaagatatttgtagagtaaatgcaactttattagagttagcaaagttaaattttaatgttgttcaagctcagcttcagaagaatcttagagaagctagtagatggtgggctaatcttggtttcgcagataatttaaagtttgctagagatagacttgtagagtgtttttcatgcgcagtaggtgtagcatttgaaccagagcattcatcttttagaatatgtttaactaaggtaattaatcttgttcttattatagatgatgtatacgatatatatggatctgaagaagagttaaaacattttacaaatgctgttgatagatgggacagtagagaaacagaacagcttcctgaatgcatgaaaatgtgttttcaagtattatataacactacttgcgaaatagcaagagagatagaagaagaaaacggttggaatcaagtattacctcaacttactaaggtttgggctgatttttgtaaggctcttttagttgaagcagagtggtacaataaatcacatattccaacattagaagaatatcttagaaacggatgtatatcaagtagtgtatctgtacttttagttcactctttcttttcaataactcatgaaggtacaaaagaaatggctgatttcttacataaaaatgaagatcttttatacaacataagtcttatagtaagattaaacaatgatttaggtacatcagctgctgaacaggaaagaggtgattctccttcttctatagtttgctatatgagagaagttaatgcttctgaagagactgcaagaaagaatataaagggaatgattgataatgcttggaaaaaggttaatggaaaatgtttcacaactaaccaagttccatttctttcatcattcatgaataatgcaactaacatggcaagagtagcacactcattatataaagacggtgatggttttggtgatcaagaaaaaggacctagaacacatattcttagtttattattccaacctttagtaaattaagctagcataaaaataagaagcctgcatttgcaggcttcttatttttatggcgcgccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggccggccgaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtgttt

SEQ ID NO: 90 - DNA, 7077 bp, synthetic construct
  misc_feature 1..7077: note = plasmid pMTL83245-Pfor-idi-ispA-FS
  source 1..7077: mol_type = other DNA
aaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcaggataaaaaaattgtagataaattttataaaatagttttatctacaatttttttatcaggaaacagctatgaccgcggccgcaccgagactagttgtatattaaaatagtagaatacataagatacttaatttaattaaagatagttaagtacttttcaatgtgcttttttagatgtttaatacaaatctttaattgtaaaagaaatgctgtactatttactgttctagtgacgggattaaactgtattaattataaataaaaaataagtacagttgtttaaaattatattttgtattaaatctaatagtacgatgtaagttattttatactattgctagtttaataaaaagatttaattatatacttgaaaaggagaggaactcgagatggcagagtatataatagcagtagatgagttcgataacgaaataggatcaatagaaaagatggaagctcatagaaaaggaacacttcatagagcattcagtattttagtttttaactcaaagaatcaacttttattacagaaaagaaatgtaaagaaatatcactctccaggattatggacaaacacttgttgtagtcacccaagatatggtgaatctcttcatgatgctatatacagaagattaaaagaagagatgggatttacttgcgaacttgaagaagtattctcattcatatataaggtaaaacttgaagataatttatttgagaatgaatatgaccatgtatttattggtaaatatgatggtgagataattgttaataaagatgaagttgatgattttaaatgggtagacattaatgaagttaaaaaggacataatagaaagacctgaggcatatacttactggtttaagtatcttgtaaataaagctgaaaataagatatttaaataaaccggtcagtaacgaatagaattagaaaaacaaaggaggcaagacaatggatttcccacaacaattagaagcatgtgtaaaacaggctaatcaggcacttagtagatttattgctcctcttccttttcaaaatacaccagtagtagaaactatgcaatacggtgcacttttaggtggtaaaagattaagaccattcttagtatatgctacaggacacatgtttggtgtatcaactaatactttagacgctccagctgctgctgttgaatgtattcatgcttattctttaatacatgatgacttaccagcaatggatgacgatgatttaagaagaggtttacctacatgtcatgttaaatttggtgaagctaatgcaattttagcaggtgacgctttacaaactttagctttttctatactttcagatgcagacatgcctgaagtttcagatagagatagaatttctatgatatcagagcttgcatctgcatcaggaatagctggaatgtgcggtggtcaagcacttgatttagatgcagaaggtaaacacgtaccacttgatgctttagagagaatacatagacataaaacaggtgctcttataagagcagcagtaagattaggtgctttaagtgctggtgacaagggtagaagagcacttccagtacttgataagtatgcagaaagtataggattagcttttcaagttcaagatgacatacttgacgttgttggtgatactgctactttaggaaaaagacagggtgcagatcagcaattaggaaaatctacataccctgctttacttggattagaacaggctagaaagaaagcaagagacttaatagatgacgcaagacaaagtcttaaacagttagctgaacaatcacttgacacaagtgcacttgaagcacttgcagattatattatacagagaaacaagtaaaagcttttaaaggaggggaaaaaatggaatttagagtacatttacaggcagacaacgaacagaaaatatttcaaaatcaaatgaaaccagagccagaagcatcatatcttataaatcaaagaagaagtgctaattataaaccaaacatttggaaaaacgattttcttgatcagtctttaatatcaaaatatgatggtgatgaatatagaaaactttcagaaaagttaatagaagaagtaaagatatacatatcagcagagactatggatttagttgctaaattagaacttatagattctgttagaaaacttggacttgctaatctttttgagaaagaaataaaggaagcattagacagtatagcagcaatagaatcagataatttaggaactagagacgatctttatggaacagctcttcattttaagattcttagacagcatggatataaggtaagtcaagatatatttggtagatttatggatgagaaaggaacattagaaaatcatcactttgcacacttaaaaggaatgttagaattatttgaggcaagtaatcttggatttgaaggtgaagacatattagatgaagctaaagcatctcttacacttgctcttagagattcaggacatatttgttatccagactcaaacttaagtagagatgtagttcatagtttagaattacctagtcatagaagagttcaatggttcgatgtaaaatggcagattaatgcatacgaaaaagatatttgtagagtaaatgcaactttattagagttagcaaagttaaattttaatgttgttcaagctcagcttcagaagaatcttagagaagctagtagatggtgggctaatcttggtttcgcagataatttaaagtttgctagagatagacttgtagagtgtttttcatgcgcagtaggtgtagcatttgaaccagagcattcatcttttagaatatgtttaactaaggtaattaatcttgttcttattatagatgatgtatacgatatatatggatctgaagaagagttaaaacattttacaaatgctgttgatagatgggacagtagagaaacagaacagcttcctgaatgcatgaaaatgtgttttcaagtattatataacactacttgcgaaatagcaagagagatagaagaagaaaacggttggaatcaagtattacctcaacttactaaggtttgggctgatttttgtaaggctcttttagttgaagcagagtggtacaataaatcacatattccaacattagaagaatatcttagaaacggatgtatatcaagtagtgtatctgtacttttagttcactctttcttttcaataactcatgaaggtacaaaagaaatggctgatttcttacataaaaatgaagatcttttatacaacataagtcttatagtaagattaaacaatgatttaggtacatcagctgctgaacaggaaagaggtgattctccttcttctatagtttgctatatgagagaagttaatgcttctgaagagactgcaagaaagaatataaagggaatgattgataatgcttggaaaaaggttaatggaaaatgtttcacaactaaccaagttccatttctttcatcattcatgaataatgcaactaacatggcaagagtagcacactcattatataaagacggtgatggttttggtgatcaagaaaaaggacctagaacacatattcttagtttattattccaacctttagtaaattaagctagcataaaaataagaagcctgcatttgcaggcttcttatttttatggcgcgccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggccggccgaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtgttt

SEQ ID NO: 91 - DNA, 10090 bp, synthetic construct
  misc_feature 1..10090: note = plasmid pMTL 8314-Prnf-MK-PMK-PMD-Pfor-idi-ispA-FS
  source 1..10090: mol_type = other DNA
aaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcaggataaaaaaattgtagataaattttataaaatagttttatctacaatttttttatcaggaaacagctatgaccgcggccgctaggtctagaatatcgatacagataaaaaaatatataatacagaagaaaaaattataaatttgtggtataatataaagtatagtaatttaagtttaaacctcgtgaaaacgctaacaaataataggaggtcaattgatgatagctgttccatttaacgctggaaaaataaaagttttaattgaggcattagaatctggaaattattcatcaataaaatcagatgtatatgacggaatgttatatgatgcaccagatcaccttaaatcattagtaaacagatttgtagaacttaataatataactgagccattagcagtaactatacagacaaatcttcctccttcaagaggtcttggatctagtgcagctgttgctgttgcttttgtaagagcaagttatgatttcttaggaaaaagtttaactaaagaagagcttatagaaaaggctaattgggctgaacaaatagctcatggaaagccatctggaatagatacacaaacaatagtatctggaaagcctgtttggtttcaaaagggacatgcagaaacacttaaaactctttcacttgatggatacatggtagtaattgatacaggtgttaaaggaagtacaagacaggctgtagaagatgttcataaactttgcgaagatcctcaatatatgagtcacgtaaaacacataggaaaacttgtacttagagcatctgatgttattgaacatcataactttgaagcacttgctgatatattcaatgaatgtcatgctgatttaaaggctcttacagtaagtcatgacaaaatagaacagttaatgaagataggaaaagaaaatggtgctatagctggtaaattaactggtgctggtagaggtggttcaatgttattacttgcaaaagacttaccaactgcaaagaatatagttaaagcagtagagaaagctggtgcagcacatacttggattgaaaatttaggtggttaagtcgacaaagacactaaaaaattataaaagtaaaggaggacattaaatgatacaagtaaaggcaccaggaaaattatatatagcaggtgaatacgctgttacagaaccaggatataaatctgttcttatagctcttgatagatttgttacagctactattgaggaagctgatcaatacaaaggaacaatacattcaaaggcattacatcacaatccagtaacttttagtagagatgaagattctattgttatatcagacccacacgcagcaaaacaacttaattatgtagtaactgctatagaaatatttgagcaatatgcaaaatcatgtgacatagcaatgaagcattttcatttaactatagattctaacttagatgatagtaatggacataagtatggacttggatcttctgctgctgttttagtttcagtaattaaagtacttaacgaattttatgatatgaaactttcaaacctttatatatataagttagcagtaattgctaatatgaaattacagagtttatcttcatgcggtgatatagcagtaagtgtttattcaggttggttagcttattctacatttgaccatgaatgggtaaaacaccagatagaagatacaacagttgaagaagtacttattaaaaattggcctggattacacatagagccacttcaagctcctgaaaatatggaagttcttataggttggacaggtagtccagctagtagtcctcattttgtttctgaagttaaaagacttaagtcagatccttcattttacggtgatttcttagaagattcacatagatgtgtagaaaaattaattcatgcattcaaaactaataatattaagggtgttcagaaaatggtaagacagaatagaactattatacaaagaatggataaggaagcaacagttgatatagagactgagaagttaaaatatttatgtgatattgctgaaaaatatcatggtgcaagtaaaacttcaggtgctggtggtggtgattgcggaataactataataaataaggatgtagacaaagagaaaatatatgatgaatggactaaacatggaataaagcctcttaagtttaatatttatcatggacaataaccatggtcaataatcttacaataaataaaagaaaggaggcaaaaatatgataaaatctggaaaagcaagagcacacactaatatagcacttataaaatattggggtaagaaagatgaggcattaataataccaatgaataactcaatatcagtaactttagaaaagttttatactgaaacaaaagttacatttaacgatcagcttactcaagatcaattttggcttaatggtgaaaaagtttctggaaaagaattagaaaagatttcaaagtatatggatattgttagaaatagagctggaatagattggtatgctgagatagaatctgataattttgttcctacagctgctggtcttgctagttctgctagtgcttatgcagcattagctgctgcatgtaaccaagcacttgatttacagttaagtgataaagacttaagtagattagctagaattggatcaggatcagcatcaagatcaatatacggtggttttgcagaatgggaaaaaggatataatgacgaaacttcttatgctgttccattagaaagtaatcactttgaagatgatcttgctatgatttttgtagtaataaaccaacattctaaaaaggttccttcaagatatggaatgtctcttacaagaaatacaagtagattctatcaatattggttagaccatattgatgaagatcttgcagaagcaaaggcagcaatacaagataaggattttaagagattaggtgaagttattgaagagaatggacttagaatgcatgctacaaatcttggatcaactccaccttttacttacttagtacaagagtcatacgatgtaatggcattagtacatgagtgtagagaagcaggatatccatgctatttcactatggatgctggacctaatgtaaaaatacttgtagagaagaaaaacaaacaacagataatagataaacttttaactcagttcgataataatcagataatagatagtgatattatagctacaggtattgaaattatagaataaactagttgtatattaaaatagtagaatacataagatacttaatttaattaaagatagttaagtacttttcaatgtgcttttttagatgtttaatacaaatctttaattgtaaaagaaatgctgtactatttactgttctagtgacgggattaaactgtattaattataaataaaaaataagtacagttgtttaaaattatattttgtattaaatctaatagtacgatgtaagttattttatactattgctagtttaataaaaagatttaattatatacttgaaaaggagaggaactcgagatggcagagtatataatagcagtagatgagttcgataacgaaataggatcaatagaaaagatggaagctcatagaaaaggaacacttcatagagcattcagtattttagtttttaactcaaagaatcaacttttattacagaaaagaaatgtaaagaaatatcactctccaggattatggacaaacacttgttgtagtcacccaagatatggtgaatctcttcatgatgctatatacagaagattaaaagaagagatgggatttacttgcgaacttgaagaagtattctcattcatatataaggtaaaacttgaagataatttatttgagaatgaatatgaccatgtatttattggtaaatatgatggtgagataattgttaataaagatgaagttgatgattttaaatgggtagacattaatgaagttaaaaaggacataatagaaagacctgaggcatatacttactggtttaagtatcttgtaaataaagctgaaaataagatatttaaataaaccggtcagtaacgaatagaattagaaaaacaaaggaggcaagacaatggatttcccacaacaattagaagcatgtgtaaaacaggctaatcaggcacttagtagatttattgctcctcttccttttcaaaatacaccagtagtagaaactatgcaatacggtgcacttttaggtggtaaaagattaagaccattcttagtatatgctacaggacacatgtttggtgtatcaactaatactttagacgctccagctgctgctgttgaatgtattcatgcttattctttaatacatgatgacttaccagcaatggatgacgatgatttaagaagaggtttacctacatgtcatgttaaatttggtgaagctaatgcaattttagcaggtgacgctttacaaactttagctttttctatactttcagatgcagacatgcctgaagtttcagatagagatagaatttctatgatatcagagcttgcatctgcatcaggaatagctggaatgtgcggtggtcaagcacttgatttagatgcagaaggtaaacacgtaccacttgatgctttagagagaatacatagacataaaacaggtgctcttataagagcagcagtaagattaggtgctttaagtgctggtgacaagggtagaagagcacttccagtacttgataagtatgcagaaagtataggattagcttttcaagttcaagatgacatacttgacgttgttggtgatactgctactttaggaaaaagacagggtgcagatcagcaattaggaaaatctacataccctgctttacttggattagaacaggctagaaagaaagcaagagacttaatagatgacgcaagacaaagtcttaaacagttagctgaacaatcacttgacacaagtgcacttgaagcacttgcagattatattatacagagaaacaagtaaaagcttttaaaggaggggaaaaaatggaatttagagtacatttacaggcagacaacgaacagaaaatatttcaaaatcaaatgaaaccagagccagaagcatcatatcttataaatcaaagaagaagtgctaattataaaccaaacatttggaaaaacgattttcttgatcagtctttaatatcaaaatatgatggtgatgaatatagaaaactttcagaaaagttaatagaagaagtaaagatatacatatcagcagagactatggatttagttgctaaattagaacttatagattctgttagaaaacttggacttgctaatctttttgagaaagaaataaaggaagcattagacagtatagcagcaatagaatcagataatttaggaactagagacgatctttatggaacagctcttcattttaagattcttagacagcatggatataaggtaagtcaagatatatttggtagatttatggatgagaaaggaacattagaaaatcatcactttgcacacttaaaaggaatgttagaattatttgaggcaagtaatcttggatttgaaggtgaagacatattagatgaagctaaagcatctcttacacttgctcttagagattcaggacatatttgttatccagactcaaacttaagtagagatgtagttcatagtttagaattacctagtcatagaagagttcaatggttcgatgtaaaatggcagattaatgcatacgaaaaagatatttgtagagtaaatgcaactttattagagttagcaaagttaaattttaatgttgttcaagctcagcttcagaagaatcttagagaagctagtagatggtgggctaatcttggtttcgcagataatttaaagtttgctagagatagacttgtagagtgtttttcatgcgcagtaggtgtagcatttgaaccagagcattcatcttttagaatatgtttaactaaggtaattaatcttgttcttattatagatgatgtatacgatatatatggatctgaagaagagttaaaacattttacaaatgctgttgatagatgggacagtagagaaacagaacagcttcctgaatgcatgaaaatgtgttttcaagtattatataacactacttgcgaaatagcaagagagatagaagaagaaaacggttggaatcaagtattacctcaacttactaaggtttgggctgatttttgtaaggctcttttagttgaagcagagtggtacaataaatcacatattccaacattagaagaatatcttagaaacggatgtatatcaagtagtgtatctgtacttttagttcactctttcttttcaataactcatgaaggtacaaaagaaatggctgatttcttacataaaaatgaagatcttttatacaacataagtcttatagtaagattaaacaatgatttaggtacatcagctgctgaacaggaaagaggtgattctccttcttctatagtttgctatatgagagaagttaatgcttctgaagagactgcaagaaagaatataaagggaatgattgataatgcttggaaaaaggttaatggaaaatgtttcacaactaaccaagttccatttctttcatcattcatgaataatgcaactaacatggcaagagtagcacactcattatataaagacggtgatggttttggtgatcaagaaaaaggacctagaacacatattcttagtttattattccaacctttagtaaattaagctagcataaaaataagaagcctgcatttgcaggcttcttatttttatggcgcgccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagttt

SEQ ID NO: 92 - DNA, 25 bp, synthetic construct
  misc_feature 1..25: note = oligonucleotide repHF
  source 1..25: mol_type = other DNA
aagaagggcgtatatgaaaacttgt

SEQ ID NO: 93 - DNA, 25 bp, synthetic construct
  misc_feature 1..25: note = oligonucleotide catR
  source 1..25: mol_type = other DNA
ttcgtttacaaaacggcaaatgtga

SEQ ID NO: 94 - DNA, 20 bp, synthetic construct
  misc_feature 1..20: note = oligonucleotide MK-RTPCR-F
  source 1..20: mol_type = other DNA
gtgctggtagaggtggttca

SEQ ID NO: 95 - DNA, 20 bp, synthetic construct
  misc_feature 1..20: note = oligonucleotide MK-RTPCR-R
  source 1..20: mol_type = other DNA
ccaagtatgtgctgcaccag

SEQ ID NO: 96 - DNA, 20 bp, synthetic construct
  misc_feature 1..20: note = oligonucleotide PMK-RTPCR-F
  source 1..20: mol_type = other DNA
atatcagacccacacgcagc

SEQ ID NO: 97 - DNA, 24 bp, synthetic construct
  misc_feature 1..24: note = oligonucleotide PMK-RTPCR-R
  source 1..24: mol_type = other DNA
aatgcttcattgctatgtcacatg

SEQ ID NO: 98 - DNA, 20 bp, synthetic construct
  misc_feature 1..20: note = oligonucleotide PMD-RTPCR-F
  source 1..20: mol_type = other DNA
gcagaagcaaaggcagcaat

SEQ ID NO: 99 - DNA, 23 bp, synthetic construct
  misc_feature 1..23: note = oligonucleotide PMD-RTPCR-R
  source 1..23: mol_type = other DNA
ttgatccaagatttgtagcatgc

SEQ ID NO: 100 - DNA, 24 bp, synthetic construct
  misc_feature 1..24: note = oligonucleotide idi-RTPCR-F
  source 1..24: mol_type = other DNA
ggacaaacacttgttgtagtcacc

SEQ ID NO: 101 - DNA, 22 bp, synthetic construct
  misc_feature 1..22: note = oligonucleotide idi-RTPCR-R
  source 1..22: mol_type = other DNA
tcaagttcgcaagtaaatccca

SEQ ID NO: 102 - DNA, 20 bp, synthetic construct
  misc_feature 1..20: note = oligonucleotide ispA-RTPCR-F
  source 1..20: mol_type = other DNA
accagcaatggatgacgatg

SEQ ID NO: 103 - DNA, 21 bp, synthetic construct
  misc_feature 1..21: note = oligonucleotide ispA-RTPCR-R
  source 1..21: mol_type = other DNA
agtttgtaaagcgtcacctgc

SEQ ID NO: 104 - DNA, 20 bp, synthetic construct
  misc_feature 1..20: note = oligonucleotide FS-RTPCR-F
  source 1..20: mol_type = other DNA
aagctagtagatggtgggct

SEQ ID NO: 105 - DNA, 20 bp, synthetic construct
  misc_feature 1..20: note = oligonucleotide FS-RTPCR-R
  source 1..20: mol_type = other DNA
aatgctacacctactgcgca

SEQ ID NO: 106 - DNA, 18 bp, synthetic construct
  misc_feature 1..18: note = oligonucleotide ermB-F
  source 1..18: mol_type = other DNA
tttgtaattaagaaggag

SEQ ID NO: 107 - DNA, 18 bp, synthetic construct
  misc_feature 1..18: note = oligonucleotide ermB-R
  source 1..18: mol_type = other DNA
gtagaatccttcttcaac

SEQ ID NO: 108 - DNA, 20 bp, synthetic construct
  misc_feature 1..20: note = oligonucleotide GnK-F
  source 1..20: mol_type = other DNA
tcaggaccttctggaactgg

SEQ ID NO: 109 - DNA, 20 bp, synthetic construct
  misc_feature 1..20: note = oligonucleotide GnK-R
  source 1..20: mol_type = other DNA
acctccccttttcttggaga

SEQ ID NO: 110 - DNA, 20 bp, synthetic construct
  misc_feature 1..20: note = oligonucleotide FoT4L-F
  source 1..20: mol_type = other DNA
caggtttcggtgctgaccta

SEQ ID NO: 111 - DNA, 20 bp, synthetic construct
  misc_feature 1..20: note = oligonucleotide FoT4L-F
  source 1..20: mol_type = other DNA
aactccgccgttgtatttca